(19) United States
(12) Patent Application Publication
(10) Pub. No.: US 2006/0276416 A1
(43) Pub. Date: Dec. 7, 2006

(54) Methods and compositions for treating flushing and drug induced weight gain

(75) Inventors: David Sinclair, West Roxbury, MA (US); Robert S. Langer, Newton, MA (US); Christoph H. Westphal, Brookline, MA (US); Michael Milburn, Cary, NC (US)

Correspondence Address: FISH & NEAVE IP GROUP;ROPES & GRAY LLP, ONE INTERNATIONAL PLACE, BOSTON, MA, 02110-2624, US

(73) Assignee: Sirtris Pharmaceuticals, Inc., Cambridge, MA (US)

(21) Appl. No.: 11/336,258

(22) Filed: Jan. 20, 2006

Related U.S. Application Data
(60) Provisional application No. 60/645,962, filed on Jan. 21, 2005.
(60) Provisional application No. 60/645,916, filed on Jan. 20, 2005.

(51) Int. Cl. A61K 31/7048; A61K 31/57; A61K 31/56; A61K 31/551; A61K 31/426; A61K 31/353; A61K 38/28; A61K 31/445; A61K 31/44; A61K 31/137

(52) U.S. Cl. 514/27; 514/456; 514/171; 514/220; 514/369; 514/342; 514/733; 514/317; 514/649; 514/3

(57) ABSTRACT

Provided herein are methods and compositions for treating and/or preventing flushing and/or weight gain. Methods may comprise modulating the activity or level of a sirtuin, such as SIRT1 or Sir2. Exemplary embodiments include methods and compositions for counteracting drug-induced weight gain and/or drug-induced flushing by administering a sirtuin-activating compound.

### RELATED APPLICATIONS

This application claims the benefit of priority to U.S. Provisional Patent Application Nos. 60/645,962, filed Jan. 21, 2005, and 60/645,916, filed Jan. 20, 2005, which applications are hereby incorporated by reference in their entirety.

### BACKGROUND

The Silent Information Regulator (SIR) family of genes represents a highly conserved group of genes present in the genomes of organisms ranging from archaebacteria to a variety of eukaryotes (Frye, 2000). The encoded SIR proteins are involved in diverse processes from regulation of gene silencing to DNA repair. The proteins encoded by members of the SIR2 gene family show high sequence conservation in a 250 amino acid core domain. A well-characterized gene in this family is S. cerevisiae SIR2, which is involved in silencing HM loci that contain information specifying yeast mating type, telomere position effects and cell aging (Guarente, 1999; Kaeberlein et al., 1999; Shore, 2000). The yeast Sir2 protein belongs to a family of histone deacetylases (reviewed in Guarente, 2000; Shore, 2000). The Sir2 homolog, CobB, in Salmonella typhimurium, functions as an NAD (nicotinamide adenine dinucleotide)-dependent ADP-ribosyl transferase (Tsang and Escalante-Semerena, 1998).

The Sir2 protein is a deacetylase which uses NAD as a cofactor (Imai et al., 2000; Moazed, 2001; Smith et al., 2000; Tanner et al., 2000; Tanny and Moazed, 2001). Unlike other deacetylases, many of which are involved in gene silencing, Sir2 is insensitive to histone deacetylase inhibitors like trichostatin A (TSA) (Imai et al., 2000; Landry et al., 2000a; Smith et al., 2000).

Deacetylation of acetyl-lysine by Sir2 is tightly coupled to NAD hydrolysis, producing nicotinamide and a novel acetyl-ADP ribose compound (1-O-acetyl-ADP-ribose) (Tanner et al., 2000; Landry et al., 2000b; Tanny and Moazed, 2001). The NAD-dependent deacetylase activity of Sir2 is essential for its functions which can connect its biological role with cellular metabolism in yeast (Guarente, 2000; Imai et al., 2000; Lin et al., 2000; Smith et al., 2000). Mammalian Sir2 homologs have NAD-dependent histone deacetylase activity (Imai et al., 2000; Smith et al., 2000). Most information about Sir2 mediated functions comes from the studies in yeast (Gartenberg, 2000; Gottschling, 2000).

Biochemical studies have shown that Sir2 can readily deacetylate the amino-terminal tails of histones H3 and H4, resulting in the formation of 1-O-acetyl-ADP-ribose and nicotinamide. Strains with additional copies of SIR2 display increased rDNA silencing and a 30% longer life span. It has recently been shown that additional copies of the C. elegans SIR2 homolog, sir-2.1, greatly extend life span in that organism. This implies that the SIR2-dependent regulatory pathway for aging arose early in evolution and has been well conserved. Yeast life span, like that of metazoans, is also extended by interventions that resemble caloric restriction. Mutations that reduce the activity of the glucose-responsive cAMP (adenosine 3′5′-monophosphate)-dependent (PKA) pathway extend life span in wild type cells but not in mutant sir2 strains, demonstrating that SIR2 is a key downstream component of the caloric restriction pathway.

Recently, a number of small molecule activators and inhibitors of the SIR proteins have been reported (see e.g., U.S. Patent Application Publication Nos. 2005/0136537 and 2005/0096256 and PCT Publication Nos. WO 2005/002555 and WO 2005/002672) and a number of uses for these compounds have been identified. For example, small molecule activators of SIR proteins were shown to extend life span in yeast and cultured human cells as well as activate SIR protein activity in human cells (supra). Additionally, the small molecule SIR activators were shown to mimic calorie restriction and extend lifespan in Caenorhabditis elegans and Drosophila melanogaster (supra). Activators of the SIR proteins may therefore be useful for mimicking the effects of calorie restriction in eukaryotic cells and treating aging-related diseases such as stroke, cardiovascular disease, arthritis, high blood pressure, or Alzheimer's disease (supra). Additionally, it has been shown that resveratrol, butein, fisetin, piceatannol, and quercetin, small molecule activators of SIR proteins, promote fat mobilization in C. elegans, prevent fat accumulation in C. elegans, stimulate fat mobilization in mammalian cells, and inhibit adipogenesis in mammalian cells (see e.g., U.S. Patent Publication No. 2005/0171027 and PCT Publication No. WO 2005/065667). Similarly, nicotinamide, an inhibitor of SIR proteins, was shown to promote fat accumulation (supra). Additionally, resveratrol was shown to at least partially restore insulin sensitivity in insulin resistant cells (supra). Activators of SIR proteins may therefore also be useful for treating or preventing insulin resistance disorders and have been suggested for uses relating to reducing weight or preventing weight gain (supra). Certain details of the methods and results presented in U.S. Patent Application Publication Nos. 2005/0136537, 2005/0096256, and 2005/0171027 and PCT Publication Nos. WO 2005/002555, WO 2005/002672 and WO 2005/065667 are provided in the figures and experimental section of this disclosure as further support for, and to illustrate, the structure and activity of SIR activators and inhibitors.

### SUMMARY

In one aspect, the invention provides methods for treating or preventing flushing and/or hot flashes. The method may comprise administering to a subject in need thereof a therapeutically effective amount of a sirtuin-activating compound. In one embodiment, the flushing may be associated with menopause. In such embodiments, the subject may be a menopausal or post-menopausal woman. In another embodiment, the flushing may be drug-induced flushing. In one embodiment, the flushing may be associated with administration of raloxifene. In another embodiment, the flushing is associated with administration of an antidepressant or anti-psychotic agent. In such embodiments, the antidepressant or anti-psychotic agent may be one or more of the following: a serotonin reuptake inhibitor, a 5HT2 receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT1A receptor antagonist, a 5HT1D receptor antagonist, a CRF antagonist, a monoamine oxidase inhibitor, or a sedative-hypnotic drug. Exemplary serotonin reuptake inhibitors that may induce flushing include, for example, a fluoxetinoid, a nefazodonoid, duloxetine, venlafaxine, milnacipran, citalopram, fluvoxamine, paroxetine or sertraline. Exemplary sedative-hypnotic drug that may induce flushing include, for example, a benzodiazepine, zolpidem, or a barbiturate. Exemplary 5-HT1A receptor antagonist that may induce flushing include, for example, buspirone, flesinoxan, gepirone or ipsapirone. Exemplary norepinephrine reuptake inhibitors that may induce flushing include, for example, a tertiary amine tricyclic (e.g., amitriptyline, clomipramine, doxepin, imipramine or trimipramine) or a secondary amine tricyclic (e.g., amoxapine, desipramine, maprotiline, nortriptyline or protriptyline). Exemplary monoamine oxidase inhibitors that may induce flushing include, for example, isocarboxazid, phenelzine, tranylcypromine, selegiline or moclobemide. In another embodiment, the flushing may be associated with administration of a chemotherapeutic agent, such as, for example, cyclophosphamide or taxmoxifen. In another embodiment, the flushing may be associated with administration of a calcium channel blocker, such as, for example, amlodipine. In another embodiment, the flushing may be associated with administration of nicotinic acid. In another embodiment, the flushing may be associated with with administration of an antibiotic, such as, for example, levofloxacin. Exemplary sirtuin-activating compounds that may be administered for treating and/or preventing flushing include, for example, resveratrol, fisetin, butein, piceatannol or quercetin. Other exemplary sirtuin-activating compounds that may be administered for treating and/or preventing flushing include, for example, a compound of formulas 1-25, 30, 32-65, or 69-76. In exemplary embodiments, the subject is a human.

In another aspect, the invention provides compositions comprising at least one sirtuin-activating compound and at least one drug that induces flushing. Exemplary sirtuin-activating compounds include, for example, resveratrol, fisetin, butein, piceatannol, quercetin or a compound of formula 1-25, 30, 32-65, or 69-76. Other sirtuin-activating compounds that may be used in such compositions include, for example, resveratrol, fisetin, butein, piceatannol or quercetin. In certain embodiments, the drug that induces flushing is at least one of the following: nicotinic acid, raloxifene, an antidepressant, an anti-psychotic, a chemotherapeutic agent (e.g., cyclophosphamide or tamoxifen), a calcium channel blocker (e.g., amlodipine), or an antibiotic (e.g., levofloxacin). Exemplary antidepressants that may induce flushing include, for example, a serotonin reuptake inhibitor (e.g., a fluoxetinoid, a nefazodonoid, duloxetine, venlafaxine, milnacipran, citalopram, fluvoxamine, paroxetine or sertraline), a 5HT2 receptor antagonist, an anticonvulsant, a norepinephrine reuptake inhibitor (e.g., a tertiary amine tricyclic (such as, for example, amitriptyline, clomipramine, doxepin, imipramine or trimipramine) or a secondary amine tricyclic (such as, for example, amoxapine, desipramine, maprotiline, nortriptyline or protriptyline)), an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT1A receptor antagonist (e.g., buspirone, flesinoxan, gepirone or ipsapirone), a 5HT1D receptor antagonist, a CRF antagonist, a monoamine oxidase inhibitor (e.g., isocarboxazid, phenelzine, tranylcypromine, selegiline or moclobemide), or a sedative-hypnotic drug (e.g., a benzodiazepine, zolpidem, or a barbiturate). In an exemplary embodiment, the composition may comprise a therapeutically effective amount of at least one sirtuin-activating compound and a therapeutically effective amount of at least one drug that induces flushing.

In another aspect, the invention provides methods for treating or preventing drug-induced weight gain. The methods may comprise administering to a subject in need thereof a therapeutically effective amount of a sirtuin-activating compound. Exemplary drugs that may induce weight gain include, for example, anti-diabetic agents, antidepressants, steroids, hormones, beta blockers, alpha blockers, and contraceptives. In an exemplary embodiment, the weight gain is associated with administration of a diabetes treatment, such as, for example, a sulfonylurea, a thiazolidinedione, a meglitinide, nateglinide, repaglinide, or insulin. In another embodiment, weight gain is associated with administration of an antidepressant, such as, for example, a tricyclic antidepressant, an irreversible monoamine oxidase inhibitor (MAOI), a selective serotonin reuptake inhibitor (SSRI), bupropion, paroxetine, or mirtazapine. In another embodiment, the weight gain is associated with administration of a steroid or a hormone. In another embodiment, the weight gain is associated with administration of a beta blocker. In another embodiment, the weight gain is associated with administration of an alpha blocker. In another embodiment, the weight gain is associated with administration of a contraceptive. Exemplary sirtuin-activating compounds that may be administered for treating and/or preventing drug-induced weight gain include, for example, resveratrol, fisetin, butein, piceatannol or quercetin. Other exemplary sirtuin-activating compounds that may be administered for treating and/or preventing drug-induced weight gain include, for example, a compound of formulas 1-25, 30, 32-65, or 69-76. In an exemplary embodiment, the subject is a human.

In another aspect, the invention provides compositions comprising at least one sirtuin-activating compound and at least one drug that induces weight gain. Exemplary sirtuin-activating compounds include, for example, resveratrol, fisetin, butein, piceatannol, quercetin or a compound of formula 1-25, 30, 32-65, or 69-76. In certain embodiments, the drug that induces weight gain is at least one of the following: an anti-diabetic (e.g., a sulfonylurea, a thiazolidinedione, a meglitinide, nateglinide, repaglinide, or insulin), an antidepressant (e.g., a tricyclic antidepressant, an irreversible monoamine oxidase inhibitor (MAOI), a selective serotonin reuptake inhibitor (SSRI), bupropion, paroxetine, or mirtazapine), a steroid, a hormone, a beta blocker, an alpha blocker, or a contraceptive. In an exemplary embodiment, the composition may comprise a therapeutically effective amount of at least one sirtuin-activating compound and a therapeutically effective amount of at least one drug that induces weight gain.

In another aspect, the invention provides methods for increasing the level or activity of a sirtuin protein, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc, using a sirtuin-activating compound in combination with nicotinic acid. The method may comprise administering to a subject in need thereof a composition comprising a sirtuin-activating compound and nicotinic acid, or pharmaceutically acceptable salts or prodrugs thereof. In one embodiment, a sirtuin activating compound and nicotinic acid may be administered as part of a combination therapy with one or more therapeutic agents for the treatment or prevention of various diseases, including, for example, cancer, diabetes, neurodegenerative diseases, cardiovascular disease, blood clotting, inflammation, flushing, obesity, ageing, stress, etc. In an exemplary embodiment, the subject is a human.

In one embodiment, the invention provides a method for promoting survival of a eukaryotic cell comprising contacting the cell with a composition comprising a sirtuin-activating compound and nicotinic acid, or pharmaceutically acceptable salts or prodrugs thereof. In certain embodiments, the composition increases at least one of the level or activity of a SIRT1 protein in the cell, such as, a mammalian SIRT1, or a human SIRT1. In certain embodiments, the composition increases the lifespan of the cell. In certain embodiments, the composition increases the cell's ability to resist stress, such as, for example, heatshock, osmotic stress, DNA damage, inadequate salt level, inadequate nitrogen level, or inadequate nutrient level. In certain embodiments, the composition mimics the effect of nutrient restriction on the cell. In certain embodiments, the composition increases deacetylase activity of the SIRT1 protein. In certain embodiments, the eukaryotic cell is a mammalian cell.

In another embodiment, the invention provides a method for treating or preventing a disease or disorder associated with cell death or aging in a subject, comprising administering to a subject in need thereof a composition comprising a therapeutically effective amount of a sirtuin-activating compound and nicotinic acid, or pharmaceutically acceptable salts or prodrugs thereof. In certain embodiments, the aging-related disease is stroke, a cardiovascular disease, arthritis, high blood pressure, or Alzheimer's disease.

In another embodiment, the invention provides a method for treating or preventing insulin resistance, a metabolic syndrome, diabetes, or complications thereof, or for increasing insulin sensitivity in a subject, comprising administering to a subject in need thereof a composition comprising a therapeutically effective amount of a sirtuin-activating compound and nicotinic acid, or pharmaceutically acceptable salts or prodrugs thereof.

In another embodiment, the invention provides a method for reducing the weight of a subject, or preventing weight gain in a subject, comprising administering to a subject in need thereof a compositions comprising a therapeutically effective amount of a sirtuin-activating compound and nicotinic acid, or pharmaceutically acceptable salts or prodrugs thereof.

In another embodiment, the invention provides a method for preventing the differentiation of a pre-adipocyte, comprising contacting the pre-adipocyte with a composition comprising a sirtuin-activating compound and nicotinic acid, or pharmaceutically acceptable salts or prodrugs thereof.

In another embodiment, the invention provides a method for prolonging the lifespan of a subject comprising administering a composition comprising a therapeutically effective amount of a sirtuin-activating compound and nicotinic acid, or pharmaceutically acceptable salts or prodrugs thereof.

In another embodiment, the invention provides a method for treating or preventing a neurodegenerative disorder in a subject, comprising administering to a subject in need thereof a composition comprising a therapeutically effective amount of a sirtuin-activating compound and nicotinic acid, or pharmaceutically acceptable salts or prodrugs thereof. Exemplary neurodegenerative disorders include, for example, Alzheimer's disease (AD), Parkinson's disease (PD), Huntington disease (HD), amyotrophic lateral sclerosis (ALS; Lou Gehrig's disease), diffuse Lewy body disease, chorea-acanthocytosis, primary lateral sclerosis, Multiple Sclerosis (MS) and Friedreich's ataxia.

In another embodiment, the invention provides a method for treating or preventing a blood coagulation disorder in a subject, comprising administering to a subject in need thereof a composition comprising a therapeutically effective amount of a sirtuin-activating compound and nicotinic acid, or pharmaceutically acceptable salts or prodrugs thereof. Exemplary blood coagulation disorders include, for example, thromboembolism, deep vein thrombosis, pulmonary embolism, stroke, myocardial infarction, miscarriage, thrombophilia associated with antithrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, disseminated intravascular coagulation, stroke, ischemic tissue injury, cardiac ischemia, cardiac reperfusion injury, thrombotic thrombocytopenic purpura, cancer metastasis, sickle cell disease, glomerular nephritis, drug induced thrombocytopenia, and re-occlusion during or after therapeutic clot lysis or procedures such as angioplasty or surgery.

In another aspect, the invention provides a composition comprising at least one sirtuin-activating compound and nicotinic acid. In certain embodiments, the invention provides a pharmaceutical composition comprising at least one sirtuin-activating compound, nicotinic acid and a therapeutic agent useful for treatment or prevention of various diseases, including, for example, cancer, diabetes, neurodegenerative diseases, cardiovascular disease, blood clotting, inflammation, flushing, obesity, ageing, stress, etc. In an exemplary embodiment, the composition may comprise a therapeutically effective amount of a sirtuin activating compound and nicotinic acid, or pharmaceutically acceptable salts or prodrugs thereof. Exemplary sirtuin activating compounds include, for example, a compound having a formula selected from the group consisting of formulas 1-25, 30, 32-65, and 69-76. Other sirtuin-activating compounds include resveratrol, fisetin, butein, piceatannol or quercetin. In certain embodiments, nicotinic acid may be one or more of the following nicotinic acid equivalents: nicotinyl alcohol tartrate, d-glucitol hexanicotinate, aluminum nicotinate, niceritrol or d,l-alpha-tocopheryl nicotinate. In certain embodiments, the composition is a sustained release formulation. In another embodiment, the invention provides a composition comprising a therapeutically effective amount of resveratrol and nicotinic acid, or pharmaceutically acceptable salts or prodrugs thereof.

In another aspect, provided is the use of a sirtuin-activating compound for the manufacture of a medicament for treating or preventing flusing.

In another aspect, provided is the use of a sirtuin-activating compound for the manufacture of a medicament for treating or preventing drug-induced weight gain.

### BRIEF DESCRIPTION OF THE DRAWINGS

FIG. 1 shows the effects of resveratrol on the kinetics of recombinant human SIRT1.a, Resveratrol dose-response of SIRT1 catalytic rate at 25 μM NAD⁺, 25 μM p53-382 acetylated peptide. Relative initial rates are the mean of two determinations, each derived from the slopes of fluorescence (arbitrary fluorescence units, AFU) vs. time plots with data obtained at 0, 5, 10 and 20 min. of deacetylation. b, SIRT1 initial rate at 3 mM NAD⁺, as a function of p53-382 acetylated peptide concentration in the presence (Δ) or absence (ν) of 100 μM resveratrol. Lines represent non-linear least-squares fits to the Michaelis-Menten equation. Kinetic constants: Kₘ (control, ν)=64 μM, Kₘ (+resveratrol, Δ)=1.8 μM; Vₘₐₓ(control, ν)=1107 AFU/min., Vₘₐₓ(+resveratrol, Δ)=926 AFU/min. c, SIRT1 initial rate at 1 mM p53-382 acetylated peptide, as a function of NAD⁺ concentration, in the presence (Δ) or absence (ν) of 100 μM resveratrol. Lines represent non-linear least-squares fits to the Michaelis-Menten equation. Kinetic constants: Kₘ (control, ν)=558 μM, Kₘ (+resveratrol, Δ)=101 μM; Vₘₐₓ (control, ν)=1863 AFU/min., Vₘₐₓ (+resveratrol, Δ)=1749 AFU/min. d, Effects of resveratrol on nicotinamide inhibition of SIRT1. Kinetic constants are shown relative to those of the control (no nicotinamide, no resveratrol) and represent the mean of two determinations. Error bars are standard errors of the mean. The variable substrate in each experiment (N=NAD⁺, P=p53 acetylated peptide), the presence/absence of nicotinamide (+/−) and the resveratrol concentration (μM) are indicated beneath each pair of Kₘ−Vₘₐₓ bars.

FIG. 2 shows the effects of polyphenols on Sir2 and S. cerevisiae lifespan. a, Initial deacetylation rate of recombinant GST-Sir2 as a function of resveratrol concentration. Rates were determined at the indicated resveratrol concentrations, either with 100 μM ‘Fluor de Lys’ acetylated lysine substrate (FdL) plus 3 mM NAD⁺ (Δ) or with 200 μM p53-382 acetylated peptide substrate plus 200 μM NAD⁺ (ν). b, Lifespan analyses were determined by micro-manipulating individual yeast cells as described³⁷ on complete 2% glucose medium with 10 μM of each compound, unless otherwise stated. Average lifespan for wild type, 22.9 generations, quercetin, 23.4; piceatannol. 24.0. c, Average lifespan for wild type, 22.9 generations; fisetin, 30.0; butein, 35.5; resveratrol, 36.8. d, Average lifespan for wild type untreated, 21.0 generations; growth on resveratrol, 10 μM, 35.7; 100 μM, 29.4; 500 μM, 29.3.

FIG. 3 shows that resveratrol extends lifespan by mimicking CR and suppressing rDNA recombination. Yeast lifespans were determined as in FIG. 2. a, Average lifespan for wild type (wt) untreated, 19.0 generations; wild type+resveratrol (wt+R) 37.8; glucose-restricted+resveratrol (CR+R), 39.9. b, Average lifespans for wild type sir2Δ, 9.9; sir2Δ+resveratrol, 10.0; pnc1Δ, 19.2; pnc1Δ+resveratrol, 33.1. c, Resveratrol suppresses the frequency of ribosomal DNA recombination in the presence and absence of nicotinamide (NAM). Frequencies were determined by loss of the ADE2 marker gene from the rDNA locus (RDN1). d, Resveratrol does not suppress rDNA recombination in a sir2 strain. e, Resveratrol and other sirtuin activators do not significantly increase rDNA silencing compared to a 2×SIR2 strain. Pre-treated cells (RDN1::URA3) were harvested and spotted as 10-fold serial dilutions on either SC or SC with 5-fluororotic acid (5-FOA). In this assay, increased rDNA silencing results in increased survival on 5-FOA medium. f, Quantitation of the effect of resveratrol on rDNA silencing by counting numbers of surviving cells on FOA/total plated.

FIG. 4 shows that resveratrol and other polyphenols stimulate SIRT1 activity in human cells. a, Method for assaying intracellular deacetylase activity with a fluorogenic, cell-permeable substrate, FdL (‘Fluor de Lys’, BIOMOL). FdL (200 μM) is added to growth media and cells incubated for 1-3 hours to allow FdL to enter the cells and the lysine-deacetylated product (deAc-FdL) to accumulate intracellularly. Cells are lysed with detergent in the presence of 1 μM TSA, 1 mM nicotinamide. Addition of the non-cell-permeable Developer (BIOMOL) releases a fluorophor, specifically from deAc-FdL. b, SIRT1 activating polyphenols can stimulate TSA-insensitive, FdL deacetylation by HeLa S3 cells. Cells were grown adherently in DMEM/10% FCS and treated for 1 hour with 200 μM FdL, 1 μM TSA and either vehicle (0.5% final DMSO, Control) or 500 μM of the indicated compound. Intracellular accumulation of deAc-FdL was then determined as described briefly in a. The intracellular deAc-FdL level for each compound (mean of six replicates) are plotted against the ratios to the control rate obtained in the in vitro SIRT1 polyphenol screen (see Table 1, Supplementary Tables 1 and 3). c, U2OS osteosarcoma cells grown to ≧90% confluence in DMEM/10% FCS were exposed to 0 or 10 grays of gamma irradiation (IR). Whole cell lysates were prepared 4 hours post-irradiation and were probed by Western blotting with indicated antibodies. d, U2OS cells cultured as above were pre-treated with the indicated amounts of resveratrol or a 0.5% DMSO blank for 4 hours after which cells were exposed to 0 or 50 J/cm² of UV radiation. Lysates were prepared and analyzed by Western blot as in c. e, Human embryonic kidney cells (HEK 293) expressing wild type SIRT1 or dominant negative SIRT1-H363Y (SIRT1-HY) protein were cultured as above, pre-treated with the indicated amounts of resveratrol or a 0.5% DMSO blank for 4 hours and exposed to 50 J/cm² of UV radiation as above. Lysates were prepared and analyzed as above.

FIG. 5 shows that intracellular deacetylation activity may be measured with a cell-permeable, fluorogenic HDAC and sirtuin substrate. HeLa S3 cells were grown to confluence in DMEM/10% FCS and then incubated with fresh medium containing 200 μM FdL for the indicated times, 37° C. Intracellular and medium levels of deacetylated substrate (deAc-FdL) were determined according to the manufacturer's instructions (HDAC assay kit, BIOMOL). All data points represent the mean of two determinations. a, Concentration ratio of intracellular ([deAc-FdL]ᵢ) to medium ([deAc-FdL]ₒ) concentrations in the presence (Δ) or absence (ν) of 1 μM trichostatin A (TSA). b, Total accumulation of deacetylated substrate (deAc-FdL) in the presence (Δ) or absence (ν) of 1 μM TSA. c, Intracellular accumulation of deacetylated substrate (deAc-FdL) in the presence (Δ) or absence (ν) of 1 μM TSA.

FIG. 6 shows that deacetylation site preferences of recombinant SIRT1. Initial rates of deacetylation were determined for a series of fluorogenic acetylated peptide substrates based on short stretches of human histone H3, H4 and p53 sequence (see key to substrate name and single letter peptide sequence below the bar graph). Recombinant human SIRT1 (1 μg, BIOMOL), was incubated 10 min, 37° C., with 25 μM of the indicated fluorogenic acetylated peptide substrate and 500 μM NAD⁺. Reactions were stopped by the addition of 1 mM nicotinamide and the deacetylation-dependent fluorescent signal was determined.

FIG. 7 is a graph representing SIRT2 activity as a function of resveratrol concentration.

FIG. 8 shows an alignment of the amino acid sequences of hSIRT2, hSIRT1 and S. cerevisiae Sir2.

FIG. 9A shows resveratrol and BML-230 dose responses of SIRT1 catalytic rate.

FIG. 9B shows the ratio of BML-230-activated to resveratrol-activated SIRT1 rates as a function of activator concentration (the ratios were calculated from data of FIG. 9A).

FIG. 10 shows the effect of polyphenolic STACs on metazoan sirtuins. a, Schematic of Sir2 polypeptides from human, yeast, C. elegans and D. melanogaster aligned to show conserved regions. Amino acids forming the NAD⁺-binding pocket (grey) and substrate binding groove (black) are indicated. Percentages refer to the homology to SIRT1. b, Effect of polyphenolic STACs (500 μM) on NAD⁺-dependent, trichostatin A (TSA)-insensitive deacetylase activity in Drosophila S2 cells. c, Fold stimulation of recombinant SIR-2.1 by STACs (10 μM). d, Fold stimulation of recombinant dSir2 by STACs (10 μM). Values are the mean of at least three determinations (+/−standard error). e, Dose-dependent activation of C. elegans SIR-2.1 by resveratrol. Rates were determined using a fluorigenic acetylated lysine substrate (Fluor de Lys). f, Dose-dependent activation of Drosophila dSir2 by resveratrol. g, SIR-2.1 initial rate at 10 μM Fluor de Lys as a function of NAD⁺ concentration, in the presence or absence of 100 μM resveratrol. AFU, arbitrary fluorescence units.

FIG. 11 shows the C. elegans survival on resveratrol. a, Survivorship of adult wild-type N2 C. elegans treated with 100 μM resveratrol fed with heat-killed OP50 E. coli. Mean lifespan relative to control (triangles, n=47) was increased by 14.5% (Log-Rank test, P<0.0001) by 100 μM resveratrol (squares, n=46). b, Survivorship of sir-2.1 mutants treated with resveratrol fed with heat-killed OP50. Adult lifespan of sir-2.1 animals does not differ significantly from N2 controls (Log-Rank, P=0.68) and the effect on lifespan of 100 □M resveratrol on sir-2.1 mutant animals was not statistically significant (5.2% extension, Log-Rank P=0.058; n=60 control, 58 treated). c, Survivorship of wild-type N2 C. elegans on 100 μM resveratrol fed with live OP50 (12.6% extension, P<0.0001; n=47 control, 67 treated). d, Survivorship of sir-2.1 mutants on 100 μM resveratrol fed with live OP50 (3.3% extension, P=0.81; n=57 control, 51 treated) e, Fecundity of adult hermaphrodites treated with 100 μM resveratrol. Controls: 106 eggs/5 worms/5 hours (s.d. 10.0); resveratrol-treated: 99 eggs/5 worms/5 hours (s.d. 13.0). f, Feeding rates of L4 larval and adult hermaphrodites treated with 100 μM resveratrol. L4 on live OP50: control 310±10.2 pumps/min, resveratrol 315±9.8; Adult on dead OP50: control 228±26.2, resveratrol 283±31.9; Adult on live OP50: control 383±16.0, resveratrol 383±22.7.

FIG. 12 shows wild-type female D. melanogaster survival with adults fed resveratrol or fisetin. a, Canton-S on 15% SY media. b, Canton-S on 5% SY media with resveratrol at two concentrations. c, Strain yw on 3% CSY media. d, Strain yw on 2% CSY media with resveratol at two concentrations. e, Strain yw on 3% CSY media with 100 μM resveratrol or fisetin. f, Strain yw on 2% CSY media with 100 μM resveratrol or fisetin. Life table statistics for this figure, for males and for additional trials are in Table 20. g, Mean daily fecundity per female (s.e.) estimated over 5-day intervals of Canton-S on 15% SY media with 0 or 10 μM resveratrol. h, Proportion (s.e.) of yw females feeding on diet with and without resveratrol in crop-filling assay. i, Mean (s.e.) body mass of Canton-S males and females feeding on diet without and with resveratrol (10 μM).

FIG. 13 shows the survivorship of D. melanogaster adults with mutant alleles of dSir2 when fed resveratrol (100 μM). Females (a) and males (b) with loss-of-function genotype dSir2^{4.5}/dSir2^{5.26}. Females (c) and males (d) with strong hypomorphic genotype dSir2¹⁷/dSir2^{KG00871}.

FIG. 14 shows the mortality rates of control and resveratrol treated adults. Mortality was estimated as ln(−ln(pₓ)) where pₓ is the survival probability at day x to x+1. a, C. elegans wild-type N2 on heat-killed OP50 E. coli. b, C. elegans wild-type N2 on live OP50 E. coli. In a and b mortality is plotted only at days with observed mortality. c, D. melanogaster wildtype females of Trial 1 at effective doses of resveratrol on 15% SY diet. d, D. melanogaster wildtype males of Trial 1 at effective doses of resveratrol on 15% SY diet. In c and d mortality is smoothed from 3-day running average of pₓ.

FIG. 15 shows the stimulation of SIRT 1 catalytic rate by 100 μM plant polyphenols (Table 1).

FIG. 16 shows the effect of 100 μM stilbenes and chalcones on SIRT 1 catalytic rate (Supplementary Table 1).

FIG. 17 shows the effect of 100 μM flavones on SIRT 1 catalytic rate (Supplementary Table 2).

FIG. 18 shows the effect of 100 μM flavones on SIRT 1 catalytic rate (Supplementary Table 3).

FIG. 19 shows the effect of 100 μM isoflavones, flavanones and anthocyanidins on SIRT 1 catalytic rate (Supplementary Table 4).

FIG. 20 shows the effect of 100 μM catechins (Flavan-3-ols) on SIRT 1 catalytic rate (Supplementary Table 5).

FIG. 21 shows the effect of 100 μM free radical protective compounds on SIRT 1 catalytic rate (Supplementary Table 6).

FIG. 22 shows the effect of 100 μM miscellaneous compounds on SIRT 1 catalytic rate (Supplementary Table 7).

FIG. 23 shows the effect of 100 μM of various modulators on SIRT 1 catalytic rate (Supplementary Table 8).

FIG. 24 shows the effect of 100 μM of new resveratrol analogs on SIRT 1 catalytic rate (Table 9).

FIG. 25 shows the effect of 100 μM of new resveratrol analogs on SIRT 1 catalytic rate (Table 10).

FIG. 26 shows the effect of 100 μM of new resveratrol analogs on SIRT 1 catalytic rate (Table 11).

FIG. 27 shows the effect of 100 μM of new resveratrol analogs on SIRT 1 catalytic rate (Table 12).

FIG. 28 shows the effect of 100 μM of new resveratrol analogs on SIRT 1 catalytic rate (Table 13).

FIG. 29 shows synthetic intermediates of resveratrol analog synthesis (Table 14).

FIG. 30 shows synthetic intermediates of resveratrol analog synthesis (Table 15).

FIG. 31 shows synthetic intermediates of resveratrol analog synthesis (Table 16).

FIG. 32 shows synthetic intermediates of resveratrol analog synthesis (Table 17).

FIG. 33 shows synthetic intermediates of resveratrol analog synthesis (Table 18).

FIG. 34 shows the effect of resveratrol on Drosophila melanogaster (Table 20).

FIGS. 35A-G shows sirtuin activators and the fold activation of SIRT1 (Table 21).

FIG. 36 shows sirtuin inhibitors and the fold inhibition of SIRT1 (Table 22).

FIG. 37 is a series of photomicrographs that depict the effect of the sirtuin-activating compound resveratrol at different concentrations to induce fat mobilization as indicated by a decrease in Nile Red staining.

FIG. 38 is a series of photomicrographs that depict the effect of resveratrol to induce fat mobilization in a mutant worm with disrupted insulin signaling.

FIG. 39 is a series of photomicrographs that depict the effect of the sirtuin-inhibiting compound nicotinamide on fat accumulation. A. Resveratrol stimulates fat mobilization in wild type animals. Worms grown in the presence of vehicle alone, or 10 μM, 50 μM, and 100 μM resveratrol in vehicle were stained with Nile Red. B. Nicotinamide promotes fat accumulation in wild type animals. Nile Red staining in the presence of PBS alone, 1 mM, 5 mM and 10 mM nicotinamide is shown. C. Lower panel, Resveratrol and Nicotinamide have opposing effects on fat content. Effect of vehicle alone, resveratrol (25 μM), Nicotinamide (5 mM) or resveratrol 25 μM and Nicotinamide 5 mM in combination, on fat accumulation as assessed by Nile Red staining.

FIG. 40a-b is a series of photomicrographs that demonstrate fat content of C. elegans wild-type treated or not with Sir2.1 RNAi and incubated in the presence or absence of resveratrol.

FIG. 41A a-d represents a series of photomicrographs of C. elegans incubated with empty RNAi vector (panel a); AMPK RNAi (panel b); COT RNAi (panel c) and DAF-16 RNAi in the presence or absence of resveratrol.

FIG. 41B represents the amount of Nile-Red staining in C. elegans shown in FIG. 41A.

FIG. 42 shows a Western Blot of proteins from C. elegans incubated in the presence or absence (control) of 500 μM AICAR, vehicle 2 (DMSO), 12.5 μM, 25 μM or 50 μM resveratrol and stained for the presence of AMPK, ACC, or tubulin.

FIG. 43 shows a Western Blot of proteins incubated in the presence or absence (control) of 500 μM AICAR, DMSO, 100 nM, 500 nM, 2.5 μM, 12.5 μM, 25 μM or 50 μM resveratrol and stained for the presence of P-ACC, P-AMPK, AMPK, or tubulin.

FIG. 44 is a Western Blot showing the phosphorylation of ACC in 3T3-L1 adipocytes treated either with ethanol or resveratrol and stained for the presence of P-ACC, SIRT1, or tubulin. In the lanes marked “SF”, cells were left in serum free media overnight before harvesting.

FIG. 45 is a Western Blot showing the phosphorylation of ACC in HEP3B human heptoma cells treated with either ethanol or resveratrol and stained for the presence of P-ACC, SIRT1, or tubulin. In the left lane, SIRT1 was knocked down. In the right four lanes, SIRT1 has been overexpressed.

FIG. 46 is a Western Blot of proteins from 3T3-L1 adipocytes infected with either a control (GFP) retrovirus, SIRT1, SIRT1 siRNA, or SIRT1 dominant negative (delta HY). Cells were incubated in the presence of AICAR, ethanol, or resveratrol and stained for the presence of P-ACC, ACC, SIRT1, P-AMPK, AMPK, tubulin, or GAPDH. A dose response curve is shown on the far right of the blot.

FIG. 47 is a Western Blot showing the effects of resveratrol in the presence or absence of AMPK kinase, LKB1. Mouse embryonic fibroblasts were incubated in the presence of AICAR, ethanol, 50, 100, 200 μM of resveratrol. Blots were stained for the presence of P-ACC, P-AMPK, AMPK, or tubulin as indicated on the left.

FIG. 48 shows that resveratrol inhibits lipid accumulation during mammalian adipogenesis. A. 3T3-L1 and NIH3T3 cells were differentiated into adipocytes in the presence of 25 μM, 12.5 μM or 0 μM resveratrol in vehicle (ethanol). After 10 days of differentiation, cells were fixed and stained with Oil red O. Oil red 0 was extracted from stained cells and quantified by measuring absorbance at 520 nm. B. Oil red 0 quantitation is shown as fold change relative to the 3T3-L1 sample treated with 0 μM resvratrol.

FIG. 49 shows that resveratrol inhibits adipogenesis, and that this is rescued by PPARγ. A marked decrease in PPARγ expression was detected in resveratrol-treated 3T3-L1 cells. In a separate experiment, 3T3-L1 cells were grown in the presence of virus encoding gfp or PPAR-gamma and 25 μM, 12.5 μM or 0 μM resveratrol in vehicle (ethanol). After 8 days of differentiation, cells were fixed and stained with Oil red O.

FIG. 50 shows that resveratrol inhibits lipid accumulation and the partial rescue by deacetylase deficient SIRT1. NIH3T3 cells were grown in the presence of virus encoding gfp, SIRT1 or deaceytlase deficient SIRT1. Cells were differentiated into adipocytes in the presence of 25 μM, 12.5 μM or 0 μM resveratrol in vehicle (ethanol). After 8 days of differentiation, cells were fixed and stained with Oil red O. Oil red O was extracted from stained cells and quantified.

FIG. 51 shows the effect of polyphenols on C. elegans fat stores. C. elegans in L1 were exposed to Nile Red stain and vehicle (A, 20% v/v DMSO in PBS buffer) or 100 μM resveratrol, butein, fisetin, piceatannol, or quercetin for 48 hours. In each image, the head is positioned towards the bottom.

FIG. 52 shows the effect of quercetin on C. elegans fat stores. C. elegans in L1 were exposed to Nile Red and vehicle (20% v/v DMSO) or quercetin at 10 μM, 50 μM and 100 μM for 48 hours. In each image, the head is positioned towards the bottom.

FIG. 53 shows the effect of fisetin on C. elegans fat stores. C. elegans in L1 stage were exposed to Nile Red and vehicle (A, 20% v/v DMSO) or fisetin at 10 μM, 50 μM and 100 μM for 48 hours. In each image, the head is positioned towards the bottom.

FIG. 54 shows the effect of 3,5-dihydroxy-4′-thiomethyl-trans-stilbene on C. elegans fat stores. Animals in L1 were treated with Nile Red stain and (A) 1% v/v DMSO or (B) 100 μM 3,5-dihydroxy-4′-thiomethyl-trans-stilbene for 24 hours. In each image, the head is positioned towards the bottom.

FIG. 55 compares the effect of resveratrol and cis-stilbene (a resveratrol analogue) on C. elegans fat stores. Animals in L1 were exposed to Nile Red stain and (A) 2.5% v/v DMSO, (B) 100 μM resveratrol or (C) cis-stilbene for 48 hours. In each image, the head is positioned towards the bottom.

FIG. 56 shows the effect of resveratrol on TNF-alpha treated adipocytes that are insulin resistant. Lane 1, no treatment; lane 2, TNF-alpha treated; lane 3, TNF-alpha plus 4 μM roziglitazone (positive control); lane 4, TNF-alpha plus 5 μM resveratrol; and Lane 5, TNF-alpha plus 15 μM resveratrol.

### DETAILED DESCRIPTION

1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

“Biologically active portion of a sirtuin” refers to a portion of a sirtuin protein having a biological activity, such as the ability to deacetylate. Biologically active portions of a sirtuin may comprise the core domain of sirtuins. Biologically active portions of SIRT1 having GenBank Accession No. NP_{—}036370 that encompass the NAD+ binding domain and the substrate binding domain, for example, may include without limitation, amino acids 62-293 of GenBank Accession No. NP_{—}036370, which are encoded by nucleotides 237 to 932 of GenBank Accession No. NM_{—}012238. Therefore, this region is sometimes referred to as the core domain. Other biologically active portions of SIRT1, also sometimes referred to as core domains, include about amino acids 261 to 447 of GenBank Accession No. NP_{—}036370, which are encoded by nucleotides 834 to 1394 of GenBank Accession No. NM_{—}012238; about amino acids 242 to 493 of GenBank Accession No. NP_{—}036370, which are encoded by nucleotides 777 to 1532 of GenBank Accession No. NM_{—}012238; or about amino acids 254 to 495 of GenBank Accession No. NP_{—}036370, which are encoded by nucleotides 813 to 1538 of GenBank Accession No. NM_{—}012238.

The term “companion animals” refers to cats and dogs. As used herein, the term “dog(s)” denotes any member of the species Canis familiaris, of which there are a large number of different breeds. The term “cat(s)” refers to a feline animal including domestic cats and other members of the family Felidae, genus Felis.

The terms “comprise” and “comprising” are used in the inclusive, open sense, meaning that additional elements may be included.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

“Diabetes” refers to high blood sugar or ketoacidosis, as well as chronic, general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

A “direct activator” of a sirtuin is a molecule that activates a sirtuin by binding to it.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “hyperinsulinemia” refers to a state in an individual in which the level of insulin in the blood is higher than normal.

The term “including” is used to mean “including but not limited to”. “Including” and “including but not limited to” are used interchangeably.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The term “livestock animals” refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus.

The term “mammal” is known in the art, and exemplary mammals include humans, primates, livestock animals (including bovines, porcines, etc.), companion animals (e.g., canines, felines, etc.) and rodents (e.g., mice and rats).

The term “naturally occurring form” when referring to a compound means a compound that is in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature. A “naturally occurring compound” refers to a compound that can be found in nature, i.e., a compound that has not been designed by man. A naturally occurring compound may have been made by man or by nature.

A “naturally occurring compound” refers to a compound that can be found in nature, i.e., a compound that has not been designed by man. A naturally occurring compound may have been made by man or by nature. For example, resveratrol is a naturally-occurring compound. A “non-naturally occurring compound” is a compound that is not known to exist in nature or that does not occur in nature.

“Obese” individuals or individuals suffering from obesity are generally individuals having a body mass index (BMI) of at least 25 or greater. Obesity may or may not be associated with insulin resistance.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject”, “individual” or “host” refers to either a human or a non-human animal.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” is art-recognized and refers to the relatively non-toxic, inorganic and organic acid addition salts of compounds, including, for example, those contained in compositions described herein.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

A “sirtuin-activating compound,” “activating compound,” or “sirtuin activator” refers to a compound that activates a sirtuin protein or stimulates or increases at least one activity of a sirtuin protein. In certain embodiments, a sirtuin-activating compound may have a formula selected from the group of formulas 1-25, 30, 32-65, and 69-76.

“Sirtuin activation” refers to increasing at least one activity of a sirtuin protein, preferably by at least about 10%, 50%, 100% or more. “Activating a sirtuin protein” refers to the action of producing an activated sirtuin protein, i.e., a sirtuin protein that is capable of performing at least one of its biological activities with an increase of activity of at least about 10%, 50%, 2 fold or more. Biological activities of sirtuin proteins include deacetylation, e.g., of histones and p53; extending lifespan; increasing genomic stability; silencing transcription; and controlling the segregation of oxidized proteins between mother and daughter cells.

“Sirtuin protein” refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 or NP_{—}036370 (or AF083106)), and human SIRT2 (GenBank Accession No. NM_{—}012237, NM_{—}030593, NP_{—}036369, NP_{—}085096, or AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, or P53685. SIRT1 proteins include polypeptides comprising all or a portion of the amino acid sequence set forth in GenBank Accession Nos. NP_{—}036370, NP 501912, NP_{—}085096, NP_{—}036369, or P53685; the amino acid sequence set forth in GenBank Accession Nos. NP_{—}036370, NP_{—}501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, or P53685, and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, or P53685.

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

The term “synthetic” is art-recognized and refers to production by in vitro chemical or enzymatic synthesis.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

“Treating” a condition or disease refers to curing as well as ameliorating at least one symptom of the condition or disease.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “structure-activity relationship” or “(SAR)” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, naphtalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 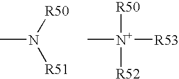
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 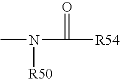
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 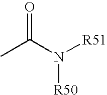
wherein R50 and R51 are as defined above. Certain embodiments of amides may not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas: 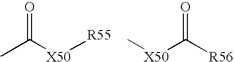
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

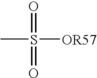

in which R57 is an electron pair, hydrogen, alkyl, cycloalkyl, or aryl.

The term “sulfate” is art recognized and includes a moiety that may be represented by the general formula: 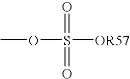
in which R57 is as defined above.

The term “sulfonamido” is art recognized and includes a moiety that may be represented by the general formula: 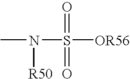
in which R50 and R56 are as defined above.

The term “sulfamoyl” is art-recognized and refers to a moiety that may be represented by the general formula: 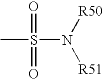
in which R50 and R51 are as defined above.

The term “sulfonyl” is art-recognized and refers to a moiety that may be represented by the general formula: 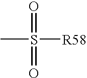
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 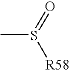
in which R58 is defined above.

The term “phosphoryl” is art-recognized and may in general be represented by the formula: 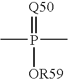
wherein Q50 represents S or O, and R59 represents hydrogen, a lower alkyl or an aryl. When used to substitute, e.g., an alkyl, the phosphoryl group of the phosphorylalkyl may be represented by the general formulas: 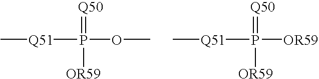
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 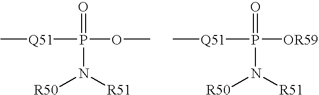
wherein Q51, R50, R51 and R59 are as defined above.

The term “phosphonamidite” is art-recognized and may be represented in the general formulas: 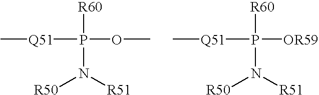
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls. The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions described herein may exist in particular geometric or stereoisomeric forms. In addition, compounds may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are encompassed herein.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. Heteroatoms such as nitrogen may have hydrogen substituents and/or any permissible substituents of organic compounds described herein which satisfy the valences of the heteroatoms. Compounds are not intended to be limited in any manner by the permissible substituents of organic compounds.

The chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonylaminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

2. Exemplary Sirtuin-Activating Compounds

In one embodiment, exemplary sirtuin-activating compounds are those described in Howitz et al. (2003) Nature 425: 191 and include, for example, resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone; 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl) ethylenediamine-N,N′-diacetic acid•H2O); Ambroxol (trans-4-(2-Amino-3,5-dibromobenzylamino)cyclohexane•HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol-2HCl). Analogs and derivatives thereof can also be used.

Other sirtuin-activating compounds may have any of formulas 1-25, 30, 32-65, and 69-76 below. In one embodiment, a sirtuin-activating compound is a stilbene or chalcone compound of formula 1: 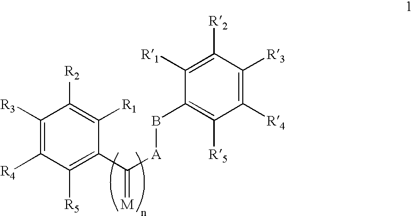
wherein, independently for each occurrence,

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents O, NR, or S;

A-B represents a bivalent alkyl, alkenyl, alkynyl, amido, sulfonamido, diazo, ether, alkylamino, alkylsulfide, hydroxylamine, or hydrazine group; and

n is 0 or 1.

In a further embodiment, a sirtuin-activating compound is a compound of formula 1 and the attendant definitions, wherein n is 0. In a further embodiment, a sirtuin-activating compound is a compound of formula 1 and the attendant definitions, wherein n is 1. In a further embodiment, a sirtuin-activating compound is a compound of formula 1 and the attendant definitions, wherein A-B is ethenyl. In a further embodiment, a sirtuin-activating compound is a compound of formula 1 and the attendant definitions, wherein A-B is —CH₂CH(Me)CH(Me)CH₂—. In a further embodiment, a sirtuin-activating compound is a compound of formula 1 and the attendant definitions, wherein M is O. In a further embodiment, the methods comprises a compound of formula 1 and the attendant definitions, wherein R₁, R₂, R₃, R₄, R₅, R′₁, R′₂, R′₃, R′₄, and R′₅ are H. In a further embodiment, a sirtuin-activating compound is a compound of formula 1 and the attendant definitions, wherein R₂, R₄, and R′₃ are OH. In a further embodiment, a sirtuin-activating compound is a compound of formula 1 and the attendant definitions, wherein R₂, R₄, R′₂ and R′₃ are OH. In a further embodiment, a sirtuin-activating compound is a compound of formula 1 and the attendant definitions, wherein R₃, R₅, R′₂ and R′₃ are OH. In a further embodiment, a sirtuin-activating compound is a compound of formula 1 and the attendant definitions, wherein R₁, R₃, R₅, R′₂ and R′₃ are OH. In a further embodiment, a sirtuin-activating compound is a compound of formula 1 and the attendant definitions, wherein R₂ and R′₂ are OH; R₄ is O-β-D-glucoside; and R′₃ is OCH₃. In a further embodiment, a sirtuin-activating compound is a compound of formula 1 and the attendant definitions, wherein R₂ is OH; R₄ is O-β-D-glucoside; and R′₃ is OCH₃.

In a further embodiment, a sirtuin-activating compound is a compound of formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, a sirtuin-activating compound is a compound of formula 1 and the attendant definitions, wherein n is 1; A-B is ethenyl; M is O; and R₁, R₂, R₃, R₄, R₅, R′₁, R′₂, R′₃, R′₄, and R′₅ are H (chalcone). In a further embodiment, a sirtuin-activating compound is a compound of formula 1 and the attendant definitions, wherein n is 0; A-B is ethenyl; R₂, R₄, and R′₃ are OH; and R₁, R₃, R₅, R′₁, R′₂, R′₄, and R′₅ are H (resveratrol). In a further embodiment, a sirtuin-activating compound is a compound of formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, a sirtuin-activating compound is a compound of formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, a sirtuin-activating compound is a compound of formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, a sirtuin-activating compound is a compound of formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, a sirtuin-activating compound is a compound of formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, a sirtuin-activating compound is a compound of formula 1 and the attendant definitions, wherein n is 0; A-B is —CH₂CH(Me)CH(Me)CH₂—; R₂, R₃, R′₂, and R′₃ are OH; and R₁, R₄, R₅, R′₁, R′₄, and R′₅ are H (NDGA).

In another embodiment, a sirtuin-activating compound is a flavanone compound of formula 2:

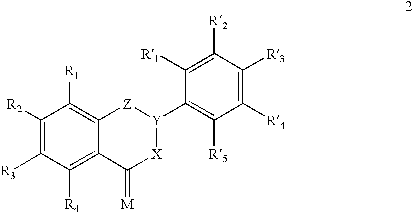

wherein, independently for each occurrence,

R₁, R₂, R₃, R₄, R′₁, R′₂, R′₃, R′₄, R′₅, and R″ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S;

X represents CR or N; and

Y represents CR or N.

In a further embodiment, a sirtuin-activating compound is a compound of formula 2 and the attendant definitions, wherein X and Y are both CH. In a further embodiment, a sirtuin-activating compound is a compound of formula 2 and the attendant definitions, wherein M is O. In a further embodiment, a sirtuin-activating compound is a compound of formula 2 and the attendant definitions, wherein M is H₂. In a further embodiment, a sirtuin-activating compound is a compound of formula 2 and the attendant definitions, wherein Z is O. In a further embodiment, a sirtuin-activating compound is a compound of formula 2 and the attendant definitions, wherein R″ is H. In a further embodiment, a sirtuin-activating compound is a compound of formula 2 and the attendant definitions, wherein R″ is OH. In a further embodiment, a sirtuin-activating compound is a compound of formula 2 and the attendant definitions, wherein R″ is an alkoxycarbonyl. In a further embodiment, a sirtuin-activating compound is a compound of formula 2 and the attendant definitions, wherein R₁ is 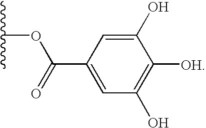
In a further embodiment, a sirtuin-activating compound is a compound of formula 2 and the attendant definitions, wherein R₁, R₂, R₃, R₄, R′₁, R′₂, R′₃, R′₄, R′₅ and R″ are H. In a further embodiment, a sirtuin-activating compound is a compound of formula 2 and the attendant definitions, wherein R₂, R₄, and R′₃ are OH. In a further embodiment, a sirtuin-activating compound is a compound of formula 2 and the attendant definitions, wherein R₄, R′₂, R′₃, and R″ are OH. In a further embodiment, a sirtuin-activating compound is a compound of formula 2 and the attendant definitions, wherein R₂, R₄, R′₂, R′₃, and R″ are OH. In a further embodiment, a sirtuin-activating compound is a compound of formula 2 and the attendant definitions, wherein R₂, R₄, R′₂, R′₃, R′₄, and R″ are OH.

In a further embodiment, a sirtuin-activating compound is a compound of formula 2 and the attendant definitions, wherein X and Y are CH; M is O; Z and 0; R″ is H; and R₁, R₂, R₃, R₄, R′₁, R′₂, R′₃, R′₄, R′₅ and R″ are H (flavanone). In a further embodiment, a sirtuin-activating compound is a compound of formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, a sirtuin-activating compound is a compound of formula 2 and the attendant definitions, wherein X and Y are CH; M is O; Z and O; R″ is OH; R₂, R₄, R′₂, and R′₃ are OH; and R₁, R₃, R′₁, R′₄, and R′₅ are H (3,5,7,3′,4′-pentahydroxyflavanone). In a further embodiment, a sirtuin-activating compound is a compound of formula 2 and the attendant definitions, wherein X and Y are CH; M is H₂; Z and 0; R″ is OH; R₂, R₄, R′₂, and R′₃, are OH; and R₁, R₃, R′₁, R′₄ and R′₅ are H (epicatechin). In a further embodiment, a sirtuin-activating compound is a compound of formula 2 and the attendant definitions, wherein X and Y are CH; M is H₂; Z and 0; R″ is OH; R₂, R₄, R′₂, R′₃, and R′₄ are OH; and R₁, R₃, R′₁, and R′₅ are H (gallocatechin). In a further embodiment, a sirtuin-activating compound is a compound of formula 2 and the attendant definitions, wherein X and Y are CH; M is H₂; Z and 0; R″ is 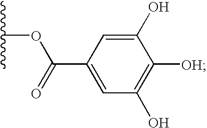
R₂, R₄, R′₂, R′₃, R′₄, and R″ are OH; and R₁, R₃, R′₁, and R′₅ are H (epigallocatechin gallate).

In another embodiment, a sirtuin-activating compound is an isoflavanone compound of formula 3:

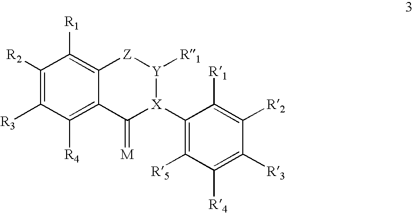

wherein, independently for each occurrence,

R₁, R₂, R₃, R₄, R′₁, R′₂, R′₃, R′₄, R′₅, and R″₁ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S;

X represents CR or N; and

Y represents CR or N.

In another embodiment, a sirtuin-activating compound is a flavone compound of formula 4:

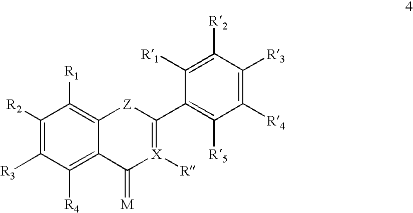

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S; and

X represents CR″ or N, wherein

R″ is H, alkyl, aryl, heteroaryl, alkaryl, heteroaralkyl, halide, NO₂, SR, OR, N(R)₂, or carboxyl.

In a further embodiment, a sirtuin-activating compound is a compound of formula 4 and the attendant definitions, wherein X is C. In a further embodiment, a sirtuin-activating compound is a compound of formula 4 and the attendant definitions, wherein X is CR. In a further embodiment, a sirtuin-activating compound is a compound of formula 4 and the attendant definitions, wherein Z is O. In a further embodiment, a sirtuin-activating compound is a compound of formula 4 and the attendant definitions, wherein M is O. In a further embodiment, a sirtuin-activating compound is a compound of formula 4 and the attendant definitions, wherein R″ is H. In a further embodiment, a sirtuin-activating compound is a compound of formula 4 and the attendant definitions, wherein R″ is OH. In a further embodiment, a sirtuin-activating compound is a compound of formula 4 and the attendant definitions, wherein R₁, R₂, R₃, R₄, R′₁, R′₂, R′₃, R′₄, and R′₅ are H. In a further embodiment, a sirtuin-activating compound is a compound of formula 4 and the attendant definitions, wherein R₂, R′₂, and R′₃ are OH. In a further embodiment, a sirtuin-activating compound is a compound of formula 4 and the attendant definitions, wherein R₂, R₄, R′₂, R′₃, and R′₄ are OH. In a further embodiment, a sirtuin-activating compound is a compound of formula 4 and the attendant definitions, wherein R₂, R₄, R′₂, and R′₃ are OH. In a further embodiment, a sirtuin-activating compound is a compound of formula 4 and the attendant definitions, wherein R₃, R′₂, and R′₃ are OH. In a further embodiment, a sirtuin-activating compound is a compound of formula 4 and the attendant definitions, wherein R₂, R₄, R′₂, and R′₃ are OH. In a further embodiment, a sirtuin-activating compound is a compound of formula 4 and the attendant definitions, wherein R₂, R′₂, R′₃, and R′₄ are OH. In a further embodiment, a sirtuin-activating compound is a compound of formula 4 and the attendant definitions, wherein R₂, R₄, and R′₃ are OH. In a further embodiment, a sirtuin-activating compound is a compound of formula 4 and the attendant definitions, wherein R₂, R₃, R₄, and R′₃ are OH. In a further embodiment, a sirtuin-activating compound is a compound of formula 4 and the attendant definitions, wherein R₂, R₄, and R′₃ are OH. In a further embodiment, a sirtuin-activating compound is a compound of formula 4 and the attendant definitions, wherein R₃, R′₁, and R′₃ are OH. In a further embodiment, a sirtuin-activating compound is a compound of formula 4 and the attendant definitions, wherein R₂ and R′₃ are OH. In a further embodiment, a sirtuin-activating compound is a compound of formula 4 and the attendant definitions, wherein R₁, R₂, R′₂, and R′₃ are OH. In a further embodiment, a sirtuin-activating compound is a compound of formula 4 and the attendant definitions, wherein R₃, R′₁, and R′₂ are OH. In a further embodiment, a sirtuin-activating compound is a compound of formula 4 and the attendant definitions, wherein R′₃ is OH. In a further embodiment, a sirtuin-activating compound is a compound of formula 4 and the attendant definitions, wherein R₄ and R′₃ are OH. In a further embodiment, a sirtuin-activating compound is a compound of formula 4 and the attendant definitions, wherein R₂ and R₄ are OH. In a further embodiment, a sirtuin-activating compound is a compound of formula 4 and the attendant definitions, wherein R₂, R₄, R′₁, and R′₃ are OH. In a further embodiment, a sirtuin-activating compound is a compound of formula 4 and the attendant definitions, wherein R₄ is OH. In a further embodiment, a sirtuin-activating compound is a compound of formula 4 and the attendant definitions, wherein R₂, R₄, R′₂, R′₃, and R′₄ are OH. In a further embodiment, a sirtuin-activating compound is a compound of formula 4 and the attendant definitions, wherein R₂, R′₂, R′₃, and R′₄ are OH. In a further embodiment, a sirtuin-activating compound is a compound of formula 4 and the attendant definitions, wherein R₁, R₂, R₄, R′₂, and R′₃ are OH.

In a further embodiment, a sirtuin-activating compound is a compound of formula 4 and the attendant definitions, wherein X is CH; Z is O; M is O; and R₁, R₂, R₃, R₄, R′₁, R′₂, R′₃, R′₄, and R′₅ are H (flavone). In a further embodiment, a sirtuin-activating compound is a compound of formula 4 and the attendant definitions, wherein X is COH; Z is O; M is O; R₂, R′₂, and R′₃ are OH; and R₁, R₃, R₄, R′₁, R′₄, and R′₅ are H (fisetin). In a further embodiment, a sirtuin-activating compound is a compound of formula 4 and the attendant definitions, wherein X is CH; Z is O; M is O; R₂, R₄, R′₂, R′₃, and R′₄ are OH; and R₁, R₃, R′₁, and R′₅ are H (5,7,3′,4′,5′-pentahydroxyflavone). In a further embodiment, a sirtuin-activating compound is a compound of formula 4 and the attendant definitions, wherein X is CH; Z is O; M is O; R₂, R₄, R′₂, and R′₃ are OH; and R₁, R₃, R′₁, R′₄, and R′₅ are H (luteolin). In a further embodiment, a sirtuin-activating compound is a compound of formula 4 and the attendant definitions, wherein X is COH; Z is O; M is O; R₃, R′₂, and R′₃ are OH; and R₁, R₂, R₄, R′₁ R′₄, and R′₅ are H (3,6,3′,4′-tetrahydroxyflavone). In a further embodiment, a sirtuin-activating compound is a compound of formula 4 and the attendant definitions, wherein X is COH; Z is O; M is O; R₂, R₄, R′₂, and R′₃ are OH; and R₁, R₃, R′₁, R′₄, and R′₅ are H (quercetin). In a further embodiment, a sirtuin-activating compound is a compound of formula 4 and the attendant definitions, wherein X is CH; Z is O; M is O; R₂, R′₂, R′₃, and R′₄ are OH; and R₁, R₃, R₄, R′₁, and R′₅ are H. In a further embodiment, a sirtuin-activating compound is a compound of formula 4 and the attendant definitions, wherein X is COH; Z is O; M is O; R₂, R₄, and R′₃ are OH; and R₁, R₃, R′₁, R′₂, R′₄, and R′₅ are H. In a further embodiment, a sirtuin-activating compound is a compound of formula 4 and the attendant definitions, wherein X is CH; Z is O; M is O; R₂, R₃, R₄, and R′₃ are OH; and R₁, R′₁, R′₂, R′₄, and R′₅ are H. In a further embodiment, a sirtuin-activating compound is a compound of formula 4 and the attendant definitions, wherein X is CH; Z is O; M is 0; R₂, R₄, and R′₃ are OH; and R₁, R₃, R′₁, R′₂, R′₄, and R′₅ are H. In a further embodiment, a sirtuin-activating compound is a compound of formula 4 and the attendant definitions, wherein X is COH; Z is O; M is O; R₃, R′₁, and R′₃ are OH; and R₁, R₂, R₄, R′₂, R′₄, and R′₅ are H. In a further embodiment, a sirtuin-activating compound is a compound of formula 4 and the attendant definitions, wherein X is CH; Z is O; M is O; R₂ and R′₃ are OH; and R₁, R₃, R₄, R′₁, R′₂, R′₄, and R′₅ are H. In a further embodiment, a sirtuin-activating compound is a compound of formula 4 and the attendant definitions, wherein X is COH; Z is O; M is O; R₁, R₂, R′₂, and R′₃ are OH; and R₁, R₂, R₄, R′₃, R′₄, and R′₅ are H. In a further embodiment, a sirtuin-activating compound is a compound of formula 4 and the attendant definitions, wherein X is COH; Z is O; M is O; R₃, R′₁, and R′₂ are OH; and R₁, R₂, R₄; R′₃, R′₄, and R′₅ are H. In a further embodiment, a sirtuin-activating compound is a compound of formula 4 and the attendant definitions, wherein X is CH; Z is O; M is O; R′₃ is OH; and R₁, R₂, R₃, R₄, R′₁, R′₂, R′₄, and R′₅ are H. In a further embodiment, a sirtuin-activating compound is a compound of formula 4 and the attendant definitions, wherein X is CH; Z is O; M is O; R₄ and R′₃ are OH; and R₁, R₂, R₃, R′₁, R′₂, R′₄, and R′₅ are H. In a further embodiment, a sirtuin-activating compound is a compound of formula 4 and the attendant definitions, wherein X is CH; Z is O; M is O; R₂ and R₄ are OH; and R₁, R₃, R′₁, R′₂, R′₃, R′₄, and R′₅ are H. In a further embodiment, a sirtuin-activating compound is a compound of formula 4 and the attendant definitions, wherein X is COH; Z is O; M is O; R₂, R₄, R′₁, and R′₃ are OH; and R₁, R₃, R′₂, R′₄, and R′₅ are H. In a further embodiment, a sirtuin-activating compound is a compound of formula 4 and the attendant definitions, wherein X is CH; Z is O; M is O; R₄ is OH; and R₁, R₂, R₃, R′₁, R′₂, R′₃, R′₄, and R′₅ are H. In a further embodiment, a sirtuin-activating compound is a compound of formula 4 and the attendant definitions, wherein X is COH; Z is O; M is O; R₂, R₄, R′₂, R′₃, and R′₄ are OH; and R₁, R₃, R′₁, and R′₅ are H. In a further embodiment, a sirtuin-activating compound is a compound of formula 4 and the attendant definitions, wherein X is COH; Z is O; M is O; R₂, R′₂, R′₃, and R′₄ are OH; and R₁, R₃, R₄, R′₁, and R′₅ are H. In a further embodiment, a sirtuin-activating compound is a compound of formula 4 and the attendant definitions, wherein X is COH; Z is O; M is O; R, R₂, R₄, R′₂, and R′₃ are OH; and R₃, R′₁, R′₄, and R′₅ are H.

In another embodiment, a sirtuin-activating compound is an isoflavone compound of formula 5:

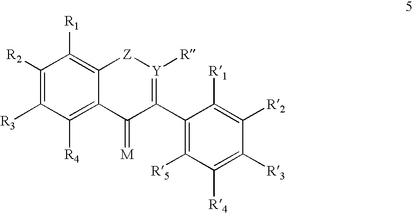

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S; and

Y represents CR″ or N, wherein

R″ represents H, alkyl, aryl, heteroaryl, alkaryl, heteroaralkyl, halide, NO₂, SR, OR, N(R)₂, or carboxyl.

In a further embodiment, a sirtuin-activating compound is a compound of formula 5 and the attendant definitions, wherein Y is CR″. In a further embodiment, a sirtuin-activating compound is a compound of formula 5 and the attendant definitions, wherein Y is CH. In a further embodiment, a sirtuin-activating compound is a compound of formula 5 and the attendant definitions, wherein Z is O. In a further embodiment, a sirtuin-activating compound is a compound of formula 5 and the attendant definitions, wherein M is O. In a further embodiment, a sirtuin-activating compound is a compound of formula 5 and the attendant definitions, wherein R₂ and R′₃ are OH. In a further embodiment, a sirtuin-activating compound is a compound of formula 5 and the attendant definitions, wherein R₂, R₄, and R′₃ are OH.

In a further embodiment, a sirtuin-activating compound is a compound of formula 5 and the attendant definitions, wherein Y is CH; Z is O; M is O; R₂ and R′₃ are OH; and R₁, R₃, R₄, R′₁, R′₂, R′₄, and R′₅ are H. In a further embodiment, a sirtuin-activating compound is a compound of formula 5 and the attendant definitions, wherein Y is CH; Z is O; M is O; R₂, R₄, and R′₃ are OH; and R₁, R₃, R′₁, R′₂, R′₄, and R′₅ are H.

In another embodiment, a sirtuin-activating compound is an anthocyanidin compound of formula 6:

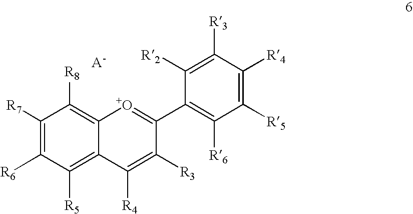

wherein, independently for each occurrence,

R₃, R₄, R₅, R₆, R₇, R₈, R′₂, R′₃, R′₄, R′₅, and R′₆ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl; and

A⁻ represents an anion selected from the following: Cl⁻, Br⁻, or I⁻.

In a further embodiment, a sirtuin-activating compound is a compound of formula 6 and the attendant definitions, wherein A⁻ is Cl⁻. In a further embodiment, a sirtuin-activating compound is a compound of formula 6 and the attendant definitions, wherein R₃, R₅, R₇, and R′₄ are OH. In a further embodiment, a sirtuin-activating compound is a compound of formula 6 and the attendant definitions, wherein R₃, R₅, R₇, R′₃, and R′₄ are OH. In a further embodiment, a sirtuin-activating compound is a compound of formula 6 and the attendant definitions, wherein R₃, R₅, R₇, R′₃, R′₄, and R′₅ are OH.

In a further embodiment, a sirtuin-activating compound is a compound of formula 6 and the attendant definitions, wherein A⁻ is Cl⁻; R₃, R₅, R₇, and R′₄ are OH; and R₄, R₆, R₈, R′₂, R′₃, R′₅, and R′₆ are H. In a further embodiment, a sirtuin-activating compound is a compound of formula 6 and the attendant definitions, wherein A⁻ is Cl⁻; R₃, R₅, R₇, R′₃, and R′₄ are OH; and R₄, R₆, R₈, R′₂, R′₅, and R′₆ are H. In a further embodiment, a sirtuin-activating compound is a compound of formula 6 and the attendant definitions, wherein A⁻ is Cl⁻; R₃, R₅, R₇, R′₃, R′₄, and R′₅ are OH; and R₄, R₆, R₈, R′₂, and R′₆ are H.

In a further embodiment, a sirtuin-activating compound is a stilbene, chalcone, or flavone compound represented by formula 7:

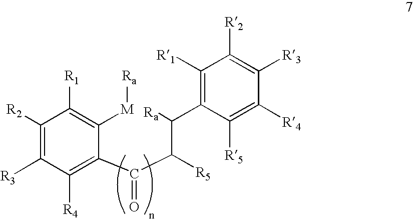

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, aryl, heteroaryl, aralkyl; and

n is 0 or 1.

In a further embodiment, a sirtuin-activating compound is an activating compound represented by formula 7 and the attendant definitions, wherein n is 0. In a further embodiment, a sirtuin-activating compound is an activating compound represented by formula 7 and the attendant definitions, wherein n is 1. In a further embodiment, a sirtuin-activating compound is an activating compound represented by formula 7 and the attendant definitions, wherein M is absent. In a further embodiment, a sirtuin-activating compound is an activating compound represented by formula 7 and the attendant definitions, wherein M is O. In a further embodiment, a sirtuin-activating compound is an activating compound represented by formula 7 and the attendant definitions, wherein Rₐ is H. In a further embodiment, a sirtuin-activating compound is an activating compound represented by formula 7 and the attendant definitions, wherein M is O and the two Rₐ form a bond.

In a further embodiment, a sirtuin-activating compound is an activating compound represented by formula 7 and the attendant definitions, wherein R₅ is H. In a further embodiment, a sirtuin-activating compound is an activating compound represented by formula 7 and the attendant definitions, wherein R₅ is OH. In a further embodiment, a sirtuin-activating compound is an activating compound represented by formula 7 and the attendant definitions, wherein R₁, R₃, and R′₃ are OH. In a further embodiment, a sirtuin-activating compound is an activating compound represented by formula 7 and the attendant definitions, wherein R₂, R₄, R′₂, and R′₃ are OH. In a further embodiment, a sirtuin-activating compound is an activating compound represented by formula 7 and the attendant definitions, wherein R₂, R′₂, and R′₃ are OH. In a further embodiment, a sirtuin-activating compound is an activating compound represented by formula 7 and the attendant definitions, wherein R₂ and R₄ are OH.

In a further embodiment, a sirtuin-activating compound is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin-activating compound is an activating compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin-activating compound is an activating compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

Other sirtuin-activating compounds include compounds having a formula selected from the group consisting of formulas 8-25 and 30 set forth below.

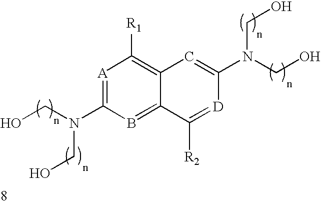

R₁, R₂ = H, aryl, heterocycle, small alkyl
A, B, C, D = CR₁, N
n = 0, 1, 2, 3

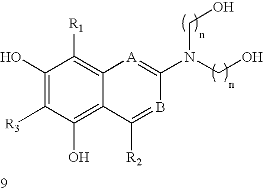

R₁, R₂ = H, aryl, heterocycle, small alkyl
R₃ = H, small alkyl
A, B = CR₁, N
n = 0, 1, 2, 3

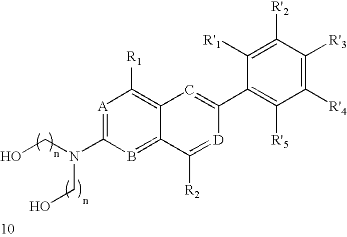

R₁, R₂ = H, aryl, heterocycle, small alkyl
R′₁-R′₅ = H, OH
A, B, C, D = CR₁, N
n = 0, 1, 2, 3

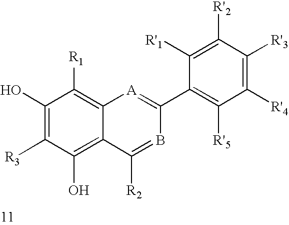

R₁, R₂ = H, aryl, heterocycle, small alkyl
R₃ = H, small alkyl
R′₁-R′₅ = H, OH
A, B = CR₁, N
n = 0, 1, 2, 3

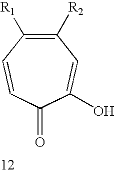

R₁, R₂ = H, alkyl alkenyl

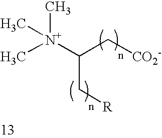

R = Heterocycle, aryl
n = 0-10

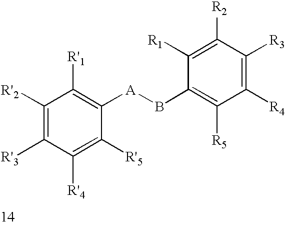

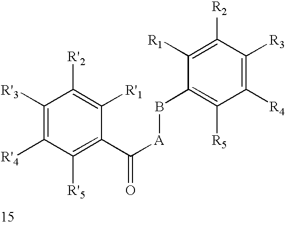

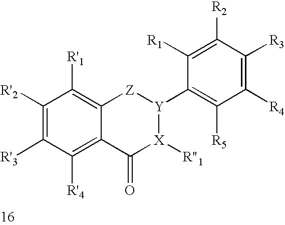

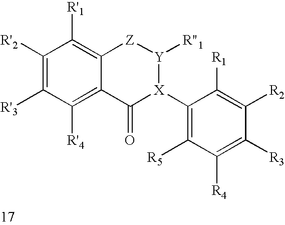

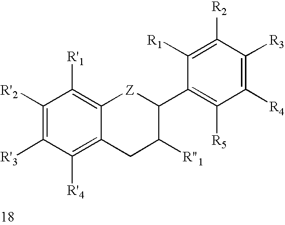

R₁ = H, halogen, NO₂, SR (R = H, alkyl, aryl), OR (R = H, alkyl, aryl),
NRR′]R, R′ = alkyl, aryl), alkyl, aryl, carboxy
R₂ = H, halogen, NO₂, SR (R = H, alkyl, aryl), OR (R = H, alkyl, aryl),
NRR′]R, R′ = alkyl, aryl), alkyl, aryl, carboxy
R₃ = H, halogen, NO₂, SR (R = H, alkyl, aryl), OR (R = H, alkyl, aryl),
NRR′]R, R′ = alkyl, aryl), alkyl, aryl, carboxy
R₄ = H, halogen, NO₂, SR (R = H, alkyl, aryl), OR (R = H, alkyl, aryl),
NRR′]R, R′ = alkyl, aryl), alkyl, aryl, carboxy
R₅ = H, halogen, NO₂, SR (R = H, alkyl, aryl), OR (R = H, alkyl, aryl),
NRR′]R, R′ = alkyl, aryl), alkyl, aryl, carboxy
R′₁ = H, halogen, NO₂, SR (R = H, alkyl, aryl), OR (R = H, alkyl, aryl),
NRR′]R, R′ = alkyl, aryl), alkyl, aryl, carboxy
R′₂ = H, halogen, NO₂, SR (R = H, alkyl, aryl), OR (R = H, alkyl, aryl),
NRR′]R, R′ = alkyl, aryl), alkyl, aryl, carboxy
R′₃ = H, halogen, NO₂, SR (R = H, alkyl, aryl), OR (R = H, alkyl, aryl),
NRR′]R, R′ = alkyl, aryl), alkyl, aryl, carboxy
R′₄ = H, halogen, NO₂, SR (R = H, alkyl, aryl), OR (R = H, alkyl, aryl),
NRR′]R, R′ = alkyl, aryl), alkyl, aryl, carboxy
R′₅ = H, halogen, NO₂, SR (R = H, alkyl, aryl), OR (R = H, alkyl, aryl),
NRR′]R, R′ = alkyl, aryl), alkyl, aryl, carboxy
R″₁ = H, halogen, NO₂, SR (R = H, alkyl, aryl), OR (R = H, alkyl, aryl),
NRR′]R, R′ = alkyl, aryl), alkyl, aryl, carboxy
A—B = ethene, ethylene, amide, sulfonamide, diazo, alkyl ether,
alkyl amine, alkyl sulfide, hydroxyamine, hydrazine
X = CR, N
Y = CR, N
Z = O, S, C(R)₂, NR
R = H, alkyl, aryl, aralkyl

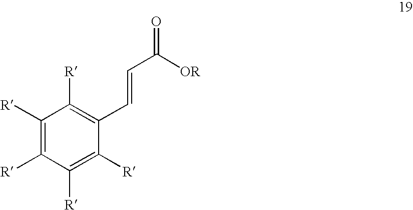

wherein, independently for each occurrence,

R=H, alkyl, aryl, heterocyclyl, heteroaryl, or aralkyl; and

R′=H, halogen, NO₂, SR, OR, NR₂, alkyl, aryl, or carboxy.

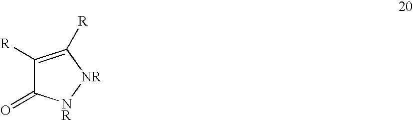

wherein, independently for each occurrence,

R=H, alkyl, aryl, heterocyclyl, heteroaryl, or aralkyl.

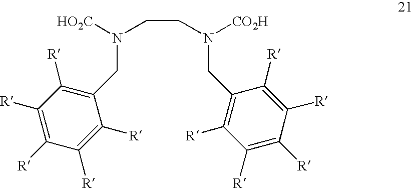

wherein, independently for each occurrence,

R′=H, halogen, NO₂, SR, OR, NR₂, alkyl, aryl, aralkyl, or carboxy; and

R=H, alkyl, aryl, heterocyclyl, heteroaryl, or aralkyl.

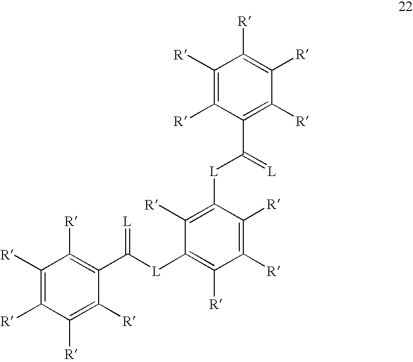

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

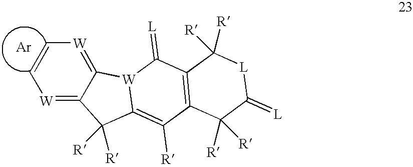

wherein, independently for each occurrence,

L represents CR₂, O, NR, or S;

W represents CR or N;

R represents H, alkyl, aryl, aralkyl, or heteroaralkyl;

Ar represents a fused aryl or heteroaryl ring; and

R′ represents H, halogen, NO₂, SR, OR, NR₂, alkyl, aryl, aralkyl, or carboxy.

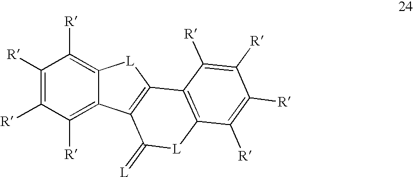

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

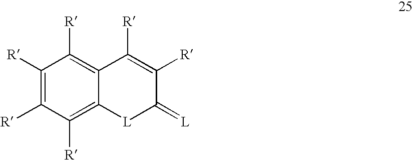

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

In a further embodiment, a sirtuin-activating compound is a stilbene, chalcone, or flavone compound represented by formula 30:

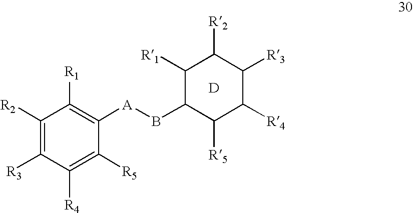

wherein, independently for each occurrence,

D is a phenyl or cyclohexyl group;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R or R′ groups taken together form a fused benzene or cyclohexyl group;

R represents H, alkyl, aryl, or aralkyl; and

A-B represents an ethylene, ethenylene, or imine group;

provided that when A-B is ethenylene, D is phenyl, and R′₃ is H: R₃ is not OH when R₁, R₂, R₄, and R₅ are H; and R₂ and R₄ are not OMe when R₁, R₃, and R₅ are H; and R₃ is not OMe when R₁, R₂, R₄, and R₅ are H.

In a further embodiment, a sirtuin-activating compound is a compound represented by formula 30 and the attendant definitions, wherein D is a phenyl group.

In a further embodiment, a sirtuin-activating compound is a compound represented by formula 30 and the attendant definitions, wherein A-B is an ethenylene or imine group.

In a further embodiment, a sirtuin-activating compound is a compound represented by formula 30 and the attendant definitions, wherein A-B is an ethenylene group.

In a further embodiment, a sirtuin-activating compound is a compound represented by formula 30 and the attendant definitions, wherein R₂ is OH.

In a further embodiment, a sirtuin-activating compound is a compound represented by formula 30 and the attendant definitions, wherein R₄ is OH

In a further embodiment, a sirtuin-activating compound is a compound represented by formula 30 and the attendant definitions, wherein R₂ and R₄ are OH.

In a further embodiment, a sirtuin-activating compound is a compound represented by formula 30 and the attendant definitions, wherein D is a phenyl group; and A-B is an ethenylene group.

In a further embodiment, a sirtuin-activating compound is a compound represented by formula 30 and the attendant definitions, wherein D is a phenyl group; A-B is an ethenylene group; and R₂ and R₄ are OH.

In a further embodiment, a sirtuin-activating compound is a compound represented by formula 30 and the attendant definitions, wherein A-B is ethenylene; D is a phenyl ring; R₂ and R₄ are OH; and R′₃ is Cl.

In a further embodiment, a sirtuin-activating compound is a compound represented by formula 30 and the attendant definitions, wherein A-B is ethenylene; D is a phenyl ring; R₂ and R₄ are OH; and R′₃ is OH.

In a further embodiment, a sirtuin-activating compound is a compound represented by formula 30 and the attendant definitions, wherein A-B is ethenylene; D is a phenyl ring; R₂ and R₄ are OH; and R′₃ is H.

In a further embodiment, a sirtuin-activating compound is a compound represented by formula 30 and the attendant definitions, wherein A-B is ethenylene; D is a phenyl ring; R₂ and R₄ are OH; and R′₃ is CH₂CH₃.

In a further embodiment, a sirtuin-activating compound is a compound represented by formula 30 and the attendant definitions, wherein A-B is ethenylene; D is a phenyl ring; R₂ and R₄ are OH; and R′₃ is F.

In a further embodiment, a sirtuin-activating compound is a compound represented by formula 30 and the attendant definitions, wherein A-B is ethenylene; D is a phenyl ring; R₂ and R₄ are OH; and R′₃ is Me.

In a further embodiment, a sirtuin-activating compound is a compound represented by formula 30 and the attendant definitions, wherein A-B is ethenylene; D is a phenyl ring; R₂ and R₄ are OH; and R′₃ is an azide.

In a further embodiment, a sirtuin-activating compound is a compound represented by formula 30 and the attendant definitions, wherein A-B is ethenylene; D is a phenyl ring; R₂ and R₄ are OH; and R′₃ is SMe.

In a further embodiment, a sirtuin-activating compound is a compound represented by formula 30 and the attendant definitions, wherein A-B is ethenylene; D is a phenyl ring; R₂ and R₄ are OH; and R′₃ is NO₂.

In a further embodiment, a sirtuin-activating compound is a compound represented by formula 30 and the attendant definitions, wherein A-B is ethenylene; D is a phenyl ring; R₂ and R₄ are OH; and R′₃ is CH(CH₃)₂.

In a further embodiment, a sirtuin-activating compound is a compound represented by formula 30 and the attendant definitions, wherein A-B is ethenylene; D is a phenyl ring; R₂ and R₄ are OH; and R′₃ is OMe.

In a further embodiment, a sirtuin-activating compound is a compound represented by formula 30 and the attendant definitions, wherein A-B is ethenylene; D is a phenyl ring; R₂ and R₄ are OH; R′₂ is OH; and R′₃ is OMe.

In a further embodiment, a sirtuin-activating compound is a compound represented by formula 30 and the attendant definitions, wherein A-B is ethenylene; D is a phenyl ring; R₂ is OH; R₄ is carboxyl; and R′₃ is OH.

In a further embodiment, a sirtuin-activating compound is a compound represented by formula 30 and the attendant definitions, wherein A-B is ethenylene; D is a phenyl ring; R₂ and R₄ are OH; and R′₃ is carboxyl.

In a further embodiment, a sirtuin-activating compound is a compound represented by formula 30 and the attendant definitions, wherein A-B is ethenylene; D is a phenyl ring; R₂ and R₄ are OH; and R′₃ and R′₄ taken together form a fused benzene ring.

In a further embodiment, a sirtuin-activating compound is a compound represented by formula 30 and the attendant definitions, wherein A-B is ethenylene; D is a phenyl ring; and R₄ is OH.

In a further embodiment, a sirtuin-activating compound is a compound represented by formula 30 and the attendant definitions, wherein A-B is ethenylene; D is a phenyl ring; R₂ and R₄ are OCH₂OCH₃; and R′₃ is SMe.

In a further embodiment, a sirtuin-activating compound is a compound represented by formula 30 and the attendant definitions, wherein A-B is ethenylene; D is a phenyl ring; R₂ and R₄ are OH; and R′₃ is carboxyl.

In a further embodiment, a sirtuin-activating compound is a compound represented by formula 30 and the attendant definitions, wherein A-B is ethenylene; D is a cyclohexyl ring; and R₂ and R₄ are OH.

In a further embodiment, a sirtuin-activating compound is a compound represented by formula 30 and the attendant definitions, wherein A-B is ethenylene; D is a phenyl ring; and R₃ and R₄ are OMe.

In a further embodiment, a sirtuin-activating compound is a compound represented by formula 30 and the attendant definitions, wherein A-B is ethenylene; D is a phenyl ring; R₂ and R₄ are OH; and R′₃ is OH.

In another embodiment, a sirtuin-activating compound is a compound of formula 32: 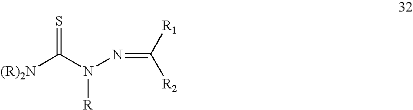
wherein, independently for each occurrence:

R is H, or a substituted or unsubstituted alkyl, aryl, aralkyl, heterocyclyl, heterocyclylalkyl, heteroaryl, or heteroaralkyl; and

R₁ and R₂ are a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 32 and the attendant definitions wherein R is H.

In a further embodiment, a sirtuin-activating compound is a compound of formula 32 and the attendant definitions wherein R₁ is 3-hydroxyphenyl.

In a further embodiment, a sirtuin-activating compound is a compound of formula 32 and the attendant definitions wherein R₂ is methyl.

In a further embodiment, a sirtuin-activating compound is a compound of formula 32 and the attendant definitions wherein R is H and R₁ is 3-hydroxyphenyl.

In a further embodiment, a sirtuin-activating compound is a compound of formula 32 and the attendant definitions wherein R is H, R₁ is 3-hydroxyphenyl, and R₂ is methyl.

In another embodiment, a sirtuin-activating compound is a compound of formula 33: 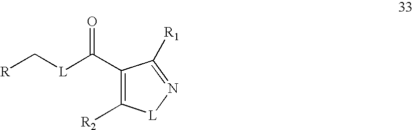
wherein, independently for each occurrence:

R is H, or a substituted or unsubstituted alkyl, alkenyl, or alkynyl;

R₁ and R₂ are a substituted or unsubstituted alkyl, aryl, aralkyl, heterocyclyl, heterocyclylalkyl, heteroaryl, or heteroaralkyl; and

L is O, S, or NR.

In a further embodiment, a sirtuin-activating compound is a compound of formula 33 and the attendant definitions wherein R is alkynyl.

In a further embodiment, a sirtuin-activating compound is a compound of formula 33 and the attendant definitions wherein R₁ is 2,6-dichlorophenyl.

In a further embodiment, a sirtuin-activating compound is a compound of formula 33 and the attendant definitions wherein R₂ is methyl.

In a further embodiment, a sirtuin-activating compound is a compound of formula 33 and the attendant definitions wherein L is O.

In a further embodiment, a sirtuin-activating compound is a compound of formula 33 and the attendant definitions wherein R is alkynyl and R₁ is 2,6-dichlorophenyl.

In a further embodiment, a sirtuin-activating compound is a compound of formula 33 and the attendant definitions wherein R is alkynyl, R₁ is 2,6-dichlorophenyl, and R₂ is methyl.

In a further embodiment, a sirtuin-activating compound is a compound of formula 33 and the attendant definitions wherein R is alkynyl, R₁ is 2,6-dichlorophenyl, R₂ is methyl, and L is O.

In another embodiment, a sirtuin-activating compound is a compound of formula 34: 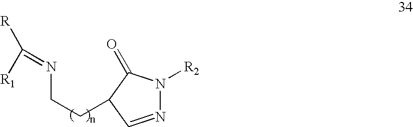
wherein, independently for each occurrence:

R, R₁, and R₂ are H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin-activating compound is a compound of formula 34 and the attendant definitions wherein R is 3,5-dichloro-2-hydroxyphenyl.

In a further embodiment, a sirtuin-activating compound is a compound of formula 34 and the attendant definitions wherein R₁ is H.

In a further embodiment, a sirtuin-activating compound is a compound of formula 34 and the attendant definitions wherein R₂ is H.

In a further embodiment, a sirtuin-activating compound is a compound of formula 34 and the attendant definitions wherein n is 1.

In a further embodiment, a sirtuin-activating compound is a compound of formula 34 and the attendant definitions wherein R is 3,5-dichloro-2-hydroxyphenyl and R₁ is H.

In a further embodiment, a sirtuin-activating compound is a compound of formula 34 and the attendant definitions wherein R is 3,5-dichloro-2-hydroxyphenyl, R₁ is H, and R₂ is H.

In a further embodiment, a sirtuin-activating compound is a compound of formula 34 and the attendant definitions wherein R is 3,5-dichloro-2-hydroxyphenyl, R₁ is H, R₂ is H, and n is 1.

In another embodiment, a sirtuin-activating compound is a compound of formula 35: 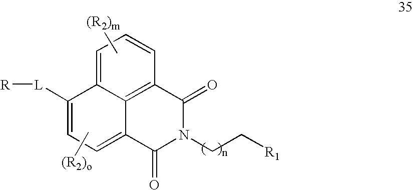
wherein, independently for each occurrence:

R is H or a substituted or unsubstituted alkyl, aryl, aralkyl, heterocyclyl, heterocyclylalkyl, heteroaryl, or heteroaralkyl;

R₁ is a substituted or unsubstituted alkyl, aryl, aralkyl, heterocyclyl, heterocyclylalkyl, heteroaryl, or heteroaralkyl;

R₂ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

L is O, NR, or S;

m is an integer from 0 to 3 inclusive;

n is an integer from 0 to 5 inclusive; and

o is an integer from 0 to 2 inclusive.

In a further embodiment, a sirtuin-activating compound is a compound of formula 35 and the attendant definitions wherein R is phenyl.

In a further embodiment, a sirtuin-activating compound is a compound of formula 35 and the attendant definitions wherein R₁ is pyridine.

In a further embodiment, a sirtuin-activating compound is a compound of formula 35 and the attendant definitions wherein L is S.

In a further embodiment, a sirtuin-activating compound is a compound of formula 35 and the attendant definitions wherein m is 0.

In a further embodiment, a sirtuin-activating compound is a compound of formula 35 and the attendant definitions wherein n is 1.

In a further embodiment, a sirtuin-activating compound is a compound of formula 35 and the attendant definitions wherein o is 0.

In a further embodiment, a sirtuin-activating compound is a compound of formula 35 and the attendant definitions wherein R is phenyl and R₁ is pyridine.

In a further embodiment, a sirtuin-activating compound is a compound of formula 35 and the attendant definitions wherein R is phenyl, R₁ is pyridine, and L is S.

In a further embodiment, a sirtuin-activating compound is a compound of formula 35 and the attendant definitions wherein R is phenyl, R₁ is pyridine, L is S, and m is 0.

In a further embodiment, a sirtuin-activating compound is a compound of formula 35 and the attendant definitions wherein R is phenyl, R₁ is pyridine, L is S, m is 0, and n is 1.

In a further embodiment, a sirtuin-activating compound is a compound of formula 35 and the attendant definitions wherein R is phenyl, R₁ is pyridine, L is S, m is 0, n is 1, and o is 0.

In another embodiment, a sirtuin-activating compound is a compound of formula 36: 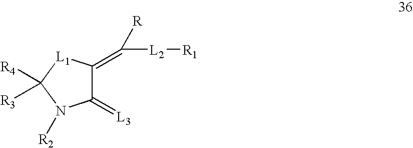
wherein, independently for each occurrence:

R, R₃, and R₄ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ and R₂ are H or a substituted or unsubstituted alkyl, aryl, aralkyl, heterocyclyl, heterocyclylalkyl, heteroaryl, heteroaralkyl;

L₁ is O, NR₁, S, C(R)₂, or SO₂; and

L₂ and L₃ are O, NR₁, S, or C(R)₂.

In a further embodiment, a sirtuin-activating compound is a compound of formula 36 and the attendant definitions wherein R is H.

In a further embodiment, a sirtuin-activating compound is a compound of formula 36 and the attendant definitions wherein R₁ is 4-chlorophenyl.

In a further embodiment, a sirtuin-activating compound is a compound of formula 36 and the attendant definitions wherein R₂ is 4-chlorophenyl.

In a further embodiment, a sirtuin-activating compound is a compound of formula 36 and the attendant definitions wherein R₃ is H.

In a further embodiment, a sirtuin-activating compound is a compound of formula 36 and the attendant definitions wherein R₄ is H.

In a further embodiment, a sirtuin-activating compound is a compound of formula 36 and the attendant definitions wherein L is SO₂.

In a further embodiment, a sirtuin-activating compound is a compound of formula 36 and the attendant definitions wherein L₂ is NH.

In a further embodiment, a sirtuin-activating compound is a compound of formula 36 and the attendant definitions wherein L₃ is O.

In a further embodiment, a sirtuin-activating compound is a compound of formula 36 and the attendant definitions wherein R is H and R₁ is 4-chlorophenyl.

In a further embodiment, a sirtuin-activating compound is a compound of formula 36 and the attendant definitions wherein R is H, R is 4-chlorophenyl, and R₂ is 4-chlorophenyl.

In a further embodiment, a sirtuin-activating compound is a compound of formula 36 and the attendant definitions wherein R is H, R is 4-chlorophenyl, R₂ is 4-chlorophenyl, and R₃ is H.

In a further embodiment, a sirtuin-activating compound is a compound of formula 36 and the attendant definitions wherein R is H, R₁ is 4-chlorophenyl, R₂ is 4-chlorophenyl, R₃ is H, and R₄ is H.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, and L₁ is SO₂.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, L₁ is SO₂, and L₂ is NH.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, L₁ is SO₂, L₂ is NH, and L₃ is O.

In another embodiment, a sirtuin-activating compound is a compound of formula 37: 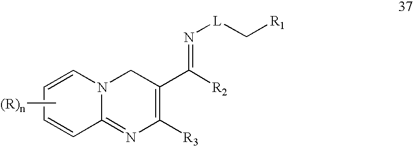
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ is H or a substituted or unsubstituted alkyl, aryl, aralkyl, heterocyclyl, heterocyclylalkyl, heteroaryl, heteroaralkyl;

R₂ and R₃ are H or a substituted or unsubstituted alkyl, aryl, aralkyl, heterocyclyl, heterocyclylalkyl, heteroaryl, heteroaralkyl;

L is O, NR₁, or S; and

n is an integer from 0 to 4 inclusive.

In a further embodiment, a sirtuin-activating compound is a compound of formula 37 and the attendant definitions wherein R is methyl.

In a further embodiment, a sirtuin-activating compound is a compound of formula 37 and the attendant definitions wherein n is 1.

In a further embodiment, a sirtuin-activating compound is a compound of formula 37 and the attendant definitions wherein R₁ is 3-fluorophenyl.

In a further embodiment, a sirtuin-activating compound is a compound of formula 37 and the attendant definitions wherein R₂ is H.

In a further embodiment, a sirtuin-activating compound is a compound of formula 37 and the attendant definitions wherein R₃ is 4-chlorophenyl.

In a further embodiment, a sirtuin-activating compound is a compound of formula 37 and the attendant definitions wherein L is O.

In a further embodiment, a sirtuin-activating compound is a compound of formula 37 and the attendant definitions wherein R is methyl and n is 1.

In a further embodiment, a sirtuin-activating compound is a compound of formula 37 and the attendant definitions wherein R is methyl, n is 1, and R₁ is 3-fluorophenyl.

In a further embodiment, a sirtuin-activating compound is a compound of formula 37 and the attendant definitions wherein R is methyl, n is 1, R₁ is 3-fluorophenyl, and R₂ is H.

In a further embodiment, a sirtuin-activating compound is a compound of formula 37 and the attendant definitions wherein R is methyl, n is 1, R₁ is 3-fluorophenyl, R₂ is H, and R₃ is 4-chlorophenyl.

In another embodiment, a sirtuin-activating compound is a compound of formula 38: 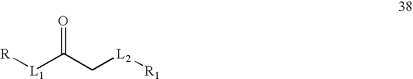
wherein, independently for each occurrence:

R and R₁ are H or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin-activating compound is a compound of formula 38 and the attendant definitions wherein R is 3-methoxyphenyl.

In a further embodiment, a sirtuin-activating compound is a compound of formula 38 and the attendant definitions wherein R₁ is 4-t-butylphenyl.

In a further embodiment, a sirtuin-activating compound is a compound of formula 38 and the attendant definitions wherein L₁ is NH.

In a further embodiment, a sirtuin-activating compound is a compound of formula 38 and the attendant definitions wherein L₂ is O.

In a further embodiment, a sirtuin-activating compound is a compound of formula 38 and the attendant definitions wherein R is 3-methoxyphenyl and R₁ is 4-t-butylphenyl.

In a further embodiment, a sirtuin-activating compound is a compound of formula 38 and the attendant definitions wherein R is 3-methoxyphenyl, R₁ is 4-t-butylphenyl, and L₁ is NH.

In a further embodiment, a sirtuin-activating compound is a compound of formula 38 and the attendant definitions wherein R is 3-methoxyphenyl, R₁ is 4-t-butylphenyl, L₁ is NH, and L₂ is O.

In another embodiment, a sirtuin-activating compound is a compound of formula 39: 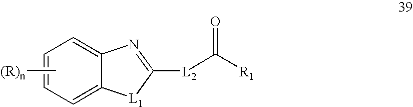
wherein, independently for each occurrence:

R is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ is H or a substituted or unsubstituted alkyl, aryl, alkaryl, heterocyclyl, heterocyclylalkyl, heteroaryl, or heteroaralkyl;

L₁ and L₂ are O, NR, or S; and

n is an integer from 0 to 4 inclusive.

In a further embodiment, a sirtuin-activating compound is a compound of formula 39 and the attendant definitions wherein R is methyl.

In a further embodiment, a sirtuin-activating compound is a compound of formula 39 and the attendant definitions wherein n is 1.

In a further embodiment, a sirtuin-activating compound is a compound of formula 39 and the attendant definitions wherein R₁ is 3,4,5-trimethoxyphenyl.

In a further embodiment, a sirtuin-activating compound is a compound of formula 39 and the attendant definitions wherein L₁ is S.

In a further embodiment, a sirtuin-activating compound is a compound of formula 39 and the attendant definitions wherein L₂ is NH.

In a further embodiment, a sirtuin-activating compound is a compound of formula 39 and the attendant definitions wherein R is methyl and n is 1.

In a further embodiment, a sirtuin-activating compound is a compound of formula 39 and the attendant definitions wherein R is methyl, n is 1, and R₁ is 3,4,5-trimethoxyphenyl.

In a further embodiment, a sirtuin-activating compound is a compound of formula 39 and the attendant definitions wherein R is methyl, n is 1, R₁ is 3,4,5-trimethoxyphenyl, and L₁ is S.

In a further embodiment, a sirtuin-activating compound is a compound of formula 39 and the attendant definitions wherein R is methyl, n is 1, R₁ is 3,4,5-trimethoxyphenyl, L₁ is S, and L₂ is NH.

In another embodiment, a sirtuin-activating compound is a compound of formula 40: 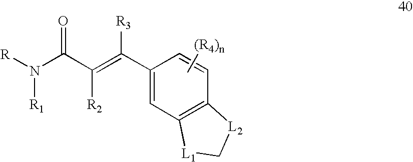
wherein, independently for each occurrence:

R, R₁, R₂, R₃ are H or a substituted or unsubstituted alkyl, aryl, alkaryl, heterocyclyl, heterocyclylalkyl, heteroaryl, or heteroaralkyl;

R₄ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR, or S; and

n is an integer from 0 to 3 inclusive.

In a further embodiment, a sirtuin-activating compound is a compound of formula 40 and the attendant definitions wherein R is H.

In a further embodiment, a sirtuin-activating compound is a compound of formula 40 and the attendant definitions wherein R₁ is perfluorophenyl.

In a further embodiment, a sirtuin-activating compound is a compound of formula 40 and the attendant definitions wherein R₂ is H.

In a further embodiment, a sirtuin-activating compound is a compound of formula 40 and the attendant definitions wherein R₃ is H.

In a further embodiment, a sirtuin-activating compound is a compound of formula 40 and the attendant definitions wherein L₁ is O.

In a further embodiment, a sirtuin-activating compound is a compound of formula 40 and the attendant definitions wherein L₂ is O.

In a further embodiment, a sirtuin-activating compound is a compound of formula 40 and the attendant definitions wherein n is 0.

In a further embodiment, a sirtuin-activating compound is a compound of formula 40 and the attendant definitions wherein R is H and R₁ is perfluorophenyl.

In a further embodiment, a sirtuin-activating compound is a compound of formula 40 and the attendant definitions wherein R is H, R₁ is perfluorophenyl, and R₂ is H.

In a further embodiment, a sirtuin-activating compound is a compound of formula 40 and the attendant definitions R is H, R₁ is perfluorophenyl, R₂ is H, and R₃ is H.

In a further embodiment, a sirtuin-activating compound is a compound of formula 40 and the attendant definitions wherein R is H, R₁ is perfluorophenyl, R₂ is H, R₃ is H, and L₁ is O.

In a further embodiment, a sirtuin-activating compound is a compound of formula 40 and the attendant definitions wherein R is H, R₁ is perfluorophenyl, R₂ is H, R₃ is H, L₁ is O, and L₂ is O.

In a further embodiment, a sirtuin-activating compound is a compound of formula 40 and the attendant definitions wherein R is H, R₁ is perfluorophenyl, R₂ is H, R₃ is H, L₁ is O, L₂ is O, and n is 0.

In another embodiment, a sirtuin-activating compound is a compound of formula 41: 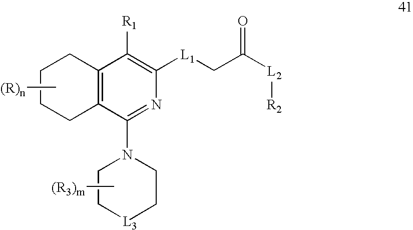
wherein, independently for each occurrence:

R, R₁, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H or a substituted or unsubstituted alkyl, aryl, aralkyl, heterocyclyl, heterocyclylalkyl, heteroaryl, or heteroaralkyl;

L₁, L₂, and L₃ are O, NR₂, or S; and

m and n are integers from 0 to 8 inclusive.

In a further embodiment, a sirtuin-activating compound is a compound of formula 41 and the attendant definitions wherein n is 0.

In a further embodiment, a sirtuin-activating compound is a compound of formula 41 and the attendant definitions wherein R₁ is cyano.

In a further embodiment, a sirtuin-activating compound is a compound of formula 41 and the attendant definitions wherein R₂ is ethyl.

In a further embodiment, a sirtuin-activating compound is a compound of formula 41 and the attendant definitions wherein m is 0.

In a further embodiment, a sirtuin-activating compound is a compound of formula 41 and the attendant definitions wherein L₁ is S.

In a further embodiment, a sirtuin-activating compound is a compound of formula 41 and the attendant definitions wherein L₂ is O.

In a further embodiment, a sirtuin-activating compound is a compound of formula 41 and the attendant definitions wherein L₃ is O.

In a further embodiment, a sirtuin-activating compound is a compound of formula 41 and the attendant definitions wherein n is 0 and R₁ is cyano.

In a further embodiment, a sirtuin-activating compound is a compound of formula 41 and the attendant definitions wherein n is 0, R₁ is cyano, and R₂ is ethyl.

In a further embodiment, a sirtuin-activating compound is a compound of formula 41 and the attendant definitions wherein n is 0, R₁ is cyano, R₂ is ethyl, and m is 0.

In a further embodiment, a sirtuin-activating compound is a compound of formula 41 and the attendant definitions wherein n is 0, R₁ is cyano, R₂ is ethyl, m is 0, and L₁ is S.

In a further embodiment, a sirtuin-activating compound is a compound of formula 41 and the attendant definitions wherein n is 0, R₁ is cyano, R₂ is ethyl, m is 0, L₁ is S, and L₂ is O.

In a further embodiment, a sirtuin-activating compound is a compound of formula 41 and the attendant definitions wherein n is 0, R₁ is cyano, R₂ is ethyl, m is 0, L₁ is S, L₂ is O, and L₃ is O.

In another embodiment, a sirtuin-activating compound is a compound of formula 42: 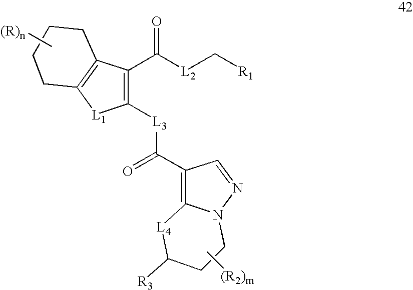
wherein, independently for each occurrence:

R and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₃ are H or a substituted or unsubstituted alkyl, aryl, aralkyl, heterocyclyl, heterocyclylalkyl, heteroaryl, or heteroaralkyl;

L₁, L₂, L₃, and L₄ are O, NR, or S;

m is an integer from 0 to 6 inclusive; and

n is an integer from 0 to 8 inclusive.

In a further embodiment, a sirtuin-activating compound is a compound of formula 42 and the attendant definitions wherein n is 0.

In a further embodiment, a sirtuin-activating compound is a compound of formula 42 and the attendant definitions wherein R₁ is methyl.

In a further embodiment, a sirtuin-activating compound is a compound of formula 42 and the attendant definitions wherein R₂ is CF₃ and m is 1.

In a further embodiment, a sirtuin-activating compound is a compound of formula 42 and the attendant definitions wherein R₃ is 4-methylphenyl.

In a further embodiment, a sirtuin-activating compound is a compound of formula 42 and the attendant definitions wherein L₁ is S.

In a further embodiment, a sirtuin-activating compound is a compound of formula 42 and the attendant definitions wherein L₂ is O.

In a further embodiment, a sirtuin-activating compound is a compound of formula 42 and the attendant definitions wherein L₃ is NR₁.

In a further embodiment, a sirtuin-activating compound is a compound of formula 42 and the attendant definitions wherein L₄ is NR₁.

In a further embodiment, a sirtuin-activating compound is a compound of formula 42 and the attendant definitions wherein n is 0 and R₁ is methyl.

In a further embodiment, a sirtuin-activating compound is a compound of formula 42 and the attendant definitions wherein n is 0, R₁ is methyl, R₂ is CF₃, and m is 1.

In a further embodiment, a sirtuin-activating compound is a compound of formula 42 and the attendant definitions wherein n is 0, R₁ is methyl, R₂ is CF₃, m is 1; and R₃ is 4-methylphenyl.

In a further embodiment, a sirtuin-activating compound is a compound of formula 42 and the attendant definitions wherein n is 0, R₁ is methyl, R₂ is CF₃, m is 1; R₃ is 4-methylphenyl; and L₁ is S.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, and L₂ is O.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, L₂ is O; and L₃ is NR₁.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, L₂ is O; L₃ is NR₁, and L₄ is NR₁.

In another embodiment, a sirtuin-activating compound is a compound of formula 43: 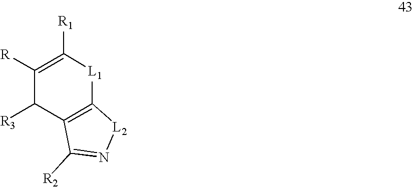
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ and R₃ are H or a substituted or unsubstituted alkyl, aryl, aralkyl, heterocyclyl, heterocyclylalkyl, heteroaryl, or heteroaralkyl; and

L₁ and L₂ are O, NR₂, or S.

In a further embodiment, a sirtuin-activating compound is a compound of formula 43 and the attendant definitions wherein R is cyano. In a further embodiment, a sirtuin-activating compound is a compound of formula 43 and the attendant definitions wherein R₁ is NH₂.

In a further embodiment, a sirtuin-activating compound is a compound of formula 43 and the attendant definitions wherein R₂ is 4-bromophenyl.

In a further embodiment, a sirtuin-activating compound is a compound of formula 43 and the attendant definitions wherein R₃ is 3-hydroxy-4-methoxyphenyl.

In a further embodiment, a sirtuin-activating compound is a compound of formula 43 and the attendant definitions wherein L₁ is O.

In a further embodiment, a sirtuin-activating compound is a compound of formula 43 and the attendant definitions wherein L₂ is NR₂.

In a further embodiment, a sirtuin-activating compound is a compound of formula 43 and the attendant definitions wherein R is cyano and R₁ is NH₂.

In a further embodiment, a sirtuin-activating compound is a compound of formula 43 and the attendant definitions wherein R is cyano, R₁ is NH₂, and R₂ is 4-bromophenyl.

In a further embodiment, a sirtuin-activating compound is a compound of formula 43 and the attendant definitions wherein R is cyano, R₁ is NH₂, R₂ is 4-bromophenyl, and R₃ is 3-hydroxy-4-methoxyphenyl.

In a further embodiment, a sirtuin-activating compound is a compound of formula 43 and the attendant definitions wherein R is cyano, R₁ is NH₂, R₂ is 4-bromophenyl, R₃ is 3-hydroxy-4-methoxyphenyl, and L₁ is O.

In a further embodiment, a sirtuin-activating compound is a compound of formula 43 and the attendant definitions wherein R is cyano, R₁ is NH₂, R₂ is 4-bromophenyl, R₃ is 3-hydroxy-4-methoxyphenyl, L₁ is O, and L₂ is NR₂.

In another embodiment, a sirtuin-activating compound is a compound of formula 44: 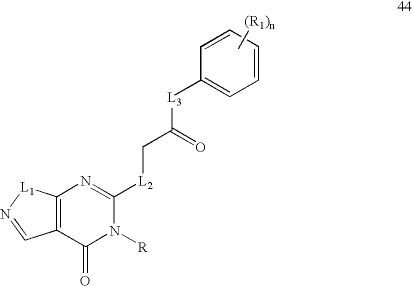
wherein, independently for each occurrence:

R is H or a substituted or unsubstituted alkyl, aryl, aralkyl, heterocyclyl, heterocyclylalkyl, heteroaryl, or heteroaralkyl;

R₁ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S; and

n is an integer from 0 to 5 inclusive.

In a further embodiment, a sirtuin-activating compound is a compound of formula 44 and the attendant definitions wherein R is 3-trifluoromethylphenyl.

In a further embodiment, a sirtuin-activating compound is a compound of formula 44 and the attendant definitions wherein R₁ is C(O)OCH₃.

In a further embodiment, a sirtuin-activating compound is a compound of formula 44 and the attendant definitions wherein L₁ is NR.

In a further embodiment, a sirtuin-activating compound is a compound of formula 44 and the attendant definitions wherein L₂ is S.

In a further embodiment, a sirtuin-activating compound is a compound of formula 44 and the attendant definitions wherein L₃ is NR.

In a further embodiment, a sirtuin-activating compound is a compound of formula 44 and the attendant definitions wherein n is 2.

In a further embodiment, a sirtuin-activating compound is a compound of formula 44 and the attendant definitions wherein R is 3-trifluoromethylphenyl and R₁ is C(O)OCH₃.

In a further embodiment, a sirtuin-activating compound is a compound of formula 44 and the attendant definitions wherein R is 3-trifluoromethylphenyl, R₁ is C(O)OCH₃, and L₁ is NR.

In a further embodiment, a sirtuin-activating compound is a compound of formula 44 and the attendant definitions wherein R is 3-trifluoromethylphenyl, R₁ is C(O)OCH₃, L₁ is NR, and L₂ is S.

In a further embodiment, a sirtuin-activating compound is a compound of formula 44 and the attendant definitions wherein R is 3-trifluoromethylphenyl, R₁ is C(O)OCH₃, L₁ is NR, L₂ is S, and L₃ is NR.

In a further embodiment, a sirtuin-activating compound is a compound of formula 44 and the attendant definitions wherein R is 3-trifluoromethylphenyl, R₁ is C(O)OCH₃, L₁ is NR, L₂ is S, L₃ is NR, and n is 2.

In another embodiment, a sirtuin-activating compound is a compound of formula 45: 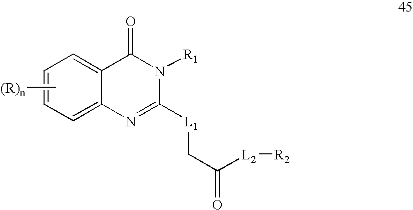
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H or a substituted or unsubstituted alkyl, aryl, aralkyl, heterocyclyl, heterocyclylalkyl, heteroaryl, or heteroaralkyl;

L₁ and L₂ are O, NR₁, or S; and

n is an integer from 0 to 4 inclusive.

In a further embodiment, a sirtuin-activating compound is a compound of formula 45 and the attendant definitions wherein n is 0.

In a further embodiment, a sirtuin-activating compound is a compound of formula 45 and the attendant definitions wherein R₁ is 2-tetrahydrofuranylmethyl.

In a further embodiment, a sirtuin-activating compound is a compound of formula 45 and the attendant definitions wherein R₂ is —CH₂CH₂C₆H₄SO₂NH₂.

In a further embodiment, a sirtuin-activating compound is a compound of formula 45 and the attendant definitions wherein L₁ is S.

In a further embodiment, a sirtuin-activating compound is a compound of formula 45 and the attendant definitions wherein L₂ is NR₁.

In a further embodiment, a sirtuin-activating compound is a compound of formula 45 and the attendant definitions wherein n is 0 and R₁ is 2-tetrahydrofuranylmethyl.

In a further embodiment, a sirtuin-activating compound is a compound of formula 45 and the attendant definitions wherein n is 0, R₁ is 2-tetrahydrofuranylmethyl, and R₂ is —CH₂CH₂C₆H₄SO₂NH₂.

In a further embodiment, a sirtuin-activating compound is a compound of formula 45 and the attendant definitions wherein n is 0, R₁ is 2-tetrahydrofuranylmethyl, R₂ is —CH₂CH₂C₆H₄SO₂NH₂, and L₁ is S.

In a further embodiment, a sirtuin-activating compound is a compound of formula 45 and the attendant definitions wherein n is 0, R₁ is 2-tetrahydrofuranylmethyl, R₂ is —CH₂CH₂C₆H₄SO₂NH₂, L₁ is S, and L₂ is NR₁.

In another embodiment, a sirtuin-activating compound is a compound of formula 46: 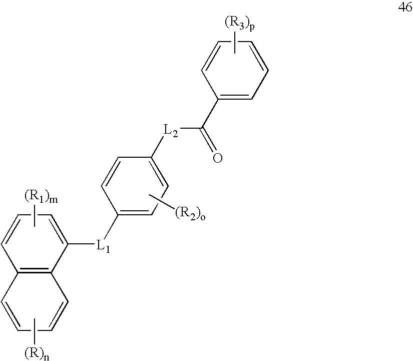
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl;

n is an integer from 0 to 4 inclusive;

m is an integer from 0 to 3 inclusive;

o is an integer from 0 to 4 inclusive; and

p is an integer from 0 to 5 inclusive.

In a further embodiment, a sirtuin-activating compound is a compound of formula 46 and the attendant definitions wherein n is 0.

In a further embodiment, a sirtuin-activating compound is a compound of formula 46 and the attendant definitions wherein m is 1.

In a further embodiment, a sirtuin-activating compound is a compound of formula 46 and the attendant definitions wherein R₁ is Cl.

In a further embodiment, a sirtuin-activating compound is a compound of formula 46 and the attendant definitions wherein o is 1.

In a further embodiment, a sirtuin-activating compound is a compound of formula 46 and the attendant definitions wherein R₂ is Cl.

In a further embodiment, a sirtuin-activating compound is a compound of formula 46 and the attendant definitions wherein p is 3.

In a further embodiment, a sirtuin-activating compound is a compound of formula 46 and the attendant definitions wherein R₃ is OH or I.

In a further embodiment, a sirtuin-activating compound is a compound of formula 46 and the attendant definitions wherein n is 0 and m is 1.

In a further embodiment, a sirtuin-activating compound is a compound of formula 46 and the attendant definitions wherein n is 0, m is 1, and o is 1.

In a further embodiment, a sirtuin-activating compound is a compound of formula 46 and the attendant definitions wherein n is 0, m is 1, o is 1, and R₁ is Cl.

In a further embodiment, a sirtuin-activating compound is a compound of formula 46 and the attendant definitions wherein n is 0, m is 1, o is 1, R₁ is Cl, and p is 3.

In a further embodiment, a sirtuin-activating compound is a compound of formula 46 and the attendant definitions wherein n is 0, m is 1, o is 1, R₁ is Cl, p is 3, and R₂ is OH or I.

In another embodiment, a sirtuin-activating compound is a compound of formula 47: 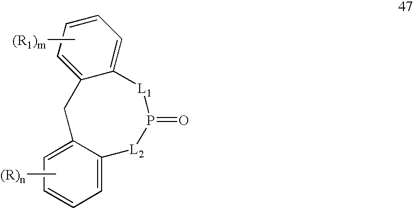
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl; and

m and n are integers from 0 to 4 inclusive.

In a further embodiment, a sirtuin-activating compound is a compound of formula 47 and the attendant definitions wherein n is 2.

In a further embodiment, a sirtuin-activating compound is a compound of formula 47 and the attendant definitions wherein R is methyl or t-butyl.

In a further embodiment, a sirtuin-activating compound is a compound of formula 47 and the attendant definitions wherein m is 2.

In a further embodiment, a sirtuin-activating compound is a compound of formula 47 and the attendant definitions wherein R₁ is methyl or t-butyl.

In a further embodiment, a sirtuin-activating compound is a compound of formula 47 and the attendant definitions wherein L₁ is O.

In a further embodiment, a sirtuin-activating compound is a compound of formula 47 and the attendant definitions wherein L₂ is O.

In a further embodiment, a sirtuin-activating compound is a compound of formula 47 and the attendant definitions wherein n is 2 and R is methyl or t-butyl.

In a further embodiment, a sirtuin-activating compound is a compound of formula 47 and the attendant definitions wherein n is 2, R is methyl or t-butyl, and m is 2.

In a further embodiment, a sirtuin-activating compound is a compound of formula 47 and the attendant definitions wherein n is 2, R is methyl or t-butyl, m is 2, and R₁ is methyl or t-butyl.

In a further embodiment, a sirtuin-activating compound is a compound of formula 47 and the attendant definitions wherein n is 2, R is methyl or t-butyl, m is 2, R₁ is methyl or t-butyl, and L₁ is O.

In a further embodiment, a sirtuin-activating compound is a compound of formula 47 and the attendant definitions wherein n is 2, R is methyl or t-butyl, m is 2, R₁ is methyl or t-butyl, L₁ is O, and L₂ is O.

In another embodiment, a sirtuin-activating compound is a compound of formula 48: 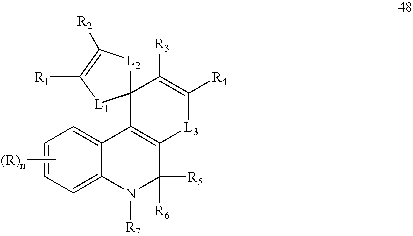
wherein, independently for each occurrence:

R, R₁, R₂, R₃, R₄, R₅, and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₇ is H or a substituted or unsubstituted alkyl, acyl, aryl, aralkyl, heterocyclyl, heterocyclylalkyl, heteroaryl, or heteroaralkyl;

L₁, L₂, and L₃ are O, NR₇, or S and

n is an integer from 0 to 4 inclusive.

In a further embodiment, a sirtuin-activating compound is a compound of formula 48 and the attendant definitions wherein n is 1.

In a further embodiment, a sirtuin-activating compound is a compound of formula 48 and the attendant definitions wherein R is methyl.

In a further embodiment, a sirtuin-activating compound is a compound of formula 48 and the attendant definitions wherein R₁ is C(O)OCH₃.

In a further embodiment, a sirtuin-activating compound is a compound of formula 48 and the attendant definitions wherein R₂ is C(O)OCH₃.

In a further embodiment, a sirtuin-activating compound is a compound of formula 48 and the attendant definitions wherein R₃ is C(O)OCH₃.

In a further embodiment, a sirtuin-activating compound is a compound of formula 48 and the attendant definitions wherein R₄ is C(O)OCH₃.

In a further embodiment, a sirtuin-activating compound is a compound of formula 48 and the attendant definitions wherein R₅ is methyl.

In a further embodiment, a sirtuin-activating compound is a compound of formula 48 and the attendant definitions wherein R₆ is methyl.

In a further embodiment, a sirtuin-activating compound is a compound of formula 48 and the attendant definitions wherein R₇ is C(O)CF₃.

In a further embodiment, a sirtuin-activating compound is a compound of formula 48 and the attendant definitions wherein L₁ is S.

In a further embodiment, a sirtuin-activating compound is a compound of formula 48 and the attendant definitions wherein L₂ is S.

In a further embodiment, a sirtuin-activating compound is a compound of formula 48 and the attendant definitions wherein L₃ is S.

In a further embodiment, a sirtuin-activating compound is a compound of formula 48 and the attendant definitions wherein n is 1 and R is methyl.

In a further embodiment, a sirtuin-activating compound is a compound of formula 48 and the attendant definitions wherein n is 1, R is methyl, and R₁ is C(O)OCH₃.

In a further embodiment, a sirtuin-activating compound is a compound of formula 48 and the attendant definitions wherein n is 1, R is methyl, R₁ is C(O)OCH₃, and R₂ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, and R₃ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, and R₄ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, and R₅ is methyl.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, and R₆ is methyl.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, and R₇ is C(O)CF₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, and L₁ is S.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, and L₂ is S.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, L₂ is S, and L₃ is S.

In another embodiment, a sirtuin-activating compound is a compound of formula 49: 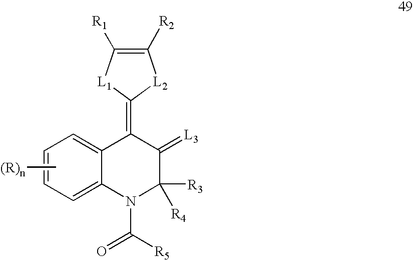
wherein, independently for each occurrence:

R, R₁, R₂, R₃, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₆, or S;

R₆ is H or a substituted or unsubstituted alkyl, aryl, aralkyl, heterocyclyl, heterocyclylalkyl, heteroaryl, or heteroaralkyl; and

n is an integer from 0 to 4 inclusive.

In a further embodiment, a sirtuin-activating compound is a compound of formula 49 and the attendant definitions wherein n is 1.

In a further embodiment, a sirtuin-activating compound is a compound of formula 49 and the attendant definitions wherein R is methyl.

In a further embodiment, a sirtuin-activating compound is a compound of formula 49 and the attendant definitions wherein R₁ is C(O)OCH₃.

In a further embodiment, a sirtuin-activating compound is a compound of formula 49 and the attendant definitions wherein R₂ is C(O)OCH₃.

In a further embodiment, a sirtuin-activating compound is a compound of formula 49 and the attendant definitions wherein R₃ is methyl.

In a further embodiment, a sirtuin-activating compound is a compound of formula 49 and the attendant definitions wherein R₄ is methyl.

In a further embodiment, a sirtuin-activating compound is a compound of formula 49 and the attendant definitions wherein R₅ is CH₂CH(CH₃)₂.

In a further embodiment, a sirtuin-activating compound is a compound of formula 49 and the attendant definitions wherein L₁ is S.

In a further embodiment, a sirtuin-activating compound is a compound of formula 49 and the attendant definitions wherein L₂ is S.

In a further embodiment, a sirtuin-activating compound is a compound of formula 49 and the attendant definitions wherein L₃ is S.

In a further embodiment, a sirtuin-activating compound is a compound of formula 49 and the attendant definitions wherein n is 1 and R is methyl.

In a further embodiment, a sirtuin-activating compound is a compound of formula 49 and the attendant definitions wherein n is 1, R is methyl, and R₁ is C(O)OCH₃.

In a further embodiment, a sirtuin-activating compound is a compound of formula 49 and the attendant definitions wherein n is 1, R is methyl, R₁ is C(O)OCH₃, and R₂ is C(O)OCH₃.

In a further embodiment, a sirtuin-activating compound is a compound of formula 49 and the attendant definitions wherein n is 1, R is methyl, R₁ is C(O)OCH₃, R₂ is C(O)OCH₃, and R₃ is methyl.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, and R₄ is methyl.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, and R₅ is CH₂CH(CH₃)₂.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, L₂ is S, and L₃ is S.

In another embodiment, a sirtuin-activating compound is a compound of formula 50: 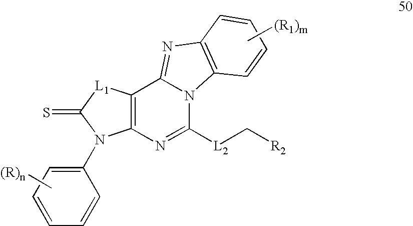
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₃, or S;

R₃ is H or a substituted or unsubstituted alkyl, aryl, aralkyl, heterocyclyl, heterocyclylalkyl, heteroaryl, or heteroaralkyl;

n is an integer from 0 to 5 inclusive; and

m is an integer from 0 to 4 inclusive.

In a further embodiment, a sirtuin-activating compound is a compound of formula 50 and the attendant definitions wherein n is 1.

In a further embodiment, a sirtuin-activating compound is a compound of formula 50 and the attendant definitions wherein R is CO₂Et.

In a further embodiment, a sirtuin-activating compound is a compound of formula 50 and the attendant definitions wherein m is 0.

In a further embodiment, a sirtuin-activating compound is a compound of formula 50 and the attendant definitions wherein R₂ is cyano.

In a further embodiment, a sirtuin-activating compound is a compound of formula 50 and the attendant definitions wherein L₁ is S.

In a further embodiment, a sirtuin-activating compound is a compound of formula 50 and the attendant definitions wherein L₂ is S.

In a further embodiment, a sirtuin-activating compound is a compound of formula 50 and the attendant definitions wherein n is 1 and R is CO₂Et.

In a further embodiment, a sirtuin-activating compound is a compound of formula 50 and the attendant definitions wherein n is 1, R is CO₂Et, and m is 0.

In a further embodiment, a sirtuin-activating compound is a compound of formula 50 and the attendant definitions wherein n is 1, R is CO₂Et, m is 0, and R₂ is cyano. In a further embodiment, a sirtuin-activating compound is a compound of formula 50 and the attendant definitions wherein n is 1, R is CO₂Et, m is 0, R₂ is cyano, and L₁ is S.

In a further embodiment, a sirtuin-activating compound is a compound of formula 50 and the attendant definitions wherein n is 1, R is CO₂Et, m is 0, R₂ is cyano, L₁ is S, and L₂ is S.

In another embodiment, a sirtuin-activating compound is a compound of formula 51: 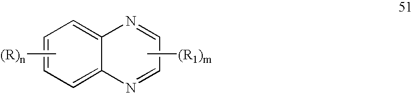
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 2 inclusive.

In a further embodiment, a sirtuin-activating compound is a compound of formula 51 and the attendant definitions wherein n is 2.

In a further embodiment, a sirtuin-activating compound is a compound of formula 51 and the attendant definitions wherein R is Cl or trifluoromethyl.

In a further embodiment, a sirtuin-activating compound is a compound of formula 51 and the attendant definitions wherein m is 2.

In a further embodiment, a sirtuin-activating compound is a compound of formula 51 and the attendant definitions wherein R₁ is phenyl.

In a further embodiment, a sirtuin-activating compound is a compound of formula 51 and the attendant definitions wherein n is 2 and R is Cl or trifluoromethyl.

In a further embodiment, a sirtuin-activating compound is a compound of formula 51 and the attendant definitions wherein n is 2, R is Cl or trifluoromethyl, and m is 2.

In a further embodiment, a sirtuin-activating compound is a compound of formula 51 and the attendant definitions wherein n is 2, R is Cl or trifluoromethyl, m is 2, and R₁ is phenyl.

In a further embodiment, a sirtuin-activating compound is a compound of formula 51 and the attendant definitions wherein n is 1.

In a further embodiment, a sirtuin-activating compound is a compound of formula 51 and the attendant definitions wherein R is F.

In a further embodiment, a sirtuin-activating compound is a compound of formula 51 and the attendant definitions wherein R₁ is 4-methylphenyl.

In a further embodiment, a sirtuin-activating compound is a compound of formula 51 and the attendant definitions wherein n is 1 and R is F.

In a further embodiment, a sirtuin-activating compound is a compound of formula 51 and the attendant definitions wherein n is 1, R is F, and m is 2.

In a further embodiment, a sirtuin-activating compound is a compound of formula 51 and the attendant definitions wherein n is 1, R is F, m is 2, and R₁ is 4-methylphenyl.

In another embodiment, a sirtuin-activating compound is a compound of formula 52: 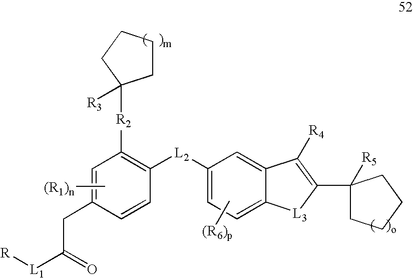
wherein, independently for each occurrence:

R is H or a substituted or unsubstituted alkyl, aryl, aralkyl, heterocyclyl, heterocyclylalkyl, heteroaryl, or heteroaralkyl;

R₁ and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkylene, alkenylene, or alkynylene;

R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S;

n and p are integers from 0 to 3 inclusive; and

m and o are integers from 0 to 2 inclusive.

In a further embodiment, a sirtuin-activating compound is a compound of formula 52 and the attendant definitions wherein R is CH₂CH₂OH.

In a further embodiment, a sirtuin-activating compound is a compound of formula 52 and the attendant definitions wherein n is 1.

In a further embodiment, a sirtuin-activating compound is a compound of formula 52 and the attendant definitions wherein R₁ is I.

In a further embodiment, a sirtuin-activating compound is a compound of formula 52 and the attendant definitions wherein R₂ is alkynylene.

In a further embodiment, a sirtuin-activating compound is a compound of formula 52 and the attendant definitions wherein m is 1.

In a further embodiment, a sirtuin-activating compound is a compound of formula 52 and the attendant definitions wherein R₃ is OH.

In a further embodiment, a sirtuin-activating compound is a compound of formula 52 and the attendant definitions wherein R₄ is C(O)OEt.

In a further embodiment, a sirtuin-activating compound is a compound of formula 52 and the attendant definitions wherein o is 1.

In a further embodiment, a sirtuin-activating compound is a compound of formula 52 and the attendant definitions wherein R₅ is OH.

In a further embodiment, a sirtuin-activating compound is a compound of formula 52 and the attendant definitions wherein p is 0.

In a further embodiment, a sirtuin-activating compound is a compound of formula 52 and the attendant definitions wherein L₁ is NH.

In a further embodiment, a sirtuin-activating compound is a compound of formula 52 and the attendant definitions wherein L₂ is O.

In a further embodiment, a sirtuin-activating compound is a compound of formula 52 and the attendant definitions wherein L₃ is O.

In a further embodiment, a sirtuin-activating compound is a compound of formula 52 and the attendant definitions wherein R is CH₂CH₂OH and n is 1.

In a further embodiment, a sirtuin-activating compound is a compound of formula 52 and the attendant definitions wherein R is CH₂CH₂OH, n is 1, and R₁ is I.

In a further embodiment, a sirtuin-activating compound is a compound of formula 52 and the attendant definitions wherein R is CH₂CH₂OH, n is 1, R₁ is I, and R₂ is alkynylene.

In a further embodiment, a sirtuin-activating compound is a compound of formula 52 and the attendant definitions wherein R is CH₂CH₂OH, n is 1, R₁ is I, R₂ is alkynylene, and m is 1.

In a further embodiment, a sirtuin-activating compound is a compound of formula 52 and the attendant definitions wherein R is CH₂CH₂OH, n is 1, R₁ is I, R₂ is alkynylene, m is 1, and R₃ is OH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, and R₄ is C(O)OEt.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, and o is 1.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, and R₅ is OH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, and p is 0.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R is C(O)OEt, o is 1, R₅ is OH, p is 0, and L₁ is NH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L1 is NH, and L₂ is O.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, L₂ is 0, and L₃ is O.

In another embodiment, a sirtuin-activating compound is a compound of formula 53: 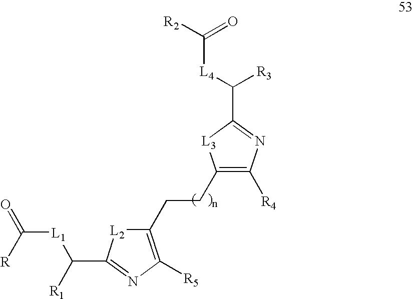
wherein, independently for each occurrence:

R, R₁, R₂, R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, L₃, and L₄ are O, NR₆, or S;

R₆ is and H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin-activating compound is a compound of formula 53 and the attendant definitions wherein R is O-t-butyl.

In a further embodiment, a sirtuin-activating compound is a compound of formula 53 and the attendant definitions wherein R₁ is t-butyl.

In a further embodiment, a sirtuin-activating compound is a compound of formula 53 and the attendant definitions wherein R₂ is O-t-butyl.

In a further embodiment, a sirtuin-activating compound is a compound of formula 53 and the attendant definitions wherein R₃ is t-butyl.

In a further embodiment, a sirtuin-activating compound is a compound of formula 53 and the attendant definitions wherein R₄ is C(O)OMe.

In a further embodiment, a sirtuin-activating compound is a compound of formula 53 and the attendant definitions wherein R₅ is C(O)OMe.

In a further embodiment, a sirtuin-activating compound is a compound of formula 53 and the attendant definitions wherein L₁ is NH.

In a further embodiment, a sirtuin-activating compound is a compound of formula 53 and the attendant definitions wherein L₂ is O.

In a further embodiment, a sirtuin-activating compound is a compound of formula 53 and the attendant definitions wherein L₃ is O.

In a further embodiment, a sirtuin-activating compound is a compound of formula 53 and the attendant definitions wherein L₄ is NH.

In a further embodiment, a sirtuin-activating compound is a compound of formula 53 and the attendant definitions wherein n is 1.

In a further embodiment, a sirtuin-activating compound is a compound of formula 53 and the attendant definitions wherein R is O-t-butyl and R₁ is t-butyl.

In a further embodiment, a sirtuin-activating compound is a compound of formula 53 and the attendant definitions wherein R is O-t-butyl, R₁ is t-butyl, and R₂ is O-t-butyl.

In a further embodiment, a sirtuin-activating compound is a compound of formula 53 and the attendant definitions wherein R is O-t-butyl, R₁ is t-butyl, R₂ is O-t-butyl, and R₃ is t-butyl.

In a further embodiment, a sirtuin-activating compound is a compound of formula 53 and the attendant definitions wherein R is O-t-butyl, R₁ is t-butyl, R₂ is O-t-butyl, R₃ is t-butyl, and R₄ is C(O)OMe.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, and R₅ is C(O)OMe.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, and L₁ is NH.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, and L₂ is O.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, and L₃ is O.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, and L₄ is NH.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, L₄ is NH, and n is 1.

In another embodiment, a sirtuin-activating compound is a compound of formula 54: 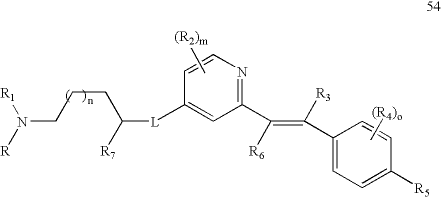
wherein, independently for each occurrence:

R and R₁ are H or a substituted or unsubstituted alkyl, aryl, aralkyl, heterocyclyl, heterocyclylalkyl, heteroaryl, or heteroaralkyl;

R₂, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR, or S;

n and o are integers from 0 to 4 inclusive; and

m is an integer from 0 to 3 inclusive.

In a further embodiment, a sirtuin-activating compound is a compound of formula 54 and the attendant definitions wherein R is ethyl.

In a further embodiment, a sirtuin-activating compound is a compound of formula 54 and the attendant definitions wherein R₁ is ethyl.

In a further embodiment, a sirtuin-activating compound is a compound of formula 54 and the attendant definitions wherein m is 0.

In a further embodiment, a sirtuin-activating compound is a compound of formula 54 and the attendant definitions wherein R₃ is H.

In a further embodiment, a sirtuin-activating compound is a compound of formula 54 and the attendant definitions wherein o is 0.

In a further embodiment, a sirtuin-activating compound is a compound of formula 54 and the attendant definitions wherein R₅ is Cl.

In a further embodiment, a sirtuin-activating compound is a compound of formula 54 and the attendant definitions wherein R₆ is H.

In a further embodiment, a sirtuin-activating compound is a compound of formula 54 and the attendant definitions wherein R₇ is methyl.

In a further embodiment, a sirtuin-activating compound is a compound of formula 54 and the attendant definitions wherein L is NH.

In a further embodiment, a sirtuin-activating compound is a compound of formula 54 and the attendant definitions wherein n is 1.

In a further embodiment, a sirtuin-activating compound is a compound of formula 54 and the attendant definitions wherein R is ethyl and R₁ is ethyl.

In a further embodiment, a sirtuin-activating compound is a compound of formula 54 and the attendant definitions wherein R is ethyl, R₁ is ethyl, and m is 0.

In a further embodiment, a sirtuin-activating compound is a compound of formula 54 and the attendant definitions wherein R is ethyl, R is ethyl, m is 0, and R₃ is H.

In a further embodiment, a sirtuin-activating compound is a compound of formula 54 and the attendant definitions wherein R is ethyl, R₁ is ethyl, m is 0, R₃ is H, and o is 0.

In a further embodiment, a sirtuin-activating compound is a compound of formula 54 and the attendant definitions wherein R is ethyl, R₁ is ethyl, m is 0, R₃ is H, o is 0, and R₅ is Cl.

In a further embodiment, a sirtuin-activating compound is a compound of formula 54 and the attendant definitions wherein R is ethyl, R is ethyl, m is 0, R₃ is H, o is 0, R₅ is Cl, and R₆ is H.

In a further embodiment, a sirtuin-activating compound is a compound of formula 54 and the attendant definitions wherein R is ethyl, R is ethyl, m is 0, R₃ is H, o is 0, R₅ is Cl, R₆ is H, and R₇ is methyl.

In a further embodiment, a sirtuin-activating compound is a compound of formula 54 and the attendant definitions wherein R is ethyl, R is ethyl, m is 0, R₃ is H, o is 0, R₅ is Cl, R₆ is H, R₇ is methyl, and L is NH.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, R₇ is methyl, L is NH, and n is 1.

In another embodiment, a sirtuin-activating compound is a compound of formula 55: 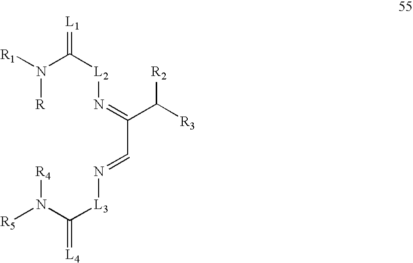
wherein, independently for each occurrence:

R, R₁, R₄, and R₅ are H or a substituted or unsubstituted alkyl, aryl, aralkyl, heterocyclyl, heterocyclylalkyl, heteroaryl, or heteroaralkyl;

R₂ and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁, L₂, L₃, and L₄ are O, NR, or S.

In a further embodiment, a sirtuin-activating compound is a compound of formula 55 and the attendant definitions wherein R is H.

In a further embodiment, a sirtuin-activating compound is a compound of formula 55 and the attendant definitions wherein R₁ is H.

In a further embodiment, a sirtuin-activating compound is a compound of formula 55 and the attendant definitions wherein R₂ is OEt.

In a further embodiment, a sirtuin-activating compound is a compound of formula 55 and the attendant definitions wherein R₃ is methyl.

In a further embodiment, a sirtuin-activating compound is a compound of formula 55 and the attendant definitions wherein R₄ is H.

In a further embodiment, a sirtuin-activating compound is a compound of formula 55 and the attendant definitions wherein R₅ is H.

In a further embodiment, a sirtuin-activating compound is a compound of formula 55 and the attendant definitions wherein L₁ is S.

In a further embodiment, a sirtuin-activating compound is a compound of formula 55 and the attendant definitions wherein L₂ is NH.

In a further embodiment, a sirtuin-activating compound is a compound of formula 55 and the attendant definitions wherein L₃ is NH.

In a further embodiment, a sirtuin-activating compound is a compound of formula 55 and the attendant definitions wherein L₄ is S.

In a further embodiment, a sirtuin-activating compound is a compound of formula 55 and the attendant definitions wherein R is H and R₁ is H.

In a further embodiment, a sirtuin-activating compound is a compound of formula 55 and the attendant definitions wherein R is H, R₁ is H, and R₂ is OEt.

In a further embodiment, a sirtuin-activating compound is a compound of formula 55 and the attendant definitions wherein R is H, R₁ is H, R₂ is OEt, and R₃ is methyl.

In a further embodiment, a sirtuin-activating compound is a compound of formula 55 and the attendant definitions wherein R is H, R is H, R₂ is OEt, R₃ is methyl, and R₄ is H.

In a further embodiment, a sirtuin-activating compound is a compound of formula 55 and the attendant definitions wherein R is H, R₁ is H, R₂ is OEt, R₃ is methyl, R₄ is H, and R₅ is H.

In a further embodiment, a sirtuin-activating compound is a compound of formula 55 and the attendant definitions wherein R is H, R₁ is H, R₂ is OEt, R₃ is methyl, R₄ is H, R₅ is H, and L₁ is S.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, and L₂ is NH. In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, L₂ is NH, and L₃ is NH.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, L₂ is NH, L₃ is NH, and L₄ is S.

In another embodiment, a sirtuin-activating compound is a compound of formula 56: 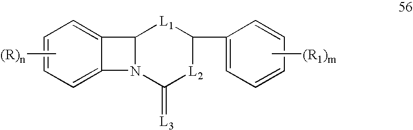
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₂, or S;

R₂ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 5 inclusive.

In a further embodiment, a sirtuin-activating compound is a compound of formula 56 and the attendant definitions wherein n is 0.

In a further embodiment, a sirtuin-activating compound is a compound of formula 56 and the attendant definitions wherein m is 0.

In a further embodiment, a sirtuin-activating compound is a compound of formula 56 and the attendant definitions wherein L₁ is NH.

In a further embodiment, a sirtuin-activating compound is a compound of formula 56 and the attendant definitions wherein L₂ is S.

In a further embodiment, a sirtuin-activating compound is a compound of formula 56 and the attendant definitions wherein L₃ is S.

In a further embodiment, a sirtuin-activating compound is a compound of formula 56 and the attendant definitions wherein m is 0 and n is 0.

In a further embodiment, a sirtuin-activating compound is a compound of formula 56 and the attendant definitions wherein m is 0, n is 0, and L₁ is NH.

In a further embodiment, a sirtuin-activating compound is a compound of formula 56 and the attendant definitions wherein m is 0, n is 0, L₁ is NH, and L₂ is S.

In a further embodiment, a sirtuin-activating compound is a compound of formula 56 and the attendant definitions wherein m is 0, n is 0, L₁ is NH, L₂ is S, and L₃ is S.

In another embodiment, a sirtuin-activating compound is a compound of formula 57: 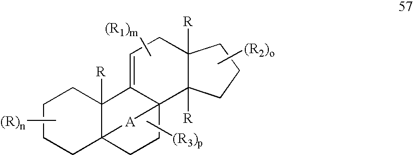
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

A is alkylene, alkenylene, or alkynylene;

n is an integer from 0 to 8 inclusive;

m is an integer from 0 to 3 inclusive;

o is an integer from 0 to 6 inclusive; and

p is an integer from 0 to 4 inclusive.

In a further embodiment, a sirtuin-activating compound is a compound of formula 57 and the attendant definitions wherein n is 2.

In a further embodiment, a sirtuin-activating compound is a compound of formula 57 and the attendant definitions wherein R is OH or methyl.

In a further embodiment, a sirtuin-activating compound is a compound of formula 57 and the attendant definitions wherein m is 1.

In a further embodiment, a sirtuin-activating compound is a compound of formula 57 and the attendant definitions wherein R₁ is methyl.

In a further embodiment, a sirtuin-activating compound is a compound of formula 57 and the attendant definitions wherein o is 1.

In a further embodiment, a sirtuin-activating compound is a compound of formula 57 and the attendant definitions wherein R₂ is C(O)CH₃.

In a further embodiment, a sirtuin-activating compound is a compound of formula 57 and the attendant definitions wherein p is 2.

In a further embodiment, a sirtuin-activating compound is a compound of formula 57 and the attendant definitions wherein R₃ is CO₂H.

In a further embodiment, a sirtuin-activating compound is a compound of formula 57 and the attendant definitions wherein A is alkenylene.

In a further embodiment, a sirtuin-activating compound is a compound of formula 57 and the attendant definitions wherein n is 2 and R is OH or methyl.

In a further embodiment, a sirtuin-activating compound is a compound of formula 57 and the attendant definitions wherein n is 2, R is OH or methyl, and m is 1.

In a further embodiment, a sirtuin-activating compound is a compound of formula 57 and the attendant definitions wherein n is 2, R is OH or methyl, m is 1, and R₁ is methyl.

In a further embodiment, a sirtuin-activating compound is a compound of formula 57 and the attendant definitions wherein n is 2, R is OH or methyl, m is 1, R₁ is methyl, and o is 1.

In a further embodiment, a sirtuin-activating compound is a compound of formula 57 and the attendant definitions wherein n is 2, R is OH or methyl, m is 1, R₁ is methyl, o is 1, and R₂ is C(O)CH₃.

In a further embodiment, a sirtuin-activating compound is a compound of formula 57 and the attendant definitions wherein n is 2, R is OH or methyl, m is 1, R₁ is methyl, o is 1, R₂ is C(O)CH₃, and p is 2.

In a further embodiment, a sirtuin-activating compound is a compound of formula 57 and the attendant definitions wherein n is 2, R is OH or methyl, m is 1, R₁ is methyl, o is 1, R₂ is C(O)CH₃, p is 2, and R₃ is CO₂H.

In a further embodiment, a sirtuin-activating compound is a compound of formula 57 and the attendant definitions wherein n is 2, R is OH or methyl, m is 1, R₁ is methyl, o is 1, R₂ is C(O)CH₃, p is 2, R₃ is CO₂H, and A is alkenylene.

In another embodiment, a sirtuin-activating compound is a compound of formula 58: 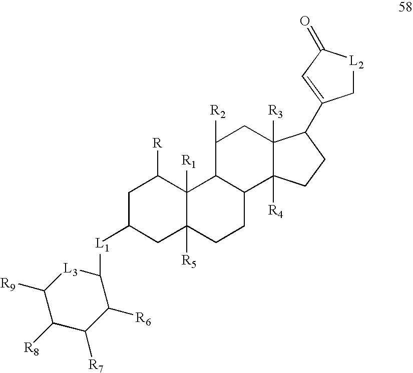
wherein, independently for each occurrence:

R, R₁, R₂, R₃, R4, R₅, R₆, R₇, R₈, and R₉ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₁₀, or S; and

R₁₀ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 58 and the attendant definitions wherein R is OH.

In a further embodiment, a sirtuin-activating compound is a compound of formula 58 and the attendant definitions wherein R₁ is CH₂OH.

In a further embodiment, a sirtuin-activating compound is a compound of formula 58 and the attendant definitions wherein R₂ is OH.

In a further embodiment, a sirtuin-activating compound is a compound of formula 58 and the attendant definitions wherein R₃ is methyl.

In a further embodiment, a sirtuin-activating compound is a compound of formula 58 and the attendant definitions wherein R₄ is OH.

In a further embodiment, a sirtuin-activating compound is a compound of formula 58 and the attendant definitions wherein R₅ is OH.

In a further embodiment, a sirtuin-activating compound is a compound of formula 58 and the attendant definitions wherein R₆ is OH.

In a further embodiment, a sirtuin-activating compound is a compound of formula 58 and the attendant definitions wherein R₇ is OH.

In a further embodiment, a sirtuin-activating compound is a compound of formula 58 and the attendant definitions wherein R₈ is OH.

In a further embodiment, a sirtuin-activating compound is a compound of formula 58 and the attendant definitions wherein R₉ is methyl.

In a further embodiment, a sirtuin-activating compound is a compound of formula 58 and the attendant definitions wherein L₁ is O.

In a further embodiment, a sirtuin-activating compound is a compound of formula 58 and the attendant definitions wherein L₂ is O.

In a further embodiment, a sirtuin-activating compound is a compound of formula 58 and the attendant definitions wherein L₃ is O.

In a further embodiment, a sirtuin-activating compound is a compound of formula 58 and the attendant definitions wherein R is OH and R₁ is CH₂OH.

In a further embodiment, a sirtuin-activating compound is a compound of formula 58 and the attendant definitions wherein R is OH, R₁ is CH₂OH, and R₂ is OH.

In a further embodiment, a sirtuin-activating compound is a compound of formula 58 and the attendant definitions wherein R is OH, R₁ is CH₂OH, R₂ is OH, and R₃ is methyl.

In a further embodiment, a sirtuin-activating compound is a compound of formula 58 and the attendant definitions wherein R is OH, R₁ is CH₂OH, R₂ is OH, R₃ is methyl, and R₄ is OH.

In a further embodiment, a sirtuin-activating compound is a compound of formula 58 and the attendant definitions wherein R is OH, R₁ is CH₂OH, R₂ is OH, R₃ is methyl, R₄ is OH, and R₅ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, and R₆ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, and R₇ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, and R₈ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, and R₉ is methyl.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, and L₁ is O.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, and L₂ is O.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, L₂ is O, and L₃ is O.

In another embodiment, a sirtuin-activating compound is a compound of formula 59: 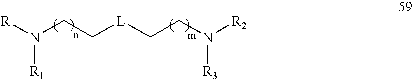
wherein, independently for each occurrence:

R, R₁, R₂, and R₃ are H or a substituted or unsubstituted alkyl, aryl, aralkyl, heterocyclyl, heterocyclylalkyl, heteroaryl, or heteroaralkyl;

L is O, NR, S, or Se; and

n and m are integers from 0 to 5 inclusive.

In a further embodiment, a sirtuin-activating compound is a compound of formula 59 and the attendant definitions wherein R is H.

In a further embodiment, a sirtuin-activating compound is a compound of formula 59 and the attendant definitions wherein R₁ is H.

In a further embodiment, a sirtuin-activating compound is a compound of formula 59 and the attendant definitions wherein R₂ is H.

In a further embodiment, a sirtuin-activating compound is a compound of formula 59 and the attendant definitions wherein R₃ is H.

In a further embodiment, a sirtuin-activating compound is a compound of formula 59 and the attendant definitions wherein L is Se.

In a further embodiment, a sirtuin-activating compound is a compound of formula 59 and the attendant definitions wherein n is 1.

In a further embodiment, a sirtuin-activating compound is a compound of formula 59 and the attendant definitions wherein m is 1.

In a further embodiment, a sirtuin-activating compound is a compound of formula 59 and the attendant definitions wherein R is H and R₁ is H.

In a further embodiment, a sirtuin-activating compound is a compound of formula 59 and the attendant definitions wherein R is H, R₁ is H, and R₂ is H.

In a further embodiment, a sirtuin-activating compound is a compound of formula 59 and the attendant definitions wherein R is H, R₁ is H, R₂ is H, and R₃ is H.

In a further embodiment, a sirtuin-activating compound is a compound of formula 59 and the attendant definitions wherein R is H, R₁ is H, R₂ is H, R₃ is H, and L is Se.

In a further embodiment, a sirtuin-activating compound is a compound of formula 59 and the attendant definitions wherein R is H, R₁ is H, R₂ is H, R₃ is H, L is Se, and n is 1.

In a further embodiment, a sirtuin-activating compound is a compound of formula 59 and the attendant definitions wherein R is H, R₁ is H, R₂ is H, R₃ is H, L is Se, n is 1, and m is 1.

In another embodiment, a sirtuin-activating compound is a compound of formula 60: 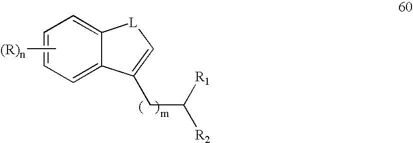
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₃, S, or SO₂;

R₃ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 1 to 5 inclusive.

In a further embodiment, a sirtuin-activating compound is a compound of formula 60 and the attendant definitions wherein n is 1.

In a further embodiment, a sirtuin-activating compound is a compound of formula 60 and the attendant definitions wherein R is Cl.

In a further embodiment, a sirtuin-activating compound is a compound of formula 60 and the attendant definitions wherein R₁ is NH₂.

In a further embodiment, a sirtuin-activating compound is a compound of formula 60 and the attendant definitions wherein R₂ is CO₂H.

In a further embodiment, a sirtuin-activating compound is a compound of formula 60 and the attendant definitions wherein L is SO₂.

In a further embodiment, a sirtuin-activating compound is a compound of formula 60 and the attendant definitions wherein m is 1.

In a further embodiment, a sirtuin-activating compound is a compound of formula 60 and the attendant definitions wherein n is 1 and R is Cl.

In a further embodiment, a sirtuin-activating compound is a compound of formula 60 and the attendant definitions wherein n is 1, R is Cl, and R₁ is NH₂.

In a further embodiment, a sirtuin-activating compound is a compound of formula 60 and the attendant definitions wherein n is 1, R is Cl, R₁ is NH₂, and R₂ is CO₂H.

In a further embodiment, a sirtuin-activating compound is a compound of formula 60 and the attendant definitions wherein n is 1, R is Cl, R₁ is NH₂, R₂ is CO₂H, and L is SO₂.

In a further embodiment, a sirtuin-activating compound is a compound of formula 60 and the attendant definitions wherein n is 1, R is Cl, R₁ is NH₂, R₂ is CO₂H, L is SO₂, and m is 1.

In another embodiment, a sirtuin-activating compound is a compound of formula 61: 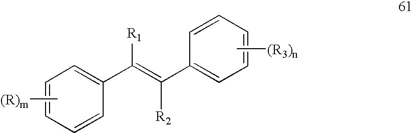
wherein, independently for each occurrence:

R, R₁, R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n and m are integers from 0 to 5 inclusive.

In a further embodiment, a sirtuin-activating compound is a compound of formula 61 and the attendant definitions wherein n is 2.

In a further embodiment, a sirtuin-activating compound is a compound of formula 61 and the attendant definitions wherein R is 3-hydroxy and 5-hydroxy.

In a further embodiment, a sirtuin-activating compound is a compound of formula 61 and the attendant definitions wherein R₁ is H.

In a further embodiment, a sirtuin-activating compound is a compound of formula 61 and the attendant definitions wherein R₂ is H.

In a further embodiment, a sirtuin-activating compound is a compound of formula 61 and the attendant definitions wherein m is 0.

In a further embodiment, a sirtuin-activating compound is a compound of formula 61 and the attendant definitions wherein m is 1.

In a further embodiment, a sirtuin-activating compound is a compound of formula 61 and the attendant definitions wherein R₃ is 4-hydroxy.

In a further embodiment, a sirtuin-activating compound is a compound of formula 61 and the attendant definitions wherein R₃ is 4-methoxy.

In a further embodiment, a sirtuin-activating compound is a compound of formula 61 and the attendant definitions wherein n is 2 and R is 3-hydroxy and 5-hydroxy.

In a further embodiment, a sirtuin-activating compound is a compound of formula 61 and the attendant definitions wherein n is 2, R is 3-hydroxy and 5-hydroxy, and R₁ is H.

In a further embodiment, a sirtuin-activating compound is a compound of formula 61 and the attendant definitions wherein n is 2, R is 3-hydroxy and 5-hydroxy, R₁ is H, and R₂ is H.

In a further embodiment, a sirtuin-activating compound is a compound of formula 61 and the attendant definitions wherein n is 2, R is 3-hydroxy and 5-hydroxy, R₁ is H, R₂ is H, and m is 0.

In a further embodiment, a sirtuin-activating compound is a compound of formula 61 and the attendant definitions wherein n is 2, R is 3-hydroxy and 5-hydroxy, R₁ is H, R₂ is H, and m is 1.

In a further embodiment, a sirtuin-activating compound is a compound of formula 61 and the attendant definitions wherein n is 2, R is 3-hydroxy and 5-hydroxy, R₁ is H, R₂ is H, m is 1, and R₃ is 4-hydroxy.

In a further embodiment, a sirtuin-activating compound is a compound of formula 61 and the attendant definitions wherein n is 2, R is 3-hydroxy and 5-hydroxy, R₁ is H, R₂ is H, m is 1, and R₃ is 4-methoxy.

In another embodiment, a sirtuin-activating compound is a compound of formula 62: 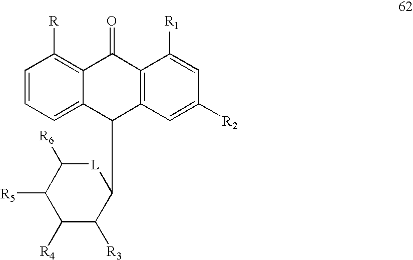
wherein, independently for each occurrence:

R, R₁, R₂, R₃, R₄, R₅, and R₆ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₇, or S; and

R₇ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 62 and the attendant definitions wherein R is OH.

In a further embodiment, a sirtuin-activating compound is a compound of formula 62 and the attendant definitions wherein R₁ is OH.

In a further embodiment, a sirtuin-activating compound is a compound of formula 62 and the attendant definitions wherein R₂ is CH₂OH.

In a further embodiment, a sirtuin-activating compound is a compound of formula 62 and the attendant definitions wherein R₃ is OH.

In a further embodiment, a sirtuin-activating compound is a compound of formula 62 and the attendant definitions wherein R₄ is OH.

In a further embodiment, a sirtuin-activating compound is a compound of formula 62 and the attendant definitions wherein R₅ is OH.

In a further embodiment, a sirtuin-activating compound is a compound of formula 62 and the attendant definitions wherein R₆ is CH₂OH.

In a further embodiment, a sirtuin-activating compound is a compound of formula 62 and the attendant definitions wherein L is O.

In a further embodiment, a sirtuin-activating compound is a compound of formula 62 and the attendant definitions wherein R is OH and R₁ is OH.

In a further embodiment, a sirtuin-activating compound is a compound of formula 62 and the attendant definitions wherein R is OH, R₁ is OH, and R₂ is CH₂OH.

In a further embodiment, a sirtuin-activating compound is a compound of formula 62 and the attendant definitions wherein R is OH, R₁ is OH, R₂ is CH₂OH, and R₃ is OH.

In a further embodiment, a sirtuin-activating compound is a compound of formula 62 and the attendant definitions wherein R is OH, R₁ is OH, R₂ is CH₂OH, R₃ is OH, and R₄ is OH.

In a further embodiment, a sirtuin-activating compound is a compound of formula 62 and the attendant definitions wherein R is OH, R₁ is OH, R₂ is CH₂OH, R₃ is OH, R₄ is OH, and R₅ is OH.

In a further embodiment, a sirtuin-activating compound is a compound of formula 62 and the attendant definitions wherein R is OH, R₁ is OH, R₂ is CH₂OH, R₃ is OH, R₄ is OH, R₅ is OH, and R₆ is CH₂OH.

In a further embodiment, a sirtuin-activating compound is a compound of formula 62 and the attendant definitions wherein R is OH, R₁ is OH, R₂ is CH₂OH, R₃ is OH, R₄ is OH, R₅ is OH, R₆ is CH₂OH, and L is O.

In another embodiment, a sirtuin-activating compound is a compound of formula 63: 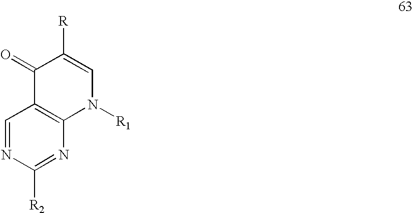
wherein, independently for each occurrence:

R, R₁, and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 63 and the attendant definitions wherein R is CO₂H.

In a further embodiment, a sirtuin-activating compound is a compound of formula 63 and the attendant definitions wherein R₁ is ethyl.

In a further embodiment, a sirtuin-activating compound is a compound of formula 63 and the attendant definitions wherein R₂ is N-1-pyrrolidine.

In a further embodiment, a sirtuin-activating compound is a compound of formula 63 and the attendant definitions wherein R is CO₂H and R₁ is ethyl.

In a further embodiment, a sirtuin-activating compound is a compound of formula 63 and the attendant definitions wherein R is CO₂H and R₂ is N-1-pyrrolidine.

In a further embodiment, a sirtuin-activating compound is a compound of formula 63 and the attendant definitions wherein R₁ is ethyl and R₂ is N-1-pyrrolidine.

In a further embodiment, a sirtuin-activating compound is a compound of formula 63 and the attendant definitions wherein R is CO₂H, R₁ is ethyl, and R₂ is N-1-pyrrolidine.

In another embodiment, a sirtuin-activating compound is a compound of formula 64: 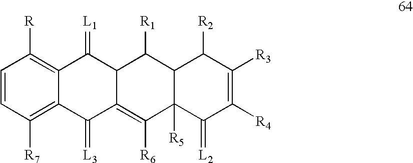
wherein, independently for each occurrence:

R, R₁, R₂, R₃, R₄, R₅, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are CH₂, O, NR₈, or S; and

R₈ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 64 and the attendant definitions wherein R is Cl.

In a further embodiment, a sirtuin-activating compound is a compound of formula 64 and the attendant definitions wherein R is H.

In a further embodiment, a sirtuin-activating compound is a compound of formula 64 and the attendant definitions wherein R₁ is OH.

In a further embodiment, a sirtuin-activating compound is a compound of formula 64 and the attendant definitions wherein R₂ is N(Me)₂.

In a further embodiment, a sirtuin-activating compound is a compound of formula 64 and the attendant definitions wherein R₃ is OH.

In a further embodiment, a sirtuin-activating compound is a compound of formula 64 and the attendant definitions wherein R₄ is C(O)NH₂.

In a further embodiment, a sirtuin-activating compound is a compound of formula 64 and the attendant definitions wherein R₅ is OH.

In a further embodiment, a sirtuin-activating compound is a compound of formula 64 and the attendant definitions wherein R₆ is OH.

In a further embodiment a sirtuin-activating compound is a compound of formula 64 and the attendant definitions wherein R₇ is OH.

In a further embodiment, a sirtuin-activating compound is a compound of formula 64 and the attendant definitions wherein L₁ is CH₂.

In a further embodiment, a sirtuin-activating compound is a compound of formula 64 and the attendant definitions wherein L₂ is O.

In a further embodiment, a sirtuin-activating compound is a compound of formula 64 and the attendant definitions wherein L₃ is O.

In a further embodiment, a sirtuin-activating compound is a compound of formula 64 and the attendant definitions wherein R is Cl and R₁ is OH.

In a further embodiment, a sirtuin-activating compound is a compound of formula 64 and the attendant definitions wherein R is Cl, R₁ is OH, and R₂ is N(Me)₂.

In a further embodiment, a sirtuin-activating compound is a compound of formula 64 and the attendant definitions wherein R is Cl, R₁ is OH, R₂ is N(Me)₂, and R₃ is OH.

In a further embodiment, a sirtuin-activating compound is a compound of formula 64 and the attendant definitions wherein R is Cl, R₁ is OH, R₂ is N(Me)₂, R₃ is OH, and R₄ is C(O)NH₂.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, and R₅ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, and R₆ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, and R₇ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, 6 is OH, R₇ is OH, and L₁ is CH₂.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L1 is CH₂, and L₂ is O.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L1 is CH₂, L₂ is O, and L₃ is O.

In a further embodiment, a sirtuin-activating compound is a compound of formula 64 and the attendant definitions wherein R is H and R₁ is OH.

In a further embodiment, a sirtuin-activating compound is a compound of formula 64 and the attendant definitions wherein R is H, R₁ is OH, and R₂ is N(Me)₂.

In a further embodiment, a sirtuin-activating compound is a compound of formula 64 and the attendant definitions wherein R is H, R₁ is OH, R₂ is N(Me)₂, and R₃ is OH.

In a further embodiment, a sirtuin-activating compound is a compound of formula 64 and the attendant definitions wherein R is H, R₁ is OH, R₂ is N(Me)₂, R₃ is OH, and R₄ is C(O)NH₂.

In a further embodiment, a sirtuin-activating compound is a compound of formula 64 and the attendant definitions wherein R is H, R₁ is OH, R₂ is N(Me)₂, R₃ is OH, R₄ is C(O)NH₂, and R₅ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, and R₆ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, and R₇ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In another embodiment, a sirtuin-activating compound is a compound of formula 65: 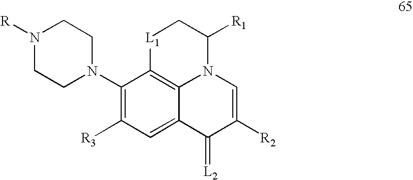
wherein, independently for each occurrence:

R is H or a substituted or unsubstituted alkyl, aryl, aralkyl, heterocyclyl, heterocyclylalkyl, heteroaryl, or heteroaralkyl;

R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin-activating compound is a compound of formula 65 and the attendant definitions wherein R is methyl.

In a further embodiment, a sirtuin-activating compound is a compound of formula 65 and the attendant definitions wherein R₁ is methyl.

In a further embodiment, a sirtuin-activating compound is a compound of formula 65 and the attendant definitions wherein R₂ is CO₂H.

In a further embodiment, a sirtuin-activating compound is a compound of formula 65 and the attendant definitions wherein R₃ is F.

In a further embodiment, a sirtuin-activating compound is a compound of formula 65 and the attendant definitions wherein L₁ is O.

In a further embodiment, a sirtuin-activating compound is a compound of formula 65 and the attendant definitions wherein L₂ is O.

In a further embodiment, a sirtuin-activating compound is a compound of formula 65 and the attendant definitions wherein R is methyl and R₁ is methyl.

In a further embodiment, a sirtuin-activating compound is a compound of formula 65 and the attendant definitions wherein R is methyl, R₁ is methyl, and R₂ is CO₂H.

In a further embodiment, a sirtuin-activating compound is a compound of formula 65 and the attendant definitions wherein R is methyl, R₁ is methyl, R₂ is CO₂H, and R₃ is F.

In a further embodiment, a sirtuin-activating compound is a compound of formula 65 and the attendant definitions wherein R is methyl, R₁ is methyl, R₂ is CO₂H, R₃ is F, and L₁ is O.

In a further embodiment, a sirtuin-activating compound is a compound of formula 65 and the attendant definitions wherein R is methyl, R₁ is methyl, R₂ is CO₂H, R₃ is F, L₁ is O, and L₂ is O.

Exemplary sirtuin-activating compounds are those listed in the appended Tables having a ratio to control rate of more than one. A preferred compound of formula 8 is Dipyridamole; a preferred compound of formula 12 is Hinokitiol; a preferred compound of formula 13 is L-(+)-Ergothioneine; a preferred compound of formula 19 is Caffeic Acid Phenol Ester; a preferred compound of formula 20 is MCI-186 and a preferred compound of formula 21 is HBED (Supplementary Table 6). Sirtuin-activating compounds may also be oxidized forms of the compounds of Table 21.

Also included are pharmaceutically acceptable addition salts and complexes of the sirtuin-activating compounds of formulas 1-25, 30, 32-65, and 69-76. In cases wherein the compounds may have one or more chiral centers, unless specified, the compounds contemplated herein may be a single stereoisomer or racemic mixtures of stereoisomers.

In one embodiment, a sirtuin-activating compound is a stilbene, chalcone, or flavone compound represented by formula 7:

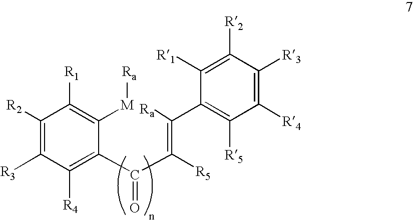

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, or aryl; and

n is 0 or 1.

In a further embodiment, a sirtuin-activating compound is a compound represented by formula 7 and the attendant definitions, wherein n is 0. In a further embodiment, a sirtuin-activating compound is a compound represented by formula 7 and the attendant definitions, wherein n is 1. In a further embodiment, a sirtuin-activating compound is a compound represented by formula 7 and the attendant definitions, wherein M is absent. In a further embodiment, a sirtuin-activating compound is a compound represented by formula 7 and the attendant definitions, wherein M is O. In a further embodiment, a sirtuin-activating compound is a compound represented by formula 7 and the attendant definitions, wherein Rₐ is H. In a further embodiment, a sirtuin-activating compound is a compound represented by formula 7 and the attendant definitions, wherein M is O and the two Rₐ form a bond. In a further embodiment, a sirtuin-activating compound is a compound represented by formula 7 and the attendant definitions, wherein R₅ is H. In a further embodiment, a sirtuin-activating compound is a compound represented by formula 7 and the attendant definitions, wherein R₅ is OH. In a further embodiment, a sirtuin-activating compound is a compound represented by formula 7 and the attendant definitions, wherein R₁, R₃, and R′₃ are OH. In a further embodiment, a sirtuin-activating compound is a compound represented by formula 7 and the attendant definitions, wherein R₂, R₄, R′₂, and R′₃ are OH. In a further embodiment, a sirtuin-activating compound is a compound represented by formula 7 and the attendant definitions, wherein R₂, R′₂, and R′₃ are OH.

In a further embodiment, a sirtuin-activating compound is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin-activating compound is a compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin-activating compound is a compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

In another embodiment, exemplary sirtuin-activating compounds are isonicotinamide analogs, such as, for example, the isonicotinamide analogs described in U.S. Pat. Nos. 5,985,848; 6,066,722; 6,228,847; 6,492,347; 6,803,455; and U.S. Patent Publication Nos. 2001/0019823; 2002/0061898; 2002/0132783; 2003/0149261; 2003/0229033; 2003/0096830; 2004/0053944; 2004/0110772; and 2004/0181063, the disclosures of which are hereby incorporated by reference in their entirety. In an exemplary emobidment, sirtuin-activating compounds may be an isonicotinamide analog having any of formulas 69-72 below. In one embodiment, a sirtuin-activating compound is an isonicotinamide analog compound of formula 69:

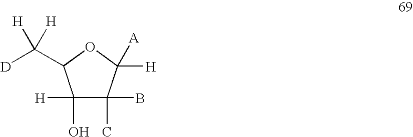

Wherein A is a nitrogen-, oxygen-, or sulfur-linked aryl, alkyl, cyclic, or heterocyclic group. The A moieties thus described, optionally have leaving group characteristics. In embodiments encompassed herein, A is further substituted with an electron contributing moiety. B and C are both hydrogen, or one of B or C is a halogen, amino, or thiol group and the other of B or C is hydrogen; and D is a primary alcohol, a hydrogen, or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge.

In one example, A is a substituted N-linked aryl or heterocyclic group, an O-linked aryl or heterocyclic group having the formula —O—Y, or an S-linked aryl or heterocyclic group having the formula —O—Y; both B and C are hydrogen, or one of B or C is a halogen, amino, or thiol group and the other of B or C is hydrogen; and D is a primary alcohol or hydrogen. Nonlimiting preferred examples of A are set forth below, where each R is H or an electron-contributing moiety and Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl.

Examples of A include i-xiv below: 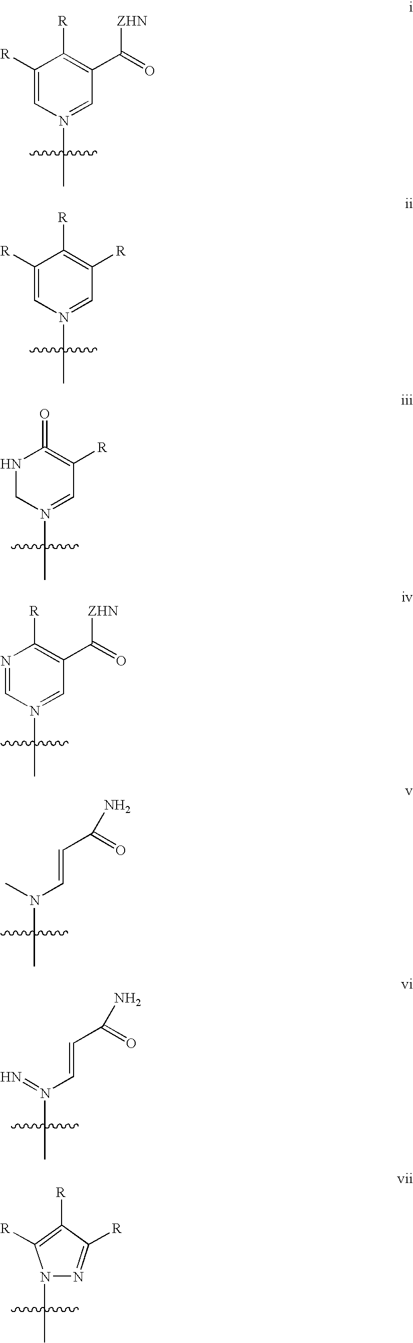 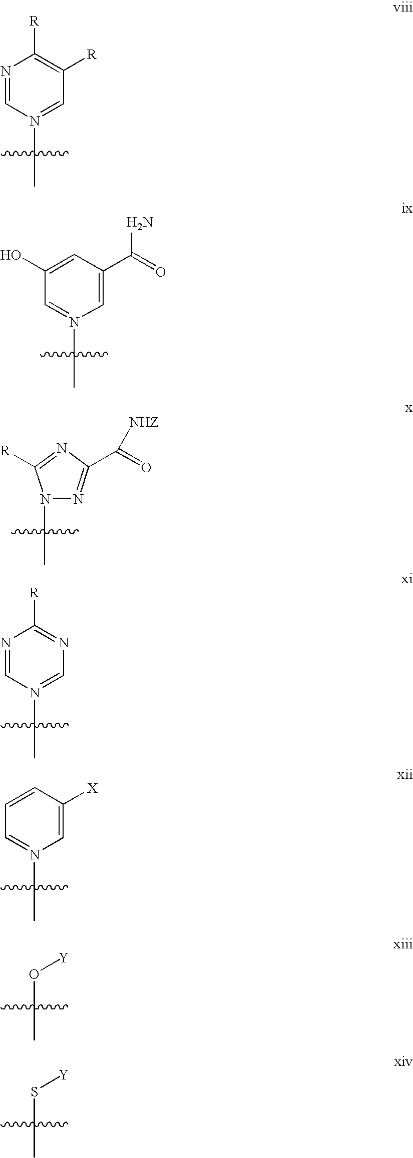
where Y=a group consistent with leaving group function.

Examples of Y include, but are not limited to, xv-xxvii below:

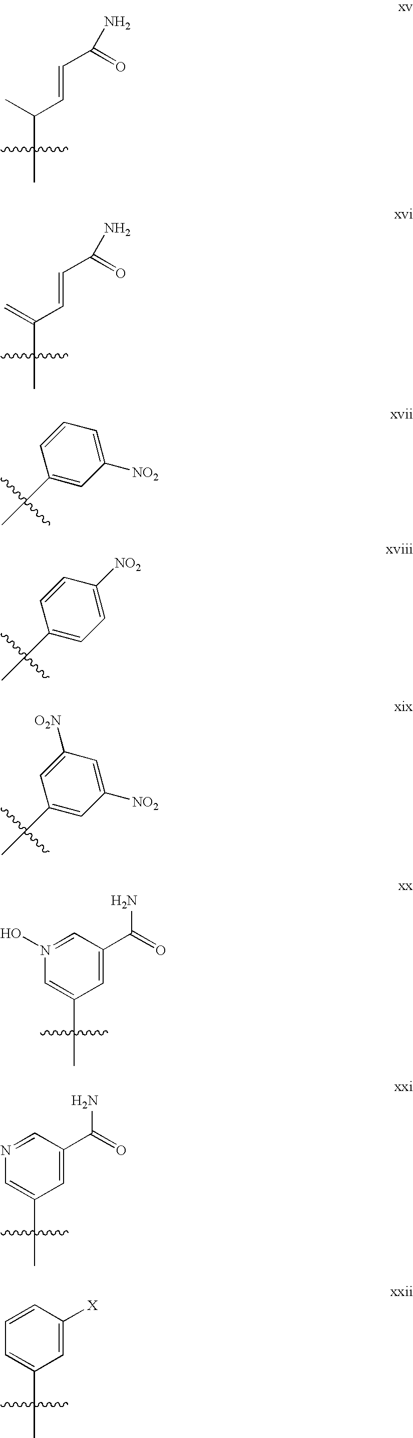

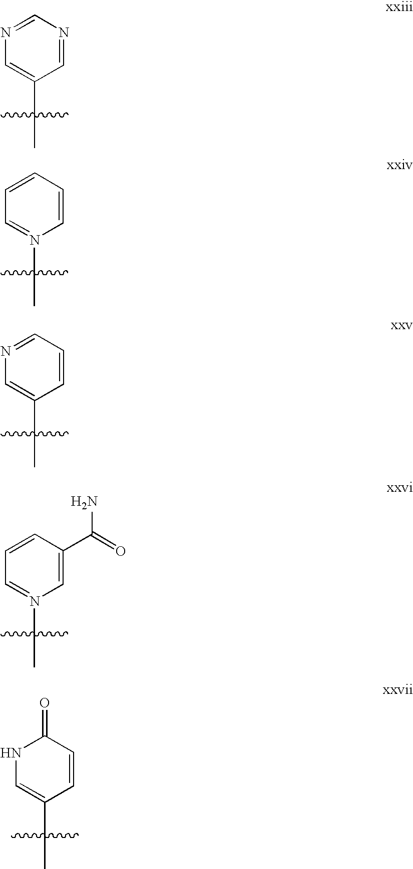

Wherein, for i-xxvii, X is halogen, thiol, or substituted thiol, amino or substituted amino, oxygen or substituted oxygen, or aryl or alkyl groups or heterocycles.

In certain embodiments, A is a substituted nicotinamide group (i above, where Z is H), a substituted pyrazolo group (vii above), or a substituted 3-carboxamid-imidazolo group (x above, where Z is H). Additionally, both B and C may be hydrogen, or one of B or C is a halogen, amino, or thiol group and the other of B or C is hydrogen; and D is a primary alcohol or hydrogen.

In other embodiments, one of B or C may be halogen, amino, or thiol group when the other of B or C is a hydrogen. Furthermore, D may be a hydrogen or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge. Analogues of adenosine monophosphate or adenosine diphosphate also can replace the adenosine monophosphate or adenosine diphosphate groups.

In some embodiments, A has two or more electron contributing moieties.

In other embodiments, a sirtuin-activating compound is an isonicotinamide analog compound of formulas 70, 71, or 72 below. 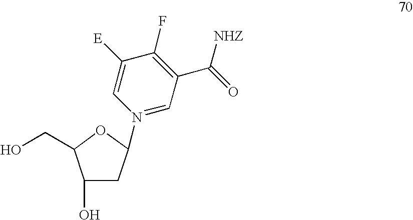
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; E and F are independently H, CH₃, OCH.sub.3, CH₂CH₃, NH₂, OH, NHCOH, NHCOCH₃, N(CH₃)₂, C(CH₃)₂, an aryl or a C₃-C₁₀ alkyl, preferably provided that, when one of of E or F is H, the other of E or F is not H; 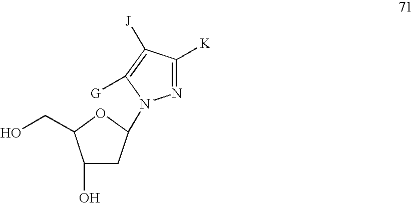
wherein G, J or K is CONHZ, Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl, and the other two of G, J and K is independently CH₃, OCH₃, CH₂CH₃, NH₂, OH, NHCOH, NHCOCH₃; 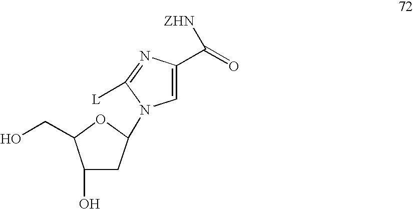
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; and L is CH₃, OCH₃, CH₂CH₃, NH₂, OH, NHCOH, NHCOCH₃.

In an exemplary embodiment, the compound is formula 70 above, wherein E and F are independently H, CH₃, OCH₃, or OH, preferably provided that, when one of E or F is H, the other of E or F is not H.

In another exemplary embodiment, the compound is β-1′-5-methyl-nicotinamide-2′-deoxyribose, β-D-1′-5-methyl-nico-tinamide-2′-deoxyribofuranoside, β-1′-4,5-dimethyl-nicotinamide-2′-de-oxyribose or β-D-1′-4,5-dimethyl-nicotinamide-2′-deoxyribofuranoside.

In yet another embodiment, the compound is β-1′-5-methyl-nicotinamide-2′-deoxyribose.

Without being bound to any particular mechanism, it is believed that the electron-contributing moiety on A stabilizes the compounds of the invention such that they are less susceptible to hydrolysis from the rest of the compound. This improved chemical stability improves the value of the compound, since it is available for action for longer periods of time in biological systems due to resistance to hydrolytic breakdown. The skilled artisan could envision many electron-contributing moieties that would be expected to serve this stabilizing function. Non-limiting examples of suitable electron contributing moieties are methyl, ethyl, O-methyl, amino, NMe₂, hydroxyl, CMe₃, aryl and alkyl groups. Preferably, the electron-contributing moiety is a methyl, ethyl, O-methyl, amino group. In the most preferred embodiments, the electron-contributing moiety is a methyl group.

The compounds of formulas 69-72 are useful both in free form and in the form of salts. The term “pharmaceutically acceptable salts” is intended to apply to non-toxic salts derived from inorganic or organic acids and includes, for example, salts derived from the following acids: hydrochloric, sulfuric, phosphoric, acetic, lactic, fumaric, succinic, tartaric, gluconic, citric, methanesulfonic, and p-toluenesulfonic acids.

Also provided are compounds of formulas 69-72 that are the tautomers, pharmaceutically-acceptable salts, esters, and pro-drugs of the inhibitor compounds disclosed herein.

The biological availability of the compounds of formulas 69-72 can be enhanced by conversion into a pro-drug form. Such a pro-drug can have improved lipophilicity relative to the unconverted compound, and this can result in enhanced membrane permeability. One particularly useful form of pro-drug is an ester derivative. Its utility relies upon the action of one or more of the ubiquitous intracellular lipases to catalyse the hydrolysis of ester groups, to release the active compound at or near its site of action. In one form of pro-drug, one or more hydroxy groups in the compound can be O-acylated, to make an acylate derivative.

Pro-drug forms of a 5-phosphate ester derivative of compounds of formulas 69-72 can also be made. These may be particularly useful, since the anionic nature of the 5-phosphate may limit its ability to cross cellular membranes. Conveniently, such a 5-phosphate derivative can be converted to an uncharged bis(acyloxymethyl)ester derivative. The utility of such a pro-drug relies upon the action of one or more of the ubiquitous intracellular lipases to catalyse the hydrolysis of ester groups, releasing a molecule of formaldehyde and a compound of the present invention at or near its site of action. Specific examples of the utility of, and general methods for making, such acyloxymethyl ester pro-drug forms of phosphorylated carbohydrate derivatives have been described (Kang et al., 1998; Jiang et al., 1998; L1 et al., 1997; Kruppa et al., 1997).

In another embodiment, exemplary sirtuin-activating compounds are O-acetyl-ADP-ribose analogs, including 2′-O-acetyl-ADP-ribose and 3′-O-acetyl-ADP-ribose, and analogs thereof. Exemplary O-acetyl-ADP-ribose analogs are described, for example, in U.S. Patent Publication Nos. 2004/0053944; 2002/0061898; and 2003/0149261, the disclosures of which are hereby incorporated by reference in their entirety. In an exemplary emobidment, sirtuin-activating compounds may be an O-acetyl-ADP-ribose analog having any of formulas 73-76 below. In one embodiment, a sirtuin-activating compound is an O-acetyl-ADP-ribose analog compound of formula 73: 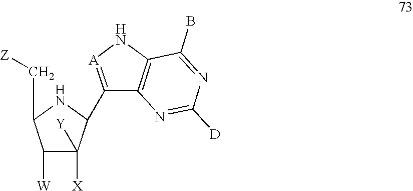
wherein:

A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups;

B is selected from OH, NH₂, NHR⁴, H and halogen, where R⁴ is an optionally substituted alkyl, aralkyl or aryl group;

D is selected from OH, NH₂, NHR⁵, H, halogen and SCH₃, where R⁵ is an optionally substituted alkyl, aralkyl or aryl group;

X and Y are independently selected from H, OH and halogen, with the proviso that when one of X and Y is hydroxy or halogen, the other is hydrogen;

Z is OH, or, when X is hydroxy, Z is selected from hydrogen, halogen, hydroxy, SQ and OQ, where Q is an optionally substituted alkyl, aralkyl or aryl group; and

W is OH or H, with the proviso that when W is OH, then A is CR where R is as defined above;

or a tautomer thereof; or a pharmaceutically acceptable salt thereof; or an ester thereof; or a prodrug thereof.

In certain embodiments, when B is NHR⁴ and/or D is NHR⁵, then R⁴ and/or R⁵ are C1-C4 alkyl.

In other embodiments, when one or more halogens are present they are chosen from chlorine and fluorine.

In another embodiment, when Z is SQ or OQ, Q is C1-C5 alkyl or phenyl.

In an exemplary embodiment, D is H, or when D is other than H, B is OH.

In another embodiment, B is OH, D is H, OH or NH₂, X is OH or H, Y is H, most preferably with Z as OH, H, or methylthio, especially OH.

In certain embodiments W is OH, Y is H, X is OH, and A is CR where R is methyl or halogen, preferably fluorine.

In other embodiments, W is H, Y is H, X is OH and A is CH.

In other embodiments, a sirtuin-activating compound is an O-acetyl-ADP-ribose analog compound of formula 74:

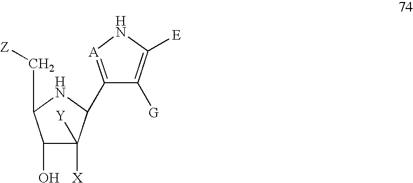

wherein A, X, Y, Z and R are defined for compounds of formula (73) where first shown above; E is chosen from CO₂H or a corresponding salt form, CO₂R, CN, CONH₂, CONHR or CONR₂; and G is chosen from NH₂, NHCOR, NHCONHR or NHCSNHR; or a tautomer thereof, or a pharmaceutically acceptable salt thereof, or an ester thereof, or a prodrug thereof.

In certain embodiments, E is CONH₂ and G is NH₂.

In other embodiments, E is CONH₂, G is NH₂, X is OH or H, is H, most preferable with Z as OH, H or methylthio, especially OH.

Exemplary sirtuin-activating compounds include the following:
(1S)-1,4-dideoxy-1-C-(4-hydroxypyrrolo[3,2-d]pyrimidin-7-yl)-1,4-imino-D-ribitol
(1S)-1-C-(2-amino-4-hydroxypyrrolo[3,2-d]pyrimidin-7-yl)-1,4-dideoxy-1,4-imino-D-ribitol
(1R)-1-C-(4-hydroxypyrrolo[3,2-d]pyrimidin-7-yl)-1,4-imino-1,2,4-trideoxy-D-erythro-pentitol
(1S)-1-C-(4-hydroxypyrrolo[3,2-d]pyrimidin-7-yl)-1,4-imino-1,4,5-trideoxy-D-ribitol
(1S)-1,4-dideoxy-1-C-(4-hydroxypyrrolo[3,2-d]pyrimidin-7-yl)-1,4-imino-5-methylthio-D-ribitol
(1S)-1,4-dideoxy-1-C-(2,4-dihydroxypyrrolo[3,2-d]pyrimidin-7-yl)-1,4-imino-D-ribitol
(1R)-1-C-(2,4-dihydroxypyrrolo[3,2-d]pyrimidin-7-yl)-1,4-imino-1,2,4-trideoxy-D-erthro-pentitol
(1S)-1-C-(2,4-dihydroxypyrrolo[3,2-d]pyrimidin-7-yl)-1,4-imino-1,4,5-trideoxy-D-ribitol
(1S)-1,4-dideoxy-1-C-(2,4-dihydroxypyrrolo[3,2-d]pyrimidin-7-yl)-1,4-imino-5-ethylthio-D-ribitol
(1R)-1-C-(2-amino-4-hydroxypyrrolo[3,2-d]pyrimidin-7-yl)-1,4-imino-1,2,4-trideoxy-D-erythro-pentitol
(1S)-1-C-(2-amino-4-hydroxypyrrolo[3,2-d]pyrimidin-7-yl)-1,4-imino-1,4,5-trideoxy-D-ribitol
(1S)-1-C-(2-amino-4-hydroxypyrrolo[3,2-d]pyrimidin-7-yl)-1,4-dideoxy-1,4-imino-5-methylthio-D-ribitol
(1S)-1,4-dideoxy-1-C-(7-hydroxypyrazolo[4,3-d]pyrimidin-3-yl)-1,4-imino-D-ribitol
(1R)-1-C-(7-hydroxypyrazolo[4,3-d]pyrimidin-3-yl)-1,4-imino-1,2,4-trideoxy-D-erythro-pentitol
(1S)-1-C-(7-hydroxypyrazolo[4,3-d]pyrimidin-3-yl)-1,4-imino-1,4,5-trideoxy-D-ribitol
(1S)-1,4-dideoxy-1-C-(7-hydroxypyrazolo[4,3-d]pyrimidin-3-yl)-1,4-imino-5-ethylthio-D-ribitol
(1S)-1,4-dideoxy-1-C-(5,7-dihydroxypyrazolo[4,3-d]pyrimidin-3-yl)-1,4-imino-D-ribitol
(1R)-1-C-(5,7-dihydroxypyrazolo[4,3-d]pyrimidin-3-yl)-1,4-imino-1,2,4-trideoxy-D-erythro-pentitol
(1S)-1-C-(5,7-dihydroxypyrazolo[4,3-d]pyrimidin-3-yl)-1,4-imino-1,4,5-trideoxy-D-ribitol
(1S)-1,4-dideoxy-1-C-(5,7-dihydroxypyrazolo[4,3-d]pyrimidin-3-yl)-1,4-imino-5-methylthio-D-ribitol
(1S)-1-C-(5-amino-7-hydroxypyrazolo[4,3-d]pyrimidin-3-yl)-1,4-dideoxy-1,4-imino-D-ribitol
(1R)-1-C-(S-amino-7-hydroxypyrazolo[4,3-d]pyrimidin-3-yl)-1,4-imino-1,2,4-trideoxy-D-erythro-pentitol
(1S)-1-C-(5-amino-7-hydroxypyrazolo[4,3-d]pyrimidin-3-yl)-1,4-imino-1,4,5-trideoxy-D-ribitol
(1S)-1-C-(5-amino-7-hydroxypyrazolo[4,3-d]pyrimidin-3-yl)-1,4-dideoxy-1,4-imino-5-methylthio-D-ribitol
(1S)-1-C-(3-amino-2-carboxamido-4-pyrroly)-1,4-dideoxy-1,4-imino-D-ribitol.
(1S)-1,4-dideoxy-1-C-(4-hydroxypyrrolo[3,2-d]pyrimidin-7-yl)-1,4-imino-D-ribitol 5-phosphate
(1S)-1-C-(2-amino-4-hydroxypyrrolo[3,2-d]pyrimidin-7-yl)-1,4-imino-D-ribitol 5-phosphate
(1S)-1-C-(3-amino-2-carboxamido-4-pyrrolyl)-1,4-dideoxy-1,4-imino-D-ribitol

In yet other embodiments, sirtuin-activating compounds are O-acetyl-ADP-ribose analog compounds of formula 75 and 76, their tautomers and pharmaceutically acceptable salts.

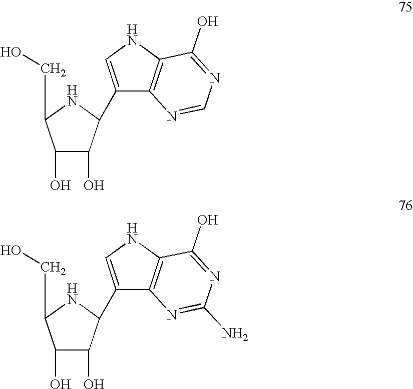

The biological availability of a compound of formula (75) or formula (76) can be enhanced by conversion into a pro-drug form. Such a pro-drug can have improved lipophilicity relative to the compound of formula (75) or formula (76), and this can result in enhanced membrane permeability. One particularly useful form of a pro-drug is an ester derivative. Its utility relies upon the action of one or more of the ubiquitous intracellular lipases to catalyse the hydrolysis of these ester group(s), to release the compound of formula (75) and formula (76) at or near its site of action.

In one form of a prodrug, one or more of the hydroxy groups in a compound of formula (75) or formula (76) can be O-acylated, to make, for example a 5-O-butyrate or a 2,3-di-O-butyrate derivative.

Prodrug forms of 5-phosphate ester derivative of a compounds of formula (75) or formula (76) can also be made and may be particularly useful, since the anionic nature of the 5-phosphate may limit its ability to cross cellular membranes. Conveniently, such a 5-phosphate derivative can be converted to an uncharged bis(acyloxymethyl)ester derivative. The utility of such a pro-drug relies upon the action of one or more of the ubiquitous intracellular lipases to catalyse the hydrolysis of these ester group(s), releasing a molecule of formaldehyde and the compound of formula (75) or formula (76) at or near its site of action.

In an exemplary embodiment, analogs of 2′-AADPR or 3′-AADPR that are designed to have increased stability from esterase action through the use of well-known substitutes for ester oxygen atoms that are subject to esterase attack. The esterase-labile oxygen atoms in 2′-AADPR and 3′-AADPR would be understood to be the ester oxygen linking the acetate group with the ribose, and the ester oxygen between the two phosphorus atoms. As is known in the art, substitution of either or both of these ester oxygen atoms with a CF₂, a NH, or a S would be expected to provide a 2′-AADPR or 3′-AADPR analog that is substantially more stable due to increased resistance to esterase action.

Thus, in some embodiments, the invention is directed to analogs 2′-O-acetyl-ADP-ribose or 3′-O-acetyl-ADP-ribose exhibiting increased stability in cells. The preferred analogs comprise a CF₂, a NH, or a S instead of the acetyl ester oxygen or the oxygen between two phosphorus atoms. The most preferred substitute is CF₂. Replacement of the acetyl ester oxygen is particularly preferred. In other preferred embodiments, both the ester oxygen and the oxygen between the two phosphorus atoms are independently substituted with a CF₂, a NH, or a S.

Also included are pharmaceutically acceptable addition salts and complexes of the sirtuin-activity compounds described herein. In cases wherein the compounds may have one or more chiral centers, unless specified, the compounds contemplated herein may be a single stereoisomer or racemic mixtures of stereoisomers.

In cases in which the sirtuin-activating compounds have unsaturated carbon-carbon double bonds, both the cis (Z) and trans (E) isomers are contemplated herein. In cases wherein the compounds may exist in tautomeric forms, such as keto-enol tautomers, such as 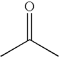
and 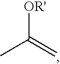
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the sirtuin-activating compounds described herein. Prodrugs are considered to be any covalently bonded carriers that release the active parent drug in vivo.

Analogs and derivatives of the sirtuin-activating compounds described herein can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

In certain embodiments, if a sirtuin-activating compound occurs naturally, it may be at least partially isolated from its natural environment prior to use. For example, a plant polyphenol may be isolated from a plant and partially or significantly purified prior to use in the methods described herein. An activating compound may also be prepared synthetically, in which case it would be free of other compounds with which it is naturally associated. In an illustrative embodiment, an activating composition comprises, or an activating compound is associated with, less than about 50%, 10%, 1%, 0.1%, 10⁻²% or 10⁻³% of a compound with which it is naturally associated.

In certain embodiments, a certain biological function (e.g., reducing flushing and/or weight gain) is modulated by a sirtuin-activating compound with the proviso that the term sirtuin-activating compound does not include one or more specific compounds. For example, in certain embodiments, a sirtuin-activating compound may be any compound that is capable of increasing the level of expression and/or activity of a sirtuin protein with the proviso that the compound is not resveratrol, flavone, or any other compound specifically cited herein. In an exemplary embodiment, a sirtuin-activating compound may be a compound of any one of formulas 1-25, 30, 32-65, and 69-76 with the proviso that the compound is not resveratrol, flavone, or any other compound specifically cited herein. In certain embodiments, a sirtuin-activating compound does not include a compound of any one of formulas 69-72, any one of formulas 73-76, or any one of formulas 69-76.

In certain embodiments, a sirtuin-activaing compound does not have any substantial ability to inhibit P13-kinase, inhibit aldoreductase and/or inhibit tyrosine protein kinases at concentrations (e.g., in vivo) effective for activating the deacetylase activity of the sirtuin, e.g., SIRT1. For instance, in preferred embodiments the sirtuin-activaing compound is chosen to have an EC₅₀ for activating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less. Methods for assaying PI3-Kinase activity, aldose reductase activity, and tyrosine kinase activity are well known in the art and kits to perform such assays may be purchased commercially. See e.g., U.S. Patent Publication No. 2003/0158212 for PI3-kinase assays; U.S. Patent Publication No. 2002/20143017 for aldose reductase assays; tyrosine kinase assay kits may be purchased commercially, for example, from Promega (Madison, Wis.; world wide web at promega.com), Invitrogen (Carlsbad, Calif.; world wide web at invitrogen.com) or Molecular Devices (Sunnyvale, Calif.; world wide web at moleculardevices.com).

In certain embodiments, a sirtuin-activaing compound does not have any substantial ability to transactivate EGFR tyrosine kinase activity at concentrations (e.g., in vivo) effective for activating the deacetylase activity of the sirtuin. For instance, in preferred embodiments the sirtuin-activaing compound is chosen to have an EC₅₀ for activating sirtuin deacetylase activity that is at least 5 fold less than the EC₅₀ for transactivating EGFR tyrosine kinase activity, and even more preferably at least 10 fold, 100 fold or even 1000 fold less. Methods for assaying transactivation of EGFR tyrosine kinase activity are well known in the art, see e.g., Pai et al. Nat. Med. 8: 289-93 (2002) and Vacca et al. Cancer Research 60: 5310-5317 (2000).

In certain embodiments, a sirtuin-activaing compound does not have any substantial ability to cause coronary dilation at concentrations (e.g., in vivo) effective for activating the deacetylase activity of the sirtuin. For instance, in preferred embodiments the sirtuin-activaing compound is chosen to have an EC₅₀ for activating sirtuin deacetylase activity that is at least 5 fold less than the EC₅₀ for coronary dilation, and even more preferably at least 10 fold, 100 fold or even 1000 fold less. Methods for assaying vasodilation are well known in the art, see e.g., U.S. Patent Publication No. 2004/0236153.

In certain embodiments, a sirtuin-activaing compound does not have any substantial spasmolytic activity at concentrations (e.g., in vivo) effective for activating the deacetylase activity of the sirtuin. For instance, in preferred embodiments the sirtuin-activaing compound is chosen to have an EC₅₀ for activating sirtuin deacetylase activity that is at least 5 fold less than the EC₅₀ for spasmolytic effects (such as on gastrointestinal muscle), and even more preferably at least 10 fold, 100 fold or even 1000 fold less. Methods for assaying spasmolytic activity are well known in the art, see e.g., U.S. Patent Publication No. 2004/0248987.

In certain embodiments, the subject sirtuin activators do not have any substantial ability to inhibit hepatic cytochrome P450 1B1 (CYP) at concentrations (e.g., in vivo) effective for activating the deacetylase activity of the sirtuin. For instance, in preferred embodiments the sirtuin-activaing compound is chosen to have an EC₅₀ for activating sirtuin deacetylase activity that is at least 5 fold less than the EC₅₀ for inhibition of P450 1B 1, and even more preferably at least 10 fold, 100 fold or even 1000 fold less. Methods for assaying cytochrome P450 activity are well known in the art and kits to perform such assays may be purchased commercially. See e.g., U.S. Pat. Nos. 6,420,131 and 6,335,428 and Promega (Madison, Wis.; world wide web at promega.com).

In certain embodiments, a sirtuin-activaing compound does not have any substantial ability to inhibit nuclear factor-kappaB (NF-κB) at concentrations (e.g., in vivo) effective for activating the deacetylase activity of the sirtuin. For instance, in preferred embodiments the sirtuin-activaing compound is chosen to have an EC₅₀ for activating sirtuin deacetylase activity that is at least 5 fold less than the EC₅₀ for inhibition of NF-κB, and even more preferably at least 10 fold, 100 fold or even 1000 fold less. Methods for assaying NF-κB activity are well known in the art and kits to perform such assays may be purchased commercially (e.g., from Oxford Biomedical Research (Ann Arbor, Mich.; world wide web at oxfordbiomed.com)).

In certain embodiments, a sirtuin-activating compound does not have any substantial ability to inhibit a histone deacetylase (HDACs) class I, a HDAC class II, or HDACs I and II, at concentrations (e.g., in vivo) effective for activating the deacetylase activity of the sirtuin. For instance, in preferred embodiments the sirtuin-activaing compound is chosen to have an EC₅₀ for activating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, a sirtuin-activaing compound does not have any substantial ability to activate SIRT1 orthologs in lower eukaryotes, particularly yeast or human pathogens, at concentrations (e.g., in vivo) effective for activating the deacetylase activity of human SIRT1. For instance, in preferred embodiments the sirtuin-activaing compound is chosen to have an EC₅₀ for activating human SIRT1 deacetylase activity that is at least 5 fold less than the EC₅₀ for activating yeast Sir2 (such as Candida, S. cerevisiae, etc), and even more preferably at least 10 fold, 100 fold or even 1000 fold less.

In certain embodiments, the sirtuin-activating compounds may have the ability to activate one or more sirtuin protein homologs, such as, for example, one or more of human SIRT1, SIRT2, SIRT3, SIRT4, SIRT5, SIRT6, or SIRT7. In other embodiments, a sirtuin-activaing compound does not have any substantial ability to activate other sirtuin protein homologs, such as, for example, one or more of human SIRT2, SIRT3, SIRT4, SIRT5, SIRT6, or SIRT7, at concentrations (e.g., in vivo) effective for activating the deacetylase activity of human SIRT1. For instance, the sirtuin-activaing compound may be chosen to have an EC₅₀ for activating human SIRT1 deacetylase activity that is at least 5 fold less than the EC₅₀ for activating one or more of human SIRT2, SIRT3, SIRT4, SIRT5, SIRT6, or SIRT7, and even more preferably at least 10 fold, 100 fold or even 1000 fold less.

In other embodiments, a sirtuin-activating compound does not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin-activaing compound is chosen to have an EC₅₀ for activating sirtuin deacetylase activity that is at least 5 fold less than the EC₅₀ for performing any of these activities, and even more preferably at least 10 fold, 100 fold or even 1000 fold less. Methods for assaying protein kinase activity, cyclo-oxygenase activity, nitric oxide synthase activity, and platelet adhesion activity are well known in the art and kits to perform such assays may be purchased commercially. See e.g., Promega (Madison, Wis.; world wide web at promega.com), Invitrogen (Carlsbad, Calif.; world wide web at invitrogen.com); Molecular Devices (Sunnyvale, Calif.; world wide web at moleculardevices.com) or Assay Designs (Ann Arbor, Mich.; world wide web at assaydesigns.com) for protein kinase assay kits; Amersham Biosciences (Piscataway, N.J.; world wide web at amershambiosciences.com) for cyclo-oxygenase assay kits; Amersham Biosciences (Piscataway, N.J.; world wide web at amershambiosciences.com) and R&D Systems (Minneapolis, Minn.; world wide web at rndsystems.com) for nitric oxide synthase assay kits; and U.S. Pat. Nos. 5,321,010; 6,849,290; and 6,774,107 for platelet adhesion assays.

In certain embodiments, a sirtuin-activating compound described herein does not have significant or detectable anti-oxidant activities, as determined by any of the standard assays known in the art. For example, a sirtuin-activating compound does not significantly scavenge free-radicals, such as O₂ radicals. A sirtuin-activating compound may have less than about 2, 3, 5, 10, 30 or 100 fold anti-oxidant activity relative to another sirtuin-activating compound, e.g., resveratrol.

In certain embodiments, a sirtuin-activating compound may have a binding affinity for a sirtuin of about 10⁻⁹ M, 10⁻¹⁰ M, 10⁻¹¹ M, 10⁻¹² M or less. A sirtuin-activaing compound may reduce the Kₘ of a sirtuin for its substrate or NAD⁺ by a factor of at least about 2, 3, 4, 5, 10, 20, 30, 50 or 100. A sirtuin-activaing compound may increase the Vₘₐₓ of a sirtuin by a factor of at least about 2, 3, 4, 5, 10, 20, 30, 50 or 100. Exemplary sirtuin-activaing compounds that may increase the Vₘₐₓ of a sirtuin include, for example, analogs of isonicotinamide, such as, for example, compounds of formulas 69-72, and/or analogs of O-acetyl-ADP-ribose, such as, for example, compounds of formulas 73-76. A sirtuin-activaing compound may have an EC₅₀ for activating the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A sirtuin-activaing compound may activate the deacetylase activity of a sirtuin by a factor of at least about 5, 10, 20, 30, 50, or 100, as measured in an acellular assay or in a cell based assay as described in the Examples. A sirtuin-activaing compound may cause at least a 10%, 30%, 50%, 80%, 2 fold, 5 fold, 10 fold, 50 fold or 100 fold greater induction of the deacetylase activity of SIRT1 relative to the same concentration of resveratrol or other compound described herein. A sirtuin-activaing compound may also have an EC₅₀ for activating SIRT5 that is at least about 10 fold, 20 fold, 30 fold, 50 fold greater than that for activating SIRT1.

In an exemplary embodiment, the methods and compositions described herein may include a combination therapy comprising (i) at least one sirtuin-activating compound that reduce the Kₘ of a sirtuin for its substrate or NAD⁺ by a factor of at least about 2, 3, 4, 5, 10, 20, 30, 50 or 100, and (ii) at least one sirtuin-activating compound that increases the Vₘₐₓ of a sirtuin by a factor of at least about 2, 3, 4, 5, 10, 20, 30, 50 or 100. In one embodiment, a combination therapy may comprise (i) at least one sirtuin-activating compound of formula 1-25, 30, and 32-65, and (ii) at least one sirtuin-activating compound of formula 69-76.

A sirtuin-activaing compound may traverse the cytoplasmic membrane of a cell. For example, a sirtuin-activaing compound may have a cell-permeability of at least about 20%, 50%, 75%, 80%, 90% or 95%.

Sirtuin-activaing compounds described herein may also have one or more of the following characteristics: the compound may be essentially non-toxic to a cell or subject; the compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a compound may be more stable in solution than resveratrol by at least a factor of about 50%, 2 fold, 5 fold, 10 fold, 30 fold, 50 fold or 100 fold; a compound may promote deacetylation of the DNA repair factor Ku70; a compound may promote deacetylation of RelA/p65; a compound may increase general turnover rates and enhance the sensitivity of cells to TNF-induced apoptosis.

3. Exemplary Uses

In one aspect, a sirtuin-activating compound may be used to treat and/or prevent the incidence and/or severity of flushing (including warmth, redness, itching and/or tingling) and/or hot flashes which are symptoms of a disorder. For instance, the subject method includes the use of a sirtuin-activating compound, alone or in combination with other agents, for reducing the incidence and/or severity of flushing and/or hot flashes in cancer patients. In other embodiments, the method provides for the use of a sirtuin-activating compound to reduce the incidence and/or severity of flushing and/or hot flashes in menopausal and post-menopausal woman.

In another aspect, a sirtuin-activating compound may be used as a therapy for reducing the incidence or severity of flushing and/or hot flashes which are side-effects of another drug therapy, e.g., drug-induced flushing. In certain embodiments, a method for treating and/or preventing drug-induced flushing comprises administering to a patient in need thereof a formulation comprising at least one flushing inducing compound and at least one sirtuin activating compound. In other embodiments, a method for treating drug induced flushing comprises separately administering one or more compounds that induce flushing and one or more sirtuin-activating compounds, e.g., wherein the sirtuin-activating compound and flushing inducing agent have not been formlulated in the same compositions. When using separate formulations, the sirtuin-activating compound may be administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, a sirtuin-activating compound may be used to reduce flushing side effects of a vasodilator or an antilipemic agent (including anticholesteremic agents and lipotropic agents). In an exemplary embodiment, a sirtuin activating compound may be used to reduce flushing associated with the administration of niacin.

Nicotinic acid, 3-pyridinecarboxylic acid or niacin, is an antilipidemic agent that is marketed under, for example, the trade names Nicolar®, SloNiacin®, Nicobide and Time Release Niacin®. Nicotinic acid has been used for many years in the treatment of lipidemic disorders such as hyperlipidemia, hypercholesterolemia and atherosclerosis. This compound has long been known to exhibit the beneficial effects of reducing total cholesterol, low density lipoproteins or “LDL cholesterol,” triglycerides and apolipoprotein a (Lp(a)) in the human body, while increasing desirable high density lipoproteins or “HDL cholesterol”.

Typical doses range from about 1 gram to about 3 grams daily. Nicotinic acid is normally administered two to four times per day after meals, depending upon the dosage form selected. Nicotinic acid is currently commercially available in two dosage forms. One dosage form is an immediate or rapid release tablet which should be administered three or four times per day. Immediate release (“IR”) nicotinic acid formulations generally release nearly all of their nicotinic acid within about 30 to 60 minutes following ingestion. The other dosage form is a sustained release form which is suitable for administration two to four times per day. In contrast to IR formulations, sustained release (“SR”) nicotinic acid formulations are designed to release significant quantities of drug for absorption into the blood stream over specific timed intervals in order to maintain therapeutic levels of nicotinic acid over an extended period such as 12 or 24 hours after ingestion.

As used herein, the term “nicotinic acid” is meant to encompass nicotinic acid or a compound other than nicotinic acid itself which the body metabolizes into nicotinic acid, thus producing essentially the same effect as nicotinic acid. Exemplary compounds that produce an effect similar to that of nicotinic acid include, for example, nicotinyl alcohol tartrate, d-glucitol hexanicotinate, aluminum nicotinate, niceritrol and d,l-alpha-tocopheryl nicotinate. Each such compound will be collectively referred to herein as “nicotinic acid.”

In another embodiment, the invention provides a method for treating and/or preventing hyperlipidemia with reduced flushing side effects. The method comprises the steps of administering to a subject in need thereof a therapeutically effective amount of nicotinic acid and a sirtuin-activating compound in an amount sufficient to reduce flushing. In an exemplary embodiment, the nicotinic acid and/or sirtuin-activating compound may be administered nocturnally.

In an exemplary embodiment, a sirtuin-activating compound may be administered as part of a combination therapy with sustained release nicotinic acid, e.g., niacin. Examples of sustained release niacin include, for example, Niaspan® and Advicor® and those described in U.S. Pat. Nos. 6,746,691; 6,676,967; 6,818,229; and 6,406,715. In one embodiment the sirtuin-activating compound may be formulated with the nicotinic acid as part of the sustained release formulation. Alternatively, the sirtuin-activating compound may be administered separately from the nicotinic acid optionally as a sustained release formulation.

In another embodiment, a sirtuin-activating compound may be used to reduce flushing side effects of raloxifene. Raloxifene acts like estrogen in certain places in the body, but is not a hormone. It helps prevent osteoporosis in women who have reached menopause. Osteoporosis causes bones to gradually grow thin, fragile, and more likely to break. Evista slows down the loss of bone mass that occurs with menopause, lowering the risk of spine fractures due to osteoporosis. A common side effect of raloxifene is hot flashes (sweating and flushing). This can be uncomfortable for women who already have hot flashes due to menopause.

In another embodiment, a sirtuin-activating compound may be used to reduce flushing side effects of antidepressants or anti-psychotic agent. For instance, a sirtuin-activating compound can be used in conjunction (administered separately or together) with a serotonin reuptake inhibitor, a 5HT2 receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT1A receptor antagonist, a 5HT1D receptor antagonist, a CRF antagonist, a monoamine oxidase inhibitor, or a sedative-hypnotic drug.

In certain embodiments, a sirtuin-activating compound may be used as part of a treatment with a serotonin reuptake inhibitor (SRI). In certain preferred embodiments, the SRI is a selective serotonin reuptake inhibitor (SSRI), such as a fluoxetinoid (fluoxetine, norfluoxetine) or a nefazodonoid (nefazodone, hydroxynefazodone, oxonefazodone). Other exemplary SSRI's include duloxetine, venlafaxine, milnacipran, citalopram, fluvoxamine, paroxetine and sertraline. The STAC can also be used as part of a treatment with sedative-hypnotic drug, such as selected from the group consisting of a benzodiazepine (such as alprazolam, chlordiazepoxide, clonazepam, chlorazepate, clobazam, diazepam, halazepam, lorazepam, oxazepam and prazepam), zolpidem, and barbiturates. In still other embodiments, a sirtuin-activating compound may be used as part of a treatment with a 5-HT1A receptor partial agonist, such as selected from the group consisting of buspirone, flesinoxan, gepirone and ipsapirone. Sirtuin-activating compounds can also be used as part of a treatment with a norepinephrine reuptake inhibitor, such as selected from tertiary amine tricyclics and secondary amine tricyclics. Exemplary tertiary amine tricyclics include amitriptyline, clomipramine, doxepin, imipramine and trimipramine. Exemplary secondary amine tricyclics include amoxapine, desipramine, maprotiline, nortriptyline and protriptyline. In certain embodiments, a sirtuin-activating compound may be used as part of a treatment with a monoamine oxidase inhibitor, including, for example, isocarboxazid, phenelzine, tranylcypromine, selegiline and moclobemide.

In still another embodiment, a sirtuin-activating compound may be used to reduce flushing side effects of chemotherapeutic agents, such as cyclophosphamide or tamoxifen.

In another embodiment, a sirtuin-activating compound may be used to reduce flushing side effects of calcium channel blockers, such as amlodipine.

In another embodiment, a sirtuin-activating compound may be used to reduce flushing side effects of antibiotics. For example, a sirtuin-activating compound can be used in combination with levofloxacin. Levofloxacin is used to treat infections of the sinuses, skin, lungs, ears, airways, bones, and joints caused by susceptible bacteria. Levofloxacin also is frequently used to treat urinary infections, including those resistant to other antibiotics, as well as prostatitis. Levofloxacin is effective in treating infectious diarrheas caused by E. coli, campylobacter jejuni, and shigella bacteria. Levofloxacin also can be used to treat various obstetric infections, including mastitis.

In another aspect, a sirtuin-activating compound may be used as a therapy for reducing the incidence or severity of weight gain which is a side-effect of another drug therapy, e.g., drug-induced weight gain. In certain embodiments, a method for treating and/or preventing drug-induced weight gain comprises administering to a patient in need thereof a formulation comprising at least one weight gain inducing compound and at least one sirtuin activating compound. In other embodiments, a method for treating drug-induced weight gain comprises separately administering one or more compounds that induce weight gain and one or more sirtuin-activating compounds, e.g., wherein the sirtuin-activating compound and weight gain inducing agent have not been formlulated in the same compositions. When using separate formulations, the sirtuin-activating compound may be administered (1) at the same as administration of the weight gain inducing agent, (2) intermittently with the weight gain inducing agent, (3) staggered relative to administration of the weight gain inducing agent, (4) prior to administration of the weight gain inducing agent, (5) subsequent to administration of the weight gain inducing agent, and (6) various combination thereof. Exemplary weight gain inducing agents include, for example, anti-diabetic agents, antidepressants, steroids, hormones, beta blockers, alpha blockers, and contraceptives.

In another embodiment, a sirtuin-activating compound may be administered to reduce drug-induced weight gain. For example, a sirtuin activating compound may be administered as a combination therapy with medications that may stimulate appetite or cause weight gain, in particular, weight gain due to factors other than water retention. Examples of medications that may cause weight gain, include for example, diabetes treatments, including, for example, sulfonylureas (such as glipizide and glyburide), thiazolidinediones (such as pioglitazone and rosiglitazone), meglitinides, nateglinide, repaglinide, sulphonylurea medicines, and insulin; antidepressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy; lithium carbonate; valproic acid; carbamazepine; chlorpromazine; thiothixene; beta blockers (such as propranolo); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, sirtuin-activating compounds may be administered as part of a smoking cessation program to prevent weight gain or reduce weight already gained.

In certain embodiments, methods for reducing, preventing or treating flushing and/or drug-induced weight gain may also comprise increasing the protein level of a sirtuin, such as SIRT1 in a human cell or a homologue of any of the sirtuins in other organisms. Increasing protein levels can be achieved by introducing into a cell one or more copies of a nucleic acid that encodes a sirtuin. For example, the level of SIRT1 can be increased in a mammalian cell by introducing into the mammalian cell a nucleic acid encoding SIRT1, e.g., having the amino acid sequence set forth in GenBank Accession No. NP_{—}036370. The nucleic acid may be under the control of a promoter that regulates the expression of the SIRT1 nucleic acid. Alternatively, the nucleic acid may be introduced into the cell at a location in the genome that is downstream of a promoter. Methods for increasing the level of a protein using these methods are well known in the art.

A nucleic acid that is introduced into a cell to increase the protein level of a sirtuin may encode a protein that is at least about 80%, 85%, 90%, 95%, 98%, or 99% identical to the sequence of a sirtuin, e.g., SIRT1 (GenBank Accession No. NP_{—}036370). For example, the nucleic acid encoding the protein may be at least about 80%, 85%, 90%, 95%, 98%, or 99% identical to the SIRT1 nucleic acid sequence (GenBank Accession No. NM_{—}012238). The nucleic acid may also be a nucleic acid that hybridizes, preferably under stringent hybridization conditions, to a nucleic acid encoding a wild-type sirtuin, e.g., SIRT1 (GenBank Accession No. NM_{—}012238). Stringent hybridization conditions may include hybridization and a wash in 0.2×SSC at 65° C. When using a nucleic acid that encodes a protein that is different from a wild-type sirtuin protein, such as a protein that is a fragment of a wild-type sirtuin, the protein is preferably biologically active, e.g., is capable of deacetylation. It is only necessary to express in a cell a portion of the sirtuin that is biologically active. For example, a protein that differs from wild-type SIRT1 (GenBank Accession No. NP_{—}036370), preferably contains the core structure thereof. The core structure sometimes refers to amino acids 62-293 of SIRT1 (GenBank Accession No. NP_{—}036370), which are encoded by nucleotides 237 to 932 of GenBank Accession No. NM_{—}012238, which encompasses the NAD binding as well as the substrate binding domains. The core domain of SIRT1 may also refer to about amino acids 261 to 447 of SIRT1 (GenBank Accession No. NP_{—}036370), which are encoded by nucleotides 834 to 1394 of GenBank Accession No. NM_{—}012238; to about amino acids 242 to 493 of SIRT1 (GenBank Accession No. NP_{—}036370), which are encoded by nucleotides 777 to 1532 of GenBank Accession No. NM_{—}012238; or to about amino acids 254 to 495 of SIRT1 (GenBank Accession No. NP_{—}036370), which are encoded by nucleotides 813 to 1538 of GenBank Accession No. NM_{—}012238. Whether a protein retains a biological function, e.g., deacetylation capabilities, can be determined according to methods known in the art.

Methods for increasing sirtuin protein levels also include methods for stimulating the transcription of genes encoding sirtuins, methods for stabilizing the corresponding mRNAs, methods, and other methods known in the art.

In another aspect, the invention provides use of a sirtuin-activating compound in combination with nicotinic acid for increasing the level or activity of a sirtuin protein, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. As described further below, the methods comprise administering to a subject in need thereof a therapeutically effective amount of a sirtuin-activating compound and nicotinic acid. In an exemplary embodiment, the methods comprise administering to a subject in need thereof a composition comprising a pharmaceutically effective amount of a sirtuin-activating compound and nicotinic acid. As described above, the term “nicotinic acid” is meant to encompass nicotinic acid or a compound other than nicotinic acid itself which the body metabolizes into nicotinic acid, thus producing essentially the same effect as nicotinic acid. Exemplary compounds that produce an effect similar to that of nicotinic acid include, for example, nicotinyl alcohol tartrate, d-glucitol hexanicotinate, aluminum nicotinate, niceritrol and d,l-alpha-tocopheryl nicotinate.

In yet another embodiment, a sirtuin activating compound and nicotinic acid may be administered as part of a combination therapy with one or more therapeutic agents for the treatment or prevention of various diseases, including, for example, cancer, diabetes, neurodegenerative diseases, cardiovascular disease, blood clotting, inflammation, flushing, obesity, ageing, stress, etc. In various embodiments, combination therapies comprising a sirtuin-activating compound, nicotinic acid, and an additiona therapeutic agent may refer to (1) pharmaceutical compositions that comprise a sirtuin-activating compound, nicotinic acid, and one or more therapeutic agents; and (2) co-administration of a sirtuin-activating compound and nicotinic acid with one or more therapeutic agents wherein the sirtuin-activating compound/nictonic acid and the therapeutic agent have not been formlulated in the same compositions. When using separate formulations, the sirtuin-activating compound/nicotinic acid may be administered at the same, intermittent, staggered, prior to, subsequent to, or combinations thereof, with the administration of another therapeutic agent. In an exemplary embodiment, a sirtuin-activating compound and/or nicotinic acid may be administered by sustained release.

In one embodiment, the invention provides a method for extending the lifespan of a cell, extending the proliferative capacity of a cell, slowing ageing of a cell, promoting the survival of a cell, delaying cellular senescence in a cell, mimicking the effects of calorie restriction, increasing the resistance of a cell to stress, or preventing apoptosis of a cell, by contacting the cell with a sirtuin-activating compound and nicotinic acid.

The methods described herein may be used to increase the amount of time that cells, particularly primary cells (i.e., cells obtained from an organism, e.g., a human), may be kept alive in a cell culture. Embryonic stem (ES) cells and pluripotent cells, and cells differentiated therefrom, may also be treated with a sirtuin-activating compound and nicotinic acid to keep the cells, or progeny thereof, in culture for longer periods of time. Such cells can also be used for transplantation into a subject, e.g., after ex vivo modification.

In one embodiment, cells that are intended to be preserved for long periods of time may be treated with a sirtuin-activating compound and nicotinic acid. The cells may be in suspension (e.g., blood cells, serum, biological growth media, etc.) or in tissues or organs. For example, blood collected from an individual for purposes of transfusion may be treated with a sirtuin-activating compound and nicotinic acid to preserve the blood cells for longer periods of time. Additionally, blood to be used for forensic purposes may also be preserved using the sirtuin-activating compounds described herein. Other cells that may be treated to extend their lifespan or protect against apoptosis include cells for consumption, e.g., cells from non-human mammals (such as meat) or plant cells (such as vegetables).

A sirtuin-activating compound and nicotinic acid may also be applied during developmental and growth phases in mammals, plants, insects or microorganisms, in order to, e.g., alter, retard or accelerate the developmental and/or growth process.

In another embodiment, a sirtuin-activating compound and nicotinic acid may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the sirtuin-activating compound and nicotinic acid prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a sirtuin-activating compound and nicotinic acid or may have a subset of cells/tissue treated locally with a sirtuin-activating compound and nicotinic acid. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, cells may be treated with a sirtuin-activating compound and nicotinic acid in vivo, e.g., to increase their lifespan or prevent apoptosis. For example, skin can be protected from aging (e.g., developing wrinkles, loss of elasticity, etc.) by treating skin or epithelial cells with a sirtuin-activating compound and nicotinic acid. In an exemplary embodiment, skin is contacted with a pharmaceutical or cosmetic composition comprising a sirtuin-activating compound and nicotinic acid. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, skin cancer and the effects of natural aging. In another embodiment, a sirtuin-activating compound and nicotinic acid may be used for the treatment of wounds and/or burns to promote healing, including, for example, first-, second- or third-degree burns and/or a thermal, chemical or electrical burns. The formulations may be administered topically, to the skin or mucosal tissue, as an ointment, lotion, cream, microemulsion, gel, solution or the like, as further described herein, within the context of a dosing regimen effective to bring about the desired result.

Topical formulations comprising a sirtuin-activating compound and nicotinic acid may also be used as preventive, e.g., chemopreventive, compositions. When used in a chemopreventive method, susceptible skin is treated prior to any visible condition in a particular individual.

A sirtuin-activating compound and nicotinic acid may be delivered locally or systemically to a subject. In one embodiment, a sirtuin-activating compound and nicotinic acid is delivered locally to a tissue or organ of a subject by injection, topical formulation, etc.

In another embodiment, a sirtuin-activating compound and nicotinic acid may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a sirtuin-activating compound and nicotinic acid may be administered to a subject in order to generally increase the lifespan of its cells and to protect its cells against stress and/or against apoptosis. It is believed that treating a subject with a sirtuin-activating compound and nicotinic acid is similar to subjecting the subject to hormesis, i.e., mild stress that is beneficial to organisms and may extend their lifespan.

A sirtuin-activating compound and nicotinic acid may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. A sirtuin-activating compound and nicotinic acid can also be administered to subjects for treatment of diseases, e.g., chronic diseases, associated with cell death, in order to protect the cells from cell death. Exemplary diseases include those associated with neural cell death or muscular cell death, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections.

In another embodiment, a sirtuin activating compound in combination with nicotinic acid may also be administered to a subject suffereing from a disease or disorder involving ischemia and/or reperfusion injury. Exemplary ischemic diseases and disorders include, for example, ischemic stroke, ischemic tissue injury, e.g. ischemic injury of organs, cardiac ischemia, cardiac reperfusion injury and complications resulting from organ transplantation, e.g. kidney, heart and liver or cardio-pulmonary bypass surgery and other disorders.

A sirtuin-activating compound and nicotinic acid can also be administered to a subject suffering from an acute disease, e.g., damage to an organ or tissue, e.g., a subject suffering from stroke or myocardial infarction or a subject suffering from a spinal cord injury. A sirtuin-activating compound and nicotinic acid may also be used to repair an alcoholic's liver.

In another embodiment, the invention provides a method for treating and/or preventing a cardiovascular disease by administering to a subject in need thereof a sirtuin-activating compound and nicotinic acid.

Cardiovascular diseases that can be treated or prevented using a sirtuin-activating compound and nicotinic acid include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems. A sirtuin-activating compound and nicotinic acid may also be used for increasing HDL levels in plasma of an individual.

Yet other disorders that may be treated with a sirtuin-activating compound and nicotinic acid include restenosis, e.g., following coronary intervention, and disorders relating to an abnormal level of high density and low density cholesterol.

In one embodiment, a sirtuin-activating compound and nicotinic acid may be administered as part of a combination therapeutic with another cardiovascular agent including, for example, an anticoagulant, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

A sirtuin-activating compound and nicotinic acid may also be administered to subjects who have recently received or are likely to receive a dose of radiation. In one embodiment, the dose of radiation is received as part of a work-related or medical procedure, e.g., working in a nuclear power plant, flying an airplane, an X-ray, CAT scan, or the administration of a radioactive dye for medical imaging; in such an embodiment, the compound is administered as a prophylactic measure. In another embodiment, the radiation exposure is received unintentionally, e.g., as a result of an industrial accident, terrorist act, or act of war involving radioactive material. In such a case, a sirtuin-activating compound and nicotinic acid is preferably administered as soon as possible after the exposure to inhibit apoptosis and the subsequent development of acute radiation syndrome.

Based at least on the discovery that certain concentrations of sirtuin-activating compounds prevent deacetylation of p53 in cells and thereby may induce apoptosis in cells, a sirtuin-activating compound and nicotinic acid can also be administed to a subject in conditions in which apoptosis of certain cells is desired. For example, cancer may be treated or prevented. Exemplary cancers are those of the brain and kidney; hormone-dependent cancers including breast, prostate, testicular, and ovarian cancers; lymphomas, and leukemias. In cancers associated with solid tumors, a sirtuin-activating compound and nicotinic acid may be administered directly into the tumor. Cancer of blood cells, e.g., leukemia, can be treated by administering a sirtuin-activating compound and nicotinic acid into the blood stream or into the bone marrow. Benign cell growth can also be treated, e.g., warts. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of compounds. Alternatively, cells can be obtained from a subject, treated ex vivo to remove certain undesirable cells, e.g., cancer cells, and administered back to the same or a different subject.

In one embodiment, a sirtuin-activating compound and nicotinic acid may be administered as part of a combination therapeutic with another chemotherapeutic agent.

In certain aspects, a sirtuin-activating compound and nicotinic acid can be used to treat patients suffering from neurodegenerative diseases, and traumatic or mechanical injury to the central nervous system (CNS) or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases evolve gradually, after a long period of normal brain function, due to progressive degeneration (e.g., nerve cell dysfunction and death) of specific brain regions. The actual onset of brain degeneration may precede clinical expression by many years. Examples of neurodegenerative diseases include, but are not limited to, Alzheimer's disease (AD), Parkinson's disease (PD), Huntington disease (HD), amyotrophic lateral sclerosis (ALS; Lou Gehrig's disease), diffuse Lewy body disease, chorea-acanthocytosis, primary lateral sclerosis, and Friedreich's ataxia. Other examples of neuronal diseases or disorders that may treated using a sirtuin-activating compound in combination with nicotinic acid are described below.

Tay-Sachs disease and Sandhoff disease are glycolipid storage diseases caused by the lack of lysosomal β-hexosamimidase (Gravel et al., in The Metabolic Basis of Inherited Disease, eds. Scriver et al., McGraw-Hill, New York, pp. 2839-2879, 1995). In both disorders, GM2 ganglioside and related glycolipidssubstrates for β-hexosamimidase accumulate in the nervous system and trigger acute neurodegeneration. In the most severe forms, the onset of symptoms begins in early infancy. A precipitous neurodegenerative course then ensues, with affected infants exhibiting motor dysfunction, seizure, visual loss, and deafness. Death usually occurs by 2-5 years of age. Neuronal loss through an apoptotic mechanism has been demonstrated (Huang et al., Hum. Mol. Genet. 6: 1879-1885, 1997).

It is well-known that apoptosis plays a role in AIDS pathogenesis in the immune system. However, HIV-1 also induces neurological disease. Shi et al. (J. Clin. Invest. 98: 1979-1990, 1996) examined apoptosis induced by HIV-1 infection of the CNS in an in vitro model and in brain tissue from AIDS patients, and found that HIV-1 infection of primary brain cultures induced apoptosis in neurons and astrocytes in vitro. Apoptosis of neurons and astrocytes was also detected in brain tissue from 10/11 AIDS patients, including 5/5 patients with HIV-1 dementia and 4/5 nondemented patients.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. A sirtuin-activating compound and nicotinic acid may be useful for treating or preventing neuronal loss due to these prior diseases.

In another embodiment, a sirtuin-activating compound and nicotinic acid may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy. The most distal portions of axons are usually the first to degenerate, and axonal atrophy advances slowly towards the nerve's cell body. If the noxious stimulus is removed, regeneration is possible, though prognosis decreases depending on the duration and severity of the stimulus. Those with distal axonopathies usually present with symmetrical stocking-glove sensori-motor disturbances. Deep tendon reflexes and autonomic nervous system (ANS) functions are also lost or diminished in affected areas.

Diabetic neuropathies are neuropathic disorders that are associated with diabetes mellitus. These conditions usually result from diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy. Clinical manifestations of diabetic neuropathy include, for example, sensorimotor polyneuropathy such as numbness, sensory loss, dysesthesia and nighttime pain; autonomic neuropathy such as delayed gastric emptying or gastroparesis; and cranial neuropathy such as oculomotor (3rd) neuropathies or Mononeuropathies of the thoracic or lumbar spinal nerves.

Peripheral neuropathy is the medical term for damage to nerves of the peripheral nervous system, which may be caused either by diseases of the nerve or from the side-effects of systemic illness. Peripheral neuropathies vary in their presentation and origin, and may affect the nerve or the neuromuscular junction. Major causes of peripheral neuropathy include seizures, nutritional deficiencies, and HIV, though diabetes is the most likely cause. Mechanical pressure from staying in one position for too long, a tumor, intraneural hemorrhage, exposing the body to extreme conditions such as radiation, cold temperatures, or toxic substances can also cause peripheral neuropathy.

In an exemplary embodiment, a sirtuin-activating compound and nicotinic acid may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

In yet another embodiment, a sirtuin-activating compound and nicotinic acid may be used to treat trauma to the nerves, including, trauma due to disease, injury (including surgical intervention), or environmental trauma (e.g., neurotoxins, alcoholism, etc.).

A sirtuin-activating compound in combination with nicotinic acid may also be useful to prevent, treat, and alleviate symptoms of various PNS disorders, such as the ones described below. The PNS is composed of the nerves that lead to or branch off from the CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

Peripheral neuropathy is a widespread disorder, and there are many underlying causes. Some of these causes are common, such as diabetes, and others are extremely rare, such as acrylamide poisoning and certain inherited disorders. The most common worldwide cause of peripheral neuropathy is leprosy. Leprosy is caused by the bacterium Mycobacterium leprae, which attacks the peripheral nerves of affected people.

Leprosy is extremely rare in the United States, where diabetes is the most commonly known cause of peripheral neuropathy. It has been estimated that more than 17 million people in the United States and Europe have diabetes-related polyneuropathy. Many neuropathies are idiopathic; no known cause can be found. The most common of the inherited peripheral neuropathies in the United States is Charcot-Marie-Tooth disease, which affects approximately 125,000 persons.

Another of the better known peripheral neuropathies is Guillain-Barré syndrome, which arises from complications associated with viral illnesses, such as cytomegalovirus, Epstein-Barr virus, and human immunodeficiency virus (HIV), or bacterial infection, including Campylobacter jejuni and Lyme disease. The worldwide incidence rate is approximately 1.7 cases per 100,000 people annually. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the PNS and the CNS, as well as other body tissues.

Other PNS diseases treatable with a sirtuin-activating compound in combination with nicotinic acid include: Brachial Plexus Neuropathies (diseases of the cervical and first thoracic roots, nerve trunks, cords, and peripheral nerve components of the brachial plexus. Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp1351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy (see Adams et al., Principles of Neurology, 6th ed, p1325); mononeuropathies (disease or trauma involving a single peripheral nerve in isolation, or out of proportion to evidence of diffuse peripheral nerve dysfunction). Mononeuropathy multiplex refers to a condition characterized by multiple isolated nerve injuries. Mononeuropathies may result from a wide variety of causes, including ischemia; traumatic injury; compression; connective tissue diseases; cumulative trauma disorders; and other conditions; Neuralgia (intense or aching pain that occurs along the course or distribution of a peripheral or cranial nerve); Peripheral Nervous System Neoplasms (neoplasms which arise from peripheral nerve tissue). This includes neurofibromas; Schwannomas; granular cell tumors; and malignant peripheral nerve sheath tumors. See DeVita Jr et al., Cancer: Principles and Practice of Oncology, 5th ed, pp1750-1); Nerve Compression Syndromes (mechanical compression of nerves or nerve roots from internal or external causes. These may result in a conduction block to nerve impulses, due to, for example, myelin sheath dysfunction, or axonal loss. The nerve and nerve sheath injuries may be caused by ischemia; inflammation; or a direct mechanical effect; Neuritis (a general term indicating inflammation of a peripheral or cranial nerve). Clinical manifestation may include pain; paresthesias; paresis; or hyperthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

In one embodiment, a sirtuin-activating compound and nicotinic acid may be administered as part of a combination therapeutic with another therapeutic agent useful for the treating or preventing neurodegenerative disorders or secondary conditions associated with these conditions. Thus, a combination drug regimen may include a sirtuin-activaing compound, nicotinic acid, and one or more anti-neurodegeneration agents. Exemplary therapeutic agents for treating or preventing neuronal diseases or disorders include, for example, L-DOPA; a dopamine agonist; an adenosine A_{2A} receptor antagonists; a COMT inhibitor; a MAO inhibitor; an NOS inhibitor; a sodium channel antagonist; a selective N-methyl D-aspartate (NMDA) receptor antagonists; an AMPA/kainate receptor antagonist; a calcium channel antagonist; a GABA-A receptor agonist; an acetyl-choline esterase inhibitor; a matrix metalloprotease inhibitor; an inhibitor of p38 MAP kinase or c-jun-N-terminal kinases; TPA; NDA antagonists; beta-interferons; growth factors; glutamate inhibitors; and/or as part of a cell therapy.

In other aspects, a sirtuin-activating compound and nicotinic acid can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. After initiation of clotting, blood coagulation proceeds through the sequential activation of certain plasma proenzymes to their enzyme forms (see, for example, Coleman, R. W. et al. (eds.) Hemostasis and Thrombosis, Second Edition, (1987)). These plasma glycoproteins, including Factor XII, Factor XI, Factor IX, Factor X, Factor VII, and prothrombin, are zymogens of serine proteases. Most of these blood clotting enzymes are effective on a physiological scale only when assembled in complexes on membrane surfaces with protein cofactors such as Factor VIII and Factor V. Other blood factors modulate and localize clot formation, or dissolve blood clots. Activated protein C is a specific enzyme that inactivates procoagulant components. Calcium ions are involved in many of the component reactions. Blood coagulation follows either the intrinsic pathway, where all of the protein components are present in blood, or the extrinsic pathway, where the cell-membrane protein tissue factor plays a critical role. Clot formation occurs when fibrinogen is cleaved by thrombin to form fibrin. Blood clots are composed of activated platelets and fibrin.

Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place. It involves a cascade of complicated and regulated biochemical reactions between circulating blood proteins (coagulation factors), blood cells (in particular platelets), and elements of an injured vessel wall.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect of the invention, the invention provides a method for reducing or inhibiting hemostasis in a subject by administering a sirtuin-activating compound in combination with nicotinic acid. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. Examples of thrombotic disorders include, but are not limited to, thromboembolism, deep vein thrombosis, pulmonary embolism, stroke, myocardial infarction, miscarriage, thrombophilia associated with antithrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, e.g., unstable angina, disseminated intravascular coagulation, thrombotic thrombocytopenic purpura, cancer metastasis, sickle cell disease, glomerular nephritis, and drug induced thrombocytopenia (including, for example, heparin induced thrombocytopenia). In addition, a sirtuin-activating compound and nicotinic acid may be administered to prevent thrombotic events or to prevent re-occlusion during or after therapeutic clot lysis or procedures such as angioplasty or surgery.

In one embodiment, a sirtuin-activating compound and nicotinic acid may be administered as part of a combination therapeutic with another therapeutic agent useful for the treatment or prevention of blood coagulation disorders or secondary conditions associated with these conditions. Thus, a combination drug regimen may include a sirtuin-activating compound, nicotinic acid, and one or more anti-coagulation or anti-thrombosis agents. Exemplary anti-coagulation or anti-thrombosis agents include, for example, aspirin, heparin, and oral Warfarin that inhibits Vit K-dependent factors, low molecular weight heparins that inhibit factors X and II, thrombin inhibitors, inhibitors of platelet GP IIbIIIa receptors, inhibitors of tissue factor (TF), inhibitors of human von Willebrand factor, inhibitors of one or more factors involved in hemostasis (in particular in the coagulation cascade). In addition, a sirtuin-activating compound and nicotinic acid can be combined with thrombolytic agents, such as t-PA, streptokinase, reptilase, TNK-t-PA, and staphylokinase.

In another aspect, a sirtuin-activating compound in combination with nicotinic acid may be used for treating or preventing weight gain or obesity in a subject. For example, a sirtuin-activating compound and nicotinic acid may be used, for example, to treat or prevent hereditary obesity, dietary obesity, hormone related obesity, obesity related to the administration of medication, to reduce the weight of a subject, or to reduce or prevent weight gain in a subject, including drug-induced weight gain. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, a sirtuin-activating compound and nicotinic acid may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Stunkard A J, Wadden T A. (Editors) Obesity: theory and therapy, Second Edition. New York: Raven Press, 1993. Finally, patients with AIDS can develop lipodystrophy or insulin resistance in response to combination therapies for AIDS.

In another embodiment, a sirtuin-activating compound and nicotinic acid may be used for inhibiting adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used for treating or preventing obesity.

In other embodiments, a sirtuin-activating compound and nicotinic acid may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

A method may further comprise monitoring the weight of the subject and/or the level of activation of sirtuins, for example, in adipose tissue.

In an exemplary embodiment, a sirtuin-activating compound and nicotinic acid may be administered as a combination therapy for treating or preventing weight gain or obesity. For example, a sirtuin-activating compound and nicotinic acid may be administered in combination with one or more anti-obesity agents. Exemplary anti-obesity agents include, for example, phenylpropanolamine, ephedrine, pseudoephedrine, phentermine, a cholecystokinin-A agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (leptin), a leptin analog, a leptin receptor agonist, a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin and ciliary neurotrophic factors such as Axokine.

In another aspect, a sirtuin-activating compound and nicotinic acid may be used for treating or preventing a metabolic disorder, such as insulin-resistance, a pre-diabetic state, type II diabetes, and/or complications thereof. Administration of a sirtuin-activating compound and nicotinic acid may increase insulin sensitivity and/or decrease insulin levels in a subject. A subject in need of such a treatment may be a subject who has insulin resistance or other precusor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, a sirtuin-activating compound and nicotinic acid may be administered as a combination therapy for treating or preventing a metabolic disorder. For example, a sirtuin-activating compound and nicotinic acid may be administered in combination with one or more anti-diabetic agents. Exemplary anti-diabetic agents include, for example, an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a peroxisome proliferator-activated receptor-γ (PPAR-γ) ligand such as troglitazone, rosaglitazone, pioglitazone or GW-1929, a sulfonylurea, glipazide, glyburide, or chlorpropamide wherein the amounts of the first and second compounds result in a therapeutic effect. Other compoundsanti-diabetic agents include a glucosidase inhibitor, a glucagon-like peptide-1 (GLP-1), insulin, a PPAR α/γ dual agonist, a meglitimide and an αP2 inhibitor. In an exemplary embodiment, an anti-diabetic agent may be a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrolidine) or MK-04301 from Merck (see e.g., Hughes et al., Biochemistry 38: 11597-603 (1999)).

In other aspects, a sirtuin-activating compound and nicotinic acid can be used to treat or prevent a disease or disorder associated with inflammation. A sirtuin-activating compound and nicotinic acid may be administered prior to the onset of, at, or after the initiation of inflammation. When used prophylactically, a sirtuin-activating compound and nicotinic acid are preferably provided in advance of any inflammatory response or symptom. Administration of a sirtuin-activating compound and nicotinic acid may prevent or attenuate inflammatory responses or symptoms.

Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, a sirtuin-activating compound and nicotinic acid may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, a sirtuin-activating compound and nicotinic acid may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, a sirtuin-activating compound and nicotinic acid may be taken in combination with other compounds useful for treating or preventing inflammation. Exemplary anti-inflammatory agents include, for example, steroids (e.g., cortisol, cortisone, fludrocortisone, prednisone, 6α-methylprednisone, triamcinolone, betamethasone or dexamethasone), nonsteroidal antiinflammatory drugs (NSAIDS (e.g., aspirin, acetaminophen, tolmetin, ibuprofen, mefenamic acid, piroxicam, nabumetone, rofecoxib, celecoxib, etodolac or nimesulide), a PDE4 inhibitor (e.g., roflumilast or rolipram), or an antihistamine (e.g., cyclizine, hydroxyzine, promethazine or diphenhydramine).

A sirtuin-activating compound in combination with nicotinic acid may be used for treating or preventing viral infections (such as infections by influenza, herpes or papilloma virus) or as antifungal agents.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, a sirtuin-activating compound and nicotinic acid may be administered to farm animals to improve their ability to withstand farming conditions longer.

A sirtuin-activating compound and nicotinic acid may also be used to increase lifespan, stress resistance, and resistance to apoptosis in plants. In one embodiment, a sirtuin-activating compound and nicotinic acid is applied to plants, e.g., on a periodic basis, or to fungi. In another embodiment, plants are genetically modified to produce a compound. In another embodiment, plants and fruits are treated with a sirtuin-activating compound and nicotinic acid prior to picking and shipping to increase resistance to damage during shipping. Plant seeds may also be contacted with a sirtuin-activating compound and nicotinic acid, e.g., to preserve them.

In other embodiments, a sirtuin-activating compound and nicotinic acid may be used for modulating lifespan in yeast cells. Situations in which it may be desirable to extend the lifespan of yeast cells include any process in which yeast is used, e.g., the making of beer, yogurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

A sirtuin-activating compound and nicotinic acid may also be used to increase lifespan, stress resistance and resistance to apoptosis in insects. In this embodiment, a sirtuin-activating compound and nicotinic acid would be applied to useful insects, e.g., bees and other insects that are involved in pollination of plants. In a specific embodiment, a sirtuin-activating compound and nicotinic acid would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of a sirtuin-activating compound and nicotinic acid may also be used as a pesticide by interfering with the regulation of silenced genes and the regulation of apoptosis during development. In this embodiment, a sirtuin-activating compound and nicotinic acid may be applied to plants using a method known in the art that ensures the compounds are bioavailable to insect larvae, and not to plants.

At least in view of the link between reproduction and longevity (Longo and Finch, Science, 2002), a sirtuin-activating compound and nicotinic acid can be applied to affect the reproduction of organisms such as insects, animals and microorganisms.

4. Assays

In certain aspects, the present invention provides screening methods for identifying compounds (agents) for treating and/or preventing flushing and/or drug-induced weight gain. Candidate compounds identified by the subject screening methods can be administered to a subject, such as a subject in need thereof. A subject in need of such a treatment may be a subject who suffers from flushing and/or drug-induced weight gain, or who has, or is, likely to have these conditions, as predicted, e.g., from family history, age, prescribed medications, etc. Exemplary agents are those described herein.

The effect of a compound on the activity of a sirtuin, such as SIRT1, may be determined as described, e.g., in Howitz et al., supra or as follows. For instance, sirtuin proteins may be contacted with a compound in vitro, e.g., in a solution or in a cell. In one embodiment, a sirtuin protein is contacted with a compound in a solution and an activity of the sirtuin, e.g., its ability to deacetylate a protein, such as a histone, p53, or portions thereof, is determined. Generally, a sirtuin is activated by a compound when at least one of its biological activities, e.g., deacetylation activity, is higher in the presence of the compound than in its absence. Activation may be by a factor of at least about 10%, 30%, 50%, 100% (i.e., a factor of two), 3, 10, 30, or 100.

Sirtuin activation can be determined, e.g., by contacting the sirtuin, or a cell or cell extract containing the sirtuin, with a deacetylation target, such as a histone, a p53 protein, or portions thereof, and determining the level of acetylation of the deacetylation target. A higher level of acetylation of the target incubated with the sirtuin that is being tested relative to the level of acetylation of a control sirtuin indicates that the sirtuin that is being tested is activated. The control sirtuin may be a recombinantly produced sirtuin that has not been contacted with a sirtuin-activating or -inhibiting compound.

Assays for determining the likelihood that a subject has or will develop flushing or drug-induced weight gain are well known in the art. For example, such assays may comprise determining the level of activity or expression (e.g., mRNA, pre-mRNA or protein) of a sirtuin such as SIRT1 in a subject. A low level of sirtuin activity or expression in a subject is likely to indicate that the subject has or is likely to develop flushing or drug-induced weight gain, or secondary conditions thereof. Alternatively, a higher level of sirtuin activity or expression in a subject is likely to indicate that the subject has or is likely to be protected from developing flushing or drug-induced weight gain. Other assays include determining the activity or level of expression of a sirtuin.

In certain embodiments, a method may comprise contacting a sirtuin with a test agent and determining the effect of the test agent on the activity of the sirtuin, e.g., SIRT1, as described, e.g., in Howitz et al., supra. The first step of the method may also comprise contacting a cell comprising a sirtuin with a test agent and determining the effect of the test agent on the activity of or expression level of the sirtuin. Expression levels of a sirtuin may be determined by measuring the mRNA, pre-mRNA or protein level of the sirtuin. Other steps of the method may comprise testing the agent in an animal model for flushing or drug-induced weight gain. Such animal models are well known in the art. Screening methods may further comprise a step to determine the toxicity or adverse effects of the agents.

5. Pharmaceutical Formulations

Pharmaceutical compositions comprising sirtuin-activating compounds may be formulated in conventional manner using one or more physiologically acceptable carriers or excipients. Thus, sirtuin-activating compounds and their physiologically acceptable salts and solvates may be formulated for administration by, for example, injection, inhalation or insufflation (either through the mouth or the nose) or oral, buccal, parenteral or rectal administration. In one embodiment, the sirtuin-activating compound is administered locally, at the site where the target cells, e.g., neuronal cells or blood cells.

In an exemplary embodiment, a pharmaceutical composition comprises one or more sirtuin-activating compounds and one or more compounds that induce flushing. Exemplary compounds that induce flushing include, for example, niacin, faloxifene, antidepressants, antipsychotics, chemotherapeutics, calcium channel blockers, and antibiotics. In another embodiment, a pharmaceutical composition comprises one or more sirtuin-activating compounds and one or more compounds that induce weight gain. Exemplary compounds that induce weight gain include, for example, anti-diabetic agents, antidepressants, steroids, hormones, beta blockers, alpha blockers, and contraceptives.

Sirtuin-activating compounds can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the sirtuin-activating compounds can be formulated in liquid solutions, preferably in physiologically compatible buffers such as Hank's solution or Ringer's solution. In addition, the sirtuin-activating compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Polyphenols such as resveratrol can oxidize and lose sirtuin-stimulatory activity, especially in a liquid or semi-solid form. To prevent oxidation and preserve the sirtuin-stimulatory activity of polyphenol-containing compounds, the compounds may be stored in a nitrogen atmosphere or sealed in a type of capsule and/or foil package that excludes oxygen (e.g., Capsugel™).

For administration by inhalation, the sirtuin-activating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

Sirtuin-activating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Sirtuin-activating compounds may also be formulated in rectal compositions such as suppositories or retention enemas, e.g., containing conventional suppository bases such as cocoa butter or other glycerides.

In addition to the formulations described previously, the sirtuin-activating compounds may also be formulated as a depot preparation. Such long acting formulations may be administered by implantation (for example subcutaneously or intramuscularly) or by intramuscular injection. Thus, for example, the sirtuin-activating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

Pharmaceutical compositions (including cosmetic preparations) may comprise from about 0.00001 to 100% such as from 0.001 to 10% or from 0.1% to 5% by weight of one or more sirtuin-activating compounds described herein. In an exemplary embodiment, pharmaceutical compositions may further comprises from about 0.00001 to 100%, such as from 0.001 to 10%, or from 0.1% to 5%, by weight of one or more compounds that induce flushing or one or more compounds that induce weight gain.

In one embodiment, a sirtuin-activating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Sirtuin-activating compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Sirtuin-activating compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor™ from Beiersdorf, Inc. (Norwalk, Conn.).

Sirtuin-activating compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Sirtuin-activating compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and diesters of PEG (e.g., oleoyl macrogol glycerides), etc.

Sirtuin-activating compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C₁₀ MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C₂-C₆ alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone™ from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprise asirtuin-activating compound-containing microemulsion as described above, and may contain alternative pharmaceutically acceptable carriers, vehicles, additives, etc. particularly suited to oral or parenteral drug administration. Alternatively, a sirtuin-activating compound-containing microemulsion may be administered orally or parenterally substantially as described above, without modification.

Administration of a sirtuin-activating compound may be followed by measuring a factor in the subject, such as measuring the activity of the sirtuin. In an illustrative embodiment, a cell is obtained from a subject following administration of an activating or inhibiting compound to the subject, such as by obtaining a biopsy, and the activity of the sirtuin or sirtuin expression level is determined in the biopsy. Alternatively, biomarkers, such as plasma biomarkers may be followed. The cell may be any cell of the subject, but in cases in which an activating compound is administered locally, the cell is preferably a cell that is located in the vicinity of the site of administration. For example, the cell may be a neuronal cell or a blood cell.

Introduction and expression of a nucleic acid encoding a sirtuin or molecules (e.g., an siRNA) that will reduced the protein level of a sirtuin in a cell may be accomplished using an expression vector. Exemplary expression vectors include adenoviral vectors or adenoviral-associated viruses (AAV). These vectors, as well as others and methods for infecting target cells are well known in the art. Alternatively, nucleic acids may also be introduced into cells using liposomes or similar technologies.

6. Kits

Also provided herein are kits, e.g., kits for therapeutic purposes, including kits for treating or preventing flushing or drug-induced weight gain, or secondary conditions thereof. A kit may comprise one or more agent that modulates sirtuin protein activity or level, e.g., sirtuin activating compounds, such as those described herein, and optionally devices for contacting cells with the agents. Devices include syringes, stents and other devices for introducing a compound into a subject or applying it to the skin of a subject.

Further, a kit may also contain components for measuring a factor, e.g., described above, such as the activity of sirtuin proteins, e.g., in tissue samples.

Other kits include kits for diagnosing the likelihood of having or developing flushing or drug-induced weight gain or secondary conditions thereof. A kit may comprise an agent for measuring the activity and or expression level of a sirtuin.

Kits for screening assays are also provided. Exemplary kits comprise one or more agents for conducting a screening assay, such as a sirtuin or a biologically active portion thereof, or a cell or cell extract comprising such. Any of the kits may also comprise instructions for use.

The present description is further illustrated by the following examples, which should not be construed as limiting in any way. The contents of all cited references (including literature references, issued patents, published patent applications and GenBank Accession numbers as cited throughout this application) are hereby expressly incorporated by reference.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXEMPLIFICATION

### Example 1

### Small Molecule Activators of SIRT1

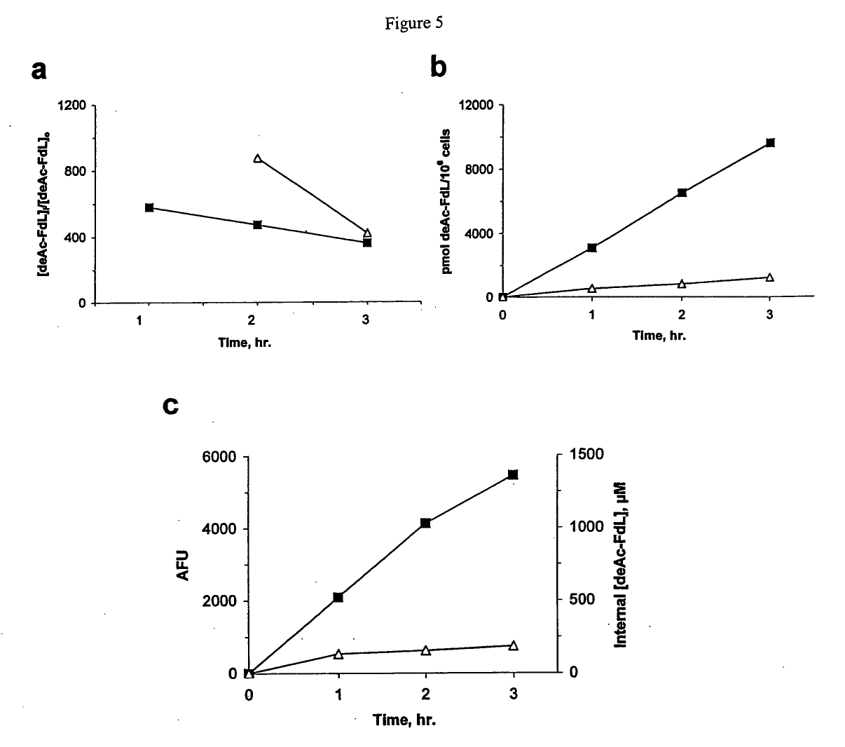

To identify compounds that modulate SIRT1 activity, a number of small molecule libraries were screened using a fluorescent deacetylation assay in 96-well plates²⁶. The substrate used in the assay was a fluorogenic peptide based on the sequence encompassing the p53-K382 acetylation site, a known target of SIRT1 in vivo^{20,21,27}. This substrate was preferred over a variety of other fluorogenic peptide substrates that were based on other known HDAC targets (FIG. 5). The small molecule libraries included analogues of nicotinamide, ε-acetyl lysine, NAD⁺, nucleotides, nucleotide analogues and purinergic ligands. From the initial screen, several sirtuin inhibitors were found (Supplementary Table 7). However, the most striking outcome was the identification of two compounds, quercetin and piceatannol, that stimulated SIRT1 activity five and eight-fold, respectively (Table 1). Both quercetin and piceatannol have been previously identified as protein kinase inhibitors^{28,29}.

Comparison of the structures of the two activating compounds suggested a possible structure-activity relationship. Piceatannol comprises two phenyl groups trans to one another across a linking ethylene moiety. The trans-stilbene ring structures of piceatannol are superimposable on the flavonoid A and B rings of quercetin, with the ether oxygen and carbon-2 of the C ring aligning with the ethylene carbons in piceatannol (see structures, Table 1). Further, the 5, 7, 3′ and 4′ hydroxyl group positions in quercetin can be aligned, respectively, with the 3, 5, 3′ and 4′ hydroxyls of piceatannol.

Given the demonstrated longevity-enhancing effects of sirtuin activity in S. cerevisiae⁷ and C. elegans¹⁹, it was naturally of interest to further explore the structure-activity relationship among compounds that stimulate SIRT1. Both quercetin and piceatannol are polyphenols, members of a large and diverse group of plant secondary metabolites that includes flavones, stilbenes, flavanones, isoflavones, catechins (flavan-3-ols), chalcones, tannins and anthocyanidins^{30,31}. Polyphenols noteworthy with respect to potential longevity-enhancing effects include resveratrol, a stilbene found in red wine and epigallocatechin gallate (EGCG) from green tea. Both have been suggested on the basis of epidemiological and mechanistic investigations to exert cancer chemopreventive and cardioprotective effects³⁰⁻³². Therefore, a secondary screen was performed that encompassed resveratrol, EGCG and additional representatives from a number of the polyphenol classes listed above. The screen emphasized flavones due to the great number of hydroxyl position variants available in this group³¹. The results of this screen are summarized in Supplementary Tables 1-6. In the tables, a “ratio to control rate” above 1 indicates that a compound with such a rate is an activator of the sirtuin tested and a number under 1 indicates that a compound is an inhibitor.

Additional potent SIRT1 activators were found among the stilbenes, chalcones and flavones (Table 1, Supplementary Tables 1 and 2). The six most active flavones had 3′ and 4′ hydroxyls (Supplementary Table 2), although it should be noted that the most active compound overall, resveratrol (3,5,4′-trihydroxystilbene), was more active than piceatannol, which differs only by its additional 3′-hydroxyl (Table 1). The importance of the 4′-hydroxyl to activity is underscored by the fact that each of the 12 most stimulatory flavones share this feature (Supplementary Tables 1 and 2).

Many, but not all of the most active compounds include hydroxyls in the two meta positions (e.g. 5,7-dihydroxylated flavones) of the ring (A ring), trans to that with the 4′ or 3′,4′ pattern (B ring, see Table 1, Supplementary Tables 1 and 2). A potentially coplanar orientation of the trans phenyl rings may be important for activity since catechins and flavanones, which lack the 2,3 double-bond, have weak activity despite having equivalent hydroxylation patterns to various stimulatory flavones (compare Supplementary Tables 2 and 3 with 4 and 5). The absence of activity in the isoflavone genistein, although hydroxylated in an equivalent way to the stimulatory compounds apigenin and resveratrol (see Supplementary Tables 1, 2 and 4), is consistent with the idea that the trans positioning and spacing of the hydroxylated rings contributes strongly to activity.

The biological effects of polyphenols are frequently attributed to antioxidant, metal ion chelating and/or free-radical scavenging activity^{30,32}. The possibility that the apparent polyphenol stimulation of SIRT1 might simply represent the repair of oxidative and/or metal-ion induced damage incurred during preparation of the recombinant protein. Two features of the results argue against this being the case. First, a variety of free-radical protective compounds, including antioxidants, chelators and radical scavengers, failed to stimulate SIRT1 (see Supplementary Table 6.). Second, among various polyphenols of equivalent antioxidant capacity diverse SIRT1 stimulating activity (e.g. compare resveratrol, quercetin and the epicatechins in Supplementary Tables 1, 2 and 5 and see³³) was observed.

### Example 2

### Resveratrol's Effects on SIRT1 Kinetics

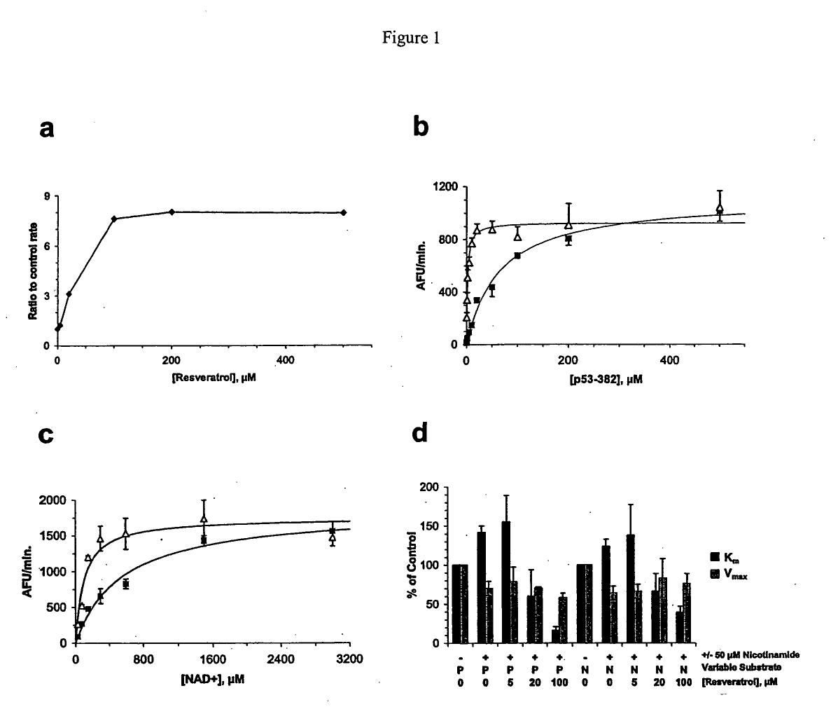

Detailed enzyme kinetic investigations were performed using the most potent activator, resveratrol. Dose-response experiments performed under the conditions of the polyphenol screening assays (25 μM NAD⁺, 25 μM p53-382 acetylated peptide), showed that the activating effect doubled the rate at ˜11 μM and was essentially saturated at 100 λM resveratrol (FIG. 1a). Initial enzyme rates, in the presence or absence of 100 μM resveratrol, were determined either as a function of acetyl-peptide concentration with high NAD⁺ (3 mM NAD⁺, FIG. 1b) or as a function of NAD⁺ concentration with high acetyl-peptide (1 mM p53-382 acetylated peptide, FIG. 1c). Although resveratrol had no significant effect on the two Vₘₐₓ determinations (FIGS. 1b, 1c), it had pronounced effects on the two apparent Kₘs. Its effect on the acetylated peptide Kₘ was particularly striking, amounting to a 35-fold decrease (FIG. 1b). Resveratrol also lowered the Kₘ for NAD⁺ over 5-fold (FIG. 1c). Since resveratrol acts only on Kₘ, it could be classified as an allosteric effector of ‘K system’ type³⁴. This can imply that only the substrate binding affinity of the enzyme has been altered, rather than a rate-limiting catalytic step.

Previous kinetic analysis of SIRT1 and Sir2²⁶ and genetic analysis of Sir2's role in yeast lifespan extension^{6,35} have implicated nicotinamide (a product of the sirtuin reaction) as a physiologically important inhibitor of sirtuin activity. Therefore the effects of resveratrol on nicotinamide inhibition were tested. In experiments similar to those of FIGS. 1b and 1c, kinetic constants in the presence of 50 μM nicotinamide were determined either by varying the concentration of NAD⁺ or that of the p53-382 acetylated peptide (FIG. 1d). Nicotinamide, in contrast to resveratrol, affects the SIRT1 Vₘₐₓ (note 30% and 36% Vₘₐₓ decreases in absence of resveratrol, FIG. 1d and see ref.²⁶). In the presence of 50 μM nicotinamide, resveratrol appears to have complex, concentration-dependent effects on the kinetics of SIRT1 (FIG. 1d). Apparent Kₘ for NAD⁺ and acetylated substrate appear to actually be raised by 5 μM resveratrol when nicotinamide is present. At 20 and 100 μM, in the presence of 50 μM nicotinamide, resveratrol lowers the Kₘ for both NAD⁺ and acetylated peptide, without reversing the nicotinamide-induced Vₘₐₓ decrease. It has been proposed that sirtuins may bind nicotinamide at a second site, known as “the C pocket”, distinct from the “B” site that interacts with the nicotinamide moiety of NAD^{+ 26}. In light of this potential complexity, further kinetic studies, supplemented by structural/crystallographic information, will likely be necessary to fully elucidate the interplay between the effects of nicotinamide and polyphenols.

### Example 3

### Activating Compounds Extend Yeast Lifespan

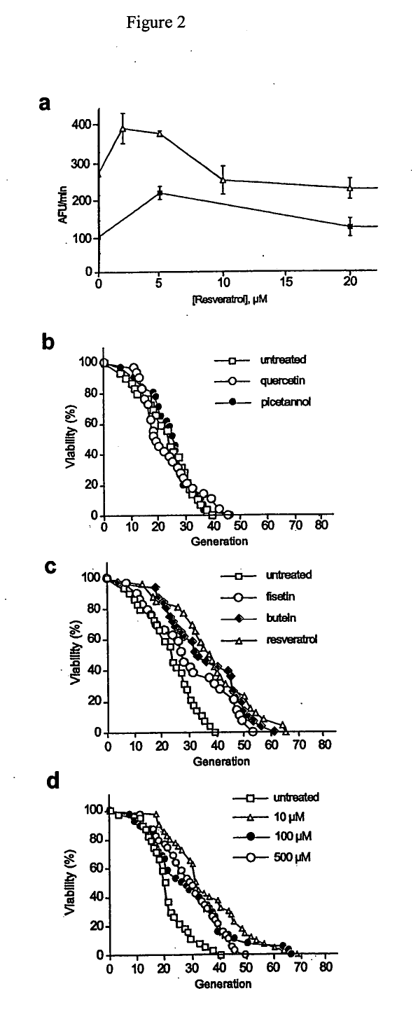

To investigate whether these compounds could stimulate sirtuins in vivo, S. cerevisiae, an organism in which the upstream regulators and downstream targets of Sir2 are relatively well understood, was used. A resveratrol dose-response study of Sir2 deacetylation rates (FIG. 2a) indeed reveals that resveratrol stimulates Sir2 in vitro, with the optimum concentration of activator being 2-5 μM. Levels of activation were somewhat lower than those for SIRT1, and unlike SIRT1, inhibition was seen at concentrations greater than ˜100 μM.

Resveratrol and four other potent sirtuin activators, representatives of the stilbene, flavone, and chalcone families, were tested for their effect on yeast lifespan. Due to the potential impediment by the yeast cell wall or plasma membrane and suspected slow oxidation of the compound in the medium, a concentration (10 μM) was chosen which is slightly higher than the optimal resveratrol concentration in vitro. As shown in FIG. 2b, quercetin and piceatannol had no significant effect on lifespan. In contrast, butein, fisetin and resveratrol increased average lifespan by 31, 55 and 70%, respectively, and all three significantly increased maximum lifespan (FIG. 2c). Concentrations of resveratrol higher than 10 μM provided no added lifespan benefit and there was no lasting effect of the compound on the lifespan of pre-treated young cells (FIG. 2d and data not shown).

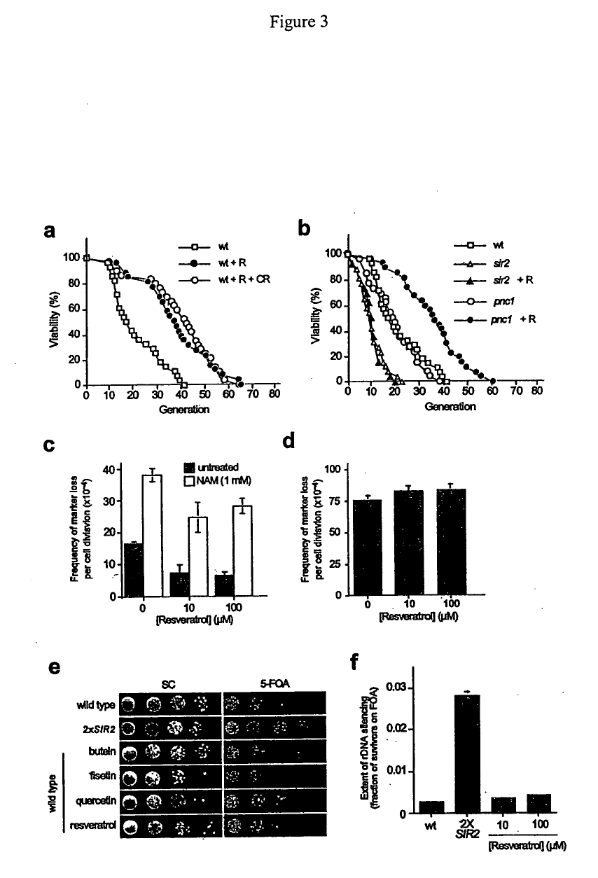

For subsequent yeast genetic experiments resveratrol was used because it was the most potent SIRT1 activator and provided the greatest lifespan extension. Glucose restriction, a form of CR in yeast, resulted in no significant extension of the long-lived resveratrol-treated cells (FIG. 3a), indicating that resveratrol likely acts via the same pathway as CR. Consistent with this, resveratrol had no effect on the lifespan of a sir2 null mutant (FIG. 3b). Given that resveratrol is reported to have fungicidal properties at high concentrations³⁶, and that mild stress can extend yeast lifespan by activating PNC1⁶, it was plausible that resveratrol was extending lifespan by inducing PNC1, rather than acting on Sir2 directly. However, resveratrol extended the lifespan of a pnc1 null mutant nearly as well as it did wild type cells (FIG. 3b). Together these data show that resveratrol acts downstream of PNC1 and requires SIR2 for its effect. Thus, the simplest explanation for these observations is that resveratrol increases lifespan by directly stimulating Sir2 activity.

A major cause of yeast aging is thought to stem from the inherent instability of the repetitive rDNA locus^{2,5,37-39}. Homologous recombination between rDNA repeats can generate an extrachromosomal circular form of rDNA (ERC) that is replicated until it reaches toxic levels in old cells. Sir2 is thought to extend lifespan by suppressing recombination at the replication fork barrier of rDNA⁴⁰. Consistent with the lifespan extension observed for resveratrol, this compound reduced the frequency of rDNA recombination by ˜60% (FIG. 3c), in a SIR2-dependent manner (FIG. 3d). In the presence of the Sir2 inhibitor nicotinamide, recombination was also decreased by resveratrol (FIG. 3c), in agreement with the kinetic data (see FIG. 1d). Interestingly, it was found that resveratrol and other sirtuin activators had only minor effects on rDNA silencing (FIGS. 3e and f). Work is underway to elucidate how these various compounds can differentially affect rDNA stability and silencing.

Another measure of lifespan in S. cerevisiae is the length of time cells can survive in a metabolically active but nutrient deprived state. Aging under these conditions (i.e. chronological aging) is primarily due to oxidative damage⁴¹. Resveratrol (10 μM or 100 μM) failed to extend chronological lifespan (not shown), indicating that the sirtuin-stimulatory effect of resveratrol may be more relevant in vivo than its antioxidant activity^{30,31}.

### Example 4

### Effects of Activators in Human Cells

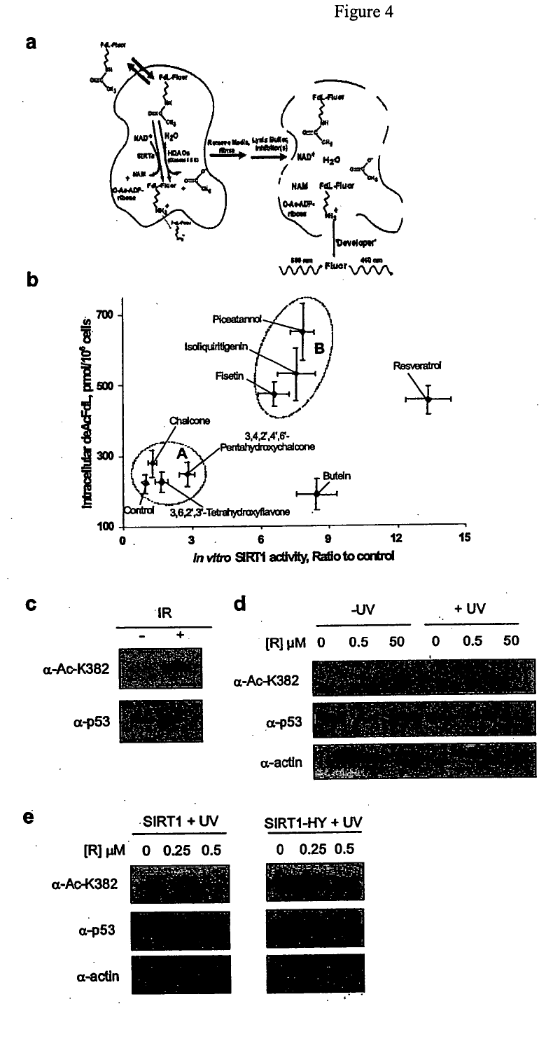

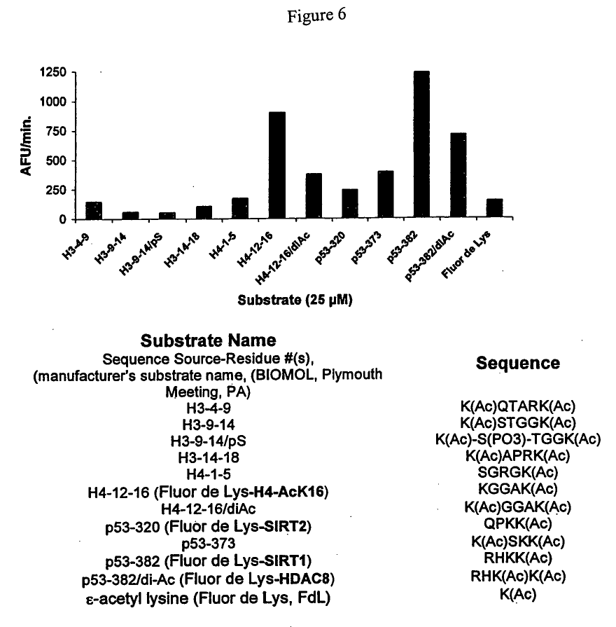

To test whether these compounds could stimulate human SIRT1 in vivo, a cellular deacetylase assay was used. A schematic of the assay procedure is depicted in FIG. 4a. Cells are incubated with media containing the fluorogenic E-acetyl-lysine substrate, ‘Fluor de Lys’ (FdL). This substrate, neutral when acetylated, becomes positively charged upon deacetylation and accumulates within cells (see FIG. 6a). Lysis of the cells and addition of the non-cell-permeable ‘Developer’ reagent releases a fluorophor specifically from those substrate molecules that have been deacetylated (FIG. 4a and see Methods). With HeLa cells growing adherently, 5-10% of the signal produced in this assay is insensitive to 1 μM trichostatin A (TSA), a potent inhibitor of class I and II HDACs but not sirtuins (class III)⁴² (FIGS. 6b and 6c).

A selection of SIRT1-stimulatory and non-stimulatory polyphenols were tested for their effects on this TSA-insensitive signal (FIG. 4b). Cellular deacetylation signals in the presence of each compound (y-axis, FIG. 4b) were plotted against their fold-stimulations of SIRT1 in vitro α-axis, FIG. 4b, data from Supplementary Tables 1-3). For most of the compounds, the in vitro activity roughly corresponded to the cellular signal. Compounds with little or no in vitro activity clustered around the negative control (Group A, FIG. 4b). Another grouping, of strong in vitro activators is clearly distanced from the low activity cluster in both dimensions (Group B, FIG. 4b). A notable outlier was butein, a potent activator of SIRT1 in vitro which had no effect on the cellular signal. With allowances for possible variation among these compounds in properties unrelated to direct sirtuin stimulation, such as cell-permeability and rates of metabolism, these data are consistent with the idea that certain polyphenols can activate native sirtuins in vivo.

One known target of SIRT1 in vivo is lysine 382 of p53. Deacetylation of this residue by SIRT1 decreases the activity and half-life of p53^{20,21,27}. To follow the acetylation status of K382 a rabbit polyclonal antibody was generated that recognizes the acetylated form of K382 (Ac-K382) on Western blots of whole cell lysates. As a control it was shown that the signal was specifically detected in extracts from cells exposed to ionizing radiation (FIG. 4c), but not in extracts from cells lacking p53 or where arginine had been substituted for lysine 382 (data not shown). U2OS osteosarcoma cells were pre-treated for 4 hours with resveratrol (0.5 and 50 μM) and exposed to UV radiation. A marked decrease in the level of Ac-K382 was consistently observed in the presence of 0.5 μM resveratrol, compared to untreated cells (FIG. 4d). At higher concentrations of resveratrol (>50 μM) the effect was reversed (FIG. 4d and data not shown), consistent with previous reports of increased p53 activity at such concentrations⁴³. The ability of low concentrations of resveratrol to promote deacetylation of p53 was diminished in cells expressing a dominant-negative SIRT1 allele (H363Y) (FIG. 4e), demonstrating that SIRT1 is necessary for this effect. This biphasic dose-response of resveratrol could explain the dichotomy in the literature regarding the effects of resveratrol on cell survival^{30,43,44}.

Thus, the first known class of small molecule sirtuin activators has been discovered, all of which are plant polyphenols. These compounds can dramatically stimulate sirtuin activity in vitro and promote effects consistent with increased sirtuin activity in vivo. In human cells, resveratrol promotes SIRT1-mediated p53 deacetylation of K382. In yeast, the effect of resveratrol on lifespan is as great as any longevity-promoting genetic manipulation⁶ and has been linked convincingly to the direct activation of Sir2. The correlation between lifespan and rDNA recombination, but not silencing, adds to the body of evidence that yeast aging is due to DNA instability^{2,5,37-39} not gene dysregulation⁴⁵.

How can the activation of the yeast and human sirtuins by so many plant metabolites be explained? Sirtuins have been found in diverse eukaryotes, including fungi, protozoans, metazoans and plants^{46,47}, and likely evolved early in life's history¹. Plants are known to produce a variety of polyphenols, including resveratrol, in response to stresses such as dehydration, nutrient deprivation, UV radiation and pathogens^{48,49}. Therefore it is plausible that these compounds may be synthesized to regulate a sirtuin-mediated plant stress response. This would be consistent with the recently discovered relationship between environmental stress and Sir2 activity in yeast⁶. Perhaps these compounds have stimulatory activity on sirtuins from fungi and animals because they mimic an endogenous activator, as is the case for the opiates/endorphins, cannabinols/endocannabinoids and various polyphenols with estrogen-like activity^{30,31}. Alternatively, animal and fungal sirtuins may have retained or developed an ability to respond to these plant metabolites because they are a useful indicator of a deteriorating environment and/or food supply.

### Example 5

### Materials and Methods for Examples 1-4

Compound Libraries and Deacetylation Assays

His₆-tagged recombinant SIRT1 and GST-tagged recombinant Sir2 were prepared as previously described²⁶. From 0.1 to 1 μg of SIRT1 and 1.5 μg of Sir2 were used per deacetylation assay (in 50 μl total reaction) as previously described²⁶. SIRT1 assays and certain of those for Sir2 employed the p53-382 acetylated substrate (‘Fluor de Lys-SIRT1’, BIOMOL) rather than FdL.

Themed compound libraries (BIOMOL) were used for primary and secondary screening. Most polyphenol compounds were dissolved at 10 mM in dimethylsulfoxide (DMSO) on the day of the assay. For water soluble compounds and negative controls, 1% v/v DMSO was added to the assay. In vitro fluorescence assay results were read in white ½-volume 96-well microplates (Corning Costar 3693) with a CytoFluor™ II fluorescence plate reader (PerSeptive Biosystems, Ex. 360 nm, Em. 460 nm, gain=85). HeLa cells were grown and the cellular deacetylation assays were performed and read, as above, but in full-volume 96-well microplates (Corning Costar 3595). Unless otherwise indicated all initial rate measurements were means of three or more replicates, obtained with single incubation times, at which point 5% or less of the substrate initially present had been deacetylated. Calculation of net fluorescence increases included subtraction of a blank value, which in the case of Sir2 was obtained by omitting the enzyme from the reaction and in the case of SIRT1 by adding an inhibitor (200 μM suramin or 1 mM nicotinamide) to the reaction prior to the acetylated substrate. A number of the polyphenols partially quenched the fluorescence produced in the assay and correction factors were obtained by determining the fluorescence increase due to a 3 μM spike of an FdL deacetylated standard (BIOMOL, catalog number KI-142). All error bars represent the standard error of the mean.

Media and Strains

All yeast strains were grown at 30° C. in complete yeast extract/bactopeptone, 2.0% (w/v) glucose (YPD) medium except where stated otherwise. Calorie restriction was induced in 0.5% glucose. Synthetic complete (SC) medium consisted of 1.67% yeast nitrogen base, 2% glucose, 40 mg/litre each of auxotrophic markers. SIR2 was integrated in extra copy and disrupted as described⁵. Other strains are described elsewhere²⁶. For cellular deacetylation assays, HeLa S3 cells were used. U2OS osteosarcoma and human embryonic kidney (HEK 293) cells were cultured adherently in Dulbecco's Modified Eagle's Medium (DMEM) containing 10% fetal calf serum (FCS) with 1.0% glutamine and 1.0% penecillin/streptomycin. HEK 293 overexpressing dominant negative SIRT1 H363Y was a gift of R. Frye (U. Pittsburgh).

Lifespan Determinations

Lifespan measurements were performed using PSY316AT MATα as previously described³⁵. All compounds for lifespan analyses were dissolved in 95% ethanol and plates were dried and used within 24 hours. Prior to lifespan analysis, cells were pre-incubated on their respective media for at least 15 hours. Following transfer to a new plate, cells were equilibrated on the medium for a minimum of 4 hours prior to micro-manipulating them. At least 30 cells were examined per experiment and each experiment was performed at least twice. Statistical significance of lifespan differences was determined using the Wilcoxon rank sum test. Differences are stated to be significant when the confidence is higher than 95%.

Silencing and Recombination Assays

Ribosomal DNA silencing assays using the URA3 reporters were performed as previously described²⁶. Ribosomal DNA recombination frequencies were determined by plating W303AR cells³⁷ on YPD medium with low adenine/histidine and counting the fraction of half-red sectored colonies using Bio-Rad Quantity One software as previously described³⁵. At least 6000 cells were analyzed per experiment and all experiments were performed in triplicate. All strains were pre-grown for 15 hours with the relevant compound prior to plating.

Proteins and Western Analyses

Recombinant Sir2-GST was expressed and purified from E. coli as previously described except that lysates were prepared using sonication²⁶. Recombinant SIRT1 from E. coli was prepared as previously described²⁶. Polyclonal antiserum against p53-AcK382 was generated using an acetylated peptide antigen as previously described²⁰, with the following modifications. Anti-Ac-K382 antibody was affinity purified using non-acetylated p53-K382 peptides and stored in PBS at −70° C. and recognized an acetylated but not a non-acetylated p53 peptide. Western hybridizations using anti-acetylated K382 or anti-actin (Chemicon) antibody were performed at 1:1000 dilution of antibody. Hybridizations with polyclonal p53 antibody (Santa Cruz Biotech.) used 1:500 dilution of antibody. Whole cell extracts were prepared by lysing cells in buffer containing 150 mM NaCl, 1 mM MgCl₂, 10% glycerol, 1% NP40, 1 mM DTT and anti-protease cocktail (Roche).

### REFERENCES FOR EXAMPLES 1-4 AND BACKGROUND

1. Kenyon, C. Cell 105, 165-168 (2001).
2. Sinclair, D. A. Mech Ageing Dev 123, 857-67 (2002).
3. Hekimi, S. & Guarente Science 299, 1351-4 (2003).
4. Guarente, L. & Kenyon, C. Nature 408, 255-62. (2000).
5. Lin et al. Science 289, 2126-8. (2000).
6. Anderson et al. Nature 423, 181-5 (2003).
7. Kaeberlein et al. Genes Dev 13, 2570-80. (1999).
8. Landry et al. Proc Natl Acad Sci USA 97, 5807-11. (2000).
9. Imai et al. Nature 403, 795-800 (2000).
10. Smith et al. Proc Natl Acad Sci USA 97, 6658-63. (2000).
11. Tanner et al. Proc Natl Acad Sci USA 97, 14178-82. (2000).
12. Tanny et al. Cell 99, 735-45. (1999).
13. Tanny et al. Proc Natl Acad Sci USA 98, 415-20. (2001).
14. Laurenson et al. Microbiol Rev 56, 543-60. (1992).
15. Smith et al. Genes Dev 11, 241-54. (1997).
16. Bryk, M. et al. Genes Dev 11, 255-69. (1997).
17. Gottlieb et al. Cell 56, 771-6. (1989).
18. Aguilaniu et al. Science (2003).
19. Tissenbaum et al. Nature 410, 227-30. (2001).
20. Vaziri et al. Cell 107, 149-59. (2001).
21. Luo et al. Cell 107, 137-48. (2001).
22. Vergnes et al. Gene 296, 139-50 (2002).
23. Holzenberger et al. Nature 421, 182-7 (2003).
24. Shimokawa et al. Faseb J 17, 1108-9 (2003).
25. Tatar et al. Science 299, 1346-51 (2003).
26. Bitterman et al. J Biol Chem 277, 45099-107. (2002).
27. Langley et al. EMBO J 21, 2383-2396 (2002).
28. Glossmann et al. Naunyn Schmiedebergs Arch Pharmacol 317, 100-2 (1981).
29. Oliver et al. J Biol Chem 269, 29697-703 (1994).
30. Ferguson et al. Mutat Res 475, 89-111 (2001).
31. Middleton et al. Pharmacol Rev 52, 673-751 (2000).
32. Jang et al. Science 275, 218-20 (1997).
33. Stojanovic et al. Arch Biochem Biophys 391, 79-89 (2001).
34. Monod et al. J. Mol. Biol. 12, 88-118 (1965).
35. Anderson et al. J Biol Chem 277, 18881-90. (2002).
36. Pont et al. J Phytopathol 130, 1-8 (1990).
37. Sinclair et al. Cell 91, 1033-42. (1997).
38. Defossez et al. Mol Cell 3, 447-55 (1999).
39. Park et al. Mol Cell Biol 19, 3848-56 (1999).
40. Benguria et al. Nucleic Acids Res 31, 893-8 (2003).
41. Longo et al. Science 299, 1342-6 (2003).
42. Denu et al. Trends Biochem Sci 28, 41-8 (2003).
43. Dong et al. Mutat Res 523-524, 145-50 (2003).
44. Nicolini et al. Neurosci Lett 302, 41-4 (2001).
45. Jazwinski, S. M. et al. Ann N Y Acad Sci 908, 21-30 (2000).
46. Pandey et al. Nucleic Acids Res 30, 5036-55 (2002).
47. Frye, R. A. Biochem Biophys Res Commun 273, 793-8. (2000).
48. Soleas et al. Clin Biochem 30, 91-113 (1997).
49. Coronado et al. Plant Physiol 108, 533-542 (1995).
50. Masoro, E. J. Exp Gerontol 35, 299-305. (2000).

### Example 6

### Localization of the Activation Domain of Sirtuins to their N-Terminus

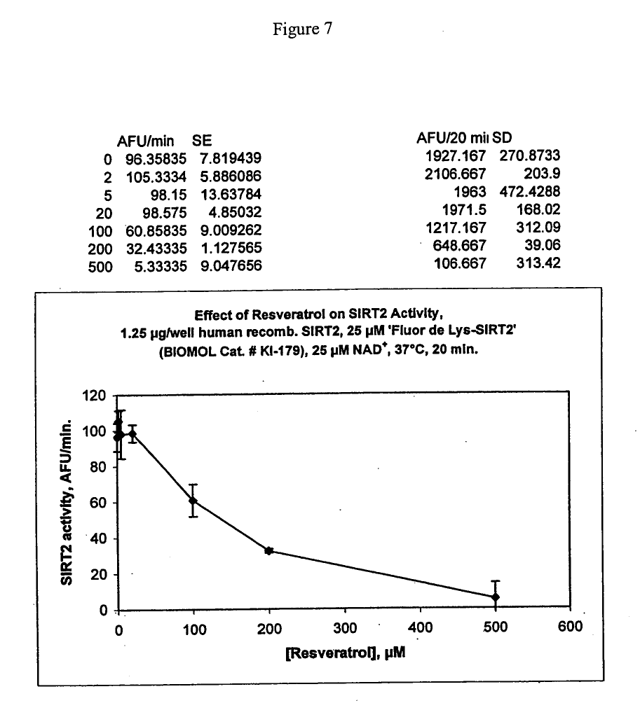

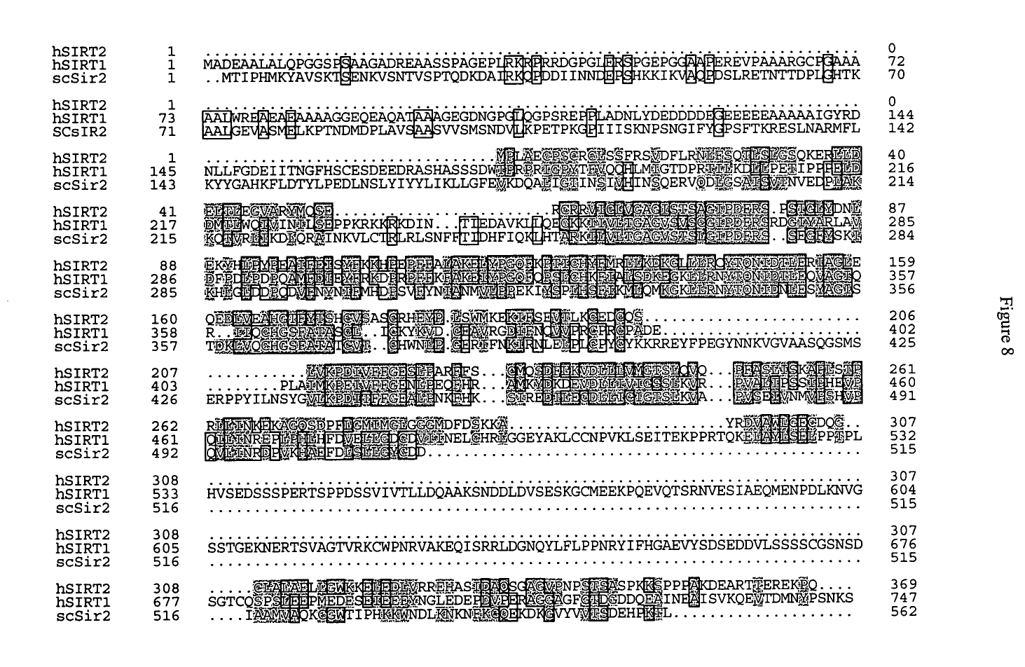

Yeast Sir2 and human SIRT1 are very homologous and differ from human SIRT2 by the addition of an N-terminal domain that is absent in SIRT2. The effect of resveratrol was assayed on human recombinant SIRT2 as follows. Human recombinant SIRT2 was incubated at a concentration of 1.25 μg/well with 25 μM of Fluor de Lys-SIRT2 (BIOMOL cat. # KI-179) and 25 μM NAD+ for 20 minutes at 37° C., as described above. The results, which are shown in FIG. 7, indicate that, in contrast to SIRT1, increasing concentrations of resveratrol decrease SIRT2 activity. Thus, based on the difference in structure of SIRT1 and SIRT2, i.e., the absence of an N-terminal domain (see FIG. 8), it is believed that the N-terminal domain of SIRT1 and Sir2 is necessary for activation by the compounds described herein. In particular, it is likely that the activator compounds described herein interact with the N-terminal portion of sirtuins. The N-terminal portion of SIRT1 that is necessary for the action of the compounds is from about amino acid 1 to about amino acid 176, and that of Sir2 is from about amino acid 1 to about amino acid 175.

### Example 7

### Resveratrol Extends the Lifespan of C. elegans

Fifty C. elegans worms (strain N2) were grown in the presence or absence of 100 μM resveratrol for 17 days. On day 17, only 5 worms in the control group without resveratrol were alive, whereas 17 worms were alive in the group that was treated with resveratrol. Thus, the presence of resveratrol in the growth media of C. elegans extends their lifespan.

### Example 8

### Identification of Additional Activators of Sirtuins

Using the screening assay described in Example 1, five more sirtuin activators have been identified. These are set forth in supplementary Table 8.

### Example 9

### Identification of Inhibitors of Sirtuins

Using the screening assay described in Example 1, more inhibitors were identified. These are set forth in the appended supplementary Table 8, and correspond to the compounds having a ratio to control rate of less than 1.

### Example 10

### Identification of Further Activators and Inhibitors of Sirtuins

Additional activators and inhibitors of sirtuins were identified, and are listed in Tables 9-13. In these Tables, “SE” stands for Standard error of the mean and N is the number of replicates used to calculate mean ratio to the control rate and standard error.

All SIRT1 rate measurements used in the calculation of “Ratio to Control Rate” were obtained with 25 μM NAD⁺ and 25 μM p53-382 acetylated peptide substrate were performed as described above and in K. T. Howitz et al. Nature (2003) 425: 191. All ratio data were calculated from experiments in which the total deacetylation in the control reaction was 0.25-1.25 μM peptide or 1-5% of the initial concentration of acetylated peptide.

Stability determinations (t_{1/2}) derived from SIRT1 rate measurements performed in a similar way to those described above, except that 5 μM p53-382 acetylated peptide substrate was used rather than 25 μM. The fold-stimulation (ratio to control) obtained with a compound diluted from an aged stock solution was compared to an identical dilution from a stock solution freshly prepared from the solid compound. “t_{1/2}” is defined as the time required for the SIRT1 fold-stimulation of the compound from the aged solution to decay to one-half of that obtained from a freshly prepared solution. Ethanol stocks of resveratrol, BML-212 and BML-221 were prepared at 2.5 mM and the compounds were assayed at a final concentration of 50 μM. The water stock of resveratrol was 100 μM and the assay performed at 10 μM. Stocks were aged by storage at room temperature, in glass vials, under a nitrogen atmosphere.

The effect of some of these compounds on lifespan was determined in yeast and C. elegans, as described above. The results are set forth below in Table 19:

| | % change in yeast | % change in C. elegans | |
|---|---|---|---|
| | replicative lifespan | lifespan relative | |
| | relative to untreated | to untreated organisms | |
| Compound | organisms (10 μM)^{a} | (100/500 μM)^{b} | |
| untreated | 100% | 100% | |
| Resveratrol | 170-180% | 110% | |
| 3,5,4′-Trihydroxy- | | | |
| trans-stilbene | | | |
| Pinosylvin | 114% | ND | |
| 3,5-Dihydroxy- | | | |
| trans-stilbene | | | |
| BML-212 | 98% | ND | |
| 3,5-Dihydroxy-4′- | | | |
| fluoro-trans- | | | |
| stilbene | | | |
| BML-217 | 90% | ND | |
| 3,5-Dihydroxy-4′- | | | |
| chloro-trans- | | | |
| stilbene | | | |
| BML-221 | 165% | >100% | (ongoing) |
| 3,4′-Dihydroxy-5- | | | |
| acetoxy-trans- | | | |
| stilbene | | | |
| BML-233 | ND | 70% | (10) |
| 3,5-Dihydroxy-4′- | | 50% | (500) |
| methoxy-trans- | | | |
| stilbene | | | |

^{a}Replicative lifespans performed using 2% (w/v) glucose standard yeast compete medium (YPD) under standard conditions.
^{b}Lifespan assays performed on N2 worms using E. coli as food under standard conditions.
ND. Not determined.

The results indicate that resveratrol significantly extends lifespan in yeast and in C. elegans. Since BML-233 was shown to be a strong activator of sirtuins (see above), the results obtained in C. elegans may indicate that the compound is toxic to the cells.

Without wanting to be limited to particular structures, it appears that the following structure/activity relationships exist. SIRT1 activation results from several of these new analogs confirmed the importance of planarity, or at least the potential for planarity, between and within the two rings of the active compounds. Reduction of the double bond of the ethylene function, between, the two rings essentially abolishes activity (compare Resveratrol, Table A and Dihydroresveratrol, Table E). Replacement of a phenyl moiety with a cyclohexyl group is nearly as detrimental to SIRT1 stimulating activity (compare Pinosylvin, Table 9 and BML-224, Table 12). Amide bonds are thought to have a partially double bond character. However, replacement of the ethylene function with a carboxamide abolished activity (compare Pinosylvin, Table 9, with BML-219, Table 13). It is possible that this effect could be due in part to the position that carbonyl oxygen must assume in the conformation that places the two rings trans to one another. If so, a compound in which the positions of the amide nitrogen and carbonyl are reversed might be expected to have greater activity.

In twelve of the analogs resveratrol's 4′-hydroxy was replaced with various functionalities (see Tables 9 and 10, BML-221 in Table 11, BML-222 in Table 12). Although none of the replacements tried led to substantial increases in SIRT1 stimulating activity, this parameter was, in general, remarkably tolerant of substitutions at this position. Small groups (Hin Pinosylvin, Cl— in BML-217, —CH₃ in BML-228) did the least to decrease activity. There is some evidence of a preference in the enzyme's stilbene binding/activation site for unbranched (ethyl in BML-225, azido in BML-232, —SCH₃ in BML-230) and hydrophobic functions (compare isopropyl in BML-231 to acetoxy in BML-221, acetamide in BML-222). Solution stability relative to resveratrol was strongly increased by one of the two 4′-substitutions (acetoxy, BML-221) tested for this so far.

Resveratrol is currently one of the most potent known activator of SIRT1. The collection of analogs described above, particularly the group entailing substitutions at the 4′ position, may be instrumental in informing the design of new SIRT1 ligands with improved pharmacological properties. One parameter that may be of interest in this regard is stability. One 4′-substituted analog, BML-221, displays a vast improvement in solution stability relative to resveratrol and although diminished in in vitro SIRT1 activating ability, retains much of resveratrol's biological activity (see lifespan data). The 4′-hydroxyl of resveratrol is thought to be of primary importance to resveratrol's free-radical scavenging reactivity (S. Stojanovic et al. Arch. Biochem. Biophys. 2001 391 79). Most of the 4′-substituted analogs have yet to be tested for solution stability, but if resveratrol's instability in solution is due to redox reactivity, many of the other analogs would be expected to also exhibit improved stability.

The results obtained with 4′-substituted analogs may indicate promising routes to explore while seeking to increase SIRT1 binding affinity. For example, the efficacy of the 4′-ethyl compound (BML-225) might indicate the presence of a narrow, hydrophobic binding pocket at the SIRT1 site corresponding to the 4′ end of resveratrol. Several new series of 4′-substituted analogs are planned, the simplest comprising straight-chain aliphatic groups of various lengths.

### Example 11

### Methods of Synthesis of the Compounds in Tables 9-13

Most of the resveratrol analogs were synthesized by the same general procedure, from a pair of intermediates, a benzylphosphonate and an aldehyde. The synthesis or sources of these intermediates are described in section II. Section III. describes the procedures for synthesizing the final compounds from any of the benzylphosphonate/aldehyde pairs. The coupling reaction (Section III. A.) is followed by one of two deprotection reactions depending on whether the intermediates contained methoxymethyl (Section III. B.) or methoxy (Section III. C.) protecting groups. Section IV corresponds to Tables 14-18, which list the particular benzylphosphonate and aldehyde used in the synthesis of particular final compounds. Seven of the compounds—Resveratrol, 3,5-Dihydroxy-4′-methoxy-trans-stilbene, Rhapontin aglycone, BML-227, BML-221, Dihydroresveratrol, BML-219—were not synthesized by the general procedure and “N/A” appears next to their entries in the table. Resveratrol was from BIOMOL and the syntheses of the remaining compounds are described in Section V.

II. Synthetic Intermediates

A. Benzylphosphonates (Synthesized)

Synthesis of Diethyl 4-Acetamidobenzylphosphonate: To diethyl 4-aminobenzylphosphonate in 1:1 methylene chloride/pyridine was added catalytic DMAP and acetic anhydride (1.1 eq.). After 3 hours, the reaction was evaporated to dryness and purified via flash chromatography (silica gel).

Synthesis of Diethyl 4-Methylthiobenzylphosphonate: 4-Methylthiobenzyl chloride was heated with triethylphosphite (as solvent) at 120° C. overnight. Excess triethyl phosphite was distilled off under high vacuum and heat. Flash chromatography (silica gel) yielded the desired product.

Synthesis of Diethyl 3,5-Dimethoxybenzylphosphonate: From 3-5-Dimethoxybenzyl bromide. See synthesis of Diethyl 4-Methylthiobenzylphosphonate.

Synthesis of Diethyl 4-Fluorobenzylphosphonate: From 4-Fluorobenzylphosphonate. See synthesis of Diethyl 4-Methylthiobenzylphosphonate.

Synthesis of Diethyl 4-azidobenzylphosphonate: To diethyl 4-aminobenzylphosphonate in acetonitrile (2.5 mL) at 0° C. was added 6M HCl (1 mL). Sodium nitrite (1.12 eq.) in water (1 mL) was added drop wise and the resulting solution stirred at 0° C. for 30 mins. Sodium azide (8 eq.) in water (1 mL) added drop wise (bubbling) and the solution stirred at 0° C. for 30 mins., then at room temperature for 1 hour. The reaction was diluted with ethyl acetate and washed with water and brine and dried over sodium sulfate. Flash chromatography (silica gel) yielded the desired product.

B. Aldehydes (Synthesized)

Synthesis of 3,5-Dimethoxymethoxybenzaldehyde: To 3,5-dihydroxybenzaldehyde in DMF at 0° C. was added sodium hydride (2.2 eq.). The reaction was stirred for 30 min. at 0° C. Chloromethylmethyl ether (2.2 eq.) was added neat, drop wise and the reaction allowed to warm to room temperature over 1.5 hrs. The reaction mixture was diluted with diethyl ether and washed with water (2×) and brine (1×) and dried over sodium sulfate. Flash chromatography (silica gel) yielded the desired product.

C. Purchased Intermediates: Unless Listed Above, All Synthetic Intermediates were Purchase from Sigma-Aldrich.

III. General Procedure for the Synthesis of Resveratrol Analogues

A. Benzylphosphonate/Aldehyde Coupling Procedure

To the appropriate benzylphosphonate (1.2 eq.) in dimethylformamide (DMF) at room temperature was added sodium methoxide (1.2 eq.). This solution was allowed to stir at room temperature for approximately 45 minutes. The appropriate aldehyde (1 eq.) was then added (neat or in a solution of dimethylformamide). The resulting solution was then allowed to stir overnight at room temperature. Thin layer chromatography (TLC) was used to determine completeness of the reaction. If the reaction was not complete, the solution was heated at 45-50° C. until complete. The reaction mixture was poured into water and extracted with ethyl acetate (2×). The combined organic layers were washed with brine and dried over sodium sulfate. Flash chromatography (silica gel) yielded the desired products.

B. General Procedure for the Deprotection of Methoxymethylresveratrol Analogues

To the appropriate methoxymethylstilbene derivative in methanol was added two drops of concentrated HCl. The resulting solution was heated overnight at 50° C. The solution was evaporated to dryness upon completion of the reaction. Flash chromatography (silica gel) yielded the desired product.

C. General Procedure for the Deprotection of Methoxyresveratrol Analogues

To the appropriate methoxystilbene derivative in methylene chloride was added tetrabutylammonium iodide (1.95 eq. per methoxy group). The reaction was cooled to 0° C. and boron trichloride (1 M in methylene chloride; 2 eq. per methoxy group) was added dropwise. Following the addition of boron trichloride, the cooling bath was removed and the reaction allowed to stir at room temperature until complete (as indicated by TLC). Saturated sodium bicarbonate solution was added and the reaction vigorously stirred for 1 hour. The reaction was poured into cold 1M HCl and extracted with ethyl acetate (3×). The combined organic layers were washed with water (1×) and brine (1×) and dried over sodium sulfate. Flash chromatography (silica gel) yielded the desired products.

V. Special Syntheses

Synthesis of BML-219 (N-(3,5-Dihydroxyphenyl)benzamide): To benzoyl chloride (1 eq.) in dry methylene chloride at room temperature was added triethylamine (1.5 eq.) and a catalytic amount of DMAP followed by 3,5-dimethoxyaniline (1 eq.). The reaction was allowed to stir overnight at room temperature. Upon completion, the reaction was diluted with ethyl acetate and washed with 1M HCl, water and brine and dried over sodium sulfate. Flash chromatography (silica gel) yielded the methoxystilbene derivative. To the methoxystilbene in dry methylene chloride at 0° C. was added tetrabutylammonium iodide (3.95 eq.) followed by boron trichloride (4 eq.; 1M in methylene chloride). Upon completion of the reaction (TLC), saturated sodium bicarbonate was added and the mixture was vigorously stirred for 1 hour. The reaction was diluted with ethyl acetate and washed with 1M HCl and brine and dried over sodium sulfate. Flash chromatography (silica gel) yielded the desired product.

Synthesis of BML-220 (3,3′,5-trihydroxy-4′-methoxystilbene): To Rhapontin in methanol was added catalytic p-toluenesulfonic acid. The reaction was refluxed overnight. Upon completion of the reaction (TLC), the reaction mixture was evaporated to dryness and taken up in ethyl acetate. The organics were washed with water and brine and dried over sodium sulfate. Flash chromatography (silica gel) yielded the desired product.

Synthesis of BML-233 (3,5-Dihydroxy-4′-methoxystilbene): To deoxyrhapontin in methanol was added catalytic p-toluenesulfonic acid. The reaction was refluxed overnight. Upon completion of the reaction (TLC), the reaction mixture was evaporated to dryness and taken up in ethyl acetate. The organics were washed with water and brine and dried over sodium sulfate. Flash chromatography (silica gel) yielded the desired product.

Synthesis of BML-221 and 227 (4′ and 3 monoacetylresveratrols): To resveratrol in tetrahydrofuran at room temperature was added pyridine (1 eq.) followed by acetic anhydride (1 eq.). After stirring for 48 hrs., another 0.25 eq. acetic anhydride added followed by 24 hrs. of stirring. The reaction was diluted with methylene chloride (reaction was not complete) and washed with cold 0.5M HCl, water and brine. Organics were dried over sodium sulfate. Flash chromatography yielded a mixture of 4′- and 3-acetyl resveratrols. Preparative HPLC yielded both monoacetyl resveratrols.

Synthesis of Dihydroresveratrol: To resveratrol in argon-purged ethyl acetate in a Parr shaker was added 10% palladium on carbon (10 wt %). The mixture was shaken under an atmosphere of hydrogen (30 psi) for 5 hours. Filtration through a pad of celite yielded the desired material.

### Example 12

### Dose-Response Analysis of SIRT1 Deacetylation by Resveratrol and BML-230

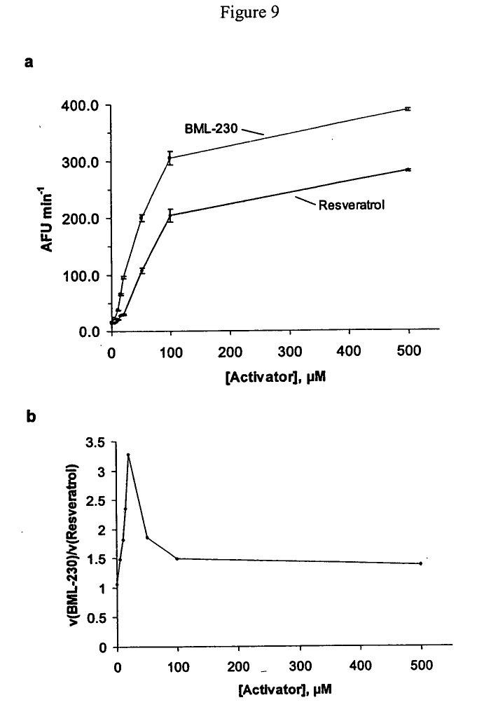

SIRT1 initial rates as a function of activator concentration were determined at 25 μM each of NAD⁺ and p53-382 acetylated peptide, with 20 minutes incubations. Plots of the dose responses of SIRT1 to BML-230 and resveratrol show that the BML-230-stimulated activity exceeds that stimulated by resveratrol at all concentrations tested (FIG. 9a). This could be due to a greater binding affinity of SIRT1 for BML-230, greater activity of the SIRT1/BML-230 complex or some combination of the two. A plot of the ratio of the rates of BML-230-stimulated enzyme to that of resveratrol-stimulated enzyme suggests that increased binding affinity does contribute to the improvement in activity of BML-230 (FIG. 9b). A simple two state model of the binding and activation process assumes that the observed rate (v) is the sum of the fractional contributions of the unliganded and liganded enzymes, where v₀ is the unstimulated rate, v, is the rate of the enzyme with bound ligand-1 (L1) and K_{L1} is the dissociation constant of the enzyme/ligand-1 complex:
v=v₀(1−[L1]/(K_{L1}+[L1]))+v₁(−[L1]/(K_{L1}+[L1])
A similar equation can be prepared for ligand-2 and the ratio (R) of the two rates calculated, an equation which will include, given the conditions of FIG. 9, the substitution [L]=[L1]=[L2]. It can be shown that if the two ligand dissociation constants were equal (K_{L1}=K_{L2}=K_{L}), this ratio would be:
R=(v₀K_{L}+v₁[L])/(v₀K_{L}+v₂[L])
If K_{L1}≠K_{L2}, this ratio would instead be:
R=(v₁[L]²+(v₀K_{L1}+v₁K_{L2})[L]+v₀K_{L1}K_{L2})/(v₂[L]²+(v₀K_{L2}+v₂K_{L1})[L]+v₀K_{L1}K_{L2})
In the first case the plot of R vs. [L] would be a simple hyperbola that monotonically approaches v₁/v₂ as [L] increases. In the second case, as in FIG. 9b, the plot would pass through a maximum before approaching v₁/v₂ at higher [L] values. The data of FIG. 9b would imply that v₁/v₂ (rate for pure SIRT1/BML-230 divided by that for pure SIRT1/resveratrol) is no more than ˜1.4 (R at 500 μM) and that the SIRT1/BML-230 complex indeed has a lower dissociation constant than SIRT1/resveratrol (K_{L1}<K_{L2}).

One of the difficulties in the use of resveratrol as a pharmacologic agent is the relatively low serum concentrations of the aglycone form that can be achieved and maintained when it is administered orally (<<1 μM; see for example D. M Goldberg et al. Clin. Biochem. 2003 36 79). Increasing the SIRT1 binding affinity of synthetic derivatives will improve this aspect of the drug. As sest forth above, various replacements of the resveratrol 4′-hydroxyl, e.g. the H— of pinosylvin or Cl— of BML-217, did not significantly diminish the SIRT1 activating effect. The results obtained with BML-230 indicate that it will be possible to actually increase SIRT1/activator binding affinity by modifications at that site. The 4′-thiomethyl of BML-230 therefore represents a new starting point in seeking further improvements in SIRT1 binding affinity by the synthesis of related derivatives (e.g. 4′-thioethyl etc.).

### Example 13

### Survival Rates

Human 293 were grown to exponential phase under standard conditions and subjected to a dose of compound (50 micromolar) for 96 hours. The number of live cells each time point was counted using a Coulter counter.

TABLE 24
Survival statistics of 293 cells:

| Time | Resvera- | Thio-Methyl | Ethyl | Methyl | Isopropyl |
|---|---|---|---|---|---|
| (h) | trol | BML-230 | BML-225 | BML-228 | BML-231 |
| 0 | 100% | 100% | 100% | 100% | 100% |
| 48 | 5% | 55% | 5% | 46% | 0% |
| 96 | 0% | 57% | 8% | 32% | 0% |

The results indicate that thiomethyl (BML-230) was the least toxic on 293 cells.

### Example 14

### Sirtuin Activators Mimic Calorie Restriction and Delay Aging in Metazoans

Caloric restriction (CR) extends lifespan in numerous species. In the budding yeast S. cerevisiae, this effect requires Sir2¹, a member of the sirtuin family of NAD⁺-dependent deacetylases^{2,3}. Sirtuin activating compounds (STACs) can promote the survival of human cells and extend the replicative lifespan of yeast⁴. Here it is shown that resveratrol and other STACs activate sirtuins from Caenorhabditis elegans and Drosophila melanogaster and extend the lifespan of these animals up to 29% without reducing fecundity. Lifespan extension is dependent on functional Sir2 and is not observed when nutrients are restricted. Together these data indicate that STACs slow metazoan ageing by mechanisms related to CR.

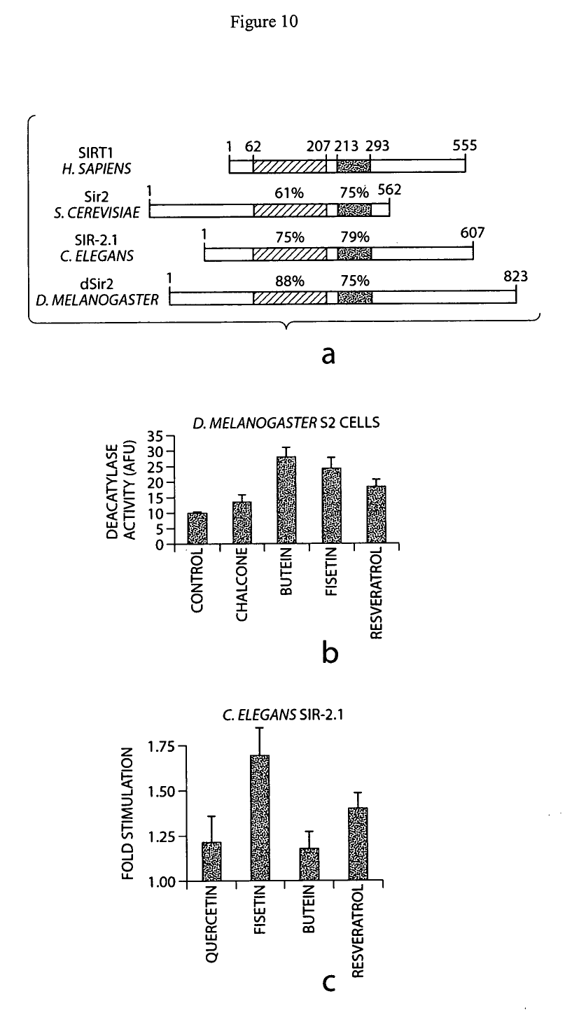

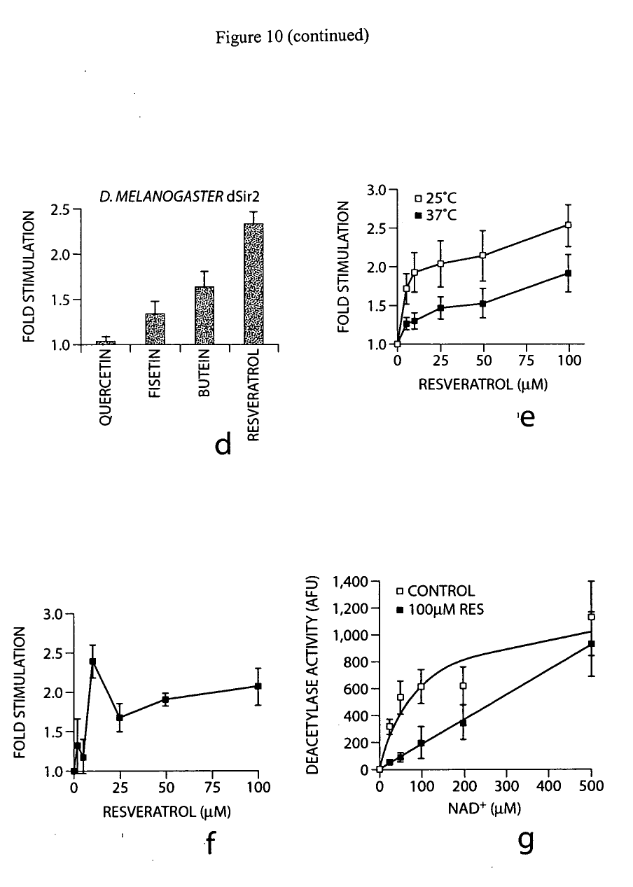

Sir2-like proteins (sirtuins) are a family of NAD⁺-dependent deacetylases conserved from E. coli to humans⁵⁻⁹ (FIG. 10a) that play important roles in gene silencing, DNA repair, rDNA recombination and ageing in model organisms^{2,10-12}. When diet is restricted (calorie restriction, CR), lifespan is extended in diverse species, suggesting there is a conserved mechanism for nutrient regulation of ageing¹³⁻¹⁷. In budding yeast, extra copies this gene extend lifespan by 30% apparently by mimicking CR^{1,18}. A group of compounds (STACs) was recently described that stimulate the catalytic activity of yeast and human sirtuins, and extend the replicative lifespan of yeast cells up to 60%⁴.

To establish whether STACs could activate sirtuins from multicellular animals, a cell-based deacetylation assay was developed for D. melanogaster S2 cells. Several classes of polyphenolic STACs, including chalcones, flavones and stilbenes, increased the rate of deacetylation in an NAD⁺-dependent manner (FIG. 10b). To determine whether this activity was due to direct stimulation of a Sir2 homolog, recombinant SIR-2.1 of C. elegans and dSir2 of D. melanogaster was purified and the effect of various STACs on enzymatic activity in vitro was determined (FIG. 10c, d). In a dose-dependent manner, resveratrol stimulated deacetylation up to 2.5-fold for SIR-2.1 (FIG. 10e) and 2.4-fold for dSir2 (FIG. 10f). As previously observed with the yeast and human Sir2 enzymes, resveratrol lowered the Kₘ of SIR-2.1 for the co-substrate NAD+(FIG. 10g).

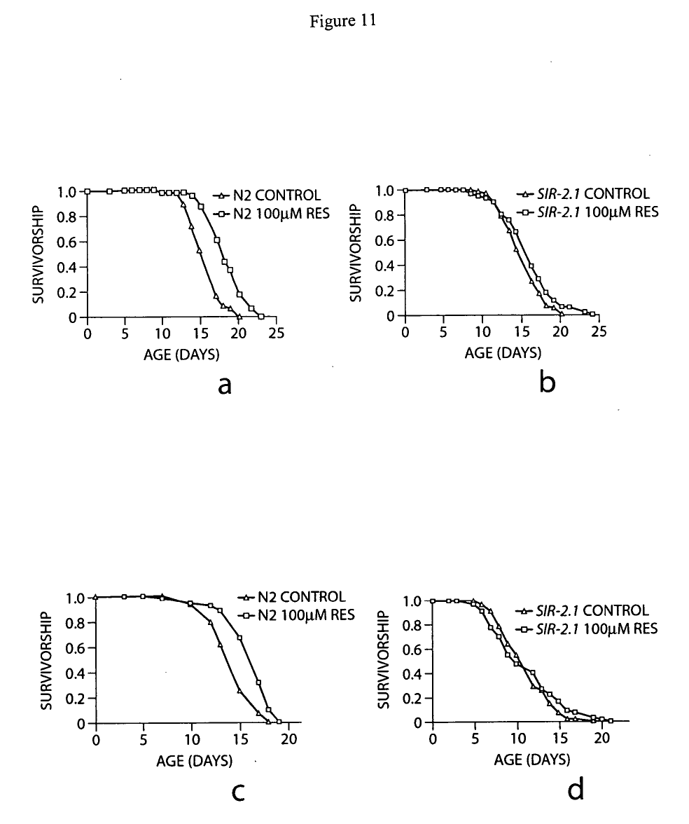

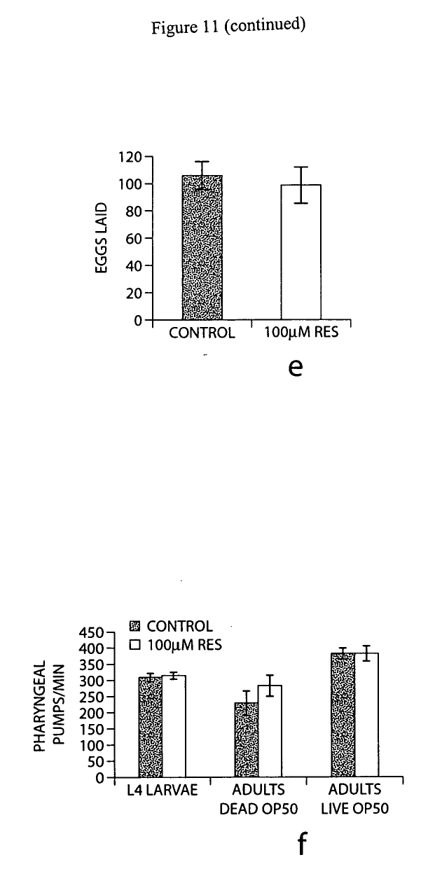

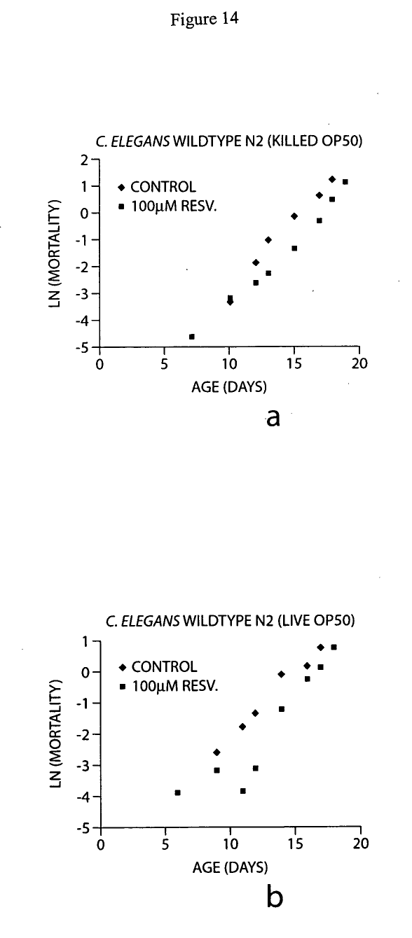

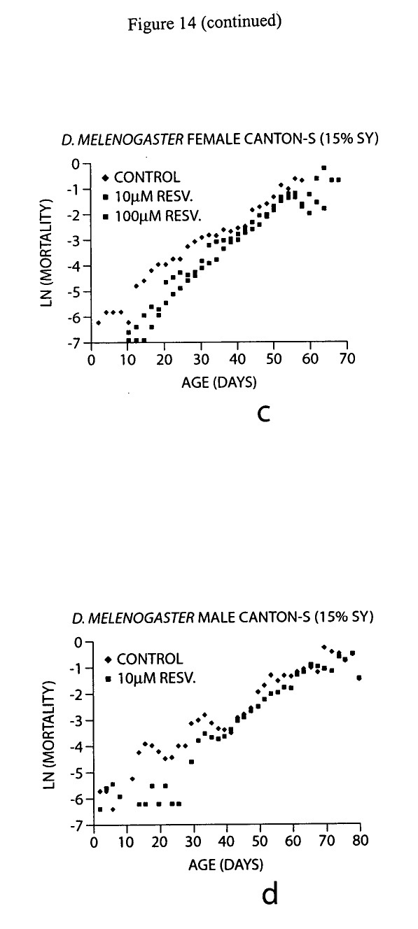

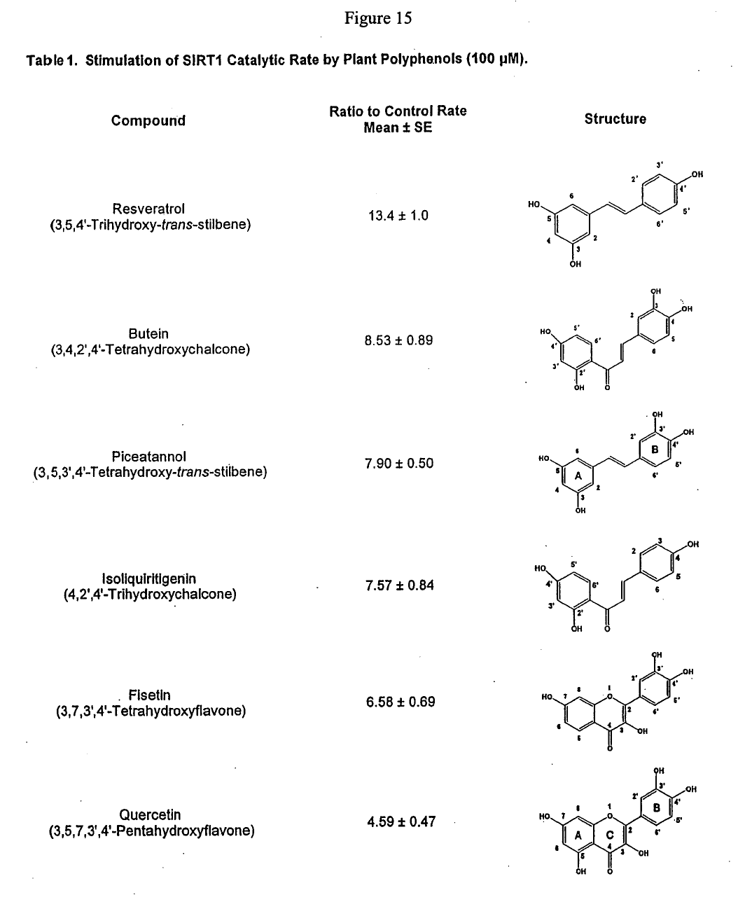

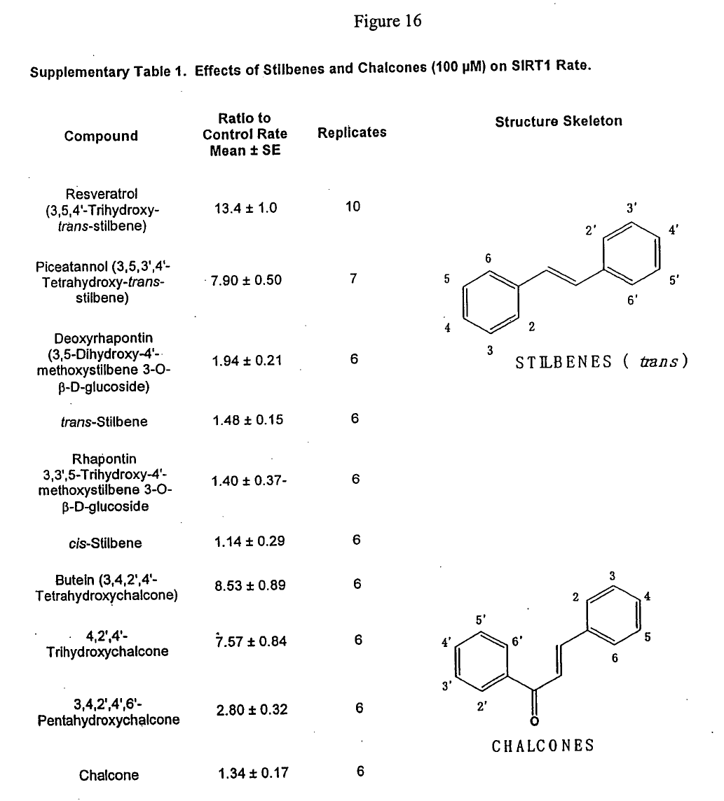

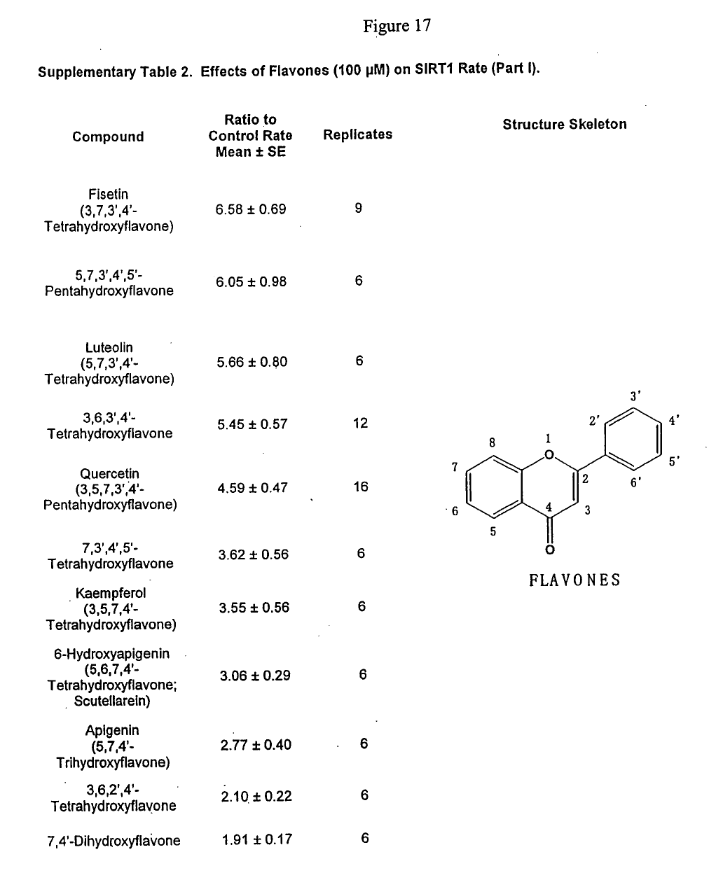

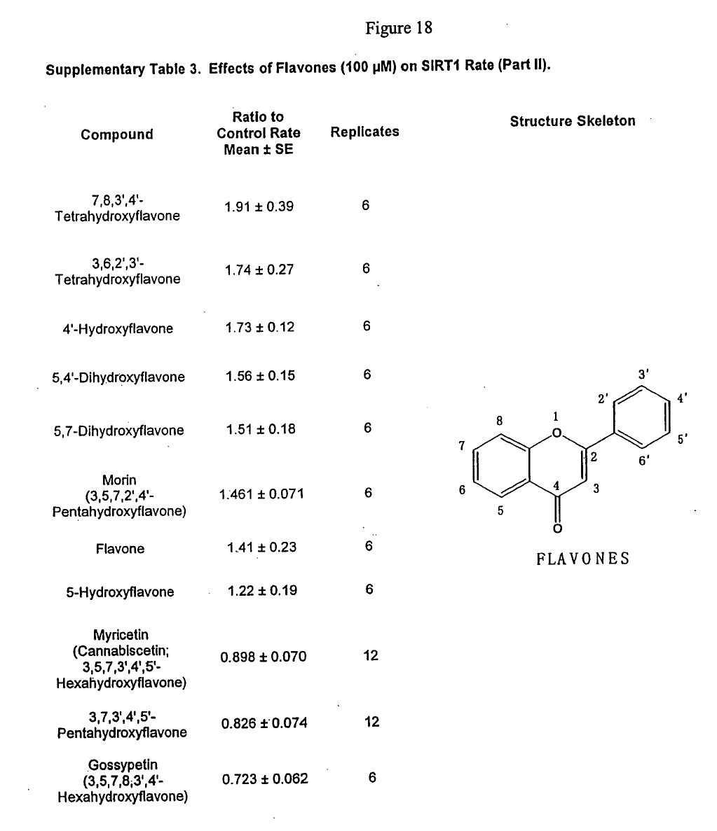

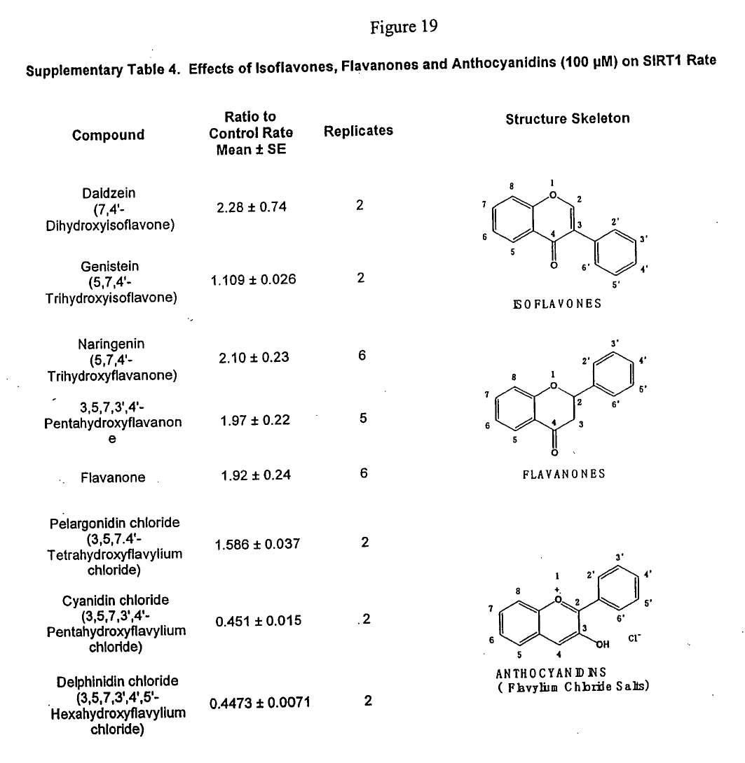

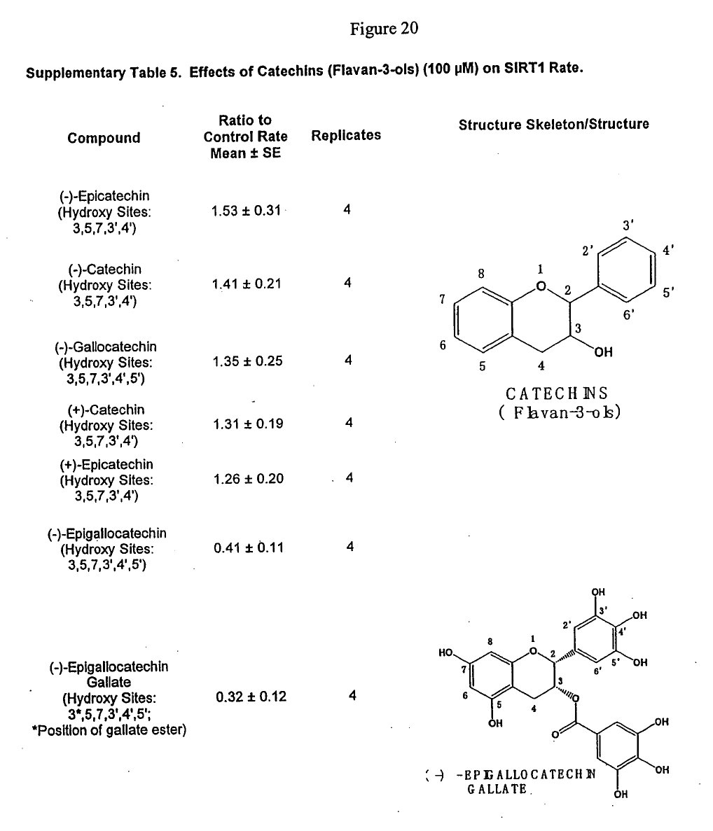

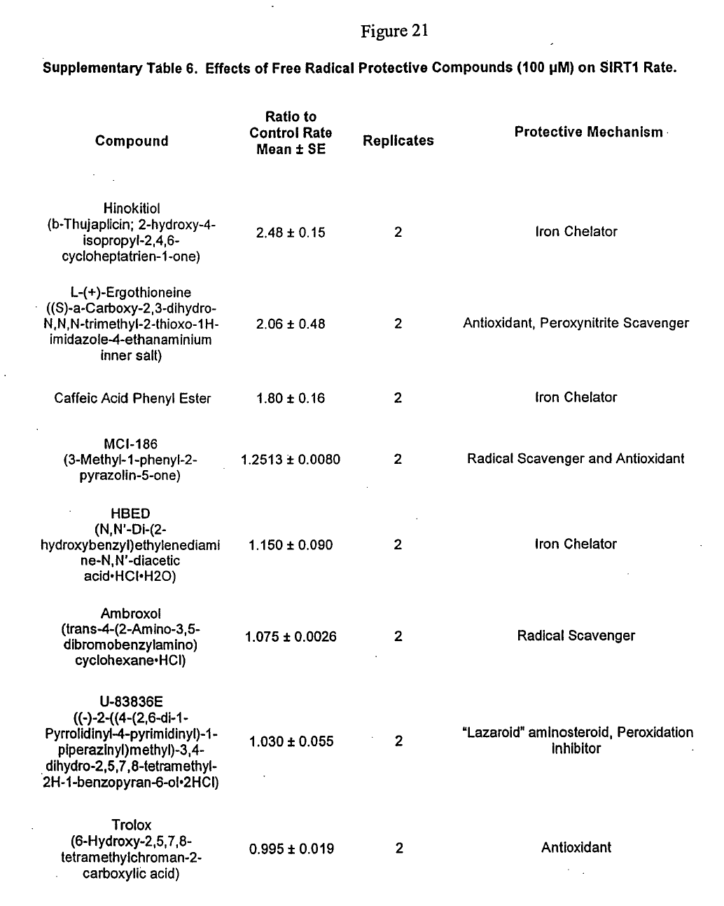

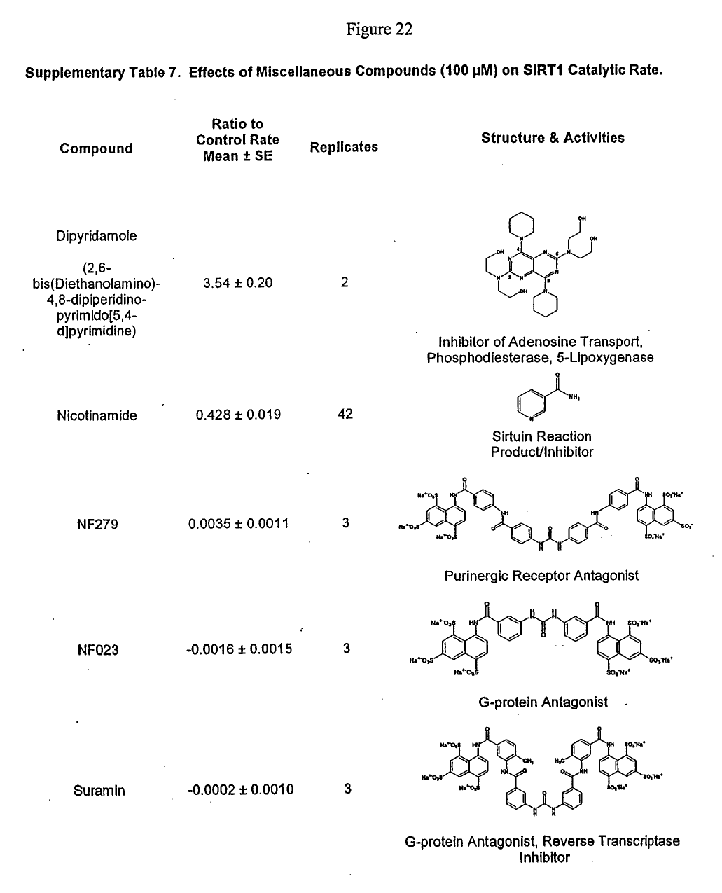

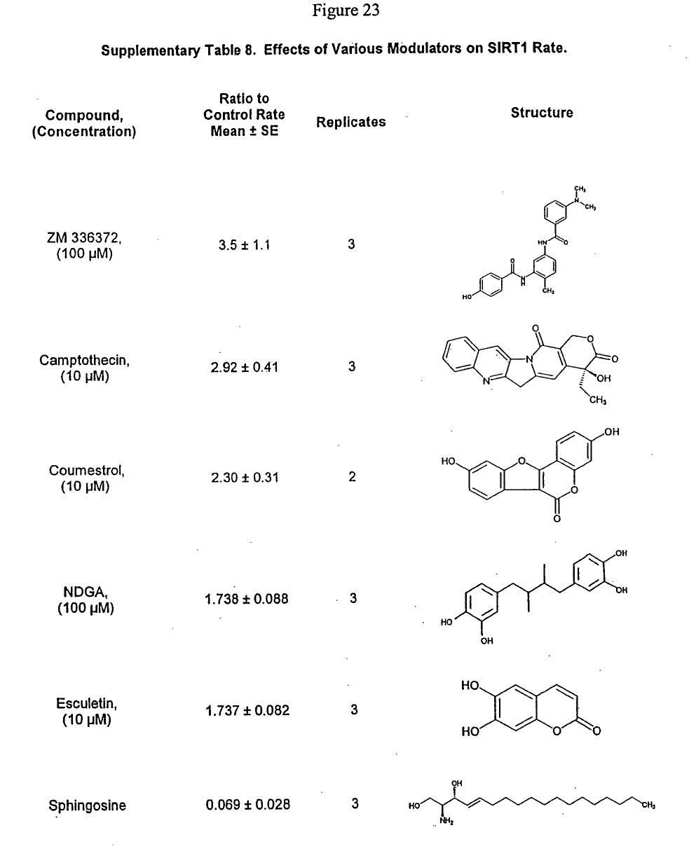

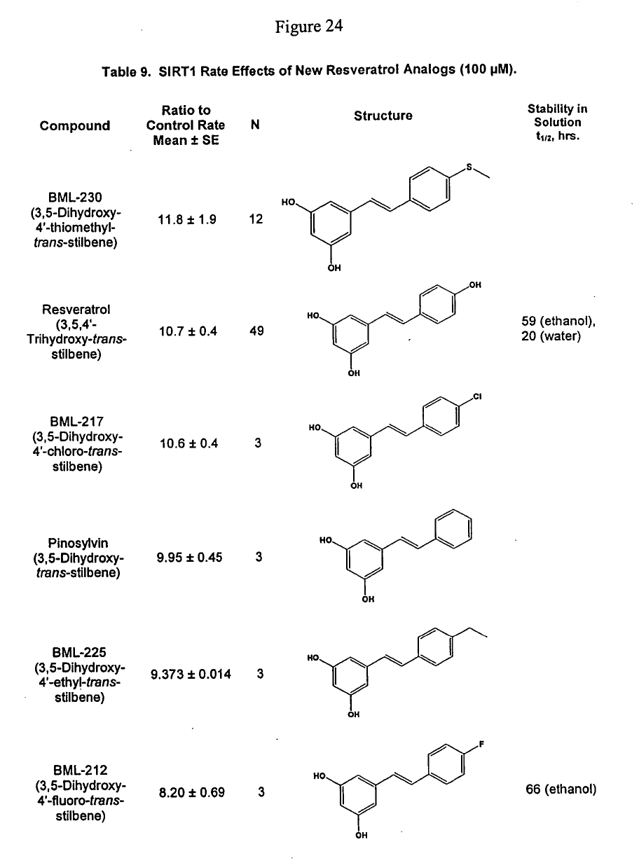

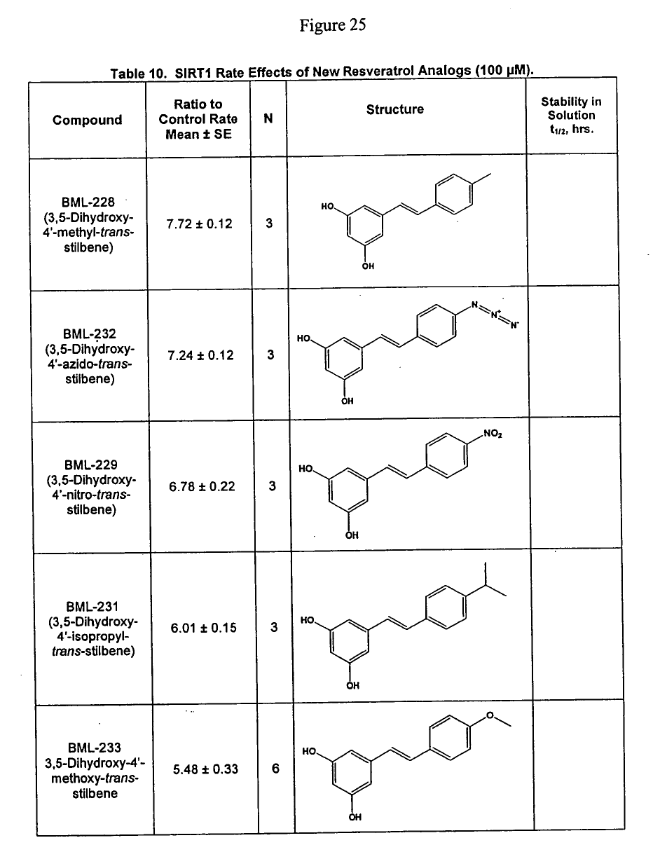

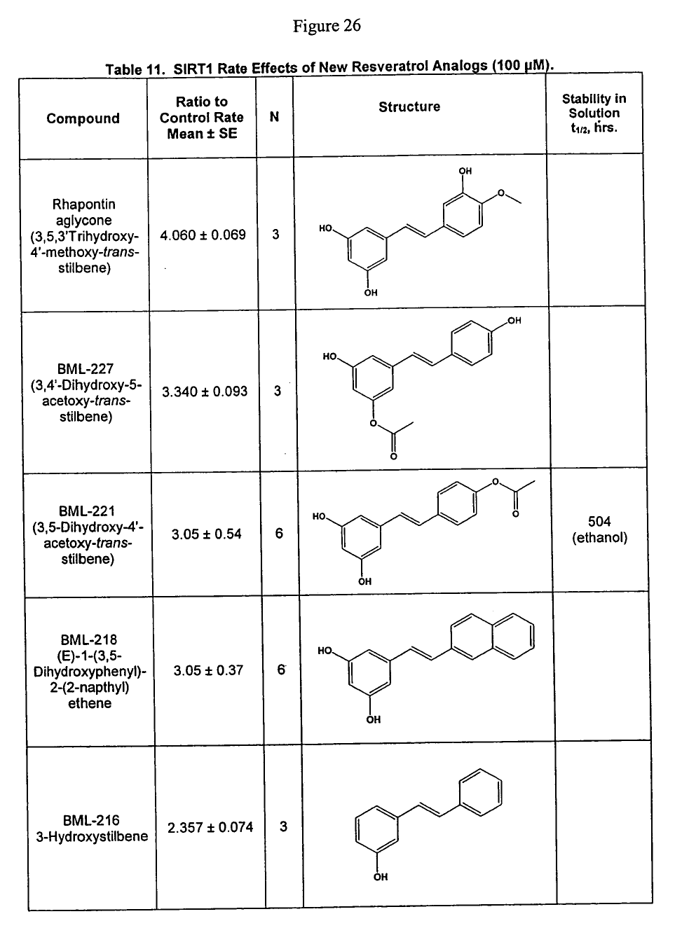

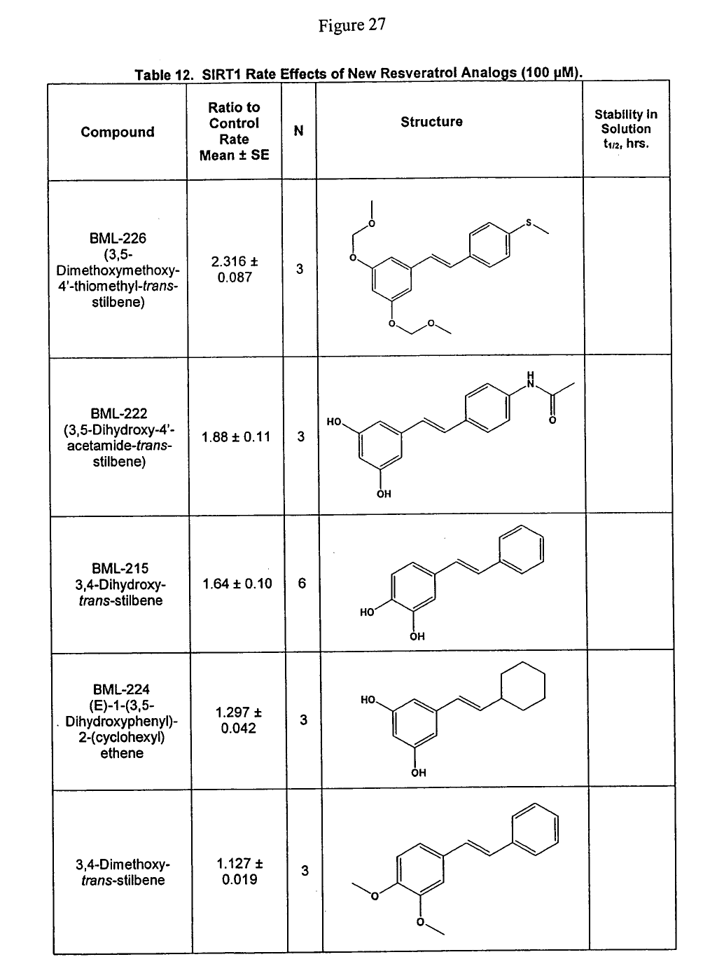

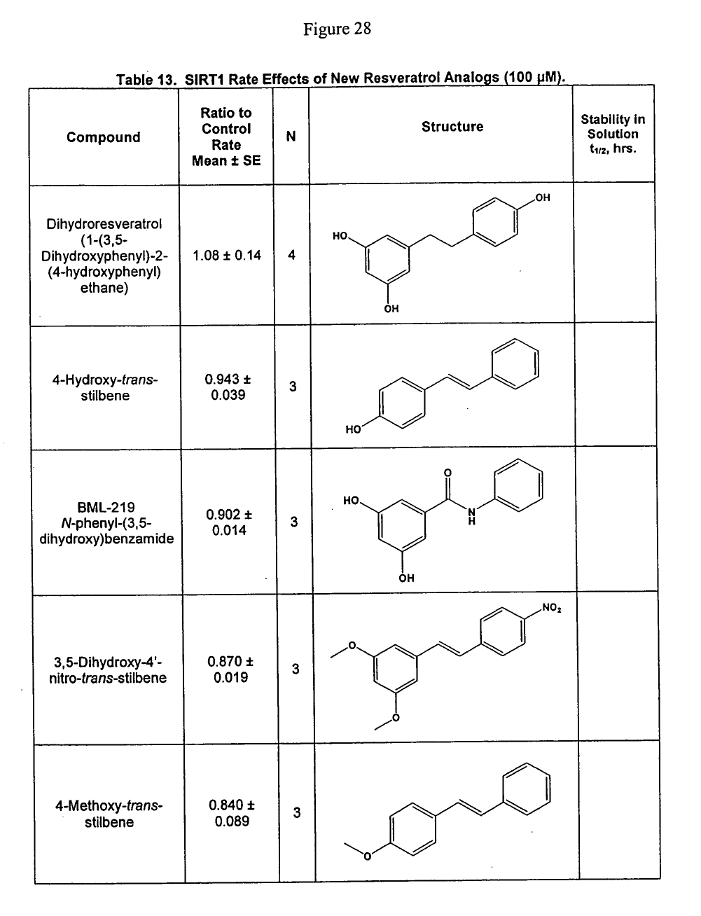

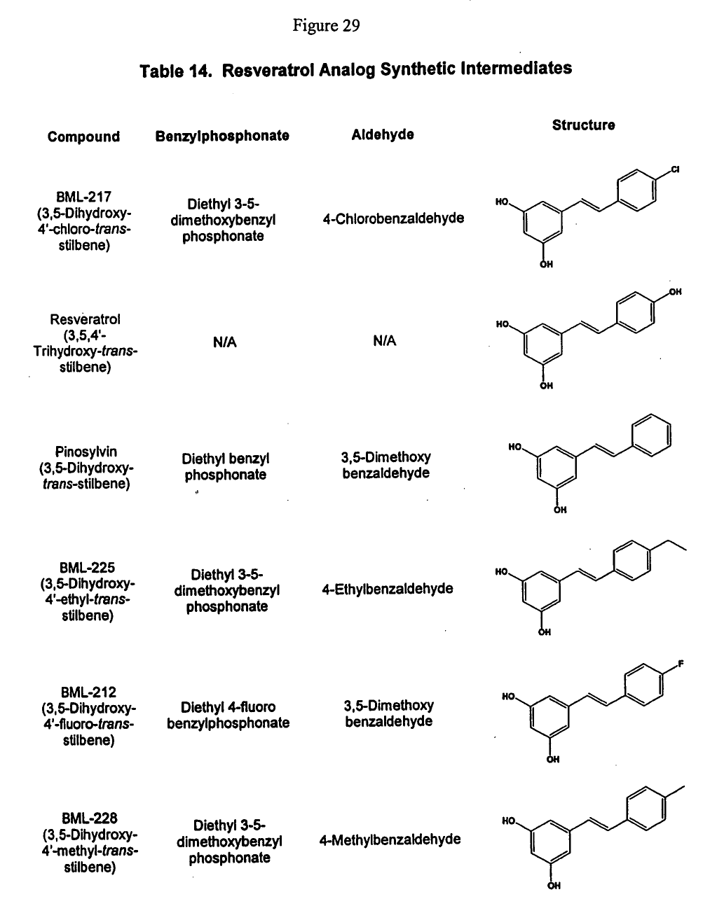

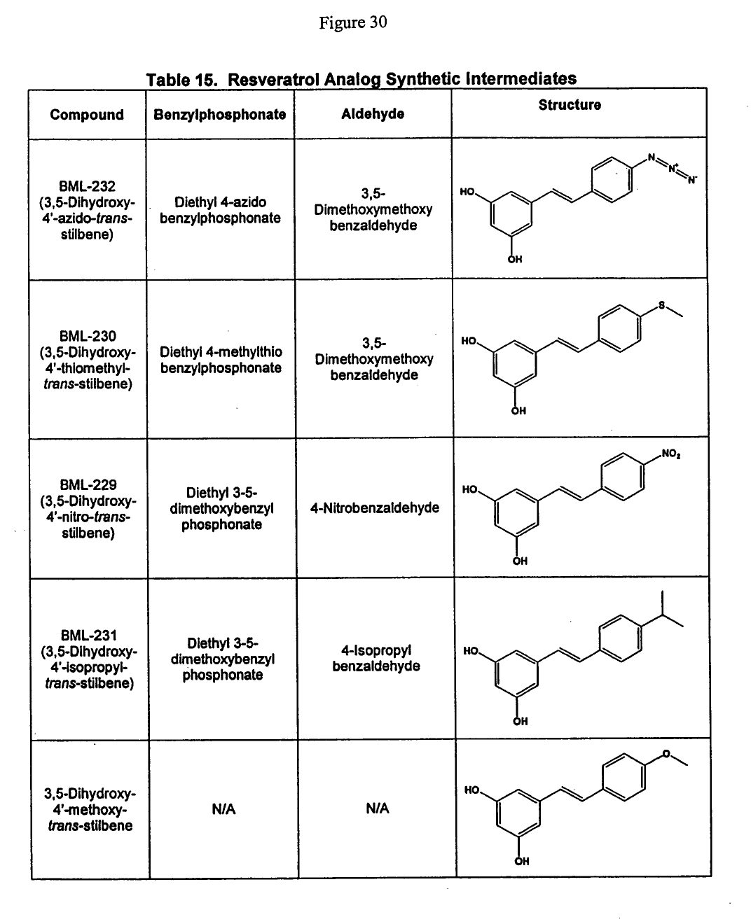

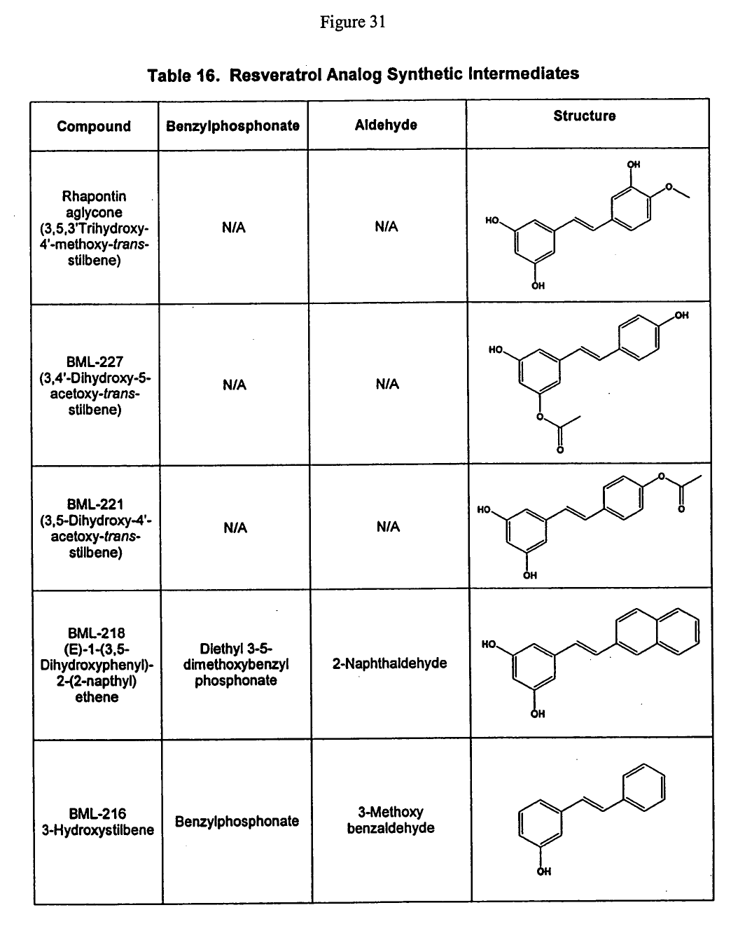

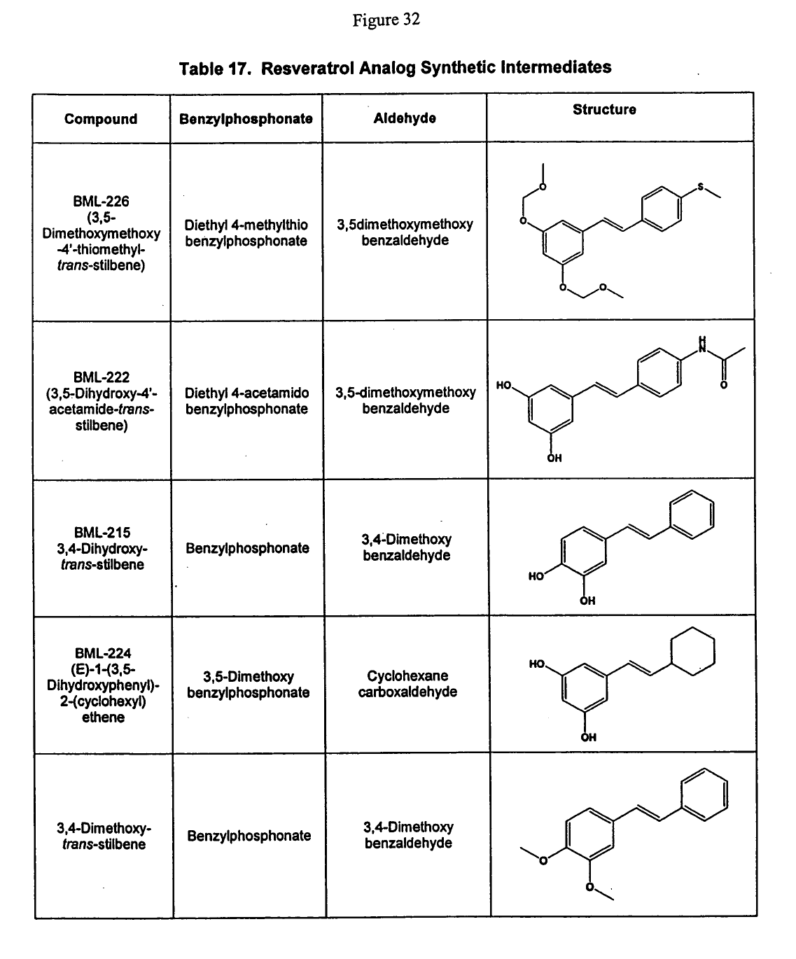

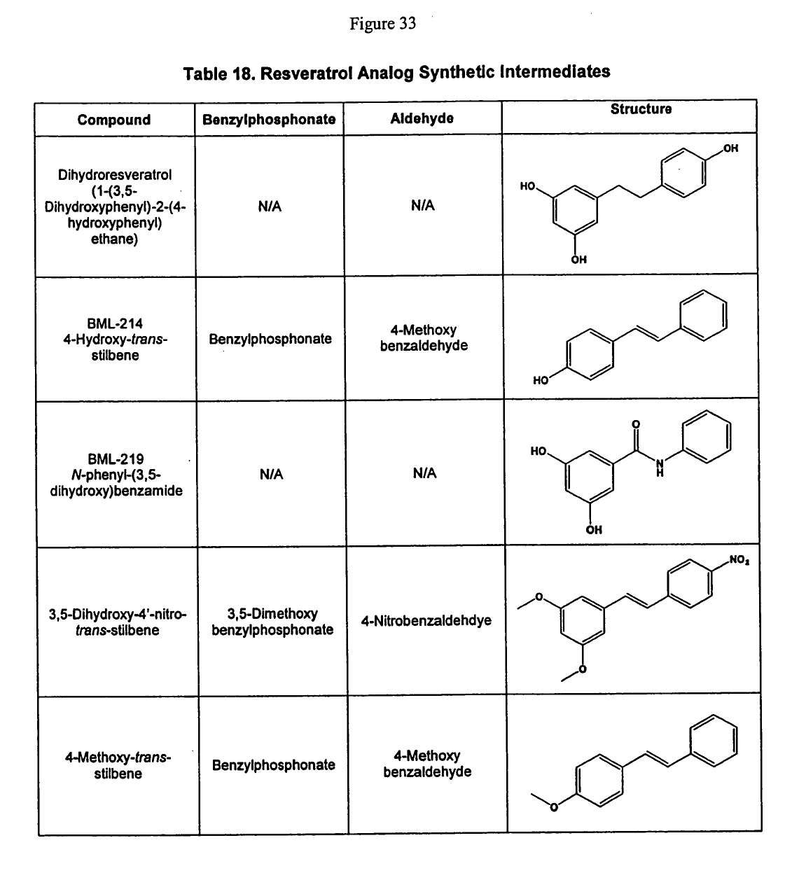

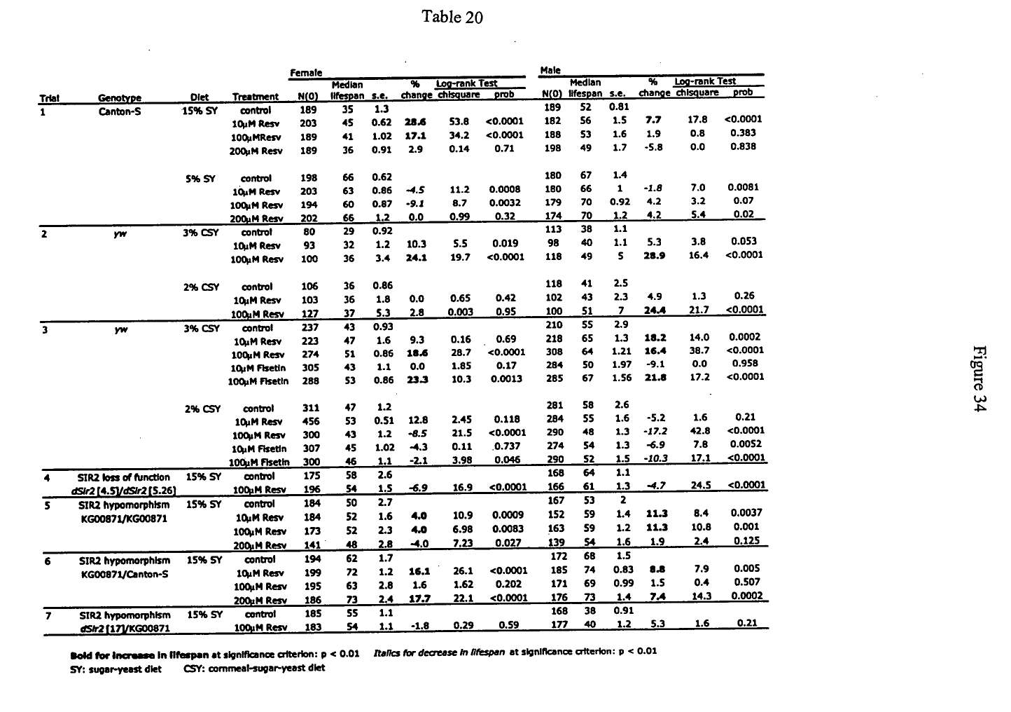

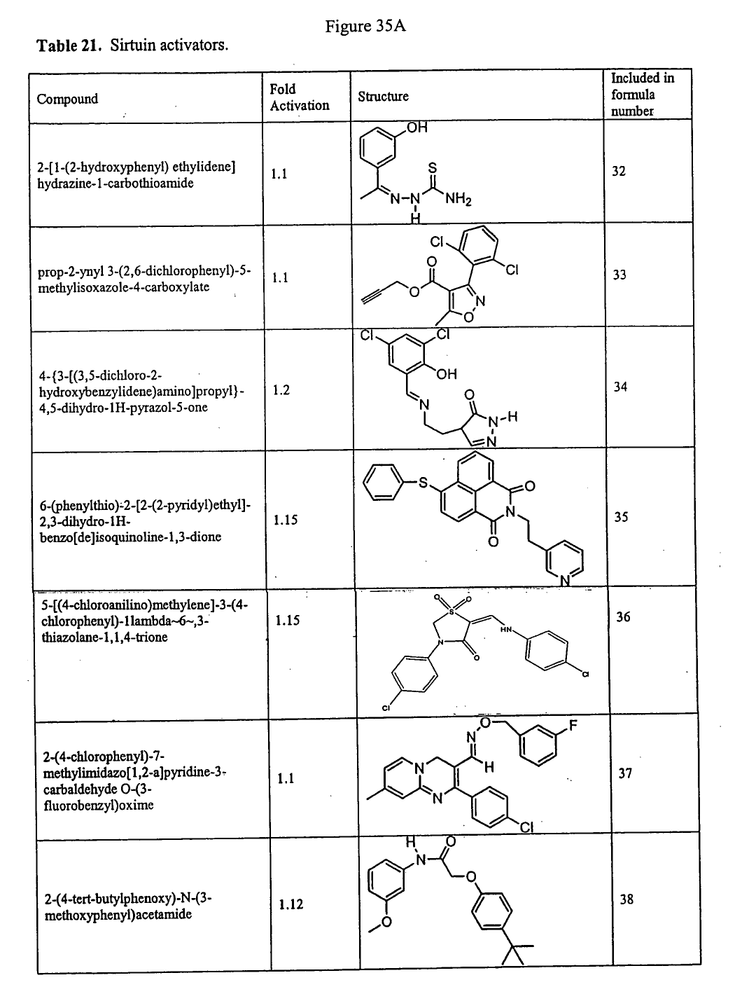

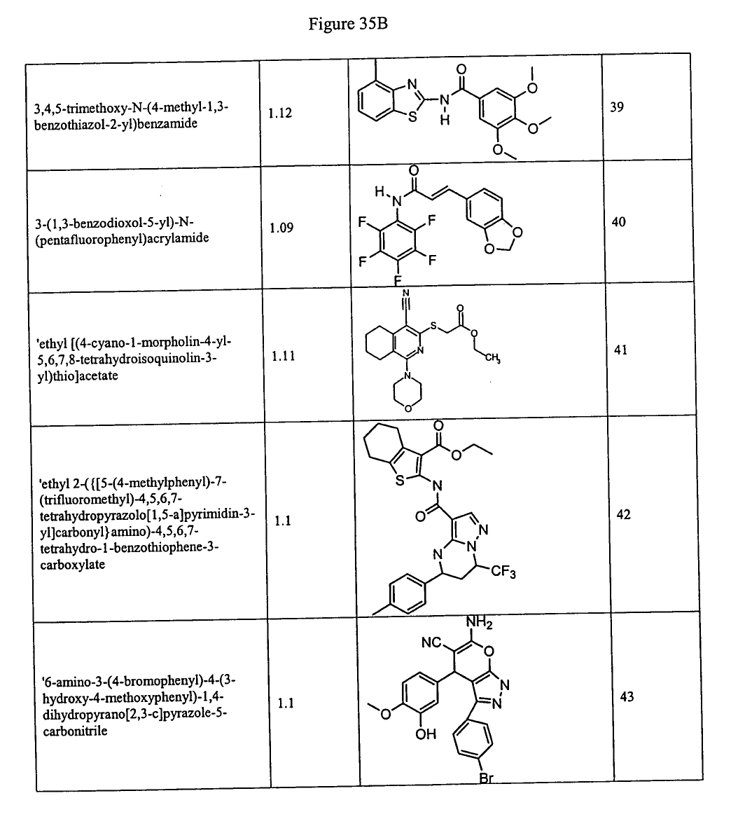

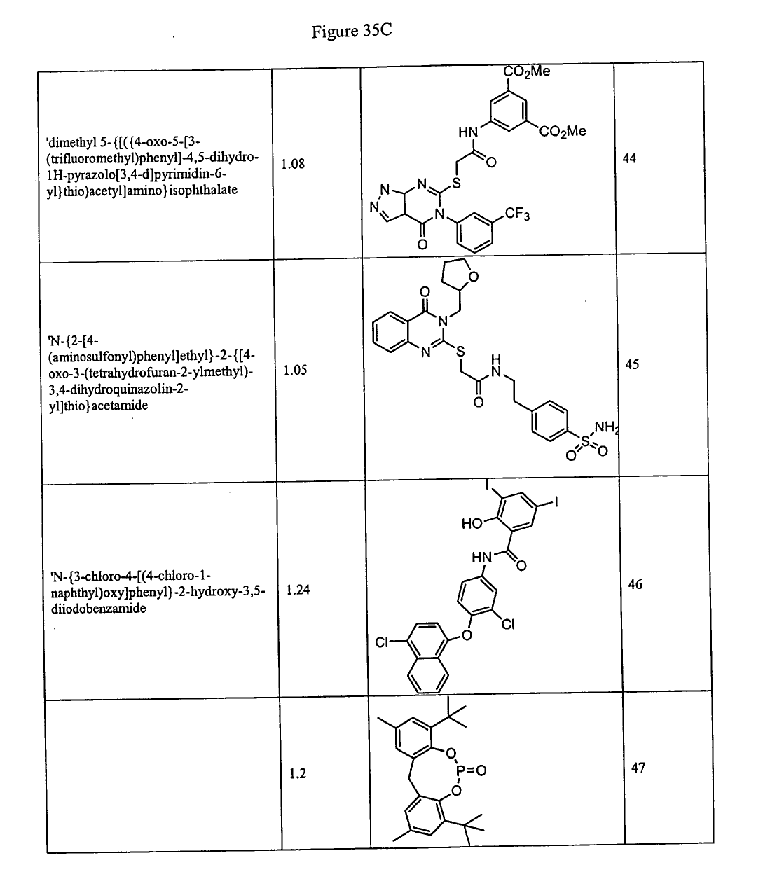

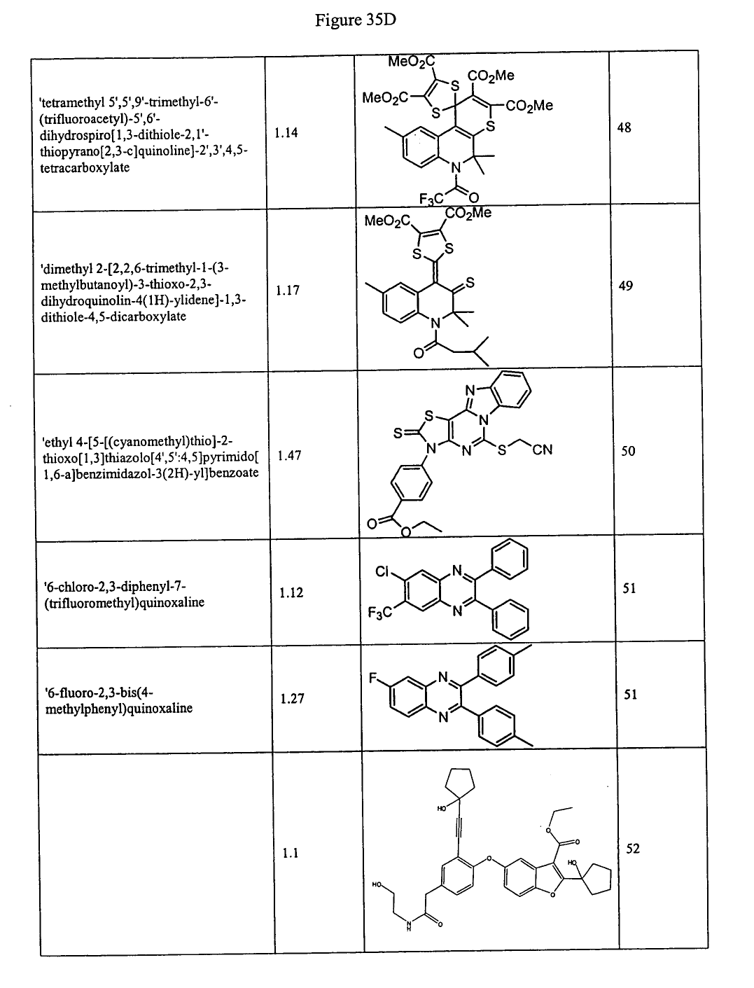

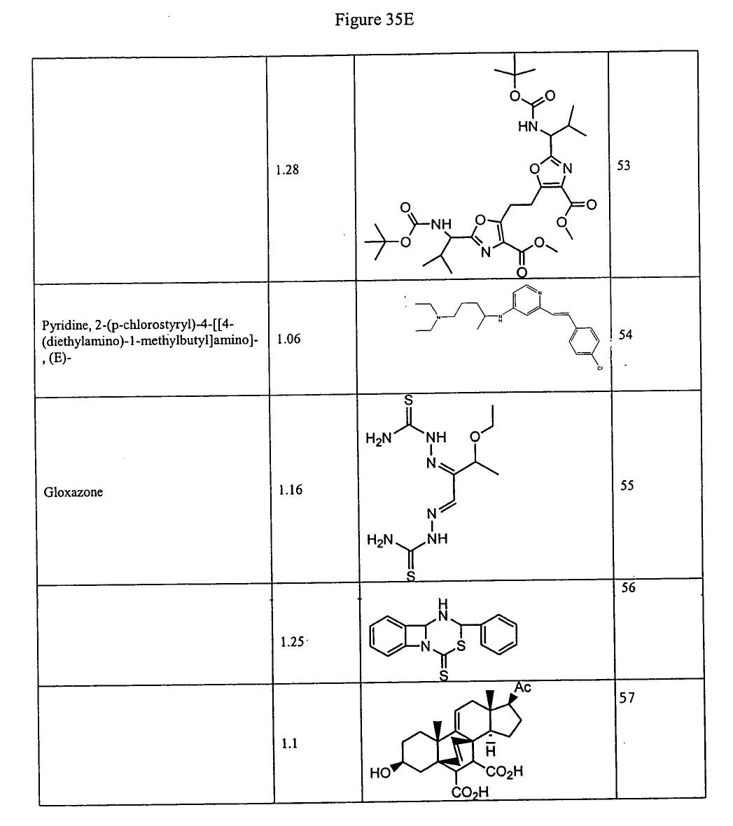

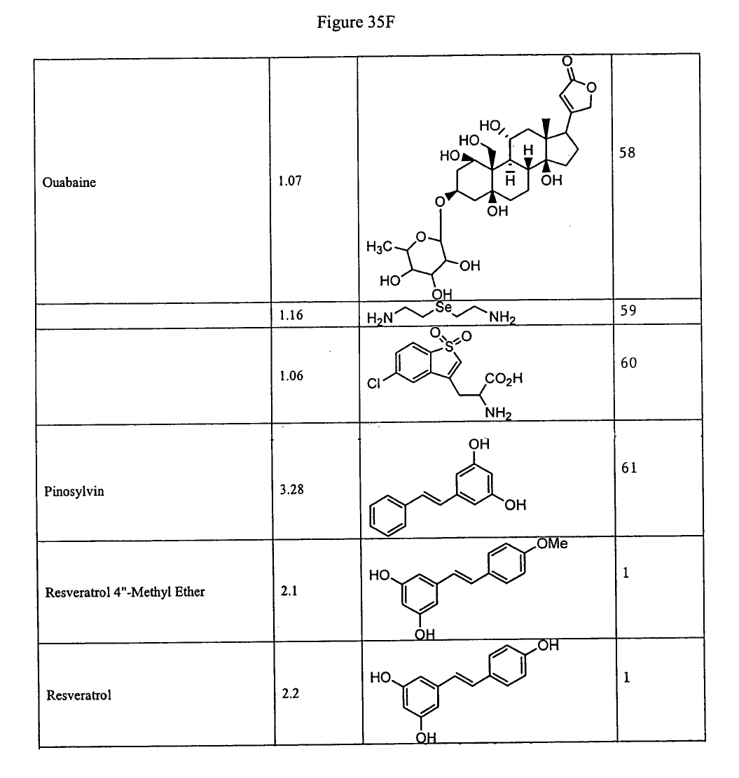

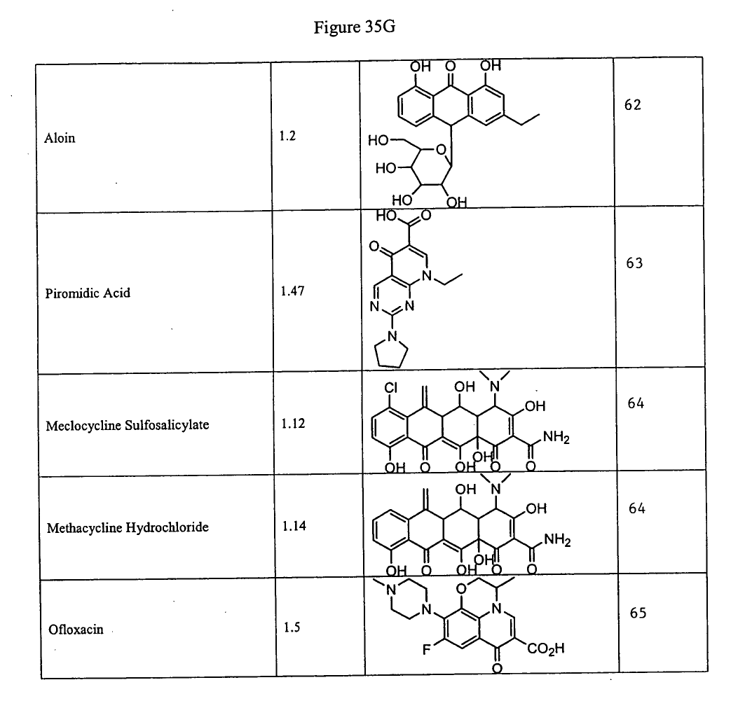

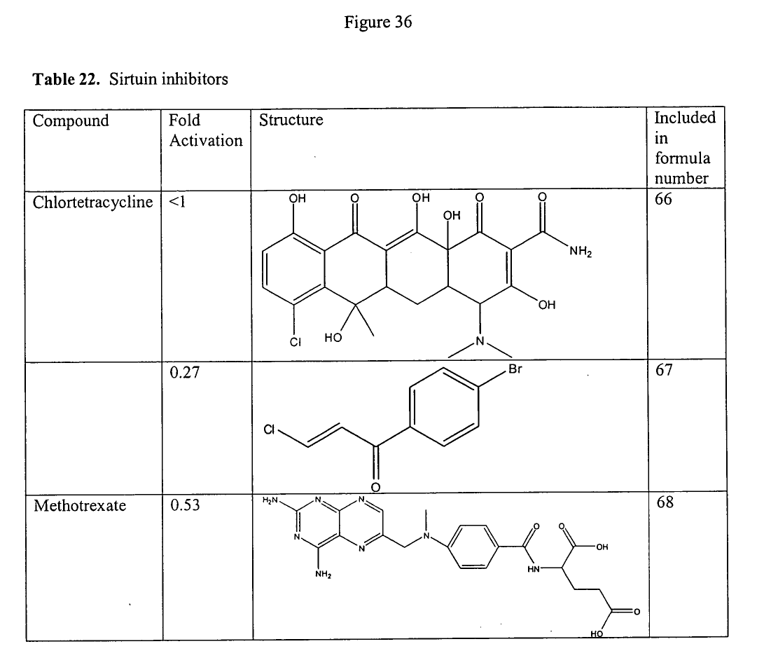

Because resveratrol can significantly extend replicative lifespan in yeast⁴, it was investigated whether STACs could also extend lifespan in the metazoans C. elegans and D. melanogaster. Wild-type worms were transferred to plates containing 0 or 100 μM of resveratrol shortly after reaching adulthood. Lifespan was reproducibly extended up to 15%, using either heat-killed or live E. coli as food supply (FIG. 11a, c respectively) and mortality was decreased across all adult ages (FIG. 14). To test whether the lifespan extension depends on functional SIR-2.1, a sir-2.1 null mutant was constructed. The lifespan of this strain was not appreciably shorter than the wildtype N2 control and adults treated with resveratrol did not exhibit a significant lifespan extension relative to untreated worms (FIG. 11b, d). There was no decrease in fecundity associated with resveratrol treatment (FIG. 11e). To rule out the possibility that resveratrol was causing the animals to eat less, thereby inducing a CR effect indirectly, feeding rates of both L4 larval and adult worms were measured with or without resveratrol and no differences were found (FIG. 11f).

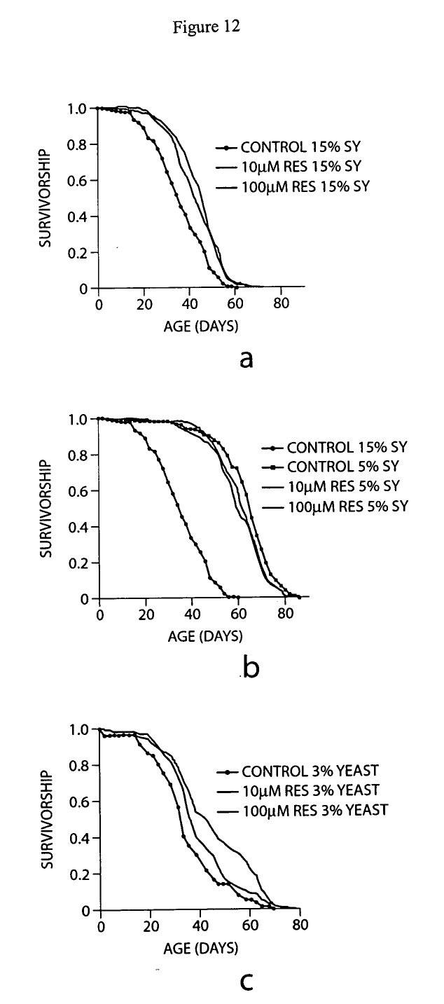

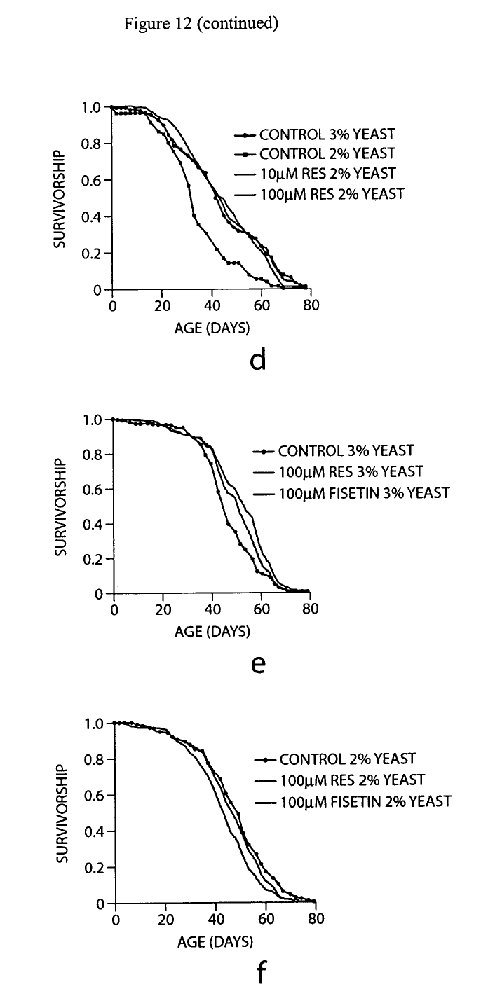

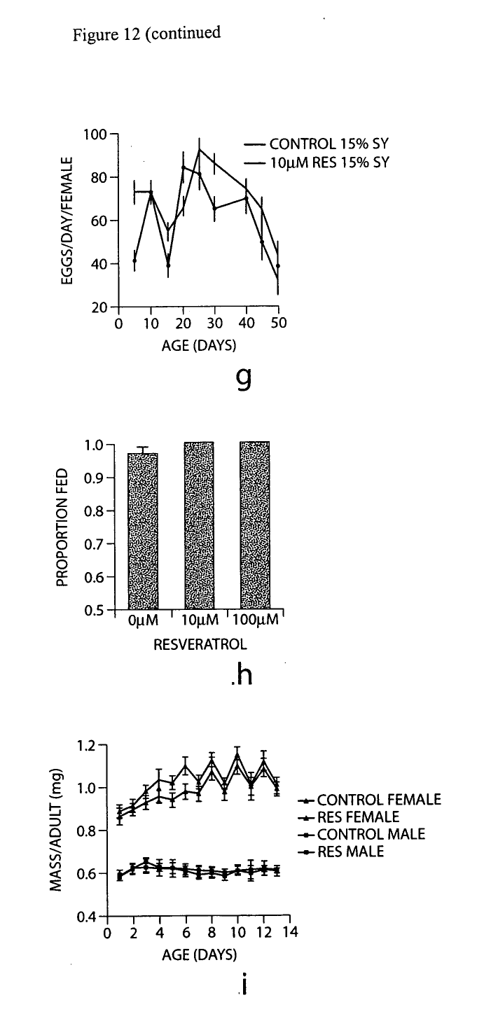

Whether STACs could extend lifespan in D. melanogaster was also tested using the standard laboratory wild type strain Canton-S and normal fly culturing conditions (vials), and a yw marked wild type strain and demographic culturing conditions (cages) (Table 20). Across independent tests in males and females, lifespan was extended up to 23% with fisetin and up to 29% with resveratrol (FIG. 12a, c, e). Increased longevity was associated with reduced mortality prior to day 40 (FIG. 14). A restricted diet increased lifespan by 40% in females and by 14% in males (averaged across trials), and under these conditions neither resveratrol nor fisetin further increased longevity (FIG. 12b, d, f), suggesting that resveratrol extends lifespan through a mechanism related to CR.

Surprisingly, while diet manipulations that extend D. melanogaster longevity typically reduce fecundity^{19,20}, longevity-extending doses of resveratrol modestly increased egg production (10 μM resveratrol: 69.8 eggs/5 days, s.e.=2.2; control: 59.9 eggs/5 days, s.e.=2.2; t=3.17, P=0.0017), particularly in the earliest days of adult life (FIG. 12g). The increase in egg production suggests that the lifespan extending effect of resveratrol in D. melanogaster was not due to CR induced by food aversion or lack of appetite. Consistent with this, no decrease in food uptake was seen with resveratrol-fed flies (FIG. 12h). Furthermore, resveratrol-fed flies maintained normal weight (FIG. 12i), except during days 3 through when resveratrol fed females were laying significantly more eggs than control fed females.

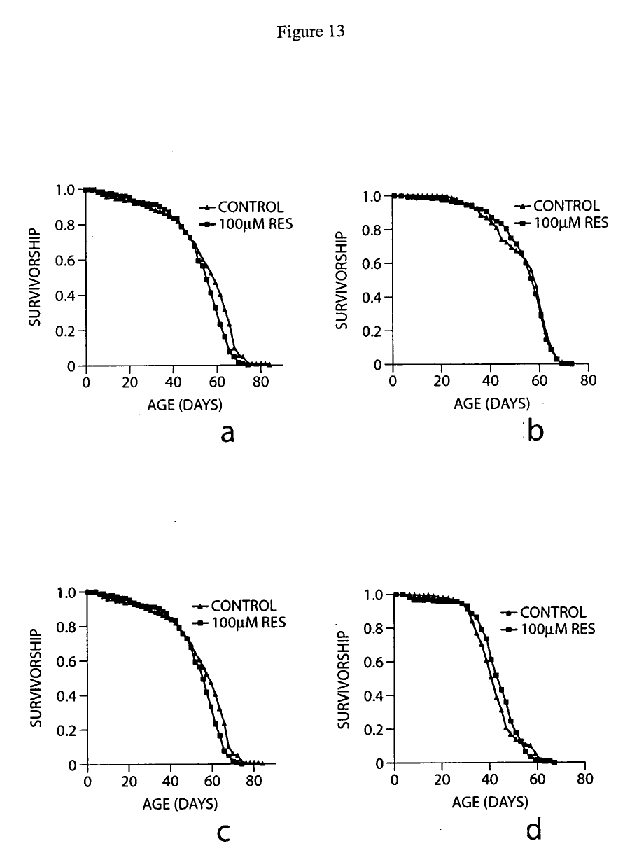

To determine whether resveratrol extends fly lifespan in a Sir2-dependent manner, a dSir2 allelic series was analyzed with increasing amounts of dSir2. Adult offspring from crosses between independently derived alleles of dSir2 were tested. Resveratrol failed to extend lifespan in flies completely lacking functional dSir2 (dSir2^{4.5}/dSir2^{5.26}) (FIG. 13a, b) or in flies in which dSir2 is severely decreased (dSir2¹⁷/dSir2^{KG00871}) (FIG. 13c, d). Resveratrol increased longevity a small but statistically significant amount in flies homozygous for a hypomorphic dSir2 allele (dSir2^{KG0087}/dSir2^{KG0087}) (Table 20, Trial 6) and increased lifespan up to 17% in flies with one copy of the hypomorphic allele and one copy of a wild-type dSir2 (Canton-S/dSir2^{KG0087}) (Table 20, Trial 7). These data demonstrate that the ability of resveratrol to extend fly lifespan requires functional Sir2.

It was previously reported that STACs extend the lifespan of replicating yeast cells by mimicking CR⁴. In yeast, chronological and reproductive aging are inseparable in the measure of replicative lifespan. Here it is shown that STACs can extend lifespan in C. elegans and D. melanogaster, both of which are comprised of primarily non-dividing (post-mitotic) cells as adults, and whose somatic and reproductive aging are independent measures of senescence. In both species, resveratrol increases lifespan in a Sir2-dependent manner and, at least for the fly, this action appears to function through a pathway common to CR.

The observation that resveratrol can increase longevity without an apparent cost of reproduction is counter to prevalent concepts of senescence evolution. However, STACs may still entail trade-offs under some environmental conditions^{21,22} or in the context of selection acting upon the network of traits that determine fitness^{23,24}. Plants synthesize STACs such as resveratrol in response to stress and nutrient limitation²⁵, possibly to activate their own sirtuin pathways⁴. These molecules may activate animal sirtuins because they serve as plant defense mechanisms against consumers or because they are ancestrally orthologous to endogenous activators within metazoans. Alternatively, animals may use plant stress molecules as a cue to prepare for a decline in their environment or food supply⁴. Understanding the adaptive significance, endogenous function, and evolutionary origin of sirtuin activators will lead to further insights into the underlying mechanisms of longevity regulation and aid in the development of interventions that provide the health benefits of CR.

### Example 15

### Materials and Methods for Example 14

Sirtuin Purification

His₆-tagged recombinant SIR-2.1 and dSir2 were purified from E. coli BL21 (DE3) plysS cells harboring either pET28a-sir-2.1 or pRSETc-dSir2 plasmids. Cells were grown in LB medium containing kanamycin (50 μg/mL) for pET28a-sir-2.1 or ampicillin (100 μg/ml) and chloramphenicol (25 μg/ml) for pRSETc-dSir2 at 30° C. (dSir2) or 37° C. (SIR-2.1) to an OD₆₀₀ of 0.6-0.8. After addition of IPTG (1 mM), flasks were shifted to 16° C. for 20 h. Cell pellets were resuspended in cold PBS buffer containing 300 mM NaCl, 0.5 mM DTT, 0.5 mM PMSF and EDTA-free protease inhibitor tablets and lysed by sonication. Ni²⁺-NTA beads were added to the clarified extract and after 1-3 hours they were loaded on a column, washed with buffer (50 mM Tris. Cl pH 7.4, 200 mM NaCl, 30 mM imidazole) then eluted with the same buffer containing 600 mM imidazole.

Deacetylation Assays

From 0.1 to 1 μg of SIR-2.1 and 1 μg of dSir2 were used per deacetylation assay as previously described with modifications (SIR-2.1: 200 μM NAD⁺, 10 μM Fluor de Lys, FdL; dSir2: 25 μM NAD⁺, 10 μM FdL)²⁶. STACs were dissolved at 10 mM in dimethylsulfoxide (DMSO) the day of the assay. In vitro fluorescence assay results were read in 96-well microplates (Corning Costar 3693) with a Wallac Victor Multilabel counter (Perkin Elmer, excitation at 360 nm, emission at 450 nm). Drosophila S2 cells were grown in Schneider media with fetal calf serum at 23-28° C., seeded at 9×10⁴ cells/well, grown overnight and then exposed to 1 μM TSA, 500 μM polyphenols, and 200 μM FdL for 2 hr. Deacetylation of FdL with lysate from whole cells was determined as described⁴. Unless otherwise indicated all initial rate measurements were means of three or more replicates obtained with single incubation times, at which point 5% or less of the substrate initially present was deacetylated.

C. elegans Media, Strains, Lifespan, and Feeding Assays

Bristol N2 (Caenorhabditis Genetics Center) was used as the wild-type strain. The sir-2.1 mutant strain was generated by backcrossing VC 199 (sir-2.1 (ok434)) to N2 four times. Cultures were grown on standard NGM media and maintained on E. coli strain OP50. For the lifespan assays, synchronized animals were transferred to treatment plates as young adults (2 d after hatching, day 0 of assay), and were transferred to fresh treatment plates every 2 days for the first 6 to 8 days of the assay. Treatment plates were standard NGM media with the reproductive suppressant FUdR (Sigma; 100 mg/L) containing resveratrol or solvent (DMSO, which does not affect lifespan) added either directly into the agar before pouring (for live OP50 trials) or diluted into PBS and added to the surface of a dry plate to the indicated final concentration (for dead OP50 trials). For some lifespan trials, heat-killed OP50 were used as a food source. OP50 cultures were heated to 65° C. for 30 minutes, then pelleted and resuspended in 1/10 volume in S Basal supplemented with 10 mM MgSO₄. In all assays, worms were monitored daily for mortality by gently probing with a platinum pick. Assays were performed at 24° C. To assay worm feeding rates, worms at the indicated stages were placed on treatment plates (no FUdR) for 4-5 hours, then videoed for 1 minute using a Pixelink PL-662 camera. The frame rate was slowed and the pumping rate of the pharynx was counted. To assay fecundity, gravid hermaphrodites (5 per plate, raised from synchronized L1s on normal or treatment plates) were allowed to lay eggs on their respective media for 5 hours, and the total number of eggs was counted.

D. melanogaster Media, Strains, Feeding Assay and Lifespan Assays

Survival assays were conducted independently with adult D. melanogaster in two laboratories. In the first laboratory, all trials used an yw marked wild-type strain. Larvae were reared on standard cornmeal-sugar-yeast (CSY) agar diet (cornmeal 5%, sucrose 10.5%, SAF yeast 2%, and agar 0.7%). Newly eclosed adults were placed in 1 L demography cages with approximately 75 males and 75 females. Three to four replicate 1 L demography cages were used for each treatment group in each trial. Every two days, dead flies were removed and scored, and food vials were replenished. Food vials contained cornmeal-sugar-yeast diet with SAF yeast as either 2% or 3% by weight. Test compounds in 100 μl of EtOH (or blank EtOH in controls) were mixed into melted aliquots of the adult food media to make a final concentration of 0, 10 or 100 μM. Fresh stock solutions and adult media were prepared weekly. In the second laboratory, lifespan trials were conducted with the wild type strain Canton-S, dSir2^{4.5} and dSir2^{5.26} (S. Smolik, University of Oregon), dSir2¹⁷ (S. Astrom, Stockholm University, Sweden), and dSir2^{KG00871} (Drosophila Stock Center, Bloomington, Ind.). Larvae for all tests were reared on standard cornmeal-sugar-yeast diet. Newly eclosed adults were incubated in plastic shell vials containing 5 ml of 15% sugar-yeast diet (15% SY) or 5% sugar-yeast (5% SY) diet (15% SY: 15% yeast, 15% sucrose, 2% agar; 5% SY: 5% yeast, 5% sucrose, 2% agar as per Ref.²⁰). In all trials, ˜20 males with ˜20 females were placed into each of 10 vials/treatment group. Every two days, flies were passed into new vials and dead flies were counted. Resveratrol in EtOH (or EtOH alone in controls) was added to the media during its preparation after it had cooled to 65° C. and mixed vigorously. Final compound concentrations were 0, 10, 100 or 200 μM. Fresh stock solution and adult media was prepared weekly.

Feeding rate was measured in yw females with the crop-filling assay²⁷. Females were held overnight with water and placed on 2% CSY diet containing food colour (FDA Blue 1) and 0, 10 or 100 μM resveratrol with EtOH. The presence of dye-marked food in the crop was scored in sets of 20 females across five 5-minute intervals. For body mass measurements, 10 vials with 20 males and 20 females each of wild type CS-5 flies were kept on 15% SY diet with EtOH or with resveratrol in EtOH (10 μM). Males and females were weighed daily.

### REFERENCES FOR EXAMPLES 14 AND 15

1. Lin, S. J., Defossez, P. A. & Guarente, L. Requirement of NAD and SIR2 for life-span extension by calorie restriction in Saccharomyces cerevisiae. Science 289, 2126-8. (2000).
2. Gasser, S. C. M. The molecular biology of the SIR proteins. Gene 279, 1-16 (2001).
3. Hekimi, S. & Guarente, L. Genetics and the specificity of the aging process. Science 299, 1351-4 (2003).
4. Howitz, K. T. et al. Small molecule activators of sirtuins extend Saccharomyces cerevisiae lifespan. Nature 425, 191-6 (2003).
5. Landry, J. et al. The silencing protein SIR2 and its homologs are NAD-dependent protein deacetylases. Proc Natl Acad Sci USA 97, 5807-11. (2000).
6. Imai, S., Armstrong, C. M., Kaeberlein, M. & Guarente, L. Transcriptional silencing and longevity protein Sir2 is an NAD-dependent histone deacetylase. Nature 403, 795-800 (2000).
7. Smith, J. S. et al. A phylogenetically conserved NAD+-dependent protein deacetylase activity in the Sir2 protein family. Proc Natl Acad Sci USA 97, 6658-63. (2000).
8. Tanner, K. G., Landry, J., Sternglanz, R. & Denu, J. M. Silent information regulator 2 family of NAD-dependent histone/protein deacetylases generates a unique product, 1-O-acetyl-ADP-ribose. Proc Natl Acad Sci USA 97, 14178-82. (2000).
9. Tanny, J. C., Dowd, G. J., Huang, J., Hilz, H. & Moazed, D. An enzymatic activity in the yeast Sir2 protein that is essential for gene silencing. Cell 99, 735-45. (1999).
10. Guarente, L. Sir2 links chromatin silencing, metabolism, and aging. Genes Dev 14, 1021-6. (2000).
11. Tissenbaum, H. A. & Guarente, L. Increased dosage of a sir-2 gene extends lifespan in Caenorhabditis elegans. Nature 410, 227-30. (2001).
12. Rogina, B., Helfand, S. L. & Frankel, S. Longevity regulation by Drosophila Rpd3 deacetylase and caloric restriction. Science 298, 1745. (2002).
13. Jiang, J. C., Jaruga, E., Repnevskaya, M. V. & Jazwinski, S. M. An intervention resembling caloric restriction prolongs life span and retards aging in yeast. Faseb J 14, 2135-7. (2000).
14. Kenyon, C. A conserved regulatory mechanism for aging. Cell 105, 165-168 (2001).
15. Masoro, E. J. Caloric restriction and aging: an update. Exp Gerontol 35, 299-305. (2000).
16. Koubova, J. & Guarente, L. How does calorie restriction work? Genes Dev 17, 313-21 (2003).
17. Sinclair, D. A. Paradigms and pitfalls of yeast longevity research. Mech Ageing Dev 123, 857-67 (2002).
18. Kaeberlein, M., McVey, M. & Guarente, L. The SIR2/3/4 complex and SIR2 alone promote longevity in Saccharomyces cerevisiae by two different mechanisms. Genes Dev 13, 2570-80. (1999).
19. Chippindale, A. K., Leroi, Armand M., Kim, Sung B., and Rose, Michael R. Phenotypic plasticity and selection in Drosophila life-history evolution. Journal of Evolutionary Biology 6, 171-193 (1993).
20. Chapman, T. & Partridge, L. Female fitness in Drosophila melanogaster: an interaction between the effect of nutrition and of encounter rate with males. Proc R Soc Lond B Biol Sci 263, 755-9 (1996).
21. Walker, D. W., McColl, G., Jenkins, N. L., Harris, J. & Lithgow, G. J. Evolution of lifespan in C. elegans. Nature 405, 296-7 (2000).
22. Marden, J. H., Rogina, B., Montooth, K. L. & Helfand, S. L. Conditional tradeoffs between aging and organismal performance of Indy long-lived mutant flies. Proc Natl Acad Sci USA 100, 3369-73 (2003).
23. Schmid-Hempel, P. On the evolutionary ecology of host-parasite interactions: addressing the question with regard to bumblebees and their parasites. Naturwissenschaften 88, 147-58 (2001).
24. Ebert, D. & Bull, J. J. Challenging the trade-off model for the evolution of virulence: is virulence management feasible? Trends Microbiol 11, 15-20 (2003).
25. Soleas, G. J., Diamandis, E. P. & Goldberg, D. M. Resveratrol: a molecule whose time has come? And gone? Clin Biochem 30, 91-113 (1997).
26. Bitterman, K. J., Anderson, R. M., Cohen, H. Y., Latorre-Esteves, M. & Sinclair, D. A. Inhibition of silencing and accelerated aging by nicotinamide, a putative negative regulator of yeast sir2 and human SIRT1. J Biol Chem 277, 45099-107. (2002).
27. Edgecomb, R. S., Harth, C. E. & Schneiderman, A. M. Regulation of feeding behavior in adult Drosophila melanogaster varies with feeding regime and nutritional state. J Exp Biol 197, 215-35 (1994).

### Example 16

### Identification of Additional Activators and Inhibitors or Sirtuins

The following high-throughput screening protocol was used to identify additional small molecule sirtuin activators and inhibitors from an ICCB library.

The following wells were designated for control reactions: a) with enzyme; DMSO blank, b) with enzyme; with resveratrol (50 μM) positive control. The reaction mixture contains (final): 0.5 units/reaction SIRT1 deacetylase (BIOMOL); 200 μM NAD⁺; 5 μM Fluor de Lys-SIRT1 substrate (BIOMOL); buffer (25 mM Tris/Cl, pH 8.0, 137 mM NaCl, 2.7 mM KCl, 1 mM MgCl₂, and 1 mg/ml BSA). In addition, a reaction mixture containing no enzyme was made so that each well receiving compound has a corresponding “no enzyme control” well. Reactions were performed in black 384 well plates (NUNC) in a final volume of 25 μl/well.

The reactions were started by combining enzyme and substrate in a reaction mixture immediately prior to aliquoting in plates (or substrate only for “no enzyme control” plates). Mixture were aliquoted to plates using Biotek μFill (Biotek Instruments). Control mixtures were manually added to designated wells. A library compound was added at a desired concentration by pin transfer to both “with enzyme” and “no enzyme” plates. Compounds were added in at least triplicate (with enzyme reaction in duplicate and no enzyme controls) at a final concentration of roughly 50 μM. The plates were incubated at 37° C. for 30-60 minutes. Then 25 μl of 1× Developer II (BIOMOL) plus 2 mM nicotinamide were added to all wells to stop the reactions. The reactions were left for at least 30 minutes at 37° C. for the signal to develop. The plates were read in a microplate-reading fluorometer capable of excitation at a wavelength in the range of 350-380 nm and detection of emitted light in the range of 440-460 nm. A read time of 0.1 sec per well was used.

The following positive controls were used: resveratrol, resveratrol 4″-methyl ether (3,5-dihydroxy-4′-methoxy-trans-stilbene, also referred to herein as BML-233, and set forth in Table 10), and pinosylvin, which activated SIRT1 2.2 fold, 2.1 fold and 3.28 fold, respectively. The activators are listed in Table 21 and the inhibitors are listed in Table 22.

### Example 17

### Resveratrol Promotes Fat Mobilization

This example shows that a compound that activates sirtuins, resveratrol, stimulates fat metabolism by reducing fat accumulation in C. elegans.

Wild-type N2 C. elegans worms were grown on OP50 bacteria and exposed overnight to vehicle (0.1% ethanol) alone or with 10, 50 or 100 μM of resveratrol (in ethanol). Fat accumulation was visualized with Nile Red staining, as described further below and in Ashrafi K, et al. Nature 421:268-27 (2003).

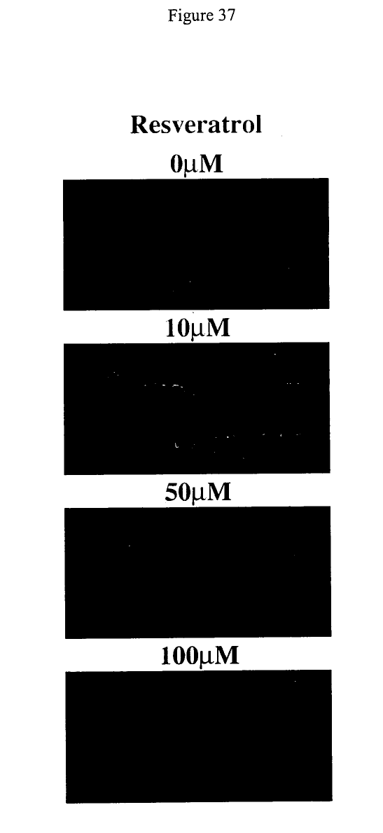

The results, which are shown in FIG. 37, indicate that resveratrol treatment with 100 μM resulted in a 90% reduction of fat accumulation. Similarly, incubation of the worms in the presence of 10 μM or 50 μM of resveratrol showed a marked decrease in fat accumulation. The decrease in fat accumulation is as or more striking than treatments with AICAR, a know activator of AMPK and fatty acid oxidation.

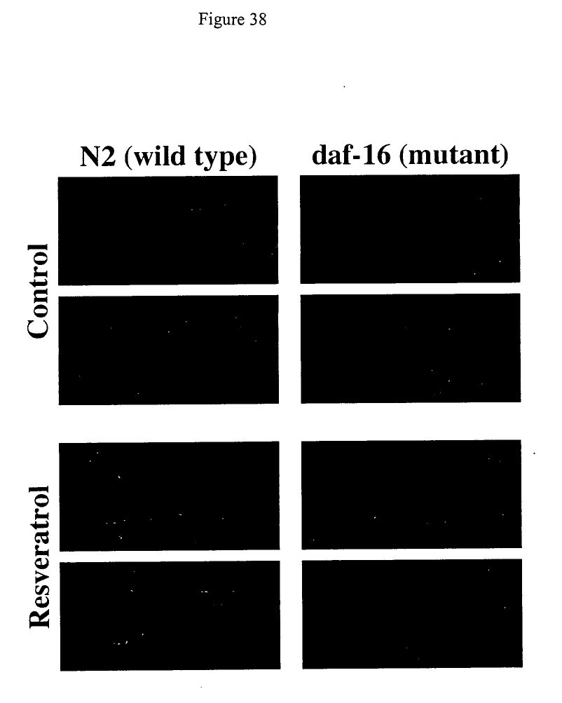

Sir2.1, which is activated by resveratrol, acts via the transcription factor DAF-16 to prolong lifespan in yeast (Tissenbaum and Guarente (2001) Nature 410:227). Similarly to the wild-type C. elegans, in DAF-16 mutant worms (mgDf47), which are defective in insulin-signaling (Wolkow, et al. Science 290:147, 2000), resveratrol stimulates fat mobilization and a decrease in fat accumulation (FIG. 38). This indicates that resveratrol signaling to fat metabolism in adult worms occurs via a pathway that is independent of DAF-16.

Accordingly, compounds in the resveratrol class that stimulate sirtuin proteins can promote fat mobilization in both wild-type and mutant C. elegans.

### Example 18

### Nicotinamide Promotes Fat Accumulation

If stimulators of sirtuin proteins decrease fat accumulation, inhibitors of sirtuin proteins, such as nicotinamide, should increase fat accumulation.

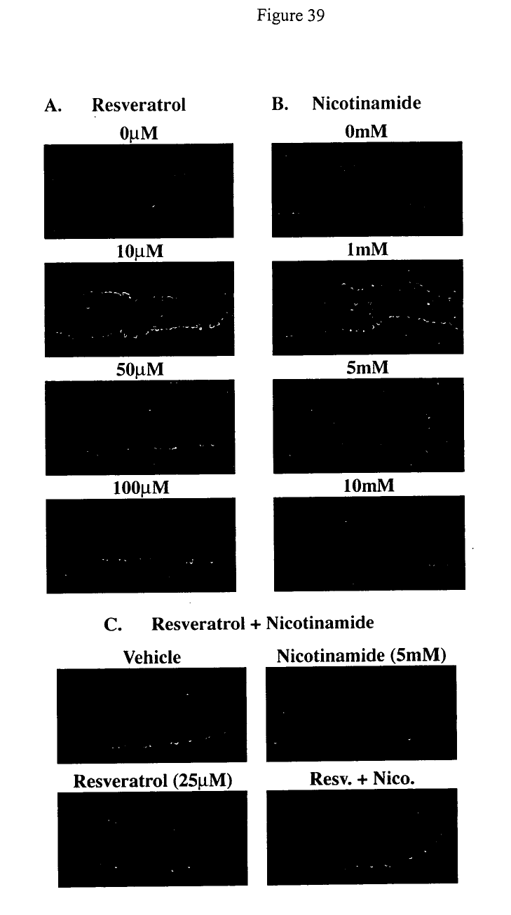

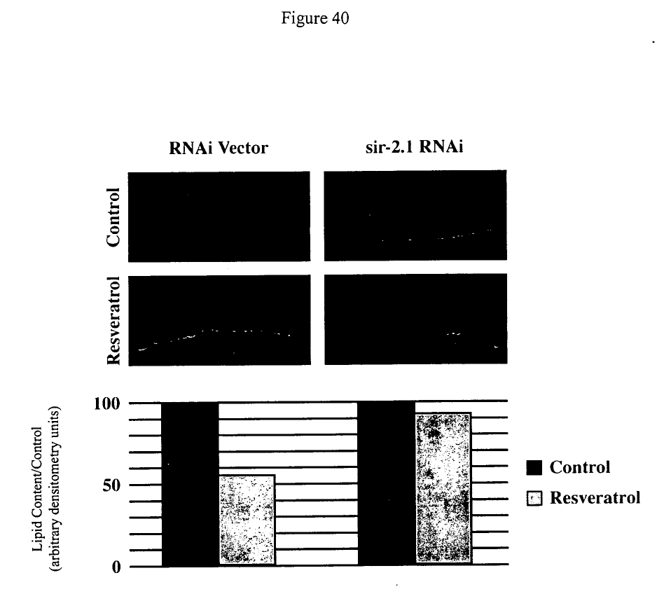

C. elegans worms were incubated overnight in the presence of 0, 1 or 10 mM nicotinamide, and stained with Nile-Red as described above. The results, which are shown in FIG. 39, indicate that the worms displayed a nicotinamide-concentration dependent increase in fat accumulation.

### Example 19

### Sir2 is Necessary for Resveratrol Mediated Fat Mobilization

The role of Sir2.1 in fat metabolism was shown in C. elegans worms in which Sir2.1 was RNA inactivated. Young adult worms were grown to adulthood in the presence of bacteria that carry RNAi vector alone or vector encoding Sir2.1 RNAi (R11A8.4), as described below. These worms were grown in the presence or absence of resveratrol, and stained with Nile-Red as described below. The results, which are shown in FIG. 4, indicate that the worms cultured in the presence of bacteria that carry Sir2.1 RNAi did not show resveratrol induced fat mobilization. These results further confirm that Sir2 is necessary for mediating the fat mobilization effect of resveratrol.

### Example 20

### AMPK is Necessary for Resveratrol Mediated Fat Mobilization

It was shown above that Sir2 is necessary for mediating the effect of resveratrol on fat mobilization. It is shown in this Example that AMPK is also necessary for mediating this effect. AMPK regulates diverse aspects of cell metabolism, glucose uptake and fatty acid oxidation. Many therapeutic agents and hormones that improve insulin sensitivity, e.g., 5-aminoimidazole-4-carboxamide-1-beta-D-ribofuranoside (AICAR) and Metformin, (decrease circulating insulin levels) are known to activate AMPK signaling to glucose uptake and fatty acid oxidation. In mammals, AMPK regulates fat metabolism by stimulating fatty acid oxidation via a series of complex steps that involve phosphorylation/inactivation of acetyl coA carboxylase, release of carnitine-palmitoyl transferase-1 (CPT-1) and camitine octanoyl transferase (COT) from end product inhibition by malonyl coA, and transport of fatty acids into the mitochondria to be oxidized.

Examination of the C. elegans database led to the finding of two gene products that are highly related to mammalian AMPK, TOC1.8 and Par2.3; five genes encoding homologs of CPT-1 and one gene encoding a homolog of COT. C. elegans worms were incubated with bacteria carrying RNAi vector alone, or interfering RNA against TOC1.8 or F41E7.3, a C. elegans homologue of COT in the presence or absence of AICAR. Fat accumulation was visualized with Nile-Red, as described below. The results indicate that RNA inactivation of TOC1.8 or COT inhibits AICAR-stimulated fat mobilization. Thus, AICAR/AMPK signaling to fatty acid oxidation is conserved in worms and mammalian cells.

The effect of TOC1.8 and COT inactivation was then investigated in C. elegans incubated with resveratrol. C. elegans worms were incubated with bacteria carrying RNAi vector alone, or a vector encoding TOC1.8 or COT interfering RNA in the presence or absence of resveratrol. Fat accumulation was visualized with Nile-Red, as described below.

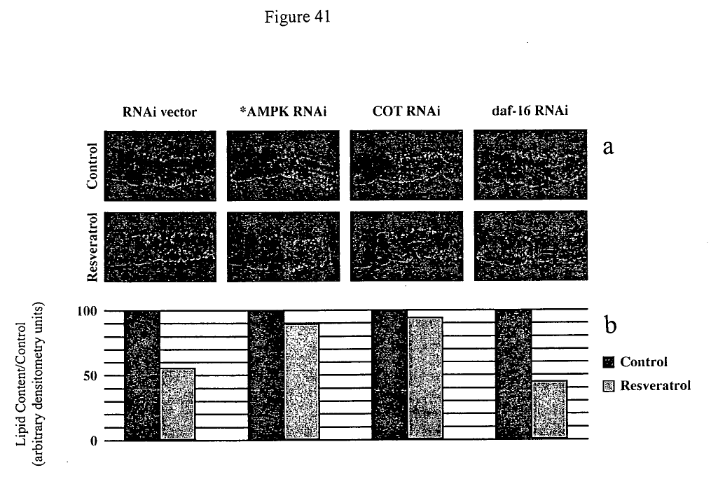

The results are shown in FIGS. 41A and B. In the presence of the RNAi vector alone, resveratrol reduces fat content in normal worms by 75% (FIG. 41A, panel a). However, RNA inactivation of TOC1.8, a homolog of mammalian AMPK or F41E7.3, a homolog of mammalian COT, blocks resveratrol-stimulated fat mobilization (see panels b and c of FIG. 41A). Thus, AMPK is necessary for resveratrol-induced fat mobilization. Thus, it seems that resveratrol, analogous to the direct AMPK activator AICAR, stimulates the AMPK signaling cascade to fat metabolism in worms. In contrast, RNA inactivation of DAF-16, the transcription factor downstream of insulin signaling to longevity, or inactivation of DAF-16 by mutation, had no effect on resveratrol-stimulated fat mobilization (see panel d of FIG. 41A).

Thus, inhibition of the resveratrol effect by RNA inactivation of AMPK and COT suggests that mobilization of fat requires activation of the AMPK signaling cascade to fatty acid oxidation.

### Example 21

### AICAR and Resveratrol Stimulate AMPK and ACC Phosphorylation

RNA inactivation of AMPK and COT suggested that the effect of resveratrol and AICAR to mobilize fat in worms is dependent on activation of the AMPK signaling cascade to fatty acid oxidation. To obtain direct evidence of AMPK activation, it was examined whether resveratrol-stimulated cells show increased phosphorylation of threonine residue 172 in AMPK or increased phosphorylation of acetyl coA carboxylase (ACC) at serine 79, modifications that correlate with activation of AMPK and inactivation of ACC, respectively.

CHO-HIR mammalian cells were washed in PBS and incubated overnight in serum-free DMEM before treatment with 500 μM AICAR (positive control) or 12.5 μM, 25 μM or 50 μM resveratrol. Cells were harvested after 30 minutes and lysates were immediately boiled in SDS and subjected to Western analysis with site-specific antibodies. Phosphorylation of AMPK at Thr172 indicates activation of the kinase. Active AMPK phosphorylates and inactivates ACC at serine 79.

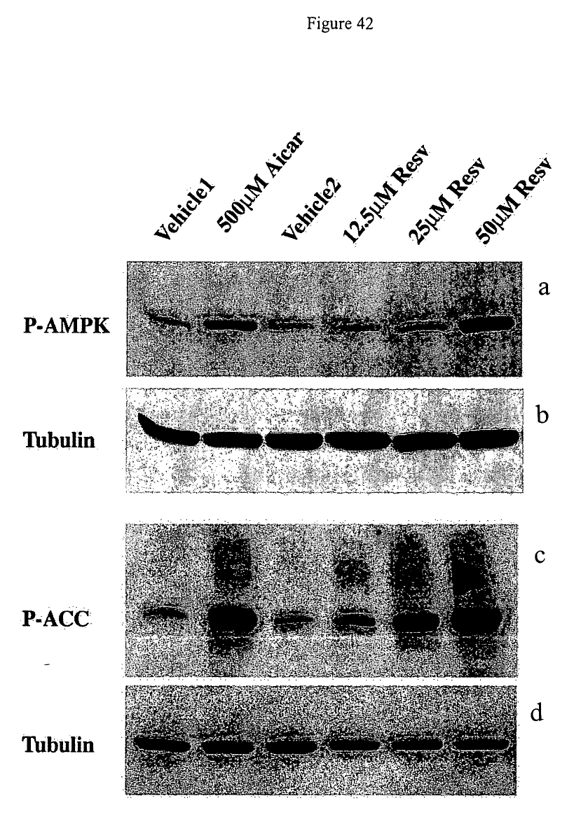

The results, which are shown in FIG. 42, indicate phosphorylation of AMPK on threonine 172 and phosphorylation of ACC on serine 79. Thus, like AICAR, resveratrol stimulates phosphorylation of AMPK and ACC. Accordingly, the ability of resveratrol to mobilize fat from lipogenic tissues is due, at least in part, to activation of AMPK signaling to fatty acid oxidation.

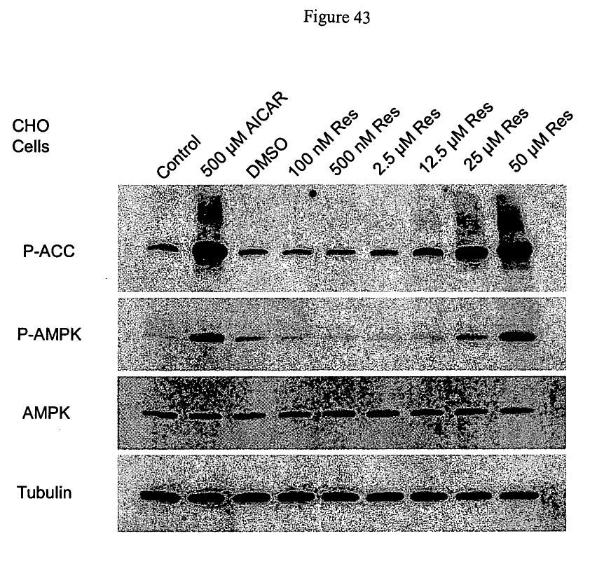

CHO cells were also treated with 500 μM AICAR (positive control), DMSO, 100 nM, 500 nM, 2.5 μM, 12.5 μM, 25 μM, or 50 μM resveratrol and subject to Western Blot analysis as described above. Western blots were stripped and re-probed for phosphorylated (active) AMPK, total AMPK, phosphorylated acetyl CoA carboxylase (ACC), which is the downstream target of AMPK, and tubulin, which served as a loading control. FIG. 43 shows activation of AMPK in CHO cells with increasing concentrations of resveratrol.

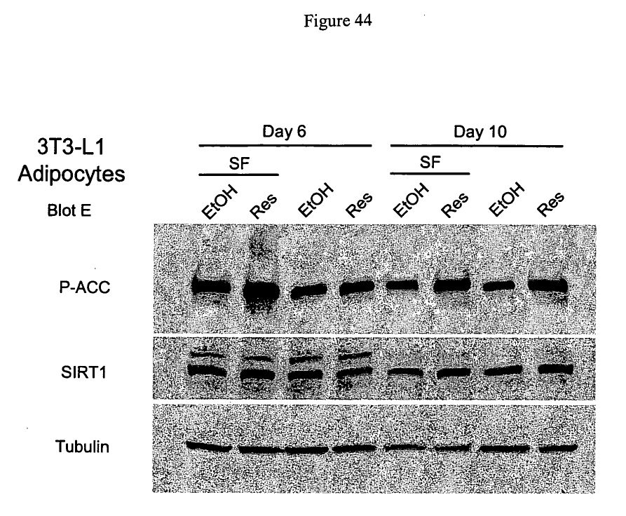

Phosphorylation of ACC, which reflects AMPK activity, was also observed in 3T3-L1 adipocytes treated with either ethanol or resveratrol. 3T3-L1 cells were incubated with either ethanol or resveratrol and then harvested either 6 or 10 days after they were induced to differentiate into adipocytes from the parent 3T3 fibroblast cell line. FIG. 44 shows that resveratrol stimulated the phosphorylation of ACC at both day 6 and day 10. ACC was also phosphorylated when the cells were incubated in serum free media overnight before harvesting (lanes marked “SF”). The reason for the extra band in the SIRT1 blot at day 6 is unknown, but it may be a modified form of SIRT1. Tubulin served as a loading control.

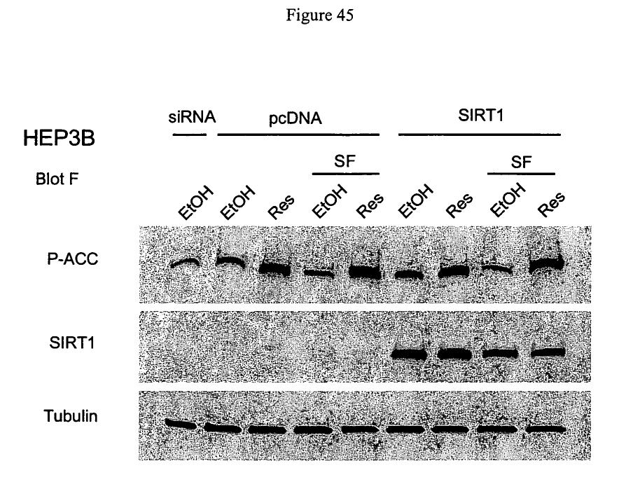

Similar results were also observed for HEP3B human hepatoma cells. In this case phosphorylation of ACC was measured in cells were SIRT1 was overexpressed (see FIG. 45, 4 right lanes) and in cells were SIRT1 was knocked down (FIG. 45, left lane). Phosphorylation of ACC was not affected indicating that resveratrol may not be working through SIRT1 in this case. Tubulin served as a loading control.

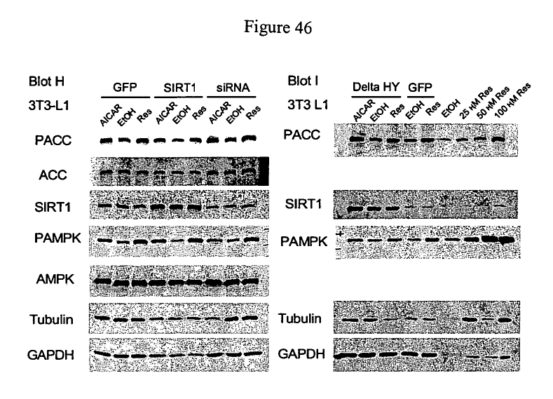

To further investigate whether resveratrol is working through SIRT1, 3T3-L1 apidocytes were infected with a control (GFP) retrovirus, SIRT1, SIRT1 siRNA, or SIRT1 dominant negative (delta HY). Cells were treated with AICAR, ethanol, or resveratrol. As described above, cells were harvested and lysates were prepared for Western blot analysis with site-specific antibodies. FIG. 46 shows phosphorylation of ACC and AMPK, which reflects AMPK activity. Total protein for each is also shown. It is also noted that the loading controls, GAPDH and tubulin, are expressed but at extremely low levels in these cells and may only reflect the presence of undifferentiated 3T3 cells. FIG. 46 also shows a separate dose-response curve on the far right.

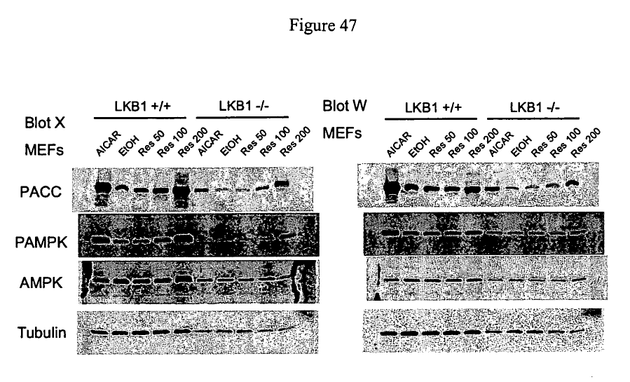

Similar results were also observed in mouse embryonic fibroblast (MEFs). FIG. 47 shows that resveratrol still has effects in the absence of the known AMPK kinase, LKB1. Cells in the left panel were incubated overnight without serum before harvesting; the cells on the right were not incubated under serum free conditions. While loading is lower for the LKB1 −/− cells, resveratrol still causes an upregulation of both AMPK and ACC phosphorylation. Tubulin served as a loading control.

### Example 22

### Resveratrol Stimulates Fat Mobilization and Inhibits Adipogenesis in Mammalian Cells

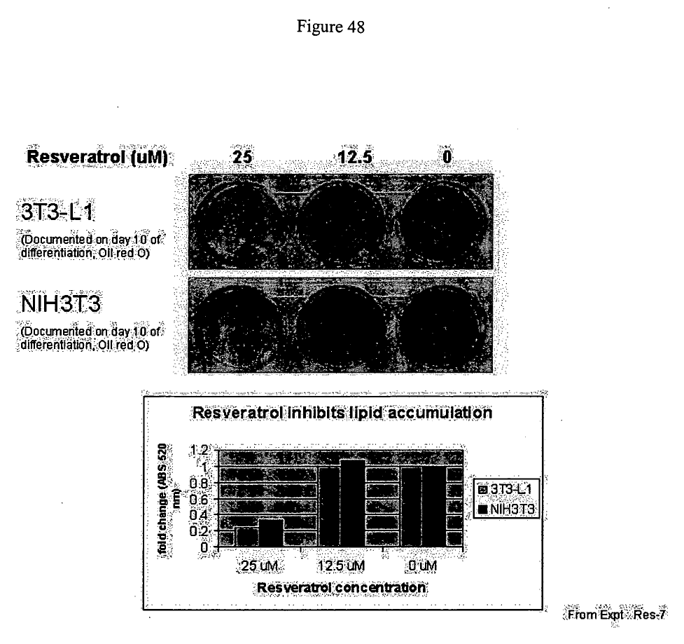

To obtain evidence that resveratrol affects fat metabolism in a physiologically relevant cell, the effect of increasing concentrations of resveratrol were examined on 3T3-L1 and NIH3T3 cell differentiation and fat content. 3T3-L1 or NIH3T3 cells were grown to confluence and allowed to pack in for 2 days at which point differentiation was initiated by addition of isobutylmethylxanthine, dexamethasone and insulin in the presence of vehicle (ethanol alone) or resveratrol at concentrations of 0, 12.5 and 25 μM. After 10 days of differentiation, fat content was assessed by Oil Red O staining, as described below. The results, which are shown in FIG. 48, indicate that concentrations of 25 μM or higher resveratrol decreased the quantity of cellular fat in 3T3-L1 and NIH3T3 cells. The results in NIH3T3 cells confirm the results obtained in C. elegans. The results indicate that resveratrol inhibits adipogenesis (or adipocyte differentiation).

AICAR stimulates AMPK signaling and inhibits adipogenesis in 3T3 cells. To distinguish whether the effect of resveratrol was to inhibit differentiation or mobilize fat from 3T3 cells, it was examined whether resveratrol inhibited the expression of adipogenic transcription regulators such as PPAR-γ. It was found that cells exposed to resveratrol did not show an increase in PPAR-γ RNA, which typically accompany differentiation of the cells into adipocytes. This suggests that resveratrol inhibits differentiation of cells into adipocytes. This may also suggest that resveratrol inhibits PPAR-γ activity or expression.

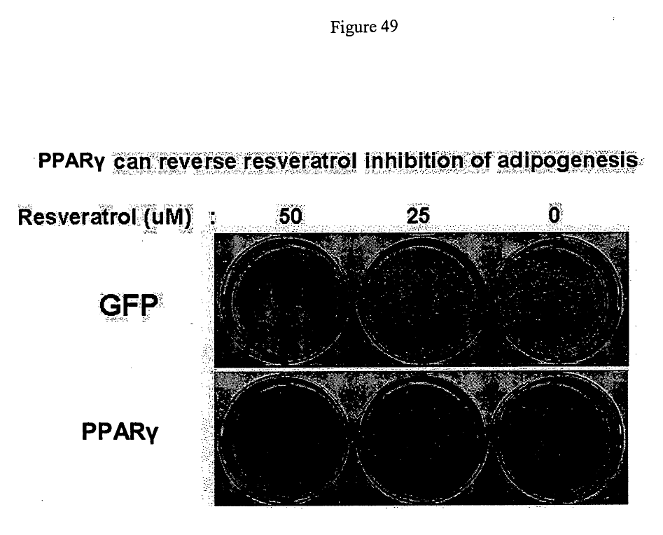

3T3 preadipoctyes/adipocytes were infected with pMX alone or pMX encoding PPAR-γ and the effect of resveratrol on 3T3 cell differentiation was examined. 3T3-L1 and NIH3T3 cells were infected with a plasmid expressing GFP or PPAR-γ and grown to confluence. Cells were differentiated into adipocytes as described below in the presence of 0 μM, 25 μM or 50 μM resveratrol in vehicle (ethanol). After eight days of differentiation, cells were fixed and stained with Oil red O. As expected, overexpression of PPAR-γ partially negated inhibition of 3T3 preadipocyte differentiation by resveratrol (FIG. 49). This observation suggests that resveratrol inhibits PPAR-γ activated fat cell differentiation.

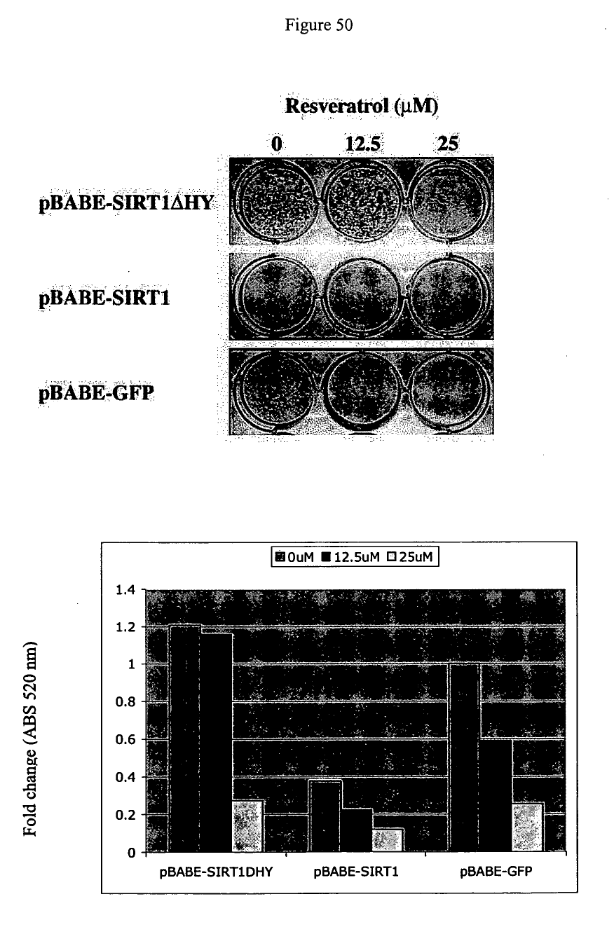

To further examine whether resveratrol activation of sir2 could promote fat mobilization or inhibition of differentiation in mammalian cells, growing cells were infected with wild-type SIRT1 or a deacetylase deficient form of SIRT1. NIH3T3 cells were grown in the presence of virus encoding GFP, SIRT1 or the deacetylase deficient form of SIRT1 (SIRT1ΔHY) (described in Vaziri et al. (2001) Cell 107:149). Cells were differentiated into adipocytes in the presence of 0 μM, 12.5 μM or 25 μM resveratrol in vehicle (ethanol). After eight days of differentiation, cells were fixed and stained with Oil red O. The results, which are shown in FIG. 50, indicate that 3T3 cells that overexpress wild-type SIRT1 show decreased fat content as compared to cells infected with virus encoding GFP (a negative control), while 3T3 cells that overexpress the deacetylase deficient form of SIRT1 show an increase in fat content. These results confirm the effect seen in worms, i.e., that SIRT activation by resveratrol appears to decrease fat content and SIRT1 inactivation by nicotinamide appears to increase fat content. Thus, sirtuins seem to play a direct role in regulating fat cell differentiation and content.

The decrease in Oil Red O staining seen with SIRT1 overexpression approaches the level seen when cells are stimulated with resveratrol. This observation raised the question whether the SIRT1 deacetylase deficient mutant would reverse the effect of resveratrol. It was found that in the SIRT1 deacetylase deficient mutant, the decrease in fat content normally induced by resveratrol was indeed partially reduced.

Thus, these results indicate that, in addition to reducing fat accumulation, resveratrol inhibits adipogenesis, and that this inhibition is also mediated at least in part by Sir2.

### Example 23

### Materials and Methods for Examples 3-6

Strains

C. elegans strains were maintained as described at 25° C., except when noted (Brenner (1974) Genetics 77:71). The wild type reference strain was N2 Bristol; the mutant strains were: sir-2.1 (ok434), T01C8.1 (ok524), and daf-16 (mgDf47). Daf-16 (mgDf47) was obatined from the Ruvkun laboratory, MGH; all other strains were obtained from the Caenorhabditis Genetics Center (from C. Elegans Gene Knockout Consortium).

Growth Conditions and Resveratrol Exposure

Synchronized starved L1 worms were grown in the presence of Nile Red. Strains were grown on NGM plates at 25° C. for approximately 48 hours until the young adult stage was reached. 20-30 young adult worms were then washed 2× with M9 buffer and transferred to new NGM/Nile red experimental plates that contained either OP50 or HTT5 E. coli carrying the L4440 RNAi control vector. For experiments comparing the effect of nicotinamide and resveratrol on fat mobilization, OP50 plates were coated with vehicle alone or Nicotinamide (in PBS), or vehicle alone and Resveratrol (in Ethanol or DMSO).

RNAi plates were seeded with HTT5 E. coli carrying either the L4440 RNAi vector control or the specific RNAi clones T01C8.1, AMPK; R11A8.4, sir-2.1; or F41E7.6 COT in the presence or absence of 100 μM resveratrol. Young adults were transferred to plates containing the appropriate vector, Nile Red stain and drug then maintained at 25° C. Nile Red staining was assessed 24 hours after resveratrol treatment by UV microscopy.

Resveratrol/Nicotinamide Dilutions

Resveratrol (Indofine #024964) was dissolved in Ethanol or DMSO to a 10 mM stock solution. Resveratrol was added to 60 mm NGM agar dishes containing either OP50 or RNAi expressing bacteria (HT115) to a final concentration of 10 μm, 50 μm, and 100 μM. Nile Red was also added to plates to a final concentration of 0.05 μg/ml. Nicotinamide (Supelco #47865-U) was diluted in PBS including Nile Red and added to 60 mM dishes containing OP50 to a final concentration of 1 mM, 10 mM, or 100 mM.

Fat Staining

Nile Red: Nile Red Powder (Sigma #N-3013) was dissolved in acetone at 500 μg/ml, diluted in 1× Phosphate Buffered Saline (PBS) including appropriate drug and applied to surface of Nematode Growth Media (NGM) plates previously seeded with OP50 or RNAi bacteria, at a final concentration of 0.05 μg/ml. Fat content was monitored and recorded by fluorescence microscopy.

Fluorescence Microscopy and Image Acquisition

Nile Red Staining was visualized by using a Nikon TE2000S microscope equipped with a CY3 filter (emission 535-685 nm). Images were captured using a SPOT RT monochrome digital camera attached to the Nikon Microscope with SPOT RT software v3.5. All Nile red images were acquired using identical settings and exposure times and then changed to red palette.

Feeding RNAi

HT115 E. Coli carrying the RNAi vector, L4440, were used for maintenance feeding. Bacteria containing experimental RNAi clones were cultured in 10 ml Luria Broth media containing 50 μg/ml ampicillin for 18 hours. 350 μl of each culture was spotted to a 60 mm dish containing NGM agar, 6 mM IPTG and 25 μg/ml carbenicillin. After overnight incubation (at room temp), Nile Red was added on top of each dish to a final concentration of 0.05 μg/ml along with the experimental compounds indicated in the figure legends. Nile Red staining was assessed after 24 hrs by UV microscopy. For each batch of RNAi clones tested, L4440 (vector alone) was included. A phenotype was assigned only if a majority of the animals displayed the phenotype. All phenotypes were confirmed by at least three additional rounds of testing.

Cell Culture and Oil Red O Staining

3T3-L1 and NIH3T3 cells were maintained in DMEM plus 10% calf serum. Adipocyte differentiation of 3T3-L1 cells was performed as described previously (MacDougald, O. A. and Lane, M. D. (1995). Transcriptional regulation of gene expression during adipocyte differentiation. Annu. Rev. Biochem. 64, 345-373). NIH3T3 cells were induced to form adipocytes under the same conditions as 3T3-L1 cells, but with 6 days of treatment with insulin, dexamethasone, and isobutylmethylxanthine in 10% fetal calf serum after cells reach confluence. The staining of adipocytes with Oil Red-O and quantitation was performed as described previously (Ramirez-Zacarias J L, Castro-Munozledo F, Kuri-Harcuch W. Histochemistry. 1992 97(6): 493-7).

Retrovirus Production and Infection

The mammalian retrovirus expression vector pMX (described in Tontonoz et al. (1994) Genes Dev. 8:1224, and provided by Gary Nolan) was used to construct and express full-length murine PPARγ2 (Tontonoz et al., supra), human SIRT1, human SIRT1ΔHY (Vaziri et al., supra) and eGFP. Recombinant retroviruses were generated by calcium phosphate transfection of the retroviral constructs into Phoenix ecotropic packaging cells (described in Tontonoz et al., supra, and provided by Gary Nolan), which were maintained in DMEM plus 10% fetal calf serum. Media was changed the next day and viral supernatant was harvested twice at 48 and 72 hr post-transfection of packaging cells. Viral supernatant was passed through a 0.2 μM syringe filter and applied to pre-confluent 3T3-L1 and NIH3T3 cells after addition of polybrene to a final concentration of 6 μg/ml. Media was changed the next day and cells were allowed to grow to confluence before differentiation to adipocytes.

### Example 24

### Additional Sirtuin Activators Stimulate Fat Mobilization

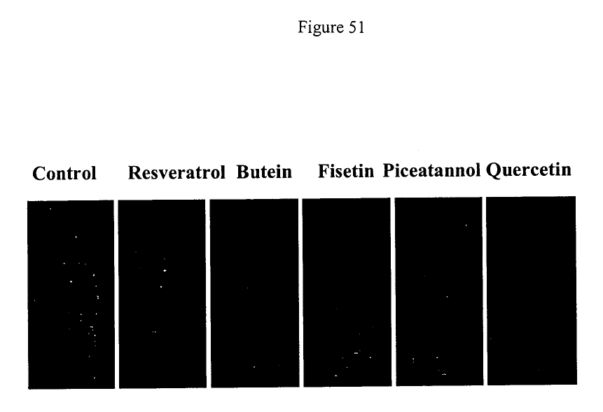

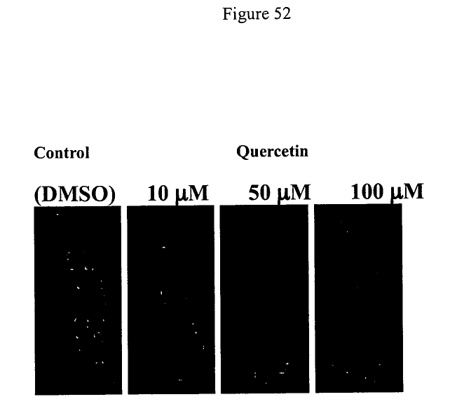

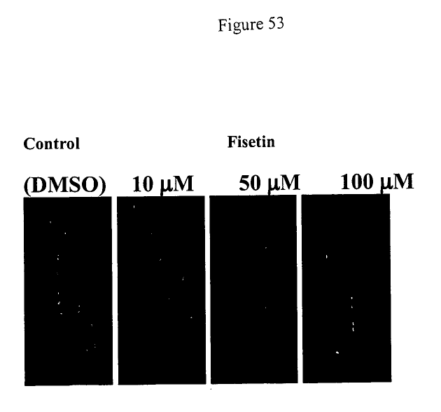

C. elegans worms were incubated in the presence or absence of 100 μM of the SIRT1 activators butein, fisetin, piceatannol and quercetin, and the fat content of the worms measured as described above. The results, which are shown in FIG. 51, indicated that these SIRT1 activators have a similar effect as resveratrol, i.e., they stimulate fat mobilization. Furthermore, as shown in FIGS. 52 and 53, quercetin and fisetin reduce fat accumulation at concentrations as low as 10 μM.

### Example 25

### Effects of Resveratrol Analogues on Fat Accumulation in C. elegans

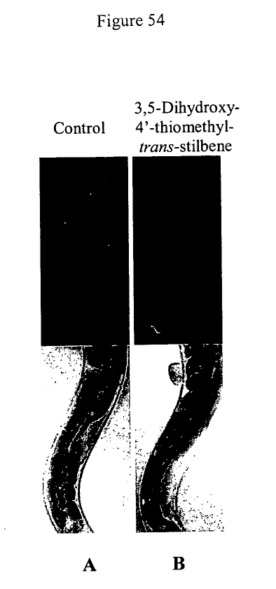

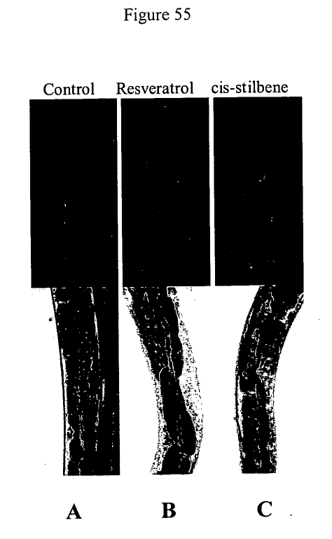

C. elegans worms were incubated in the absence (1% v/v DMSO) or presence of 100 μM 3,5-dihydroxy-4′-thiomethyl-trans-stilbene for 24 hours. Significant reduction of fat staining by 3,5-dihydroxy-4′-thiomethyl-trans-stilbene was observed (FIG. 54). Animals in L1 were also incubated in the absence (2.5% v/v DMSO) or presence of 100 μM resveratrol or 100 μM cis-stilbene for 48 hours. Significant reduction of fat staining by resveratrol is observed. No significant effect on worm fat staining is observed with cis-stilbene compared to the control (FIG. 55). Fat accumulation was visualized with Nile Red, a lipophilic stain, as described in Ashrafi et al., Nature 421: 268-27 (2003).

### Example 26

### Effects of Resveratrol on TNF-Alpha Treated Adipocytes that are Insulin Resistant

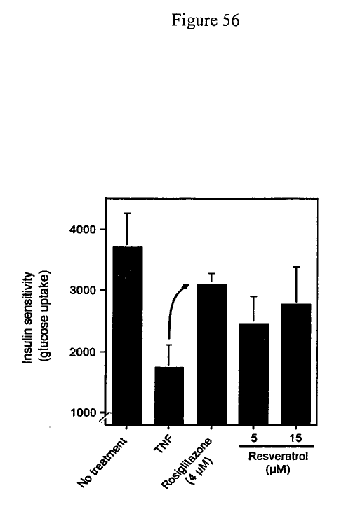

This example shows that resveratrol boosts insulin sensitivity of adipocytes. Adipocytes were treated with TNF-alpha to induce insulin resistance as described in Kabayama et al., Glycobiology 15: 21-29 (2005) and Wu et al., Mol. Cell 3: 151-8 (1999). Treatment with roziglitazone, a positive control, increases the uptake of radioactive glucose indicating increased insulin senstivity of the TNF-alpha treated adipocytes. As shown in FIG. 56, treatment with 5 μM or 15 μM resveratrol partially rescued the TNF-alpha treated adipocytes restoring insulin sensitivity in the treated cells. The arrow in FIG. 56, shows the desired effect of increased radioactive-glucose uptake.

### Example 27

### Resveratrol, like Other AMPK Activators, can Stimulate Fatty Acid Oxidation in Lipogenic Cells

Insulin is the major hormone charged with promoting storage of excess energy as fat. In cells with lipogenic capacity, insulin signaling promotes fat deposition. When fat stores become excessive this process is referred to as dyslipogenesis. Dyslipogenesis, is associated with insulin resistance and the progressive increase in circulating insulin and triglycerides levels, propensity to hypertension, and atherosclerosis that is characteristic of metabolic syndrome (Muller-Wieland, D. et al. Ann N Y Acad Sci 967: 19-27 (2002)). Insulin sensitizers, such as AICAR (5-aminoimidazole-4-carboxamide-1-beta-D-ribofuranoside) and metformin, activate AMP kinase and mobilize fat from non-adipose cells thereby reducing insulin resistance and serum lipid levels (Lin, H. Z., et al. Nat Med, 2000. 6(9): 998-1003; Bergeron, R., et al., Diabetes (2001) 50(5): 1076-82). Ample evidence exists that polyphenolic compounds derived from wine reduce serum lipid levels and atherosclerotic plaque (Waddington, E., et al., Am J Clin Nutr (2004) 79(1): 54-61). The observation that resveratrol activates AMP kinase suggested that this drug, analogous to AICAR and metformin, might be effective in reducing dyslipogenesis and increasing insulin sensitivity.

A plethora of reports indicate that AICAR and metformin activate AMPK, which in turn phosphorylates and inhibits acetyl coA carboxylase (ACC) (reviews by Kemp, B. E., et al., Trends Biochem Sci (1999) 24(1): 22-5; Kemp, B. E., et al., Biochem Soc Trans (2003) 31(Pt 1): 162-8; Viollet, B., et al., J Clin Invest (2003) 111(1): 91-8; Viollet, B., et al., Biochem Soc Trans (2003) 31(Pt 1): 216-9; Ruderman, N. B., et al., Am J Physiol (1999) 276(1 Pt 1): E1-E18; Mu, J., et al. Biochem Soc Trans (2003) 31(Pt 1): 236-41; and Zhou, G., et al., J Clin Invest (2001) 108(8): 1167-74). Inactivating ACC has the dual effect of inhibiting de novo fat biosynthesis and releasing fatty acid transferases carnitine-palmatoyl transferase-1 (CPT-1) and carnitine octanloyl transferase (COT) from end product inhibition by malonyl coA (Morillas, M., et al., FEBS Lett (2000) 466(1): p. 183-6). The result is decreased de novo fat biosynthesis and increased fatty acid oxidation FAO with a consequent decrease in cellular fat content.

Having shown that resveratrol increase phosphorylation of AMP kinase and ACC, see FIG. 43, it was confirmed that resveratrol stimulates CO₂ production from palmitate in two hepatoma cell lines (Table 23). The 3- to 6-fold increase in CO₂ production mirrors the stimulation achieved with AICAR. In sum, the data suggests that resveratrol can stimulate fat mobilization by activating AMPK signaling to the lipogenic enzyme ACC, reducing production of malonyl coA. The latter event inhibits the flow of substrate into de novo fat biosynthesis and stimulates fatty acid oxidation.

Table 23: Resveratrol, like other AMPK activators, can stimulate fatty acid oxidation. Oxidation of ¹⁴C-palmitate in hepatoma cells stimulated with vehicle control (1% DMSO or H₂O as appropriate), resveratrol (10 μM in 1% DMSO), AICAR (500 μM in H₂O), or metformin (1 mM in H₂O) for 4 hours as described in Methods. The fold effect of resveratrol on CO₂ production is shown.

¹⁴C—CO₂ production (nmol/hr/10⁶ cells)

| (Fold Effect) | | | | |
|---|---|---|---|---|
| Compound | Vehicle | Resveratrol | AICAR | Metformin |
| H4IIEC3 cells | 1 | 2.3 | 2.3 | 2 |
| HepG2 cells | 1 | 6 | 5 | 3.5 |

Methods:

Oxidation of ¹⁴C-palmitate to acid-soluble products (modified from H4IIEC3 cells (Witters, L. A. and B. E. Kemp, J Biol Chem (1992) 267(5): 2864-7) and HepG2 cells were maintained as described above. Cells (10⁶ cells/T25) were seeded in a T25 flask one day prior to the experiment. On the day of the experiment cells were washed with assay buffer (114 mM NaCl, 4.7 mM KCL, 1.2 mM KH₂PO₄, 1.2 mM MgSO₄, 11 mM glucose) before labeling with ¹⁴C-palmitate (0.4 μCi/ml) in presence of vehicle, or resveratrol (10 μM), or AICAR (500 μM) for 4 hours.

At the end of incubation, the cap of each T25 flask was replaced with a stopper and a 1′×1.5″ Whatman filter paper soaked with 250 μl 2N NaOH. Each flask was injected with 2 ml of 6N HCL, placed in a horizontal position for 10 minutes and left standing overnight. The next morning, 1 ml H₂O and 61 μl NaOH were added to a glass scintillation vial and the filter papers from each T25 flask were transferred to their respective vial. 10 ml Aquasol was added to each vial and allowed to stand for 2 hours, after which the vials were vortexed to dissolve the NaH¹⁴CO₂ and counted in the scintillation counter. The results were expressed as nmols/h/10⁶ cells and shown as the fold effect. ¹⁴CO₂ production ranged from 0.3 to 1.8 nmols/h/10⁶ cells. The experiment was repeated three times.

Equivalents

The present invention provides among other things sirtuin-activating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: PCT Publications WO 2005/002672; 2005/002555; and 2004/016726; and U.S. Pat. No. 6,746,691.

1. A method for treating or preventing drug-induced weight gain, comprising administering to a subject in need thereof a therapeutically effective amount of a sirtuin-activating compound.

2. The method of claim 1, wherein the weight gain is associated with administration of a diabetes treatment.

3. The method of claim 2, wherein the diabetes treatment is at least one of the following: a sulfonylurea, a thiazolidinedione, a meglitinide, nateglinide, repaglinide, or insulin.

4. The method of claim 1, wherein the weight gain is associated with administration of an antidepressant.

5. The method of claim 4, wherein the antidepressant is at least one of the following: a tricyclic antidepressant, an irreversible monoamine oxidase inhibitor (MAOI), a selective serotonin reuptake inhibitor (SSRI), bupropion, paroxetine, or mirtazapine.

6. The method of claim 1, wherein the weight gain is associated with administration of a steroid or a hormone.

7. The method of claim 1, wherein the weight gain is associated with administration of a beta blocker.

8. The method of claim 1, wherein the weight gain is associated with administration of an alpha blocker.

9. The method of claim 1, wherein the weight gain is associated with administration of a contraceptive.

10. The method of claim 1, wherein the sirtuin-activating compound comprises a formula selected from the group consisting of formulas 1-25, 30, 32-65, and 69-76.

11. The method of claim 1, wherein the sirtuin-activating compound is resveratrol, fisetin, butein, piceatannol or quercetin.

12. The method of claim 1, wherein the subject is a human.

13. A composition comprising at least one sirtuin-activating compound and at least one drug that induces weight gain.

14. A method for treating or preventing flushing, comprising administering to a subject in need thereof a therapeutically effective amount of a sirtuin-activating compound.

15. The method of claim 14, wherein the flushing is drug-induced flushing.

16. The method of claim 15, wherein the flushing is associated with administration of a chemotherapeutic agent.

17. The method of claim 15, wherein the flushing is associated with administration of nicotinic acid.

18. The method of claim 14, wherein the sirtuin-activating compound comprises a formula selected from the group consisting of formulas 1-25, 30, 32-65, and 69-76.

19. The method of claim 14, wherein the sirtuin-activating compound is resveratrol, fisetin, butein, piceatannol or quercetin.

20. The method of claim 14, wherein the subject is a human.


SEQUENCE LISTING (14 sequences)

SEQ ID NO: 1 - protein, 8 aa, Artificial Sequence
  peptide substrate for sirtuins derived from human histone H3
  VARIANT 2, 8: Xaa = acetylated lysine
KXQTARKX

SEQ ID NO: 2 - protein, 8 aa, Artificial Sequence
  peptide substrate for sirtuins derived from human histone H3
  VARIANT 2, 8: Xaa = acetylated lysine
KXSTGGKX

SEQ ID NO: 3 - protein, 9 aa, Artificial Sequence
  peptide substrate for sirtuins derived from human histone H3
  VARIANT 2, 9: Xaa = acetylated lysine
  VARIANT 4: Xaa = phosphorylated serine
KXSXTGGKX

SEQ ID NO: 4 - protein, 7 aa, Artificial Sequence
  peptide substrate for sirtuins derived from human histone H3
  VARIANT 2, 7: Xaa = acetylated lysine
KXAPRKX

SEQ ID NO: 5 - protein, 6 aa, Artificial Sequence
  peptide substrate for sirtuins derived from human histone H4
  VARIANT 6: Xaa = acetylated lysine
SGRGKX

SEQ ID NO: 6 - protein, 6 aa, Artificial Sequence
  peptide substrate for sirtuins derived from human histone H4
  VARIANT 6: Xaa = acetylated lysine
KGGAKX

SEQ ID NO: 7 - protein, 7 aa, Artificial Sequence
  peptide substrate for sirtuins derived from human histone H4
  VARIANT 2, 7: Xaa = acetylated lysine
KXGGAKX

SEQ ID NO: 8 - protein, 5 aa, Artificial Sequence
  peptide substrate for sirtuins derived from human P53
  VARIANT 5: Xaa = acetylated lysine
QPKKX

SEQ ID NO: 9 - protein, 6 aa, Artificial Sequence
  peptide substrate for sirtuins derived from human P53
  VARIANT 2, 6: Xaa = acetylated lysine
KXSKKX

SEQ ID NO: 10 - protein, 5 aa, Artificial Sequence
  peptide substrate for sirtuins derived from human P53
  VARIANT 5: Xaa = acetylated lysine
RHKKX

SEQ ID NO: 11 - protein, 6 aa, Artificial Sequence
  peptide substrate for sirtuins derived from human P53
  VARIANT 4, 6: Xaa = acetylated lysine
RHKXKX

SEQ ID NO: 12 - protein, 369 aa, Homo sapiens [sequence omitted]

SEQ ID NO: 13 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 14 - protein, 562 aa, Saccharomyces cerevisiae
MTIPHMKYAVSKTSENKVSNTVSPTQDKDAIRKQPDDIINNDEPSHKKIKVAQPDSLRETNTTDPLGHTKAALGEVASMELKPTNDMDPLAVSAASVVSMSNDVLKPETPKGPIIISKNPSNGIFYGPSFTKRESLNARMFLKYYGAHKFLDTYLPEDLNSLYIYYLIKLLGFEVKDQALIGTINSIVHINSQERVQDLGSAISVTNVEDPLAKKQTVRLIKDLQRAINKVLCTRLRLSNFFTIDHFIQKLHTARKILVLTGAGVSTSLGIPDFRSSEGFYSKIKHLGLDDPQDVFNYNIFMHDPSVFYNIANMVLPPEKIYSPLHSFIKMLQMKGKLLRNYTQNIDNLESYAGISTDKLVQCHGSFATATCVTCHWNLPGERIFNKIRNLELPLCPYCYKKRREYFPEGYNNKVGVAASQGSMSERPPYILNSYGVLKPDITFFGEALPNKFHKSIREDILECDLLICIGTSLKVAPVSEIVNMVPSHVPQVLINRDPVKHAEFDLSLLGYCDDIAAMVAQKCGWTIPHKKWNDLKNKNFKCQEKDKGVYVVTSDEHPKTL